(19) United States
(12) Patent Application Publication
(10) Pub. No.: US 2022/0162191 A1
(43) Pub. Date: May 26, 2022

(54) SMALL MOLECULE MODULATORS OF IL-17

(71) Applicant: LEO PHARMA A/S, Ballerup (DK)

(72) Inventors: Xifu LIANG, Ballerup (DK); Kevin Neil DACK, Ballerup (DK); Peter ANDERSEN, Ballerup (DK); Morten Dahl SØRENSEN, Ballerup (DK); Mark ANDREWS, Ballerup (DK); Alan Stuart JESSIMAN, Ballerup (DK)

(21) Appl. No.: 17/436,885

(22) PCT Filed: Mar. 6, 2020
(86) PCT No.: PCT/EP2020/056038

§ 371 Date: Sep. 7, 2021

(30) Foreign Application Priority Data
Mar. 8, 2019 (EP) 19161676.2

(51) Int. Cl. C07D 403/12; C07D 401/14; C07D 401/12; C07D 413/14; C07D 471/08; C07D 417/14; C07D 491/107; C07D 491/048; C07D 403/14; C07D 405/14

(52) U.S. Cl. CPC C07D 403/12; C07D 401/14; C07D 401/12; C07D 413/14; C07D 405/14; C07D 417/14; C07D 491/107; C07D 491/048; C07D 403/14; C07D 471/08

(57) ABSTRACT

The present invention relates to a compound according to formula I

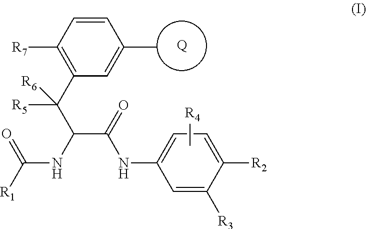

and pharmaceutically acceptable salts, hydrates, or solvates thereof. The invention further relates to, to said compounds for use in therapy, to pharmaceutical compositions comprising said compounds, to methods of treating diseases, e.g. dermal diseases, with said compounds, and to the use of said compounds in the manufacture of medicaments.

### FIELD OF THE INVENTION

This invention relates to novel amino-acid anilides and derivatives thereof, to said compounds for use in therapy and to pharmaceutical compositions comprising said compounds.

### BACKGROUND OF THE INVENTION

IL-17 (also known as IL-17A or CTLA8) is a pro-inflammatory cytokine involved in antimicrobial defense at epithelial surfaces. IL-17 is comprised of two covalently joined IL-17A subunits (IL-17AA) with an approximate mass of 32 kDa, and signals through a receptor comprising IL17RA and IL17RC subunits. This receptor is predominantly expressed in epithelial and mesenchymal cells. The IL17RA/IL17RC receptor is also used by IL-17 variants IL-17AF and IL-17FF, which both are successively weaker, partial agonists on this receptor (Monin, L., Gaffen, S. L.; 2018, Cold Spring Harb. Perspect. Biol. 10. doi:10.1101/cshperspect.a028522). Crucial for signaling is the assembly of signaling complexes containing the multifunctional protein ACT1/CIKS, which in turn can recruit TRAF and other proteins.

Via these signaling complexes IL-17 induces cytokines, chemokines, antimicrobial peptides and growth factors via activation of transcription factor NFkB or via MAP kinase-dependent pathways (e.g. IL-6, IL-8, CXCL1, CXCL2, CXCL5, CCL20, G-CSF, BD4) and stabilizes the mRNAs of certain inflammatory cytokines, such as CXCL1. This leads to amplification of their effects. Further IL-17 acts in concert with IL-1beta, IL-22 and IFNgamma (Amatya, N. et al., Trends in Immunology, 2017, 38, 310-322. doi:10.1016/j.it.2017.01.006; Onishi, R. M., Gaffen, S. L. Immunology, 2010, 129, 311-321. doi:10.1111/j.1365-2567.2009.03240.x).

IL-17 is secreted by a variety of immune cells, such as Th17 helper cells, Tc17 cytotoxic cells, ILC3 innate cells, NKT cells, TCRbeta+ natural T cells and gamma-deltaT-cells (Monin, L., Gaffen, S. L.; 2018, Cold Spring Harb. Perspect. Biol. 10. doi:10.1101/cshperspect.a028522). Increased, disease-provoking levels of IL-17 are observed in several autoimmune diseases, such as psoriasis, ankylosing spondylitis, spondyloarthritis and psoriatic arthritis. Other diseases where deregulation of IL-17 is observed are rheumatoid arthritis, systemic lupus erythematosus, asthma, inflammatory bowel disease, autoimmune uveitis, multiple sclerosis and certain cancers (Gaffen, S. L. et al., Nat Rev Immunol., 2014, 14, 585-600. doi:10.1038/nri3707; Monin, L., Gaffen, S. L.; 2018, Cold Spring Harb. Perspect. Biol. 10. doi:10.1101/cshperspect.a028522). Hence, IL-17 is a significant therapeutic target.

Therapeutic, neutralizing antibodies against IL-17A (Secukinumab, Ixekizumab) or receptor IL17RA (Brodalumab) have shown high efficacy in the treatment of psoriasis, ankylosing spondylitis and psoriatic arthritis. These antibodies have long half-lives in the body.

Although various antibodies against IL-17A or IL-17RA are approved, only very few small molecule orally available modulators of IL-17 are known.

WO2013116682 discloses Macrocyclic Compounds for Modulating IL-17;

WO2014066726 discloses Compounds for Modulating IL-17;

WO2018229079 discloses compounds for modulating IL-17,

WO2019223718 discloses Compounds for Modulating IL-17;

WO2019138017 discloses Compounds for Modulating IL-17;

WO2020011731 discloses Compounds for Modulating IL-17;

Scientific Reports (2016) 6, 30859 discloses Macrocyclic IL-17A antagonists.

Leslie Dakin, 12^{th} Swiss Course on Medicinal Chemistry, Leysin, Oct. 9-14, 2016 discloses ‘Hit Identification, binding site elucidation and structure guided design of novel macrocyclic IL-17A antagonists’.

Orally available, highly efficacious small molecule IL-17 modulators which bind to IL-17 to decrease its functional ability to activate the IL-17 receptor complex may have a number of advantages compared to monoclonal antibodies. Oral administration and flexible treatment regimen may be two significant aspects in favor of patient convenience and the compounds may exhibit improved safety due to the possibility of faster withdrawal of the drug should adverse events occur.

Therefore, there is a continuous need to develop small molecule modulators of IL-17, particularly small molecules suitable for oral administration.

In addition, some patients may be treated by topical application of small molecule modulators of IL-17. This can be particularly suitable for patients with skin lesions that are readily accessible and limited in body surface area. Topical treatment may also be prescribed for certain patients who could benefit from avoiding systemic modulation of the IL-17 pathway, for example when undergoing treatment for infections or gastrointestinal problems.

### SUMMARY OF THE INVENTION

The inventors have surprisingly found that novel compounds of the present invention exhibit modulating effect on the IL-17 signalling pathway.

Compounds of the present invention may have advantageous properties such as high metabolic stability and/or membrane permeability properties that make them suitable for oral administration. Other compounds of the present invention may have advantageous properties for local topical therapy, such as high skin permeability and high metabolic instability.

Compounds of the present invention may be beneficial in preventing, treating or ameliorating a variety of diseases which involve up-regulation or de-regulation of IL-17, such as for example psoriasis, ankylosing spondylitis and psoriatic arthritis

Accordingly, the present invention relates to a compound according to formula (I)

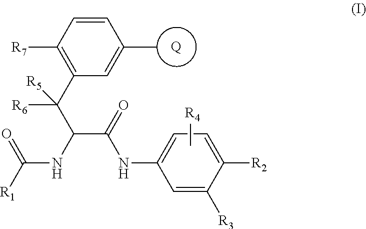

wherein

R₁ is selected from the group consisting of 5- or 6-membered heteroaryl, 9- or 10-membered bicyclic heteroaryl, phenyl, 4-6-membered heterocycloalkyl, (C₁-C₆)alkoxy, (C₃-C₇)cycloalkoxy, (C₃-C₇)cycloalkyl(C₁-C₆)alkyl, (C₃-C₇)cycloalkyl(C₁-C₆)alkoxy, (C₁-C₆)alkyl, phenyl-(C₁-C₄)alkyl, (C₃-C₇)cycloalkyl and —NR_{c}R_{d}, wherein said 5- or 6-membered heteroaryl, 9- or 10-membered bicyclic heteroaryl, phenyl, 4-6-membered heterocycloalkyl, (C₁-C₆)alkoxy, (C₃-C₇)cycloalkoxy, (C₃-C₇)cycloalkyl(C₁-C₆)alkyl, (C₃-C₇)cycloalkyl(C₁-C₆)alkoxy, (C₁-C₆)alkyl, phenyl-(C₁-C₄)alkyl and (C₃-C₇)cycloalkyl is optionally substituted with one or more substituents independently selected from Rₐ;

Rₐ represents deuterium, halogen, hydroxy —NR_{c}R_{d}, (C₁-C₆)alkyl, (C₁-C₆)alkylcarbonyl, (C₃-C₇)cycloalkyl, phenyl, 4-6-membered heterocycloalkyl or 5- or 6-membered heteroaryl, wherein said (C₁-C₆)alkyl, (C₁-C₆)alkylcarbonyl, (C₃-C₇)cycloalkyl, phenyl, 4-6-membered heterocycloalkyl or 5- or 6-membered heteroaryl is optionally substituted with one or more substituents independently selected from deuterium, halogen, hydroxy, (C₁-C₄)alkyl, (C₃-C₇)cycloalkyl, (C₁-C₄)alkoxy, —SO₂—(C₁-C₄)alkyl and —NR_{c}R_{d};

R₂ is 5-membered heteroaryl, wherein said 5-membered heteroaryl is optionally substituted with one or more substituents independently selected from R_{b};

R_{b} represents deuterium, halogen, cyano, hydroxy, —NR_{c}R_{d}, (C₁-C₆)alkyl, (C₁-C₆)alkoxy or (C₃-C₇)cycloalkyl, wherein said (C₁-C₆)alkyl, (C₁-C₆)alkoxy or (C₃-C₇)cycloalkyl is optionally substituted with one or more substituents independently selected from deuterium, halogen, hydroxy, —NR_{c}R_{d} and (C₁-C₄)alkoxy;

R_{c} and R_{d} each independently are selected from the group consisting of hydrogen and (C₁-C₄)alkyl;

R₃ is selected from the group consisting of hydrogen, deuterium, hydroxy, (C₁-C₆)alkoxy and halogen;

or R₂ and R₃ together with the phenyl to which they are attached form a 9- or 10-membered bicyclic heteroaromatic ring system, wherein said 9- or 10-membered bicyclic heteroaromatic ring system is optionally substituted with one or more substituents independently selected from Rₑ;

Rₑ represents deuterium, halogen, (C₁-C₄)alkyl, hydroxy(C₁-C₄)alkyl, (C₃-C₇)cycloalkyl or —NR_{c}R_{d};

R₄ is selected from the group consisting of hydrogen, deuterium and halogen;

R₅ and R₆ each independently are selected from the group consisting of hydrogen, deuterium, (C₁-C₄)alkyl, (C₃-C₇)cycloalkyl and phenyl, with the proviso that at least one of R₅ and R₆ is selected from hydrogen or deuterium;

R₇ is selected from the group consisting of hydrogen and halogen, with the proviso that R₇ is not hydrogen when both R₅ and R₆ are selected from hydrogen or deuterium;

or R₆ and R₇ together with the phenyl to which R₇ is attached form a fused bicyclic ring system selected from the group consisting of tetralin and indane, wherein said tetralin or indane is optionally substituted with one more substituents independently selected from the group consisting of deuterium, halogen and (C₁-C₄)alkyl;

Q represents phenyl, 4-8 membered heterocycloalkyl, 5- or 6-membered heteroaryl, 9- or 10-membered bicyclic heteroaryl, pyridonyl, indolinyl, isoindolinyl, tetrahydroquinolinyl or tetrahydroisoquinolinyl, wherein said pyridonyl, phenyl, 4-8 membered heterocycloalkyl, 5- or 6-membered heteroaryl, 9- or 10-membered bicyclic heteroaryl, indolinyl, isoindolinyl, tetrahydroquinolinyl or tetrahydroisoquinolinyl is optionally substituted with one or more substituents independently selected from R₈;

R₈ represents deuterium, oxo, hydroxy, —S(O)₂R_{f}, —S(O)R_{f}, —NR_{g}Rₕ, —C(O)NR_{g}Rₕ, —C(O)R_{f}, cyano, (C₁-C₆)alkyl, (C₃-C₇)cycloalkyl, 4-8 membered heterocycloalkyl, (C₃-C₇)cycloalkyl(C₁-C₄)alkyl, (C₃-C₇)cycloalkyl(C₁-C₄)alkoxy, (C₁-C₄)alkoxy(C₁-C₄)alkyl, (C₁-C₄)alkoxy, (C₃-C₇)cycloalkoxy, 5- or 6-membered heteroaryl, (5- or 6-membered heteroaryl)-(C₁-C₄)alkyl and (4-8 membered heterocycloalkyl)-(C₁-C₄)alkyl wherein said (C₁-C₆)alkyl, (C₃-C₇)cycloalkyl, 4-8 membered heterocycloalkyl, (C₃-C₇)cycloalkyl(C₁-C₄)alkyl, (C₃-C₇)cycloalkyl(C₁-C₄)alkoxy, (C₁-C₄)alkoxy(C₁-C₄)alkyl, (C₁-C₄)alkoxy, (C₃-C₇)cycloalkoxy, 5- or 6-membered heteroaryl, (5- or 6-membered heteroaryl)-(C₁-C₄)alkyl and (4-8 membered heterocycloalkyl)-(C₁-C₄)alkyl is optionally substituted with one or more substituents independently selected from hydroxy, deuterium, halogen, cyano, oxo, (C₁-C₄)alkyl, (C₃-C₇)cycloalkyl, (C₁-C₄)alkoxy, hydroxy(C₁-C₄)alkyl, (C₁-C₄)alkoxy-(CH₂CH₂O)₀₋₂(C₁-C₄)alkyl, 4-7 membered heterocycloalkyl, —NR_{g}Rₕ, R_{g}RₕN—(C₁-C₄)alkyl and —CO(O)Rᵢ;

R_{f} represents (C₁-C₄)alkyl, or (C₃-C₇)cycloalkyl;

R_{g} and Rₕ each independently represents hydrogen, (C₁-C₄)alkyl, (C₃-C₇)cycloalkyl, (C₃-C₇)cycloalkyl(C₁-C₄)alkyl or hydroxy(C₁-C₄)alkyl;

or R_{g} and Rₕ together with the nitrogen to which they are attached form a 4-8 membered heterocycloalkyl wherein said 4-8 membered heterocycloalkyl is optionally substituted with one or more substituents independently selected from deuterium, halogen, hydroxy, (C₁-C₄)alkyl, hydroxy(C₁-C₄)alkyl, halo(C₁-C₄)alkyl;

Rᵢ represents hydrogen or (C₁-C₄)alkyl;

or pharmaceutically acceptable salts, hydrates, solvates or prodrugs thereof.

In another aspect, the invention relates to a pharmaceutical composition comprising a compound of general formula (I) as defined herein together with a pharmaceutically acceptable vehicle or excipient or pharmaceutically acceptable carrier(s), optionally together with one or more other therapeutically active compound(s).

In yet another aspect, the invention relates to the use of a compound according to formula I as defined herein for use in therapy, for example for use in treatment of a disease, disorder or condition, which disease, disorder or condition is responsive of modulation of IL-17, for example for use in treatment of autoimmune diseases, such as psoriasis, ankylosing spondylitis, spondyloarthritis or psoriatic arthritis.

### DETAILED DESCRIPTION OF THE INVENTION

### Definitions

The term “(Cₐ-C_{b})alkyl” is intended to indicate a hydrocarbon radical obtained when one hydrogen atom is removed from a branched or linear hydrocarbon. Said alkyl comprises (a-b) carbon atoms, such as 1-6, such as 1-4, such as 1-3, such as 2-3 or such as 1-2 carbon atoms. The term includes the subclasses normal alkyl (n-alkyl), secondary and tertiary alkyl, such as methyl, ethyl, n-propyl, isopropyl, n-butyl, isobutyl, sec.-butyl, tert.-butyl, n-pentyl, isopentyl, neopentyl, n-hexyl and isohexyl.

The terms “(Cₐ-C_{b})alkyloxy” and “(Cₐ-C_{b})alkoxy” are intended to indicate a radical of the formula —OR′, wherein R′ is (Cₐ-C_{b})alkyl as indicated herein, wherein the (Cₐ-C_{b})alkyl group is appended to the parent molecular moiety through an oxygen atom, e.g. methoxy (—OCH₃), ethoxy (—OCH₂CH₃), n-propoxy, isopropoxy, butoxy, tert-butoxy, and the like.

The term “cyano” is intended to indicate a —CN group attached to the parent molecular moiety through the carbon atom.

The term “(Cₐ-C_{b})cycloalkyl” is intended to indicate a saturated (Cₐ-C_{b})cycloalkane hydrocarbon radical, including polycyclic radicals such as bicyclic or tricyclic radicals, including spirocyclic radicals, comprising a-b carbon atoms, such as 3-10 carbon atoms, such as 3-8 carbon atoms, such as 3-7 carbon atoms, such as 3-6 carbon atoms, such as 3-5 carbon atoms or such as 3-4 carbon atoms, e.g. cyclopropyl, cyclobutyl, cyclopentyl, cyclohexyl, cycloheptyl, cyclooctanyl, adamantly, cubanyl, bicyclo[1,1,1]pentanyl and spiro[2.5]octanyl, spiro[2.3]hexanyl, bicyclo[3,1,0]hexanyl, bicyclo[4,1,0]heptanyl and bicyclo[2,2,2]octanyl.

The term “(Cₐ-C_{b})cycloalkoxy” is intended to indicate a radical of the formula —OR′, wherein R′ is (Cₐ-C_{b})cycloalkyl as indicated herein, wherein the (Cₐ-C_{b})cycloalkyl group is appended to the parent molecular moiety through an oxygen atom, e.g. cyclopentyloxy or cyclobutyloxy.

The term “(Cₐ-C_{b})cycloalkyl(Cₐ-C_{b})cycloalkyl is intended to indicate an (Cₐ-C_{b})alkyl group as defined herein substituted with a (Cₐ-C_{b})cycloalkyl group as defined herein, e.g cyclopropylmethyl.

The term “(Cₐ-C_{b})cycloalkyl(Cₐ-C_{b})cycloalkoxy is intended to indicate an (Cₐ-C_{b})alkoxy group as defined herein substituted with a (Cₐ-C_{b})cycloalkyl group as defined herein, e.g cyclopropylmethyloxy.

The term “halo(Cₐ-C_{b})alkyl” is intended to indicate an (Cₐ-C_{b})alkyl group as defined herein substituted with one or more halogen atoms as defined herein, e.g. fluoro or chloro, such as difluoromethyl or trifluoromethyl.

The terms “halo(Cₐ-C_{b})alkyloxy” and “halo(Cₐ-C_{b})alkoxy” are intended to indicate an halo(Cₐ-C_{b})alkyl group as defined herein which is appended to the parent molecular moiety through an oxygen atom, such as difluoromethoxy or trifluoromethoxy.

The term “halogen” is intended to indicate a substituent from the 7^{th} main group of the periodic table, such as fluoro, chloro and bromo.

The term “5- or 6-membered heteroaryl” is intended to indicate radicals of monocyclic heteroaromatic rings comprising 5- or 6-membered ring which contains from 1-5 carbon atoms and from 1-4 heteroatoms selected from oxygen, sulphur and nitrogen; such as 2-5 carbon atoms and 1-3 heteroatoms, such as 3-5 carbon atoms and 1-2 heteroatoms, such as 4-5 carbon atoms and 1-2 heteroatoms selected from oxygen, sulphur and nitrogen, such as furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, quinolyl, tetrazolyl, thiadiazolyl and thiazolyl. The term “5- or 6-membered heteroaryl” includes compounds wherein a ring member of said “5- or 6-membered heteroaryl” is a C(O) or carbonyl group.

The term “5-membered heteroaryl” is intended to indicate radicals of 5-membered monocyclic heteroaromatic ring which contains from 1-4 carbon atoms and from 1-4 heteroatoms selected from oxygen, sulphur and nitrogen; such as 2-4 carbon atoms and 1-3 heteroatoms, such as 3-4 carbon atoms and 1-2 heteroatoms, such as 4 carbon atoms and 1 heteroatom selected from oxygen, sulphur and nitrogen; such as furanyl, imidazolyl, isothiazolyl, isoxazolyl, oxadiazolyl, oxazolyl, pyrazolyl, pyrrolyl, quinolyl, tetrazolyl, thiadiazolyl, thiazolyl and triazolyl. The term “5- or 6-membered heteroaryl” includes compounds wherein a ring member of said “5- or 6-membered heteroaryl” is a C(O) or carbonyl group.

The term “9- or 10-membered bicyclic heteroaryl” is intended to indicate fused bicyclic heteroaromatic radicals comprising 9- or 10-carbon or heteroatoms, which for example contains from 3-9 carbon atoms and 1-7 heteroatoms selected from oxygen, sulphur and nitrogen, such as 1-5 heteroatoms and 5-9 carbon atoms, such as 1-3 heteroatoms and 7-9 carbon atoms, such as 1-2 heteroatoms and 8-9 carbon atoms, such as 1 heteroatom and 8 carbon atoms, such as 1 heteroatom and 9 carbon atoms, such as 2 heteroatom and 7 carbon atoms, such as 2 heteroatom and 8 carbon atoms. Said bicyclic heteroaromatic radicals comprise a 5- or 6-membered heteroaromatic ring fused to phenyl or a 5- or 6-membered heteroaromatic ring fused to another 5- or 6-membered heteroaromatic ring, as defined herein. The heteroaryl radical may be connected to the parent molecular moiety through a carbon atom or a nitrogen atom contained anywhere within the heteroaryl group. Representative examples of 9- or 10-membered bicyclic heteroaryl include, but are not limited to azaindolyl, benzofuranyl, benzothiophenyl, benzimidazolyl, benzooxazolyl, benzothiazolyl, benzothienyl, cinnolyl, imidazopyridinyl, imidazopyrimidinyl, imidazothiazolyl, indazolyl, indolyl, isobenzofuranyl, isoquinolyl, pyrrolopyrimidinyl, thienopyridinyl, pyrrolo[2,3]pyridinyl, pyrazolo[1,5]pyridinyl, pyrazolo[1,5]pyridazinyl, imidazo[1,2]pyrimidinyl, pyrrolo[2,3-c]pyridinyl, pyrrolo[2,3-b]pyridinyl, pyrazolo[1,5-a]pyridinyl, pyrazolo[1,5-b]pyridazinyl and imidazo[1,2-a]pyrimidinyl.

The term (5- or 6-membered heteroaryl)-(Cₐ-C_{b})alkyl is intended to indicate a 5- or 6-membered heteroaryl appended to the parent molecular moiety through a (Cₐ-C_{b})alkyl group, as defined herein.

The term “(a-b) membered heterocycloalkyl” is intended to indicate a cycloalkane radical as described herein, including polycyclic radicals such as bicyclic or tricyclic radicals, including spirocyclic radicals, wherein one or more carbon atoms of said cycloalkane radical are replaced by heteroatoms, i.e. the a-b membered heterocycloalkyl comprise from a to b carbon- or hetero-atoms. Such a-b membered heterocycloalkyl could comprise for example 2-9 carbon atoms and 1-6 heteroatoms selected from O, N, or S, such as 3-8 carbon atoms and 1-4 heteroatoms, such as 3-7 carbon atoms and 1-3 heteroatoms, such as 3-6 carbon atoms and 1-2 heteroatom. The heterocycloalkyl radical may be connected to the parent molecular moiety through a carbon atom or a nitrogen atom contained anywhere within the heterocycloalkyl group. Representative examples of heterocycloalkyl groups include, but are not limited to azepanyl, azetidinyl, aziridinyl, dioxolanyl, dioxolyl, imidazolidinyl, morpholinyl, thiomorpholinyl, oxetanyl, piperazinyl, piperidinyl, pyrrolidinyl, tetrahydrofuranyl, tetrahydropyranyl, thietanyl, 2,6-diazaspiro[3.3]heptanyl, 2,5-diazabicyclo[2.2.1]heptanyl, 2-oxa-5-aza-[2.2.1]heptanyl, 2-oxa-8-azaspiro[3.5]nonanyl, 2-oxa-7-azaspiro[3.5]nonanyl, 2-oxa-8-azaspiro[3.5]nonanyl, 6-oxa-2-azaspiro[3.3]heptanyl, 2-oxa-7-azaspiro[3,4]octanyl, and 1, 3, 3a, 4, 6, 6a-hexahydrofuro[3,4-c]pyrrolyl. The term includes compounds wherein a ring member of said “(a-b) membered heterocycloalkyl” is a C(O) or carbonyl group and S(O) group.

The term “(a-b membered heterocycloalkyl)-(C_{c}-Cₐ)alkyl” is intended to indicate a a-b membered heterocycloalkyl radical appended to the parent molecular moiety through an (C_{c}-C_{d})alkyl group, as defined herein.

The term “hydrocarbon radical” is intended to indicate a radical containing only hydrogen and carbon atoms, it may contain one or more double and/or triple carbon-carbon bonds, and it may comprise cyclic moieties in combination with branched or linear moieties. Said hydrocarbon comprises 1-6 carbon atoms, e.g. 1-5, e.g. 1-4, e.g. 1-3, e.g. 1-2 carbon atoms. The term includes alkyl and cycloalkyl as indicated herein.

The term “hydroxy(Cₐ-C_{b})alkyl” is intended to indicate an (Cₐ-C_{b})alkyl group as defined above substituted with one or more hydroxy, e.g. hydroxymethyl, hydroxyethyl, hydroxypropyl.

The term “oxo” is intended to indicate an oxygen atom which is connected to the parent molecular moiety via a double bond (═O).

The term “phenyl-(Cₐ-C_{b})alkyl” is intended to indicate a phenyl group appended to the parent molecular moiety through an (Cₐ-C_{b})alkyl group, as defined herein.

When two or more of the above defined or similar terms are used in combination, such as cycloalkylalkyl or phenyl-(Cₐ-C_{b})alkyl and the like, it is to be understood that the first mentioned radical is a substituent on the latter mentioned radical, where the point of attachment to the parent molecular moiety is on the latter radical.

The group C(O) is intended to represent a carbonyl group (C═O)

If substituents are described as being independently selected from a group, each substituent is selected independent of the other. Each substituent may therefore be identical or different from the other substituent(s).

The term “optionally substituted” means “unsubstituted or substituted”, and therefore the general formulas described herein encompasses compounds containing the specified optional substituent(s) as well as compounds that do not contain the optional substituent(s).

The term “pharmaceutically acceptable salt” is intended to indicate salts prepared by reacting a compound of formula I, which comprise a basic moiety, with a suitable inorganic or organic acid, such as hydrochloric, hydrobromic, hydroiodic, sulfuric, nitric, phosphoric, formic, acetic, 2,2-dichloroacetic, adipic, ascorbic, L-aspartic, L-glutamic, galactaric, lactic, maleic, L-malic, phthalic, citric, propionic, benzoic, glutaric, gluconic, D-glucuronic, methanesulfonic, salicylic, succinic, malonic, tartaric, benzenesulfonic, ethane-1,2-disulfonic, 2-hydroxy ethanesulfonic acid, toluenesulfonic, sulfamic or fumaric acid. Pharmaceutically acceptable salts of compounds of formula I comprising an acidic moiety may also be prepared by reaction with a suitable base such as sodium hydroxide, potassium hydroxide, magnesium hydroxide, calcium hydroxide, zinc hydroxide, barium hydroxide, ammonia or the like, or suitable non-toxic amines, such as lower alkylamines (such as diethylamine, tetraalkylammonium hydroxide), hydroxy-lower alkylamines (such as diethanolamine, 2-(diethylamino)-ethanol, ethanolamine, triethanolamine, tromethamine, deanol), cycloalkylamines, ethylene diamine, or benzylamines, (such as benethamine and benzathine), betaine, choline hydroxide, N-methyl-glucamine, hydrabamine, 1H-imidazole, 4-(2-hydroxyethyl)-morpholine, piperazine, 1-(2-hydroxyethyl)-pyrrolidine, L-arginine or L-lysine. Further examples of pharmaceutical acceptable salts are listed in Berge, S. M.; J. Pharm. Sci.; (1977), 66(1), 1-19, and Stahl, P. H. and in Wermuth, C. G, Handbook of Pharmaceutical Salts, Properties, Selection and Use, 2^{nd} Edition, Wiley-VCH, 2011 both of which is incorporated herein by reference.

The term ‘prodrug’ is intended to indicate compounds which are drug-precursors which, upon administration, are converted to the parent drug in vivo by enzymatic and/or chemical reactions. Generally, the pro-drug is less biologically active than its parent drug. The prodrug may have improved physical-chemical properties compared to the parent drug, such as improved aqueous solubility, thereby facilitating the absorption and consequently the bioavailability of the parent compound upon administration.

The term “solvate” is intended to indicate a species formed by interaction between a compound, e.g. a compound of formula I, and a solvent, e.g. alcohol, glycerol or water, wherein said species are in a crystalline form. When water is the solvent, said species is referred to as a hydrate.

The term “treatment” as used herein means the management and care of a patient for the purpose of combating a disease, disorder or condition. The term is intended to include the delaying of the progression of the disease, disorder or condition, the amelioration, alleviation or relief of symptoms and complications, and/or the cure or elimination of the disease, disorder or condition. The term may also include prevention of the condition, wherein prevention is to be understood as the management and care of a patient for the purpose of combating the disease, condition or disorder and includes the administration of the active compounds to prevent the onset of the symptoms or complications. Nonetheless, prophylactic (preventive) and therapeutic (curative) treatments are two separate aspects.

All references, including publications, patent applications and patents, cited herein are hereby incorporated by reference in their entirety and to the same extent as if each reference were individually and specifically indicated to be incorporated by reference, regardless of any separately provided incorporation of particular documents made elsewhere herein.

### Embodiments of the Invention

In an embodiment the invention relates to a compound of general formula (Ia)

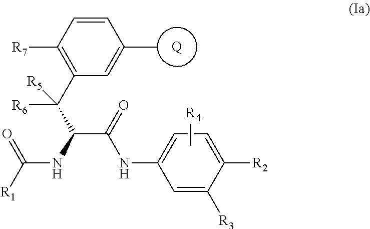

wherein R₁, R₂, R₃, R₄, R₅, R₆, R₇ and Q are as defined above.

In one embodiment the invention relates to compounds of formula (I) or (Ia) wherein

R₁ is selected from (C₃-C₇)cycloalkyl, (C₃-C₇)cycloalkyl(C₁-C₆)alkyl, (C₃-C₇)cycloalkyl(C₁-C₆)alkoxy, (C₃-C₇)cycloalkoxy and 5- or 6 membered heteroaryl wherein said (C₃-C₇)cycloalkyl, (C₃-C₇)cycloalkyl(C₁-C₆)alkyl, (C₃-C₇)cycloalkyl(C₁-C₆)alkoxy, (C₃-C₇)cycloalkoxy and 5- or 6 membered heteroaryl is optionally substituted with one or more substituents independently selected from Rₐ;

R₂ is selected from the group consisting of 5-membered heteroaryl, wherein said 5-membered heteroaryl is optionally substituted with one or more substituents independently selected from R_{b};

R₅ and R₆ each independently are selected from the group consisting of hydrogen, deuterium, (C₁-C₄)alkyl, (C₃-C₇)cycloalkyl and phenyl, with the proviso that at least one of R₅ and R₆ is selected from hydrogen or deuterium;

R₇ is selected from the group consisting of hydrogen and halogen, with the proviso that R₇ is not hydrogen when both R₅ and R₆ are selected from hydrogen or deuterium;

or R₆ and R₇ together with the phenyl to which R₇ is attached form a fused bicyclic ring system selected from the group consisting of tetralin and indane, wherein said tetralin or indane is optionally substituted with one more substituents independently selected from the group consisting of deuterium, halogen and (C₁-C₄)alkyl;

Q represents phenyl, 4-8 membered heterocycloalkyl, 5- or 6-membered heteroaryl, 9- or 10-membered bicyclic heteroaryl, pyridonyl, indolinyl, isoindolinyl, tetrahydroquinolinyl or tetrahydroisoquinolinyl, wherein said pyridonyl, 4-8 membered heterocycloalkyl, phenyl, 5- or 6-membered heteroaryl, 9- or 10-membered bicyclic heteroaryl, indolinyl, isoindolinyl, tetrahydroquinolinyl or tetrahydroisoquinolinyl is optionally substituted with one or more substituents independently selected from R₈;

and wherein Rₐ, R_{b} and R₈ are as defined above

and pharmaceutically acceptable salts, hydrates, solvates or prodrugs thereof.

In one embodiment the invention relates to a compound of general formula (I) or (Ia) as wherein

R₁ is 5- or 6 membered heteroaryl optionally substituted with one or more substituents independently selected from Rₐ;

R₂ is 5-membered heteroaryl optionally substituted with one or more substituents independently selected from R_{b};

R₆ and R₇ together with the phenyl to which R₇ is attached form a fused bicyclic ring system selected from tetralin and indane, wherein said tetralin or indane is optionally substituted with one or more substituents independently selected from the group consisting of deuterium, halogen and C₁₋₄ alkyl;

Q is selected from phenyl, 5- or 6-membered heteroaryl, pyridonyl, pyrrolidinyl, indolinyl, isoindolinyl, tetrahydroisoquinolinyl and tetrahydroquinolinyl, wherein said phenyl, 5- or 6-membered heteroaryl, pyridonyl, pyrrolidinyl, indolinyl, isoindolinyl, tetrahydroisoquinolinyl or tetrahydroquinolinyl is optionally substituted with one or more substituents independently selected from R₈;

and wherein R₈, Rₐ and R_{b} are as defined above.

In one embodiment the invention relates to a compound of general formula (I) or (Ia) wherein

R₁ is (C₃-C₇)cycloalkyl, (C₃-C₇)cycloalkyl(C₁-C₆)alkyl, (C₃-C₇)cycloalkyl(C₁-C₆)alkoxy or (C₃-C₇)cycloalkoxy wherein said (C₃-C₇)cycloalkyl, (C₃-C₇)cycloalkyl(C₁-C₆)alkyl, (C₃-C₇)cycloalkyl(C₁-C₆)alkoxy and (C₃-C₇)cycloalkoxy is optionally substituted with one or more substituents independently selected from Rₐ;

R₂ is 5-membered heteroaryl optionally substituted with one or more substituents independently selected from R_{b};

R₆ and R₇ together with the phenyl to which R₇ is attached form a fused bicyclic ring system selected from tetralin and indane, wherein said tetralin or indane is optionally substituted with one or more substituents independently selected from the group consisting of deuterium, halogen and C₁₋₄ alkyl;

Q is selected from phenyl, 5- or 6-membered heteroaryl, pyridonyl, indolinyl, isoindolinyl, tetrahydroisoquinolinyl and tetrahydroquinolinyl, wherein said phenyl, 5- or 6-membered heteroaryl, pyridonyl, indolinyl, isoindolinyl, tetrahydroisoquinolinyl or tetrahydroquinolinyl is optionally substituted with one or more substituents independently selected from R₈;

and wherein R₈, Rₐ and R_{b} are as defined above.

In one embodiment the invention relates to a compound of general formula (I) or (Ia) wherein

R₁ is 5- or 6 membered heteroaryl optionally substituted with one or more substituents independently selected from Rₐ;

R₂ is 5-membered heteroaryl optionally substituted with one or more substituents independently selected from R_{b};

R₅ and R₆ are each independently are selected from the group consisting of hydrogen, deuterium, (C₁-C₄)alkyl, (C₃-C₇)cycloalkyl and phenyl, with the proviso that at least one of R₅ and R₆ is selected from hydrogen or deuterium and at least one of R₅ and R₆ is selected from (C₁-C₄)alkyl, (C₃-C₇)cycloalkyl and phenyl;

R₇ is hydrogen or halogen;

Q is selected from phenyl, 5- or 6-membered heteroaryl, pyridonyl, indolinyl, isoindolinyl, tetrahydroisoquinolinyl and tetrahydroquinolinyl wherein said phenyl, 5- or 6-membered heteroaryl, pyridonyl, indolinyl, isoindolinyl, tetrahydroisoquinolinyl or tetrahydroquinolinyl is optionally substituted with one or more substituents independently selected from R₈;

and wherein Rₐ, R_{b} and R₈, are as defined above.

In one embodiment the invention relates to a compound of general formula (I) or (Ia) wherein

R₁ is (C₃-C₇)cycloalkyl, (C₃-C₇)cycloalkyl(C₁-C₆)alkyl, (C₃-C₇)cycloalkyl (C₁-C₆)alkoxy or (C₃-C₇)cycloalkoxy wherein said (C₃-C₇)cycloalkyl, (C₃-C₇)cycloalkyl(C₁-C₆)alkyl, (C₃-C₇)cycloalkyl (C₁-C₆)alkoxy or (C₃-C₇)cycloalkoxy is optionally substituted with one or more substituents selected independently from Rₐ;

R₂ is 5-membered heteroaryl optionally substituted with one or more substituents independently selected from R_{b};

R₅ and R₆ each independently are selected from the group consisting of hydrogen, deuterium, (C₁-C₄)alkyl, (C₃-C₇)cycloalkyl and phenyl, with the proviso that at least one of R₅ and R₆ is selected from hydrogen or deuterium and at least one of R₅ and R₆ is selected from (C₁-C₄)alkyl, (C₃-C₇)cycloalkyl and phenyl;

R₇ is hydrogen or halogen;

Q is selected from phenyl, 5- or 6-membered heteroaryl, pyridonyl, indolinyl, isoindolinyl, yl and tetrahydroquinolinyl, wherein said phenyl, 5- or 6-membered heteroaryl, pyridonyl, indolinyl, isoindolinyl, tetrahydroisoquinolinyl or tetrahydroquinolinyl is optionally substituted with one or more substituents independently selected from R₈;

and wherein Rₐ, R_{b}, and R₈, are as defined above.

In one embodiment the invention relates to a compound of general formula (I) or (Ia) wherein

R₁ is pyrazolyl or isoxazolyl wherein said pyrazolyl and isoxazolyl is optionally substituted with one or more substituents independently selected from Rₐ;

R₂ is pyrazolyl or imidazolyl wherein said pyrazolyl or imidazolyl is optionally substituted with one or more substituents independently selected from R_{b};

Q is selected from phenyl, 5- or 6-membered heteroaryl, pyridonyl, indolinyl, isoindolinyl, tetrahydroisoquinolinyl and tetrahydroquinolinyl, wherein said phenyl, 5- or 6-membered heteroaryl, pyridonyl, indolinyl, isoindolinyl, tetrahydroisoquinolinyl or tetrahydroquinolinyl is optionally substituted with one or more substituents independently selected from R₈;

and wherein Rₐ, R_{b}, and R₈, are as defined above.

In one embodiment the invention relates to a compound of general formula (I) or (Ia) wherein

R₁ is pyrazol-3-yl or isoxazol-4-yl wherein said pyrazol-3-yl or isoxazol-4-yl is optionally substituted with one or more substituents independently selected from Rₐ;

R₂ is pyrazol-4-yl, pyrazol-3-yl or imidazo-4-yl wherein said pyrazol-4-yl, pyrazol-3-yl and imidazo-4-yl is optionally substituted with one or more substituents independently selected from R_{b};

Q is selected from phenyl, 5- or 6-membered heteroaryl, pyridonyl, indolinyl, isoindolinyl, tetrahydroisoquinolinyl and tetrahydroquinolinyl, wherein said phenyl, 5- or 6-membered heteroaryl, pyridonyl, indolinyl, isoindolinyl, tetrahydroisoquinolinyl or tetrahydroquinolinyl is optionally substituted with one or more substituents independently selected from R₈;

and wherein Rₐ, R_{b}, and R₈, are as defined above.

In one embodiment the invention relates to a compound of general formula (I) or (Ia) wherein

R₁ is (C₃-C₇)cycloalkyl wherein said (C₃-C₇)cycloalkyl is optionally substituted with one or more substituents independently selected from Rₐ;

R₂ is pyrazolyl or imidazolyl wherein said pyrazolyl or imidazolyl is optionally substituted with one or more substituents independently selected from R_{b};

Q is selected from phenyl, 5- or 6-membered heteroaryl, pyridonyl, indolinyl, isoindolinyl, tetrahydroisoquinolinyl and tetrahydroquinolinyl, wherein said phenyl, 5- or 6-membered heteroaryl, pyridonyl, indolinyl, isoindolinyl, tetrahydroisoquinolinyl or tetrahydroquinolinyl is optionally substituted with one or more substituents independently selected from R₈;

and wherein Rₐ, R_{b} and R₈ are as defined above.

In one embodiment the invention related to a compound of general formula (I) or (Ia) wherein

R₁ is cyclopropyl optionally substituted with one or more substituents independently selected from Rₐ;

R₂ is pyrazol-4-yl or imidazo-4-yl wherein said pyrazol-4-yl or imidazo-4-yl is optionally substituted with one or more substituents independently selected from R_{b}:

Q is selected from phenyl, 5- or 6-membered heteroaryl, pyridonyl, indolinyl, isoindolinyl, tetrahydroisoquinolinyl and tetrahydroquinolinyl, wherein said phenyl, 5- or 6-membered heteroaryl, pyridonyl, indolinyl, isoindolinyl, tetrahydroisoquinolinyl or tetrahydroquinolinyl is optionally substituted with one or more substituents independently selected from R₈;

and wherein Rₐ, R_{b} and R₈ are as defined above.

In one embodiment the invention related to a compound as in any of the embodiments above wherein

R₁ is unsubstituted or Rₐ is deuterium or (C₁-C₆)alkyl;

R₂ is unsubstituted or R_{b} is deuterium or (C₁-C₆)alkyl;

Q is unsubstituted or R₈ is deuterium or (C₁-C₆)alkyl;

wherein when Rₐ, R_{b}, and/or R₈ is (C₁-C₆)alkyl said (C₁-C₆)alkyl is optionally substituted with one or more substituents independently selected from hydroxy, (C₁-C₄)alkyl, (C₃-C₇)cycloalkyl and —NR_{g}Rₕ wherein R_{g} and Rₕ are independently selected from hydrogen, (C₁-C₄)alkyl, (C₃-C₇)cycloalkyl, (C₃-C₇)cycloalkyl(C₁-C₄)alkyl and hydroxy(C₁-C₄)alkyl.

In another embodiment the invention relates to a compound according to any of the embodiments above wherein

R₁ is unsubstituted or Rₐ is deuterium or (C₁-C₆)alkyl;

R₂ is unsubstituted or R_{b} is deuterium or (C₁-C₆)alkyl;

R₈ is —NR_{g}Rₕ, or (C₁-C₆)alkyl substituted with —NR_{g}Rₕ wherein R_{g} and Rₕ together with the nitrogen to which they are attached form a 4-8 membered heterocycloalkyl wherein said 4-8 membered heterocycloalkyl is optionally substituted with one or more substituents independently selected from deuterium, halogen, hydroxy, (C₁-C₄)alkyl, hydroxy(C₁-C₄)alkyl and halo(C₁-C₄)alkyl.

In yet another embodiment the invention relates to a compound selected from:
N-[(1S)-2-[4-(3,5-dimethyl-1H-pyrazol-4-yl)anilino]-1-[(1R)-6-isoindolin-5-ylindan-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-6-[4-(morpholinomethyl)phenyl]indan-1-yl]-2-oxo-ethyl]-2-methyl-pyrazole-3-carboxamide;
N-[(1S)-1-[(1R)-6-(1-tert-butylpyrazol-4-yl)indan-1-yl]-2-[4-(3,5-dimethyl-1H-pyrazol-4-yl)anilino]-2-oxo-ethyl]-2-methyl-pyrazole-3-carboxamide;
N-[(1S)-1-[(1R)-6-(1-cyclobutylpyrazol-4-yl)indan-1-yl]-2-[4-(3,5-dimethyl-1H-pyrazol-4-yl)anilino]-2-oxo-ethyl]-2-methyl-pyrazole-3-carboxamide;
N-[(1S)-2-[4-(3,5-dimethyl-1H-pyrazol-4-yl)anilino]-1-[(1R)-6-(1-isopropyl-6-oxo-3-pyridyl)indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-(1-isopropylpyrazol-4-yl)indan-1-yl]-2-oxo-ethyl]-2-methyl-pyrazole-3-carboxamide;
N-[(1S)-1-[(1R)-6-(1-cyclopropylpyrazol-4-yl)indan-1-yl]-2-[4-(3,5-dimethyl-1H-pyrazol-4-yl)anilino]-2-oxo-ethyl]-2-methyl-pyrazole-3-carboxamide;
N-[(1S)-2-[4-(3,5-dimethyl-1H-pyrazol-4-yl)anilino]-2-oxo-1-[(1R)-6-(2-piperazin-1-yl-4-pyridyl)indan-1-yl]ethyl]-1-fluoro-cyclopropanecarboxamide;
N-[(1S)-2-[4-(3,5-dimethyl-1H-pyrazol-4-yl)anilino]-1-[(1R)-6-(2-methylisoindolin-5-yl)indan-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-6-[1-(2-hydroxy-1,1-dimethyl-ethyl)pyrazol-4-yl]indan-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-6-[1-(2-hydroxy-2-methyl-propyl)pyrazol-4-yl]indan-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-6-(2-isopropyl-4-pyridyl)indan-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-6-(2-methyl-3,4-dihydro-1H-isoquinolin-6-yl)indan-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-6-[2-(1-hydroxy-1-methyl-ethyl)-4-pyridyl]indan-1-yl]-2-oxo-ethyl]-2-methyl-pyrazole-3-carboxamide;
N-[(1S)-2-[4-(3,5-dimethyl-1H-pyrazol-4-yl)anilino]-2-oxo-1-[(1R)-6-(1,2,3,4-tetrahydroisoquinolin-6-yl)indan-1-yl]ethyl]-2-methyl-pyrazole-3-carboxamide;
N-[(1S)-2-[4-(3,5-dimethyl-1H-pyrazol-4-yl)anilino]-1-[(1R)-6-[3-methyl-4-(morpholinomethyl)phenyl]indan-1-yl]-2-oxo-ethyl]-2-methyl-pyrazole-3-carboxamide;
N-[(1S)-2-[4-(3,5-dimethyl-1H-pyrazol-4-yl)anilino]-2-oxo-1-[(1R)-6-[4-(pyrrolidin-1-ylmethyl)phenyl]indan-1-yl]ethyl]-2-methyl-pyrazole-3-carboxamide;
N-[(1S)-1-[(1R)-6-[4-(azetidin-1-ylmethyl)phenyl]indan-1-yl]-2-[4-(3,5-dimethyl-1H-pyrazol-4-yl)anilino]-2-oxo-ethyl]-2-methyl-pyrazole-3-carboxamide;
N-[(1S)-1-[(1R)-6-[2-[(1S,4S)-2,5-diazabicyclo[2.2.1]heptan-2-yl]-4-pyridyl]indan-1-yl]-2-[4-(3,5-dimethyl-1H-pyrazol-4-yl)anilino]-2-oxo-ethyl]-1-fluoro-cyclopropanecarboxamide;
N-[(1S)-1-[(1R)-7-(1-cyclopropylpyrazol-4-yl)tetralin-1-yl]-2-[4-(3,5-dimethyl-1H-pyrazol-4-yl)anilino]-2-oxo-ethyl]-2-methyl-pyrazole-3-carboxamide;
N-[(1S)-2-[4-(3,5-dimethyl-1H-pyrazol-4-yl)anilino]-1-[(1R)-7-(1-isopropyl-6-oxo-3-pyridyl)tetralin-1-yl]-2-oxo-ethyl]-1-fluoro-cyclopropanecarboxamide;
N-[(1S)-1-[(1R)-7-(1-isopropyl-6-oxo-3-pyridyl)tetralin-1-yl]-2-[4-(3-methylimidazol-4-yl)anilino]-2-oxo-ethyl]-2-methyl-pyrazole-3-carboxamide;
cyclopropyl N-[(1S)-2-[3-hydroxy-4-(3-methylimidazol-4-yl)anilino]-1-[(1R)-7-(1-isopropyl-6-oxo-3-pyridyl)tetralin-1-yl]-2-oxo-ethyl]carbamate;
N-[(1S)-2-[4-(3,5-dimethyl-1H-pyrazol-4-yl)-3-hydroxy-anilino]-1-[(1R)-7-(1-isopropyl-6-oxo-3-pyridyl)tetralin-1-yl]-2-oxo-ethyl]-2-methyl-pyrazole-3-carboxamide;
N-[(1S)-1-[(1R)-7-(1-tert-butylpyrazol-4-yl)tetralin-1-yl]-2-[4-(3,5-dimethyl-1H-pyrazol-4-yl)anilino]-2-oxo-ethyl]-2-methyl-pyrazole-3-carboxamide;
N-[(1S)-2-[4-(3,5-dimethyl-1H-pyrazol-4-yl)anilino]-1-[(1R)-7-isoindolin-5-yltetralin-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-7-(2-methyl-3,4-dihydro-1H-isoquinolin-6-yl)tetralin-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-7-(2-methylisoindolin-5-yl)tetralin-1-yl]-2-oxo-ethyl]-2-methyl-pyrazole-3-carboxamide;
N-[(1S)-2-[4-(3,5-dimethyl-1H-pyrazol-4-yl)anilino]-2-oxo-1-[(1R)-7-(1,2,3,4-tetrahydroisoquinolin-6-yl)tetralin-1-yl]ethyl]-2-methyl-pyrazole-3-carboxamide;
N-[(1S)-2-[4-(3,5-dimethyl-1H-pyrazol-4-yl)anilino]-2-oxo-1-[(1R)-7-(2-piperazin-1-yl-4-pyridyl)tetralin-1-yl]ethyl]-1-fluoro-cyclopropanecarboxamide;
N-[(1S)-1-[(1R)-7-[2-[(1S,4S)-2,5-diazabicyclo[2.2.1]heptan-2-yl]-4-pyridyl]tetralin-1-yl]-2-[4-(3,5-dimethylimidazol-4-yl)anilino]-2-oxo-ethyl]-1-fluoro-cyclopropanecarboxamide;
N-[(1S)-2-[4-(3,5-dimethylimidazol-4-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]-1-fluoro-cyclopropanecarboxamide;
N-[(1S)-2-[4-(3,5-dimethylimidazol-4-yl)anilino]-1-[(1R)-7-isoindolin-5-yltetralin-1-yl]-2-oxo-ethyl]-2-methyl-pyrazole-3-carboxamide;
N-[(1S)-2-[4-(3,5-dimethyl-1H-pyrazol-4-yl)-3-hydroxy-anilino]-1-[(1R)-7-(1-isopropyl-6-oxo-3-pyridyl)tetralin-1-yl]-2-oxo-ethyl]-2-methyl-pyrazole-3-carboxamide;
N-[(1S)-2-[4-(3,5-dimethyl-1H-pyrazol-4-yl)-3-hydroxy-anilino]-1-[(1R)-7-(1-isopropyl-6-oxo-3-pyridyl)tetralin-1-yl]-2-oxo-ethyl]-1-fluoro-cyclopropanecarboxamide;
N-[(1S)-2-[3-hydroxy-4-(3-methylimidazol-4-yl)anilino]-1-[(1R)-7-(1-isopropyl-6-oxo-3-pyridyl)tetralin-1-yl]-2-oxo-ethyl]-2-methyl-pyrazole-3-carboxamide;
N-[(1S)-1-[(1R)-7-[3-(1-amino-1-methyl-ethyl)phenyl]tetralin-1-yl]-2-[4-(3,5-dimethylimidazol-4-yl)anilino]-2-oxo-ethyl]-2-methyl-pyrazole-3-carboxamide;
N-[(1S)-1-[[2-chloro-5-[3-(1-hydroxy-1-methyl-ethyl)phenyl]phenyl]methyl]-2-[4-(3,5-dimethyl-1H-pyrazol-4-yl)anilino]-2-oxo-ethyl]-2-methyl-pyrazole-3-carboxamide;
N-[(1S)-1-[[5-[3-(1-amino-1-methyl-ethyl)phenyl]-2-chloro-phenyl]methyl]-2-[4-(3,5-dimethyl-1H-pyrazol-4-yl)anilino]-2-oxo-ethyl]-2-methyl-pyrazole-3-carboxamide;
N-[(1S)-1-[[2-chloro-5-[3-(dimethylaminomethyl)phenyl]phenyl]methyl]-2-[4-(3,5-dimethyl-1H-pyrazol-4-yl)anilino]-2-oxo-ethyl]-2-methyl-pyrazole-3-carboxamide;
N-[(1S)-1-[[2-chloro-5-(1-isopropyl-6-oxo-3-pyridyl)phenyl]methyl]-2-[3-hydroxy-4-(2-methylpyrazol-3-yl)anilino]-2-oxo-ethyl]-3-methyl-isoxazole-4-carboxamide;
N-[1-[[2-chloro-5-(1-isopropyl-6-oxo-3-pyridyl)phenyl]methyl]-2-[4-(3,5-dimethyl-1H-pyrazol-4-yl)anilino]-2-oxo-ethyl]-2-methyl-pyrazole-3-carboxamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]-3-methyl-isoxazole-4-carboxamide;
N-[(1S)-1-[[2-chloro-5-[2-[cyclopropyl(hydroxy)methyl]-4-pyridyl]phenyl]methyl]-2-[4-(3-methylimidazol-4-yl)anilino]-2-oxo-ethyl]-3-methyl-isoxazole-4-carboxamide;
N-[(1S)-1-[[2-chloro-5-(1-isopropyl-6-oxo-3-pyridyl)phenyl]methyl]-2-[3-hydroxy-4-(3-methylimidazol-4-yl)anilino]-2-oxo-ethyl]-3-methyl-isoxazole-4-carboxamide;
N-[(1S)-1-[[2-chloro-5-(1-isopropyl-6-oxo-3-pyridyl)phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[2-chloro-5-[2-(1-cyclopropyl-1-hydroxy-ethyl)-4-pyridyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-[2-(1-cyclopropyl-1-hydroxy-ethyl)-4-pyridyl]phenyl]methyl]-2-[4-(3-methylimidazol-4-yl)anilino]-2-oxo-ethyl]-1-fluoro-cyclopropanecarboxamide;
N-[1-[[2-chloro-5-[2-[cyclopropyl(methyl)amino]-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-[isopropyl(methyl)amino]-4-pyridyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-(1-isopropyl-6-oxo-3-pyridyl)phenyl]methyl]-2-[3-hydroxy-4-(3-methyl-1H-pyrazol-4-yl)anilino]-2-oxo-ethyl]-2-methyl-pyrazole-3-carboxamide;
N-[(1S)-1-[[2-chloro-5-(1-isopropyl-6-oxo-3-pyridyl)phenyl]methyl]-2-[3-hydroxy-4-(1H-pyrazol-4-yl)anilino]-2-oxo-ethyl]-2-methyl-pyrazole-3-carboxamide;
N-[1-[[5-[3-(1-amino-1-methyl-ethyl)phenyl]-2-chloro-phenyl]methyl]-2-[4-(3-methylimidazol-4-yl)anilino]-2-oxo-ethyl]-2-methyl-pyrazole-3-carboxamide;
N-[(1S)-1-[[2-chloro-5-(1-isopropyl-6-oxo-3-pyridyl)phenyl]methyl]-2-[4-(3,5-dimethyl-1H-pyrazol-4-yl)-3-hydroxy-anilino]-2-oxo-ethyl]-2-methyl-pyrazole-3-carboxamide;
N-[1-[[2-chloro-5-[3-(1-hydroxy-1-methyl-ethyl)phenyl]phenyl]methyl]-2-[4-(3-methylimidazol-4-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-[4-(3-chloro-1H-pyrazol-4-yl)anilino]-2-oxo-ethyl]-2-methyl-pyrazole-3-carboxamide;
N-[(1S)-1-[[2-chloro-5-[3-(1-hydroxy-1-methyl-ethyl)phenyl]phenyl]methyl]-2-[4-(3,5-dimethylimidazol-4-yl)anilino]-2-oxo-ethyl]-2-methyl-pyrazole-3-carboxamide;
N-[(1S)-1-[[5-(1-tert-butylpyrazol-4-yl)-2-chloro-phenyl]methyl]-2-[4-(3,5-dimethylimidazol-4-yl)anilino]-2-oxo-ethyl]-2-methyl-pyrazole-3-carboxamide;
N-[(1S)-1-[[5-(1-tert-butylpyrazol-4-yl)-2-chloro-phenyl]methyl]-2-[4-(3,5-dimethyl-1H-pyrazol-4-yl)anilino]-2-oxo-ethyl]-2-methyl-pyrazole-3-carboxamide;
N-[(1S)-1-[[2-chloro-5-[2-[(2R)-2-methylpiperazin-1-yl]-4-pyridyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-[2-[(1R,4R)-2-oxa-5-azabicyclo[2.2.1]heptan-5-yl]-4-pyridyl]phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[2-chloro-5-[2-[(1S,4S)-2,5-diazabicyclo[2.2.1]heptan-2-yl]-4-pyridyl]phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[2-chloro-5-(2-morpholino-4-pyridyl)phenyl]methyl]-2-[4-(3,5-dimethylimidazol-4-yl)anilino]-2-oxo-ethyl]-1-fluoro-cyclopropanecarboxamide;
N-[(1S)-1-[[2-chloro-5-(2-morpholino-4-pyridyl)phenyl]methyl]-2-[4-(3,5-dimethyl-1H-pyrazol-4-yl)anilino]-2-oxo-ethyl]-1-fluoro-cyclopropanecarboxamide;
N-[1-[[3-(1-isopropyl-6-oxo-3-pyridyl)phenyl]-phenyl-methyl]-2-[4-(3-methyl-1H-pyrazol-4-yl)anilino]-2-oxo-ethyl]-2-methyl-pyrazole-3-carboxamide;
cyclopropyl N-[1-[[3-(1-isopropyl-6-oxo-3-pyridyl)phenyl]-phenyl-methyl]-2-[4-(3-methyl-1H-pyrazol-4-yl)anilino]-2-oxo-ethyl]carbamate;
N-[(1S)-2-[4-(3,5-dimethyl-1H-pyrazol-4-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-7-[2-[(1S,4S)-2-oxa-5-azabicyclo[2.2.1]heptan-5-yl]-4-pyridyl]tetralin-1-yl]-2-oxo-ethyl]-1-fluoro-cyclopropanecarboxamide;

or pharmaceutically acceptable salts, hydrates or solvates thereof.

In one or more embodiments of the present invention, the compounds of general formula I have an (EC₅₀) value in IL-8 release assay of less than 1 micromolar, or of less than 100 nanomolar.

The compounds of formula I may be obtained in crystalline form either directly by concentration from an organic solvent or by crystallisation or recrystallisation from an organic solvent or mixture of said solvent and a cosolvent that may be organic or inorganic, such as water. The crystals may be isolated in essentially solvent-free form or as a solvate, such as a hydrate. The invention covers all crystalline forms, such as polymorphs and pseudopolymorphs, and also mixtures thereof.

Compounds of formula I comprise asymmetrically substituted (chiral) carbon atoms which give rise to the existence of isomeric forms, e.g. enantiomers and possibly diastereomers. The present invention relates to all such isomers, either in optically pure form or as mixtures thereof (e.g. racemic mixtures or partially purified optical mixtures). Pure stereoisomeric forms of the compounds and the intermediates of this invention may be obtained by the application of procedures known in the art. The various isomeric forms may be separated by physical separation methods such as selective crystallization and chromatographic techniques, e.g. high pressure liquid chromatography using chiral stationary phases. Enantiomers may be separated from each other by selective crystallization of their diastereomeric salts which may be formed with optically active amines, or with optically active acids. Optically purified compounds may subsequently be liberated from said purified diastereomeric salts. Enantiomers may also be resolved by the formation of diastereomeric derivatives. Alternatively, enantiomers may be separated by chromatographic techniques using chiral stationary phases. Pure stereoisomeric forms may also be derived from the corresponding pure stereoisomeric forms of the appropriate starting materials, provided that the reaction occur stereoselectively or stereospecifically. Preferably, if a specific stereoisomer is desired, said compound will be synthesized by stereoselective or stereospecific methods of preparation. These methods will advantageously employ chiral pure starting materials. Furthermore, when a double bond or a fully or partially saturated ring system is present in the molecule geometric isomers may be formed. Any geometric isomer, as separated, pure or partially purified geometric isomers or mixtures thereof are included within the scope of the invention.

In the compounds of general Formula I, the atoms may exhibit their natural isotopic abundances, or one or more of the atoms may be artificially enriched in a particular isotope having the same atomic number, but an atomic mass or mass number different from the atomic mass or mass number found in nature. The present invention includes all suitable isotopic variations of the compounds of general Formula I. For example, different isotopic forms of hydrogen include ¹H, ²H and ³H, different isotopic forms of carbon include ¹²C, ¹³C and ¹⁴C and different isotopic forms of nitrogen include ¹⁴N and ¹⁵N. Enriching for deuterium (²H) may for example increase in-vivo half-life or reduce dosage regiments, or may provide a compound useful as a standard for characterization of biological samples. Isotopically enriched compounds within general formula I can be prepared by conventional techniques well known to a person skilled in the art or by processes analogous to those described in the general procedures and examples herein using appropriate isotopically enriched reagents and/or intermediates.

Prodrugs of the compounds of formula (I) form part of the invention.

Solvates and hydrates form part of the invention.

In an embodiment the invention relates to the use of a compound of general formula (I) as defined above, in the manufacture of a medicament for the prophylaxis, treatment or amelioration of autoimmune diseases, such as psoriasis, ankylosing spondylitis, spondyloarthritis or psoriatic arthritis.

The compounds of the present invention may be useful for preventing, treating or ameliorating any of the following diseases: psoriasis, ankylosing spondylitis, spondyloarthritis or psoriatic arthritis, lichen planus, lupus nephritis, Sjögren's syndrome, acne, vitiligo, alopecia areata, ichthyosis, acute and chronic liver diseases, gout, osteoarthritis, SLE (besides LN and DLE), multiple sclerosis, plaque psoriasis, pustular psoriasis, psoriatic arthritis, rheumatoid arthritis, pityriasis rubra pilaris, pyoderma gangrenosum, hidradenitis suppurativa, discoid lupus erythematosus, Papulopustolar rosacea, atopic dermatitis, Ichthyosis, bullous pemphigoid, scleroderma, rheumatoid arthritis, tendinopathy, chronic wounds and cancer.

In an embodiment the invention relates to the use of a compound of general formula (I) as defined above, in the manufacture of a medicament for the prophylaxis, treatment or amelioration of any of the following diseases: psoriasis, ankylosing spondylitis, spondyloarthritis or psoriatic arthritis, lichen planus, lupus nephritis, Sjögren's syndrome, acne, vitiligo, alopecia areata, ichthyosis, acute and chronic liver diseases, gout, osteoarthritis, SLE (besides LN and DLE), multiple sclerosis, plaque psoriasis, pustular psoriasis, psoriatic arthritis, rheumatoid arthritis, pityriasis rubra pilaris, pyoderma gangrenosum, hidradenitis suppurativa, discoid lupus erythematosus, Papulopustolar rosacea, atopic dermatitis, Ichthyosis, bullous pemphigoid, scleroderma, rheumatoid arthritis, tendinopathy, chronic wounds and cancer.

In an embodiment the invention relates to the use of a compound of general formula (I) as defined above, in the manufacture of a medicament for the prophylaxis, treatment or amelioration of autoimmune diseases, such as psoriasis, ankylosing spondylitis, spondyloarthritis or psoriatic arthritis.

In an embodiment the invention relates to a method of preventing, treating or ameliorating autoimmune diseases, such as psoriatic arthritis, lichen planus, lupus nephritis, Sjögren's syndrome, acne, vitiligo, alopecia areata, ichthyosis, acute and chronic liver diseases, gout, osteoarthritis, SLE (besides LN and DLE), multiple sclerosis, plaque psoriasis, pustular psoriasis, psoriatic arthritis, rheumatoid arthritis, pityriasis rubra pilaris, pyoderma gangrenosum, hidradenitis suppurativa, discoid lupus erythematosus, Papulopustolar rosacea, atopic dermatitis, Ichthyosis, bullous pemphigoid, scleroderma, rheumatoid arthritis, tendinopathy, chronic wounds and cancer, the method comprising administering to a person suffering from at least one of said diseases an effective amount of one or more compounds according to according to general formula (I), optionally together with a pharmaceutically acceptable carrier or one or more excipients, optionally in combination with other therapeutically active compounds.

In an embodiment the invention relates to a method of preventing, treating or ameliorating autoimmune diseases, such as psoriasis, ankylosing spondylitis, spondyloarthritis or psoriatic arthritis, the method comprising administering to a person suffering from at least one of said diseases an effective amount of one or more compounds according to according to general formula (I), optionally together with a pharmaceutically acceptable carrier or one or more excipients, optionally in combination with other therapeutically active compounds.

Besides being useful for human treatment, the compounds of the present invention may also be useful for veterinary treatment of animals including mammals such as horses, cattle, sheep, pigs, dogs, and cats.

Pharmaceutical Compositions of the Invention

For use in therapy, compounds of the present invention are typically in the form of a pharmaceutical composition. The invention therefore relates to a pharmaceutical composition comprising a compound of formula I, optionally together with one or more other therapeutically active compound(s), together with a pharmaceutically acceptable excipient, vehicle or carrier(s). The excipient must be “acceptable” in the sense of being compatible with the other ingredients of the composition and not deleterious to the recipient thereof.

Conveniently, the active ingredient comprises from 0.0001-99.9% by weight of the formulation.

In the form of a dosage unit, the compound may be administered one or more times a day at appropriate intervals, always depending, however, on the condition of the patient, and in accordance with the prescription made by the medical practitioner. Conveniently, a dosage unit of a formulation contain between 0.001 mg and 1000 mg, preferably between 0.01 mg and 300 mg of a compound of formula I.

A suitable dosage of the compound of the invention will depend, inter alia, on the age and condition of the patient, the severity of the disease to be treated and other factors well known to the practising physician. The compound may be administered either orally, parenterally, topically, transdermally or interdermally and other routes according to different dosing schedules, e.g. daily, weekly or with monthly intervals. In general a single dose will be in the range from 0.001 to 400 mg/kg body weight.

If the treatment involves administration of another therapeutically active compound it is recommended to consult Goodman & Gilman's The Pharmacological Basis of Therapeutics, 9^{th} Ed., J. G. Hardman and L. E. Limbird (Eds.), McGraw-Hill 1995, for useful dosages of said compounds.

The administration of a compound of the present invention with one or more other active compounds may be either concomitantly or sequentially.

The formulations include e.g. those in a form suitable for oral, rectal, parenteral transdermal, intradermal, ophthalmic, topical, nasal, sublingual or buccal administration.

The formulations may conveniently be presented in dosage unit form and may be prepared by but not restricted to any of the methods well known in the art of pharmacy, e.g. as disclosed in Remington, The Science and Practice of Pharmacy, 21ed ed., 2005. All methods include the step of bringing the active ingredient into association with the carrier, which constitutes one or more accessory ingredients. In general, the formulations are prepared by uniformly and intimately bringing the active ingredient into association with a liquid carrier, semisolid carrier or a finely divided solid carrier or combinations of these, and then, if necessary, shaping the product into the desired formulation.

Formulations of the present invention suitable for oral and buccal administration may be in the form of discrete units as capsules, sachets, tablets, chewing gum or lozenges, each containing a predetermined amount of the active ingredient.

A tablet may be made by compressing, moulding or freeze drying the active ingredient optionally with one or more accessory ingredients. Compressed tablets may be prepared by compressing, in a suitable machine, the active ingredient(s) in a free-flowing form; for example with a lubricant; a disintegrating agent or a dispersing agent. Moulded tablets may be made by moulding, in a suitable machine, a mixture of the powdered active ingredient and suitable carrier. Freeze dried tablets may be formed in a freeze-dryer from a solution of the drug substance.

Formulations suitable for parenteral administration conveniently comprise a sterile oily or aqueous preparation of the active ingredients, which is preferably isotonic with the blood of the recipient, e.g. isotonic saline, isotonic glucose solution or buffer solution. Liposomal formulations are also suitable for parenteral administration.

Transdermal formulations may be in the form of a plaster, patch, microneedles, liposomal or nanoparticulate delivery systems or other cutaneous formulations applied to the skin.

Formulations suitable for ophthalmic administration may be in the form of a sterile aqueous preparation of the active ingredients. Liposomal formulations or biodegradable polymer systems may also be used to present the active ingredient for ophthalmic administration.

Formulations suitable for topical, such as dermal, intradermal or ophthalmic administration include liquid or semi-solid preparations, solutions or suspensions.

Formulations suitable for nasal or buccal administration include powder, self-propelling and spray formulations, such as aerosols and atomisers.

Methods of Preparation

The compounds of the present invention can be prepared in a number of ways well known to those skilled in the art of synthesis. The compounds of the invention could for example be prepared using the reactions and techniques outlined below together with methods known in the art of synthetic organic chemistry, or variations thereof as appreciated by those skilled in the art. Preferred methods include, but are not limited to, those described below. The reactions are carried out in solvents appropriate to the reagents and materials employed and suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognized by one skilled in the art. Not all compounds falling into a given class may be compatible with some of the reaction conditions required in some of the methods described. Such restrictions to the substituents which are compatible with the reaction conditions will be readily apparent to one skilled in the art and alternative methods can be used.

The compounds of the present invention or any intermediate could be purified, if required, using standard methods well known to a synthetic organist chemist, e.g. methods described in “Purification of Laboratory Chemicals”, 6^{th} ed. 2009, W. Amarego and C. Chai, Butterworth-Heinemann.

Starting materials are either known or commercially available compounds, or may be prepared by routine synthetic methods well known to a person skilled in the art. Unless otherwise noted, reagents and solvents were used as received from commercial suppliers. The organic solvents used were usually anhydrous. The solvent ratios indicated refer to vol:vol unless otherwise noted. Thin layer chromatography was performed using Merck 6OF254 silica-gel TLC plates. Visualisation of TLC plates was performed using UV light (254 nm) or by an appropriate staining technique.

Proton nuclear magnetic resonance spectra were obtained at the stated frequencies in the solvents indicated. Tetramethylsilane was used as an internal standard for proton spectra. The value of a multiplet, either defined doublet (d), triplet (t), quartet (q) or (m) at the approximate midpoint is given unless a range is quoted. (br) indicates a broad peak, whilst (s) indicates a singlet.

Mass spectra were obtained using the following methods. LCMS Method 1 was used, unless otherwise stated.

LCMS Method 1:

Column: Acquity UPLC HSS T3 1.8 μm; 2.1×50 mm

Flow: 0.7 ml/min

Column temp: 30° C.

Mobile phases: A: 10 mM Ammonium acetate+0.1% formic acid
B: 100% Acetonitrile+0.1% formic acid

UV: 240-400 nm

Injection volume: 1 μl

Gradient:

| Time | A % | B % |
|---|---|---|
| 0.0 | 99% | 1% |
| 0.5 | 94% | 6% |
| 1.0 | 94% | 6% |
| 2.6 | 5% | 95% |
| 3.8 | 5% | 95% |
| 3.81 | 99% | 1% |
| 4.8 | 99% | 1% |

UPLC (inlet method): XEV Metode 1 CM

MS-method: Pos_50_1000 or Neg_50_1000

Instruments: Waters Acquity UPLC, Waters XEVO G2-XS QTof, Waters PDA (Photodiode Array)

LCMS Method 2:

Column: Acquity UPLC BEH 1.7 μm; 2.1×50 mm

Flow: 0.7 ml/min

Column temp.: 30° C.

Mobile phases: A: 10 mM Ammonium bicarbonate
B: 100% Acetonitrile

UV: 240-400 nm

Injection volume: 1 μl

Gradient:

| | | |
|---|---|---|
| 0.0 min. | 99% A | 1% B |
| 0.5 min. | 94% A | 6% B |
| 1.0 min. | 94% A | 6% B |
| 2.6 min. | 5% A | 95% B |
| 3.8 min. | 5% A | 95% B |
| 3.81 min. | 99% A | 1% B |
| 4.8 min. | 99% A | 1% B |

UPLC (inlet method): XEV Metode 1 CM_BASIC

MS-method: Pos_50_1000 or Neg_50_1000

Instruments: Waters Acquity UPLC Waters, XEVO G2-XS QTof

LCMS Method 3:

Column: Waters Acquity UPLC HSS T3 1.8 μm, 2.1×50 mm.

Column temperature: 60° C.

UV: PDA 210-400 nm.

Injection volume: 2 μl.

Eluents: A: 10 mM Ammonium acetate with 0.1% formic acid.
B: 100% Acetonitrile with 0.1% formic acid.

Gradient:

| Time | A % | B % | Flow |
|---|---|---|---|
| 0.0 | 95 | 5 | 1.2 |
| 0.9 | 5 | 95 | 1.2 |
| 0.91 | 5 | 95 | 1.3 |
| 1.2 | 5 | 95 | 1.3 |
| 1.21 | 5 | 95 | 1.2 |
| 1.4 | 95 | 5 | 1.2 |

MS: Electrospray switching between positive and negative ionisation.

Instruments: Waters Acquity UPLC, Waters SQD, Waters PDA (Photodiode array)

LCMS Method 4:

Column: Waters Acquity UPLC BEH 1.7 μm, 2.1×50 mm.

Column temperature: 60° C.

UV: PDA 210-400 nm.

Injection volume: 2 μl.

Eluents: A: 10 mM Ammonium Bicarbonate
B: 100% Acetonitrile

Gradient:

| Time | % A | % B | Flow |
|---|---|---|---|
| 0.0 | 95 | 5 | 1.2 |
| 0.9 | 5 | 95 | 1.2 |
| 0.91 | 5 | 95 | 1.3 |
| 1.2 | 5 | 95 | 1.3 |
| 1.21 | 5 | 95 | 1.2 |
| 1.4 | 95 | 5 | 1.2 |

MS: Electrospray positive or negative ionisation.

Instrument: Waters Acquity UPLC, Waters QDa (MS detector), Waters PDA (Photodiode Array)

LCMS Method 5:

Column: Waters Acquity UPLC BEH 1.7 μm, 2.1×50 mm.

Column temperature: 35° C.

UV: PDA 190-400 nm.

Injection volume: 0.5 μl.

Eluents: A: 0.1% Formic acid in H₂O
B: 0.1% Formic acid in MeCN

Gradient:

| Time | % A | % B | Flow |
|---|---|---|---|
| 0.0 | 97 | 3 | 0.6 |
| 0.3 | 97 | 3 | 0.6 |
| 2.7 | 2 | 98 | 0.6 |
| 3.5 | 2 | 98 | 0.6 |
| 3.51 | 97 | 3 | 0.6 |

MS: Electrospray positive or negative ionisation.

Instrument: Waters Acquity UPLC, Waters Xevo TQ-S, Waters PDA (Photodiode Array)

Basic Preparative HPLC Conditions:

Column: XBridge Prep C18 5 μm OBD, 19×150 mm

Eluents: Ammonium formate (50 mM)/acetonitrile, 10-100% acetonitrile

Flow: 30 mL/min

Acidic Preparative HPLC Conditions:

Column: XTerra® RP-18 5 μm OBD, 19×150 mm

Eluents: 0.1% formic acid in water/acetonitrile, 10-100% acetonitrile

Flow: 30 mL/min

The following abbreviations have been used throughout:
ABPR automated back pressure regulator
AcOH acetic acid
Boc tert-butoxycarbonyl
DEA diethylamine
DCM dichloromethane
DIPEA diisopropylethylamine
DMF N,N-dimethylformamide
DMSO dimethylsulfoxide
dppf 1,1′-bis(diphenylphosphino)ferrocene
EtOAc ethyl acetate
EtOH ethanol
HATU 1-[Bis(dimethylamino)methylene]-1H-1,2,3-triazolo[4,5-b]pyridinium 3-oxid hexafluorophosphate
HPLC high-performance liquid chromatography
LCMS liquid chromatography-mass spectrometry
Me methyl
MeCN acetonitrile
MeOH methanol
MHz megahertz
NCS N-chlorosuccinimide
NMP N-methyl-2-pyrrolidinone
NMR nuclear magnetic resonance
ppm parts per million
SFC supercritical fluid chromatography
T3P 1-Propanephosphonic anhydride
TBAB tetra-n-butylammonium bromide
TBAF tetra-n-butylammonium fluoride
TBME tert-butyl methyl ether
TFA trifluoroacetic acid
THF tetrahydrofuran
TMS trimethylsilyl
TLC thin layer chromatography

General Methods

Compounds of the invention may be prepared according to the following non-limiting general methods and examples.

Synthesis of a compound of general formula (I), wherein R¹, R², R³, R⁴, R⁵, R⁶, R⁷ and Q are as previously defined:

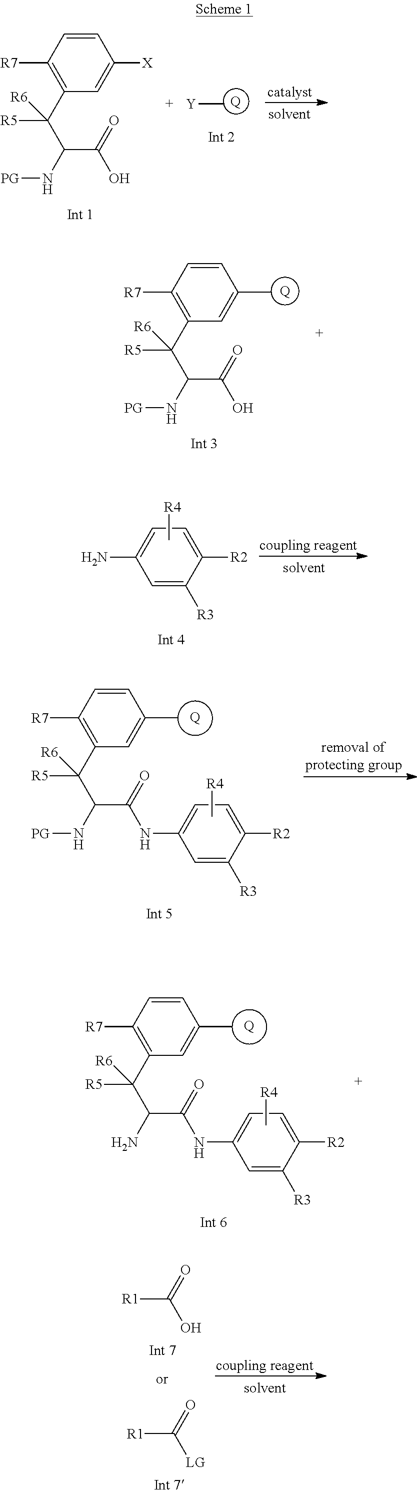

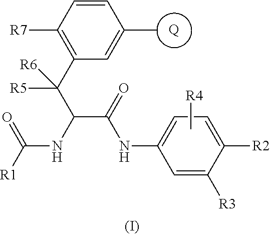

Compounds of general formula (I) can be prepared, as shown in Scheme 1. Compounds of general formula (Int 1), where X is a Br, I, phenolic triflate or boronic ester, that are either commercially available or are synthesised in a racemic form or an enantiomerically pure form, can be reacted with compounds of formula (Int 2), where Y is boronic ester or acid, Br, Cl or I, that are commercially available or are synthesised. Compounds of formula (Int 2) may contain protecting groups that can be removed or selectively removed to those skilled in the art. The reaction takes place in the presence of a catalyst such as [1,1′-bis(diphenylphosphino)ferrocene]palladium(II) dichloride PdCl₂(dppf), or bis(triphenylphosphine)palladium(II) dichloride, PdCl₂(PPh₃)₂, in the presence of an aqueous base, such as K₂CO₃ or Na₂CO₃, in a suitable solvent, such as DMF or toluene to form compounds of formula (Int 3). Those skilled in the art will appreciate other metal mediated coupling reaction will give rise to compounds of general formula (Int 3). Compounds of general formula (Int 3) are coupled with amines of general formula (Int 4), which are either commercially available or synthesised, in the presence of a coupling reagent such as HATU, HBTU, PyBOP, BOP, DCC or EDC and in most of the cases in the presence of a base, such as DIPEA or triethylamine, in a suitable solvent, such as DMF or acetonitrile to form compounds of formula (Int 5). Protecting groups (PGs), such as tert-butoxycarbonyl (Boc), benzyloxycarbonyl (Cbz), on compounds of general formula (Int 5) can be removed or selectively removed by methods known to those skilled in the art. Compounds of general formula (Int 6) are coupled with acids of general formula (Int 7), which are either commercially available or synthesised, in the presence of a coupling reagent such as HATU, HBTU, PyBOP, BOP, DCC or EDC and in most of the cases in the presence of a base, such as DIPEA or triethylamine, in a suitable solvents, such as DMF or acetonitrile, or compounds of general formula (Int 7′) that are commercial or synthesised, react in the presence of a base, such as DIPEA or triethylamine, in a suitable solvents, such as THF to form compounds of general formula (I). Leaving groups (LG) on compounds of general formula (Int 7′) such as chloro or 4-nitrophenoxy have been used. Where the compounds of general formula (I) contain protecting groups, those protecting groups can be removed by methods known to those skilled in the art. Racemic compounds of general formula (Int 3), (Int 5), (Int 6) or (I) can be separated by chiral SFC, to give the S-enantiomers of compounds of general formula (Int 3), (Int 5), (Int 6) or (I).

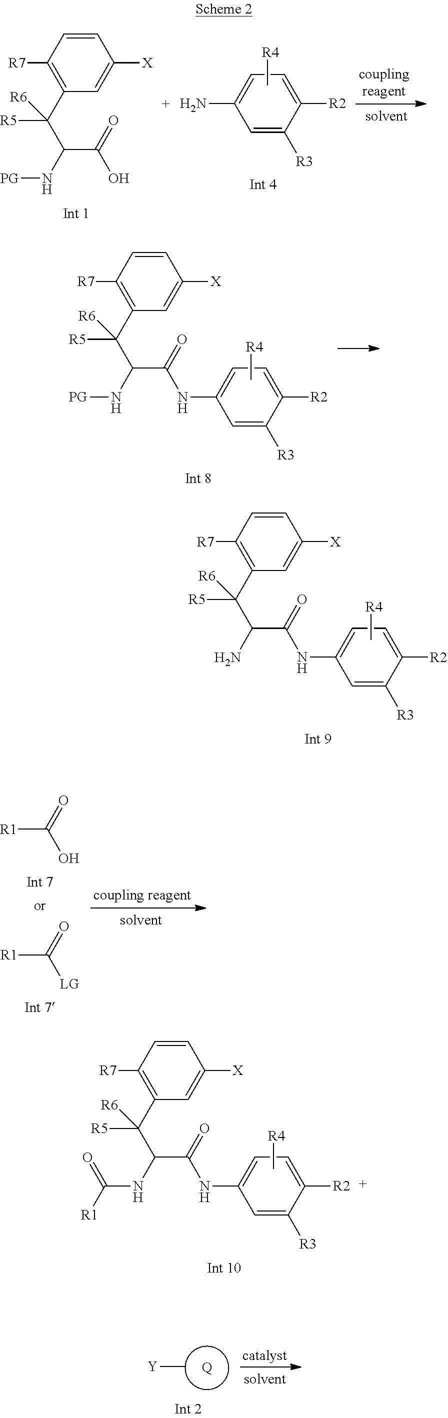

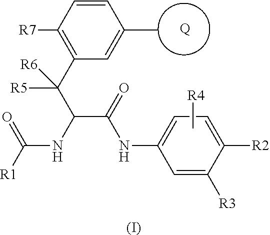

Compounds of general formula (I) can also be prepared, as shown in Scheme 2. Compounds of general formula (Int 1), where X is a Br, I, phenolic triflate or boronic ester, that are either commercially available or are synthesised in a racemic form or an enantiomerically pure form, are coupled with amines of general formula (Int 4), which are either commercially available or synthesised, in the presence of a coupling reagent such as HATU, HBTU, PyBOP, BOP, DCC or EDC and in most of the cases in the presence of a base, such as DIPEA or triethylamine, in a suitable solvent, such as DMF or acetonitrile to form compounds of formula (Int 8). Protecting groups (PGs), such as tert-butoxycarbonyl (Boc), benzyloxycarbonyl (Cbz), on compounds of general formula (Int 8) can be removed or selectively removed by methods known to those skilled in the art. Compounds of general formula (Int 9) are coupled with acids of general formula (Int 7), which are either commercially available or synthesised, in the presence of a coupling reagent such as HATU, HBTU, PyBOP, BOP, DCC or EDC and in most of the cases in the presence of a base, such as DIPEA or triethylamine, in a suitable solvents, such as DMF or acetonitrile, or compounds of general formula (Int 7′) that are commercial or synthesised, react in the presence of a base, such as DIPEA or triethylamine, in a suitable solvents, such as THF to form compounds of general formula (Int 10). Compounds of general formula (Int 10) can be reacted with compounds of formula (Int 2), where Y is boronic ester or acid, Br, Cl or I, that are commercially available or are synthesised. Compounds of formula (Int 2) may contain protecting groups that can be removed or selectively removed to those skilled in the art. The reaction takes place in the presence of a catalyst such as [1,1′-bis(diphenylphosphino)ferrocene]palladium(II) dichloride PdCl₂(dppf), or bis(triphenylphosphine)palladium(II) dichloride, PdCl₂(PPh₃)₂, in the presence of an aqueous base, such as K₂CO₃ or Na₂CO₃, in a suitable solvent, such as DMF or toluene to form compounds of formula (I). Racemic compounds of general formula (Int 8), (Int 9), (Int 10) or (I) can be separated by chiral SFC, to give the S-enantiomers of compounds of general formula (Int 8), (Int 9), (Int 10) or (I).

Synthesis of a compound of formula (Int 1), wherein R⁵, R⁶, R⁷ and X are as previously defined:

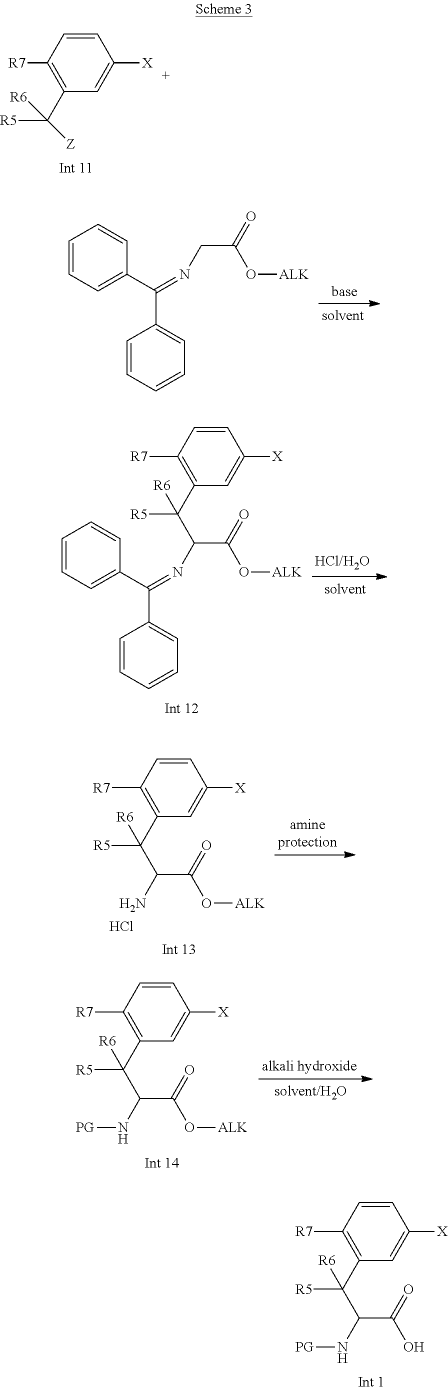

Compounds of general formula (Int 1) can be prepared, as shown in Scheme 3. Compounds of formula (Int 11), where Z is a halogen such as Br, an OH or OTs, are reacted with a commercially available compound in the presence of an alkali carbonate, such as sodium carbonate, potassium carbonate or caesium carbonate in a suitable solvent such as DMSO, DMF or acetonitrile to form compounds of formula (Int 12). Hydrolysis of compound of formula (Int 12) can be performed by using aqueous HCl in a suitable solvent, such as THF, to give compounds of general formula (Int 13). The amines of formula (Int 13) can be protected by methods known to those skilled in the art. The esters of formula (Int 14) are readily converted to Formula (Int 1) in the presence of an alkali hydroxide such as sodium hydroxide, potassium hydroxide or lithium hydroxide.

Preparation of an enantiomerically pure compound of formula (Int 1′), wherein R⁵, R⁶, R⁷ and X are as previously defined:

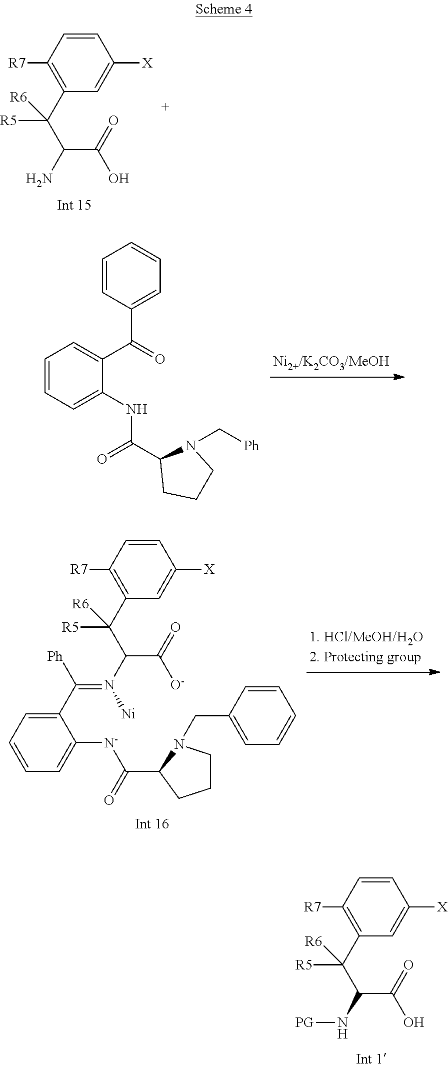

Compounds of formula (Int 1′) can be prepared, as shown in Scheme 4. Compounds of formula (Int 15) and a commercially available ligand are mixed in the presence of Ni²⁺/K₂CO₃ in a protic solvent, such as methanol, to form nickel complexes of formula (Int 16) (for dynamic kinetic resolution of α-amino acids, see: Angew. Chem. Int. Ed. 2015, 54, 12918-12922). Compounds of formula (Int 1′) are prepared by hydrolysis of compounds of formula (Int 16) in the presence of aq. HCl in a suitable protic solvent such as methanol and protecting amino functions by using, for example, CbzCl or Boc anhydride.

Preparation of an enantiomerically pure compound of formula (Int 21):

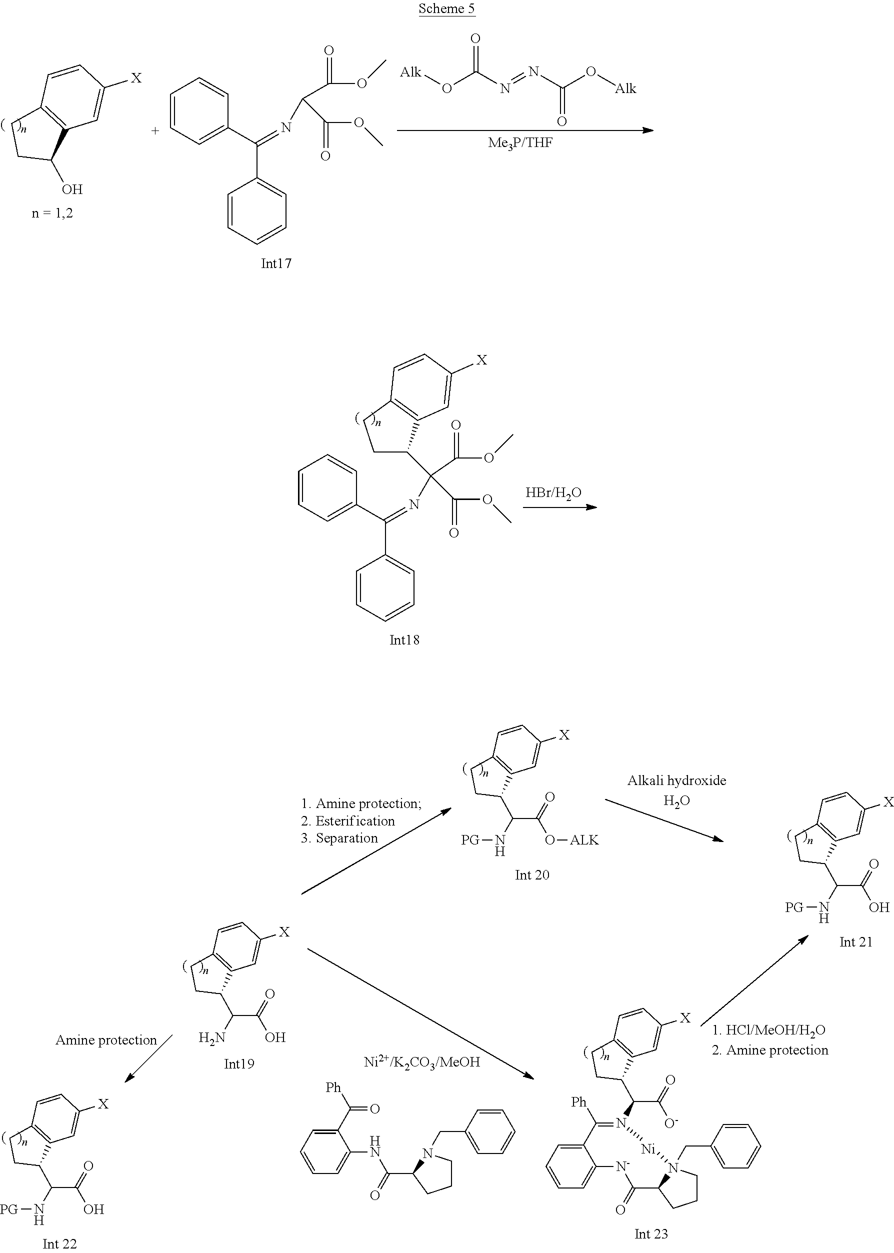

Compounds of formula (Int 21) can be prepared, as shown in Scheme 5. The compound of Formula (Int 17) is prepared according to a literature procedure (J. Org. Chem. 2017, 82, 12849-12856). Compounds of formula (Int 18) can be formed by reaction of chiral alcohols with compounds of formula (Int 17) under Mitsunobu reaction conditions (Me₃P, DEAD or DIAD) in a suitable solvent such as toluene (for a similar Mitsunobu reaction, see: Org. Lett. 2004, 6, 573-576). Compounds of formula (Int 18) can be treated with aqueous HBr under reflux, giving compounds of formula (Int 19). Compounds of formula (Int 19) can be converted to N-protected amino acid esters which are a mixture of two diastereomers. Compounds of formula (Int 20) can be isolated by silica gel flash chromatography.

Alternatively, compounds of formula (Int 19) and a commercially available ligand are mixed in the presence of Ni²⁺/K₂CO₃ in a protic solvent, such as methanol, to form nickel complexes of formula (Int 23) (for dynamic kinetic resolution of α-amino acids, see: Angew. Chem. Int. Ed. 2015, 54, 12918-12922; see also: scheme 3). Compounds of formula (Int 21) can be prepared by hydrolysis of compounds of formula (Int 23) in the presence of aq. HCl in a suitable protic solvent such as methanol and subsequently protecting the amino function by using, for example, CbzCl or Boc anhydride.

Still alternatively, diastereomeric mixtures of formula (Int 22) can be synthesised by protecting the amino function of compounds of formula (Int 19) by using, for example, CbzCl or Boc anhydride.

Preparation of compounds of formula (Int 28).

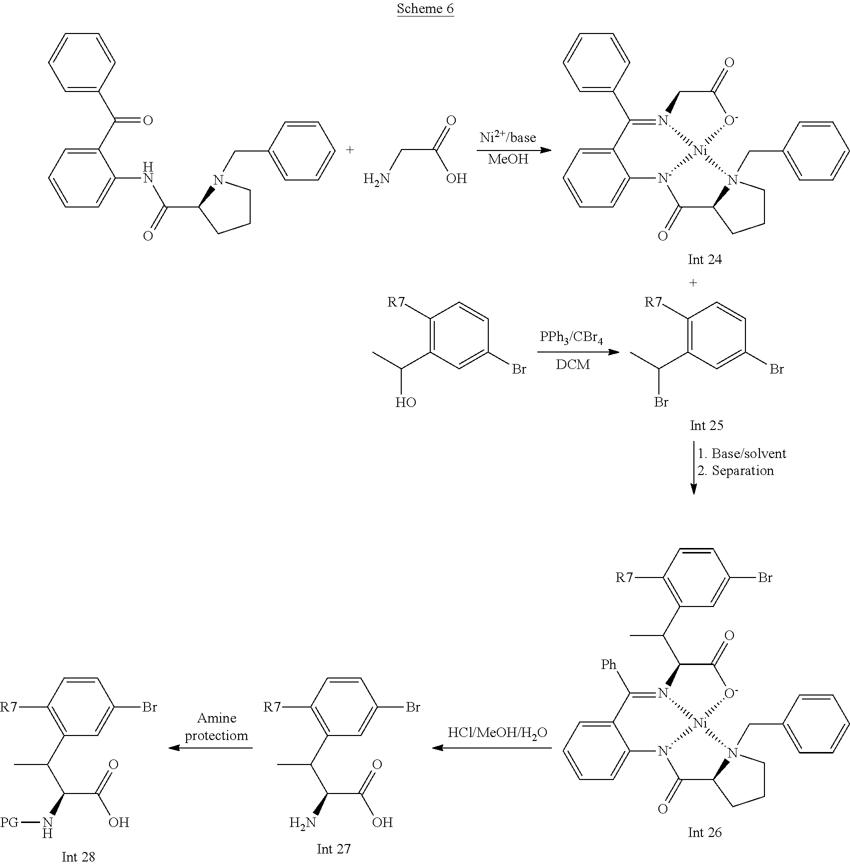

Compounds of formula (Int 24) can be prepared, as shown in Scheme 6. Commercially available 2-aminoacetic acid and a commercially available ligand are mixed in the presence of Ni²⁺ source, such as nickelous nitrate hexahydrate, with a base such as KOH or NaH, in a protic solvent, such as methanol or propan-2-ol, to form nickel complexes of formula (Int 24). Compounds of formula (Int 25) can be prepared from commercial secondary alcohols in reaction with PPh₃ and CBr₄ in a suitable solvent such as DCM or carbon tetrachloride. Compounds of formula (Int 26) are formed by mixing compounds of formula (Int 25) with the nickel complex (Int 24) in the presence of a base such as KOH, NaOH or NaH in a suitable solvent such as DMF. Compounds of formula (Int 26) can be isolated by silica gel flash chromatography. Compounds of formula (Int 27) can be prepared by hydrolysis of compounds of formula (Int 26) in the presence of aq. HCl in a suitable protic solvent such as methanol and subsequently protecting the amino function by using, for example, CbzCl or Boc anhydride to afford compounds of formula (Int 28).

Preparation of compounds of formula (Int 32).

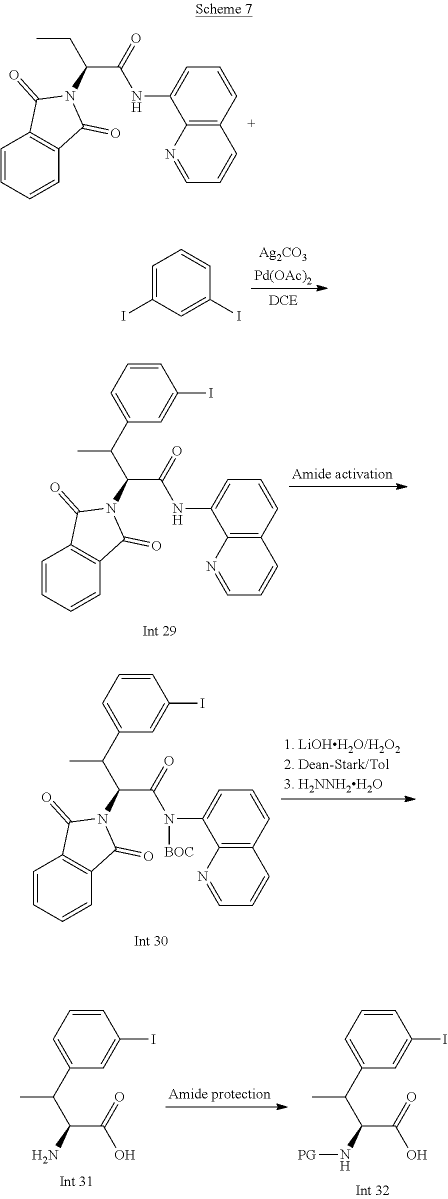

Compounds of formula (Int 32) can be prepared, as shown in Scheme 7. Compounds of formula (Int 29) can be formed by the Pd catalysed aminoquinoline directed C—H arylation reaction between the commercially available N-phthalimido-(8-quinolyl)butanamide and an aryl halide such as 1,3-diiodobenzene in the presence of Pd(OAc)₂ and Ag₂CO₃ in DCE. The amide of formula (Int 29) can be protected by methods known to those skilled in the art to form compounds of formula (Int 30). Compounds of formula (Int 31) are prepared by hydrolysis of compounds of formula (Int 30) in the presence of LiO₂H in a suitable solvent such as THF/H₂O, followed by phthalimide deprotection with hydrazine monohydrate in a suitable solvent such as Toluene. The amine of formula (Int 31) can be protected by methods known to those skilled in the art to form compounds of formula (Int 32).

Preparation of compounds of formula (Int 32).

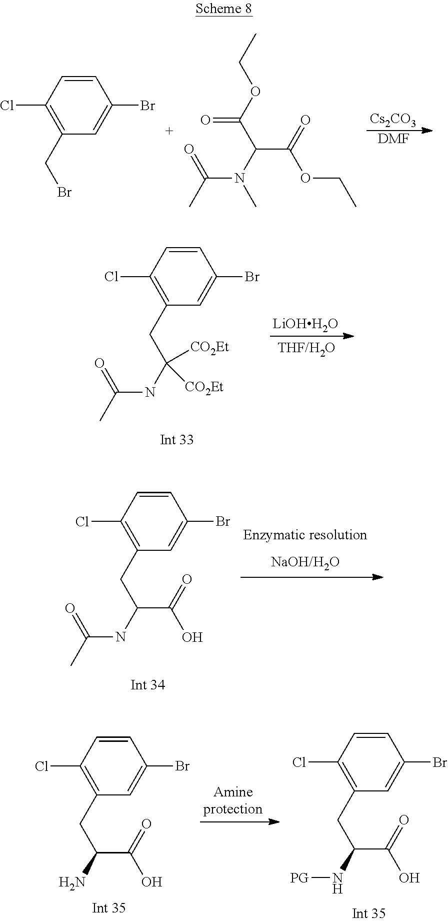

Compounds of formula (Int 36) can be prepared, as shown in Scheme 8. Compounds of formula (Int 33) can be prepared from reaction of commercial benzyl bromide with diethyl acetamidomalonate in the presence of an appropriate base such as Cs₂CO₃ in a suitable solvent such as DMF. Compounds of formula (Int 34) can be prepared by hydrolysis of compounds of formula (Int 33) in the presence of aq. LiOH in a suitable solvent such as aq. THF. Compounds of formula (Int 34) can be converted to chiral unprotected amines of formula (Int 35) using Acylase I (Aspergillus melleus) in a pH buffered aqueous solution. Compounds of formula (Int 36) are accessed by protecting the amino function by using, for example, CbzCl or Boc anhydride.

### PREPARATIONS AND EXAMPLES

### Preparation 1

### 2-[[3,5-dimethyl-4-(4-nitrophenyl)pyrazol-1-yl]methoxy]ethyl-trimethyl-silane

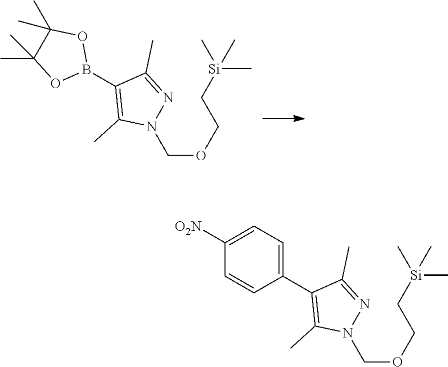

K₂CO₃ (2.12 g, 15.3 mmol) in water (11.5 mL) and Pd(dppf)Cl₂ (313 mg, 0.383 mmol) were added to a mixture of 2-[[3,5-dimethyl-4-(4,4,5,5-tetramethyl-1,3,2-dioxaborolan-2-yl)pyrazol-1-yl]methoxy]ethyl-trimethyl-silane (2.70 g, 7.66 mmol) and 4-bromonitrobenzene (1.55 g, 7.66 mmol) in THF (23 mL) and MeOH (3.83 mL). The mixture was placed in two 20 mL microwave vials which were degassed with argon for 10 min, capped and stirred for 20 min at 90° C. in a heating block. The combined reactions were diluted with with EtOAc (100 mL), washed with water (2×20 mL) and brine (20 mL), dried (MgSO₄) and concentrated in vacuo to give a dark oil. The crude product was purified by column chromatography (silica gel, loaded in DCM, eluting with 0-30% EtOAc in heptane) to give the title compound (1.885 g, 71%). ¹H NMR (300 MHz, DMSO-d6) δ 8.39-8.19 (m, 2H), 7.69-7.40 (m, 2H), 5.39 (s, 2H), 3.64-3.53 (m, 2H), 2.33 (s, 3H), 2.21 (s, 3H), 0.92-0.76 (m, 2H), −0.03 (s, 9H); LCMS (ES): m/z 384.3 [M+H]⁺, RT=0.95 min.

### Preparation 2

### 4-[3,5-dimethyl-1-(2-trimethylsilylethoxymethyl)pyrazol-4-yl]aniline

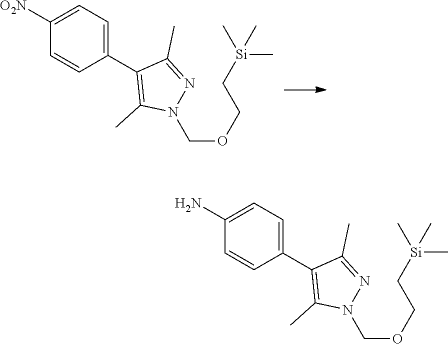 [SMILES: Cc1nn(COCC[Si](C)(C)C)c(C)c1-c1ccc([N+](=O)[O-])cc1 ; Cc1nn(COCC[Si](C)(C)C)c(C)c1-c1ccc(N)cc1]

10% Pd/C (188 mg) was added to a solution of the compound of Preparation 1 (1.88 g, 5.41 mmol) in MeOH (30 mL) and placed under hydrogen at atmospheric pressure. After 1 hour the catalyst was filtered off, washing with MeOH, and the filtrate was concentrated in vacuo to give the title compound (1.67 g, 97%) as a colourless solid. ¹H NMR (300 MHz, DMSO-d6) δ 6.96-6.85 (m, 2H), 6.65-6.57 (m, 2H), 5.30 (s, 2H), 5.03 (s, 2H), 3.59-3.48 (m, 2H), 2.20 (s, 3H), 2.08 (s, 3H), 0.83 (dd, J=8.4, 7.4 Hz, 2H), −0.04 (s, 9H); LCMS (ES): m/z 318.4 [M+H]⁺, RT=0.80 min.

### Preparation 3

### 3-chloro-4-iodo-1H-pyrazole

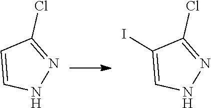 [SMILES: Clc1cc[nH]n1 ; Clc1n[nH]cc1I]

NIS (5.29 g, 23.5 mmol) was added portionwise to a solution of 3-chloro-1H-pyrazole (2.0 g, 19.6 mmol) in DMF (20 mL) at 0° C. The reaction mixture was stirred at room temperature o/n. The reaction mixture was diluted with H₂O (100 mL) and extracted with EtOAc (2×100 mL). The combined organic phases were successively washed with H₂O (50 mL) and brine (100 mL) then dried over Na₂SO₄, filtered and concentrated in vacuo. The obtained crude compound was purified by silica column chromatography (230-400 mesh), eluting with 15% EtOAc in hexane, to afford title compound as an off-white solid. (3.2 g, 71% yield); LCMS (Method 5) (ES): m/z 228.8 [M+H]⁺, RT=2.54 min.

### Preparation 4

### tert-butyl N-[4-(3-chloro-1H-pyrazol-4-yl)phenyl]carbamate

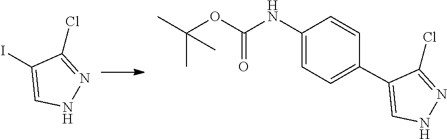 [SMILES: Clc1n[nH]cc1I ; CC(C)(C)OC(=O)Nc1ccc(-c2c[nH]nc2Cl)cc1]

Na₂CO₃ (1.4 g, 13.2 mmol) was added to a solution of the compound from Preparation 3 (1.0 g, 4.40 mmol) and [4-(tert-butoxycarbonylamino)phenyl]boronic acid (1.14 g, 4.84 mmol) in 1,4-Dioxane/H₂O (9:1, 10 mL). The reaction mixture was purged with argon for 15 min. PdCl2(dppf).DCM (0.36 g, 0.44 mmol) was added and the reaction mixture was heated under microwave irradiation at 110° C. for 1 h. The reaction mixture was diluted with H₂O (100 mL) and extracted with EtOAc (2×50 mL). The combined organic phases were successively washed with H₂O (50 mL) and brine (50 mL) then dried over Na₂SO₄, filtered and concentrated in vacuo. The obtained crude compound was purified by silica column chromatography (230-400 mesh), eluting with 1-2% MeOH in DCM, to afford title compound as an off-white solid. (150 mg, 11% yield); LCMS (Method 5) (ES): m/z 294.08 [M+H]⁺, RT=1.90 min.

### Preparation 5

### 4-(3-chloro-1H-pyrazol-4-yl)aniline

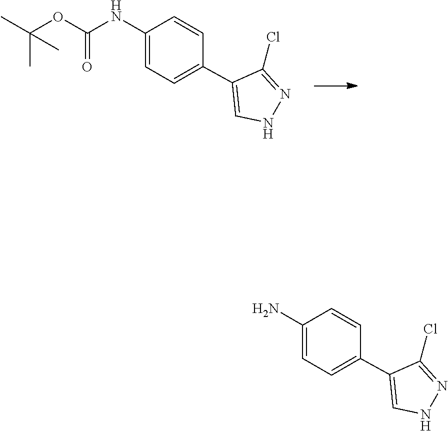 [SMILES: CC(C)(C)OC(=O)Nc1ccc(-c2c[nH]nc2Cl)cc1 ; Nc1ccc(-c2c[nH]nc2Cl)cc1]

Hydrogen chloride (4M in 1, 4-Dioxane, 1 mL) was added to a solution of the compound from Preparation 4 (70.0 mg, 0.24 mmol) at 0° C. The reaction mixture was stirred at 5° C. for 2 h. The reaction mixture was concentrated under reduced pressure and diluted with MeOH (5 mL). Amberlyst A21 base resin was added to adjust to pH 8. The resulting suspension was filtered and the filtrate was concentrated in vacuo to afford title compound as an off-white solid. (50 mg, 91% yield); LCMS (Method 5) (ES): m/z 194.13 [M+H]⁺, RT=0.95 min.

### Preparation 6

### 2-[(2-bromo-5-nitro-phenoxy)methoxy]ethyl-trimethyl-silane

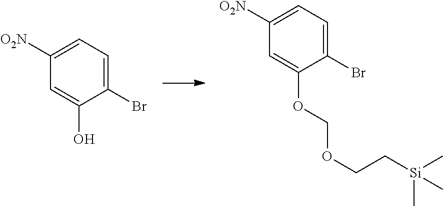 [SMILES: O=[N+]([O-])c1ccc(Br)c(O)c1 ; C[Si](C)(C)CCOCOc1cc([N+](=O)[O-])ccc1Br]

2-(chloromethoxy)ethyl-trimethyl-silane (1.38 mL, 7.80 mmol) was added to a solution of 2-bromo-5-nitro-phenol (1.0 g, 4.59 mmol) and TEA (1.28 mL, 9.17 mmol) in DCM (10 mL) at room temperature and the reaction mixture was stirred o/n. The reaction mixture was concentrated in vacuo and the residue diluted with EtOAc (30 mL). The organic phase was successively washed with H₂O (2×10 mL) and brine (10 mL) then dried over MgSO₄, filtered and concentrated in vacuo. The obtained crude compound was purified by silica column chromatography (230-400 mesh), eluting with 0-30% EtOAc in heptane, to afford title compound. (1.37 g, 85% yield); ¹H NMR (300 MHz, DMSO-d6) δ 8.05 (d, J=2.6 Hz, 1H), 7.97 (d, J=8.7 Hz, 1H), 7.85 (dd, J=8.7, 2.6 Hz, 1H), 5.55 (s, 2H), 3.88-3.72 (m, 2H), 1.03-0.85 (m, 2H), 0.00 (s, 9H).

### Preparation 7

### 2-[[3,5-dimethyl-4-[4-nitro-2-(2-trimethylsilylethoxymethoxy)phenyl]pyrazol-1-yl]methoxy]ethyl-trimethyl-silane

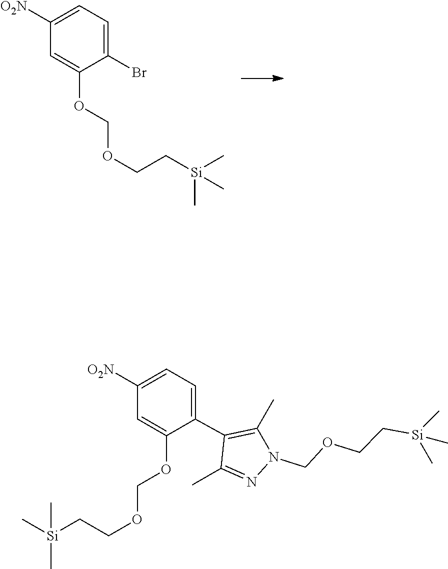

According to the method of Preparation 1 the compound of Preparation 6 (494 mg, 1.42 mmol) was reacted to afford the title compound (580 mg, 82% yield). LCMS (METHOD 3) (ES): m/z 494.5 [M+H]⁺, RT=1.10 min.

### Preparation 8

### 4-[3,5-dimethyl-1-(2-trimethylsilylethoxymethyl)pyrazol-4-yl]-3-(2-trimethylsilylethoxymethoxy)aniline

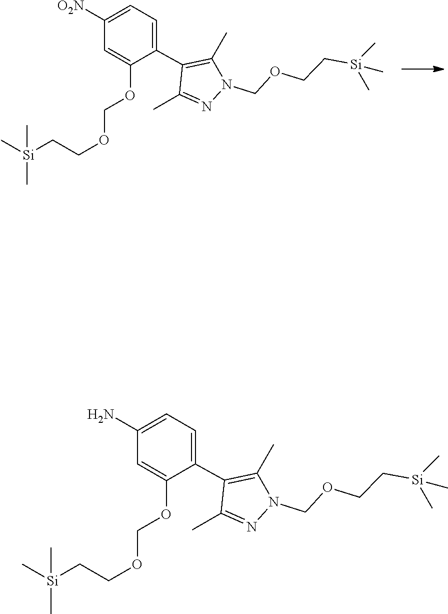

According to the method of Preparation 2 the compound of Preparation 7 (580 mg, 1.20 mmol) was reacted to afford the title compound (495 mg, 91% yield). LCMS (METHOD 3) (ES): m/z 464.5 [M+H]⁺, RT=1.00 min.

### Preparation 9

### Dimethyl 2-(benzhydrylideneamino)propanedioate

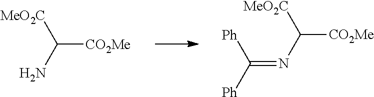 [SMILES: COC(=O)C(N)C(=O)OC ; COC(=O)C(N=C(c1ccccc1)c1ccccc1)C(=O)OC]

A mixture of benzophenone (25.0 g, 138 mmol) and dimethyl 2-aminopropanedioate hydrochloride (25.0 g, 136 mmol) in DCM (300 mL) was stirred at room temperature for 3 days. The solid material was filtered off and the filtrate was concentrated in vacuo, The residue was re-dissolved in TBME and washed with water, dried (MgSO₄) and concentrated in vacuo. The residue was purified by column chromatography (silica gel, eluting with 15-100% EtOAc in heptane) to give the title compound (27.0 g, 64%) as a colourless oil, which solidified on standing. ¹H NMR (300 MHz, Chloroform-d) δ 7.78-7.63 (m, 2H), 7.51-7.28 (m, 6H), 7.24-7.11 (m, 2H), 4.90 (s, 1H), 3.78 (s, 6H); LCMS (METHOD 4) (ES): m/z 312.2 [M+H]⁺, RT=0.73 min.

### Preparation 10

### Dimethyl 2-(benzhydrylideneamino)-2-[(1R)-6-bromoindan-1-yl]propanedioate

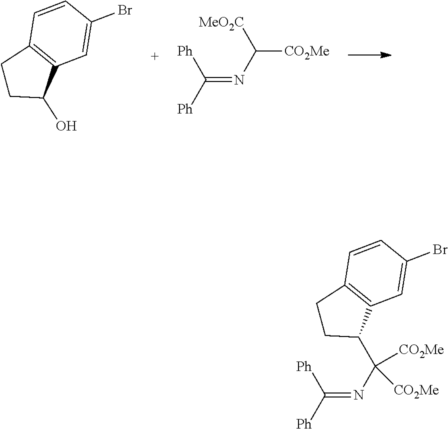 [SMILES: O[C@H]1CCc2ccc(Br)cc21 ; COC(=O)C(N=C(c1ccccc1)c1ccccc1)C(=O)OC ; COC(=O)C(N=C(c1ccccc1)c1ccccc1)(C(=O)OC)[C@@H]1CCc2ccc(Br)cc21]

A solution of (1S)-6-bromoindan-1-ol (6.5 g, 31 mmol), the compound of Preparation 9 (14.0 g, 46 mmol) and trimethylphosphine (1M solution in THF, 46 mL, 46 mmol) in toluene (100 g) was cooled down to −75° C. Diethyl azodicarboxylate (40 wt % solution in toluene, 21 g, 48 mmol) was added dropwise over 30 minutes and the solution was stirred at −75° C. for 1.5 hours and at room temperature for 3 hours to give a dark brown solution. The solution was concentrated in vacuo and the residue was purified by column chromatography (silica gel, eluting with heptane/ethyl acetate 4:1) to give the title compound (9.6 g, 62%) as a yellow oil. ¹H NMR (300 MHz, Chloroform-d) δ 7.84 (dd, J=1.9, 0.9 Hz, 1H), 7.64-7.49 (m, 2H), 7.47-7.22 (m, 7H), 7.22-7.14 (m, 2H), 7.05 (dd, J=8.0, 1.1 Hz, 1H), 4.26-4.03 (m, 1H), 3.44 (s, 3H), 3.25 (s, 3H), 2.94 (ddd, J=15.6, 9.2, 6.0 Hz, 1H), 2.76 (ddd, J=15.6, 9.1, 5.9 Hz, 1H), 2.55-2.17 (m, 2H); LCMS (METHOD 3) (ES): m/z 506.3, 508.3 [M+H]⁺, RT=1.03 min.

### Preparation 11

### 2-Amino-2-[(1R)-6-bromoindan-1-yl]acetic Acid Hydrobromide

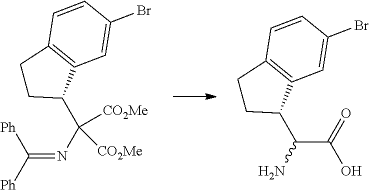 [SMILES: COC(=O)C(N=C(c1ccccc1)c1ccccc1)(C(=O)OC)[C@@H]1CCc2ccc(Br)cc21 ; NC(C(=O)O)[C@@H]1CCc2ccc(Br)cc21]

To a solution of the compound of Preparation 10 (9.6 g, 19 mmol) in THF (30 mL) at room temperature was added conc. HCl (10 mL) (exothermic). The solution was stirred at room temperature for 30 min and then diluted with TBME (70 mL) and water (30 mL). The phases were separated, the aqueous phase was extracted twice with TBME and then concentrated in vacuo, giving crude dimethyl 2-amino-2-[(1R)-6-bromoindan-1-yl]propanedioate hydrochloride as a colourless oil, which was used without further purification.

To a solution of dimethyl 2-amino-2-[(1R)-6-bromoindan-1-yl]propanedioate hydrochloride in water (20 mL) at room temperature was added conc. HBr (48% HBr, 20 mL). The solution was heated at reflux for 3 hours and the product precipitated. The suspension was cooled to room temperature and filtered. The filter cake was washed with TBME and dried in vacuo, giving the title compound (5.2 g, 78%) as a white solid as a mixture of diastereomers. ¹H NMR (300 MHz, Deuterium Oxide+1 drop DCl) δ 7.38 (s, 0.33H), 7.34-7.21 (m, 1.67H), 7.15-6.96 (m, 1H), 4.41 (d, J=4.0 Hz, 0.33H), 4.32 (d, J=3.7 Hz, 0.67H), 3.91-3.81 (m, 0.33H), 3.75 (dt, J=9.3, 4.8 Hz, 0.67H), 3.07-2.54 (m, 2H), 2.50-2.01 (m, 1H), 1.99-1.64 (m, 1H); LCMS (METHOD 3) (ES): m/z 270.2, 272.2 [M+H]⁺, RT=0.38 min.

### Preparation 12

### Nickelous (2S)-2-[(E)-[[2-[(2S)-1-benzylpyrrolidine-2-carbonyl]azanidylphenyl]-phenyl-methylene]amino]-2-[(1R)-6-bromoindan-1-yl]acetate

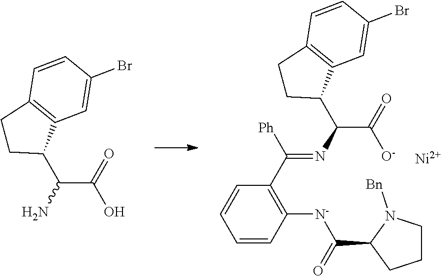

A mixture of (2S)—N-(2-benzoylphenyl)-1-benzyl-pyrrolidine-2-carboxamide (3.85 g, 10.0 mmol), the compound of Preparation 11 (3.2 g, 9.1 mmol), nickel (II) acetate hydrate (2.5 g, 8.6 mmol) and K₂CO₃ (7.0 g, 51 mmol) in MeOH (50 mL) was stirred at 80° C. for 18 h. The reaction was concentrated in vacuo and the residue was taken up in DCM and water. After separating the phases, the aqueous phase was extracted twice with DCM. The combined organic phases were dried (MgSO₄) and concentrated in vacuo. The crude product was purified by column chromatography (silica gel, eluting with EtOAc/DCM 1:3) to give a red foam. This was taken up in TBME (50 mL) and the mixture was shaken for 10 min. The solid material was filtered and washed with TBME (2×20 mL), giving the title compound (4.2 g, 66%) as a red solid). ¹H NMR (300 MHz, Chloroform-d) δ 8.56-8.34 (m, 1H), 8.12-7.93 (m, 2H), 7.60-7.45 (m, 3H), 7.41-7.23 (m, 5H), 7.19-7.07 (m, 3H), 6.71-6.57 (m, 3H), 4.36 (d, J=12.6 Hz, 1H), 4.26 (d, J=4.9 Hz, 1H), 3.49 (d, J=12.6 Hz, 1H), 3.45-3.34 (m, 2H), 3.29-3.18 (m, 1H), 3.12-2.94 (m, 2H), 2.91-2.75 (m, 1H), 2.75-2.36 (m, 3H), 2.30-2.09 (m, 1H), 2.07-1.93 (m, 1H), 1.81 (ddt, J=15.5, 8.0, 3.7 Hz, 1H); LCMS (METHOD 3) (ES): m/z 692.5, 694.5 [M+H]⁺, RT=0.90 min.

### Preparation 13

### (2S)-2-[(1R)-6-bromoindan-1-yl]-2-(tert-butoxycarbonylamino)acetic Acid

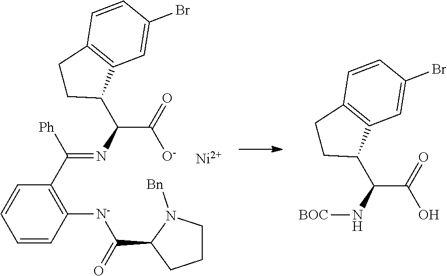

To a suspension of the compound of Preparation 12 (4.8 g, 6.9 mmol) in MeOH (40 mL) at room temperature was added 6M HCl (10 mL, 60 mmol). The suspension was heated at 60° C. for 30 minutes, by which time the dark red suspension became a blue solution. The solution was concentrated in vacuo, giving a solid residue. The residue was taken up in water (100 mL) and filtered, washing the solid with water (20 mL). The filtrate was carefully neutralised to pH 6 using sat. aq. NaHCO₃. The precipitate was collected by filtration and washed with water (2×20 mL) and DCM (2×25 mL), giving crude (2S)-2-amino-2-[(1R)-6-bromoindan-1-yl]acetic acid which was used without further purification.

The crude (2S)-2-amino-2-[(1R)-6-bromoindan-1-yl]acetic acid (6.9 mmol) was taken up in water (50 mL) and the suspension was basified to pH 12. Dioxane (40 mL) was added followed by tert-butoxycarbonyl tert-butyl carbonate (7.53 g, 34.5 mmol, 4 portions over 4 hours). After addition, the reaction mixture was stirred at room temperature for 4 hours. The reaction mixture was washed with DCM (2×50 mL), EtOAc (50 mL) was added and the mixture was acidified with 4N HCl to pH 2. The layers were separated and the aqueous phase was extracted with further EtOAc (30 mL). The combined organic phases were dried (MgSO₄) and concentrated in vacuo, giving the title compound (2.014 g, 79%) as a white solid. ¹H NMR (300 MHz, DMSO-d6) δ 7.65-6.95 (m, 4H), 4.61 (br s, 1H), 3.67 (br s, 1H), 2.93-2.60 (m, 2H), 2.22-1.81 (m, 2H), 1.32 (s, 9H); LCMS (METHOD 3) (ES): m/z 370.4, 372.4 [M+H]⁺, RT=0.76 min.

### Preparation 14

### tert-butyl N-[(1S)-1-[(1R)-6-bromoindan-1-yl]-2-[4-[3,5-dimethyl-1-(2-trimethylsilylethoxymethyl)pyrazol-4-yl]anilino]-2-oxo-ethyl]carbamate

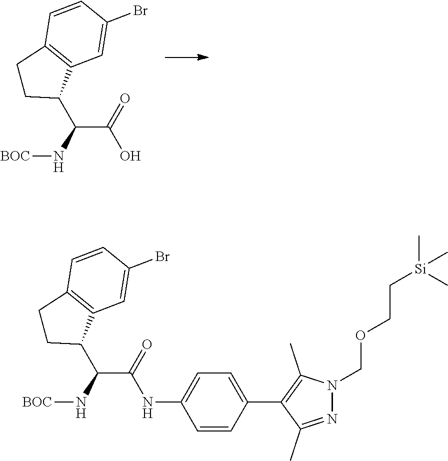 [SMILES: CC(C)(C)OC(=O)N[C@H](C(=O)O)[C@@H]1CCc2ccc(Br)cc21 ; Cc1nn(COCC[Si](C)(C)C)c(C)c1-c1ccc(NC(=O)[C@@H](NC(=O)OC(C)(C)C)[C@@H]2CCc3ccc(Br)cc32)cc1]

4-[3,5-dimethyl-1-(2-trimethylsilylethoxymethyl)pyrazol-4-yl]aniline (270.1 mg, 0.85 mmol) was added to a mixture of the compound from Preparation 13 (300 mg, 0.81 mmol), DIPEA (0.155 mL, 0.89 mmol) and HATU (323.5 mg, 0.85 mmol) in DMF (5 mL) at room temperature. The reaction mixture was stirred for 1 h. The reaction mixture was passed through a PTFE filter and purified by acidic HPLC directly to afford the title compound as an off-white solid. (484 mg, 89% yield); LCMS (METHOD 3) (ES): m/z 671.6 [M+H]⁺, RT=1.04 min.

### Preparation 15

### N-[(1S)-1-[(1R)-6-bromoindan-1-yl]-2-[4-[3,5-dimethyl-1-(2-trimethylsilylethoxymethyl)pyrazol-4-yl]anilino]-2-oxo-ethyl]-2-methyl-pyrazole-3-carboxamide

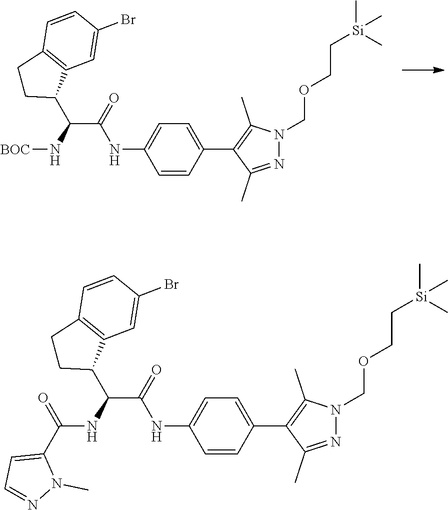 [SMILES: Cc1nn(COCC[Si](C)(C)C)c(C)c1-c1ccc(NC(=O)[C@@H](NC(=O)OC(C)(C)C)[C@@H]2CCc3ccc(Br)cc32)cc1 ; Cc1nn(COCC[Si](C)(C)C)c(C)c1-c1ccc(NC(=O)[C@@H](NC(=O)c2ccnn2C)[C@@H]2CCc3ccc(Br)cc32)cc1]

Hydrogen chloride (4M in dioxan, 2 mL) was added to a solution of the compound from Preparation 14 (480 mg, 0.71 mmol) in MeOH (4 mL) at room temperature for 1.5 h. The reaction mixture was diluted with MeOH (10 mL) concentrated in vacuo to give crude intermediate (2S)-2-amino-2-[(1R)-6-bromoindan-1-yl]-N-[4-[3,5-dimethyl-1-(2-trimethylsilylethoxymethyl)pyrazol-4-yl]phenyl]acetamide hydrochloride. Assume quantitative yield. LCMS (METHOD 3) (ES): m/z 571.5 [M+H]⁺, RT=0.82 min. DIPEA (0.5 mL, 2.87 mmol) was added to a solution of the crude (2S)-2-amino-2-[(1R)-6-bromoindan-1-yl]-N-[4-[3,5-dimethyl-1-(2-trimethylsilylethoxymethyl)pyrazol-4-yl]phenyl]acetamide hydrochloride (434 mg, 0.71 mmol) and 2-methylpyrazole-3-carboxylic acid (99.4 mg, 0.79 mmol) in DMF (5 mL) at room temperature. HATU (300 mg, 0.79 mmol) was added and the reaction mixture stirred for 30 min. The reaction mixture was purified directly by basic HPLC. The relevant fractions were combined and concentrated in vacuo. The residue was dissolved in MeOH (5 mL) and poured into H₂O (50 mL). The solid was collected and dried under reduced pressure to afford title compound as an colourless solid. (414 mg, 85% yield) LCMS (METHOD 3) (ES): m/z 679.6 [M+H]⁺, RT=0.95 min.

### Preparation 16

### tert-butyl 5-[(3R)-3-[(1S)-2-[4-[3,5-dimethyl-1-(2-trimethylsilylethoxymethyl)pyrazol-4-yl]anilino]-1-[(2-methylpyrazole-3-carbonyl)amino]-2-oxo-ethyl]indan-5-yl]isoindoline-2-carboxylate

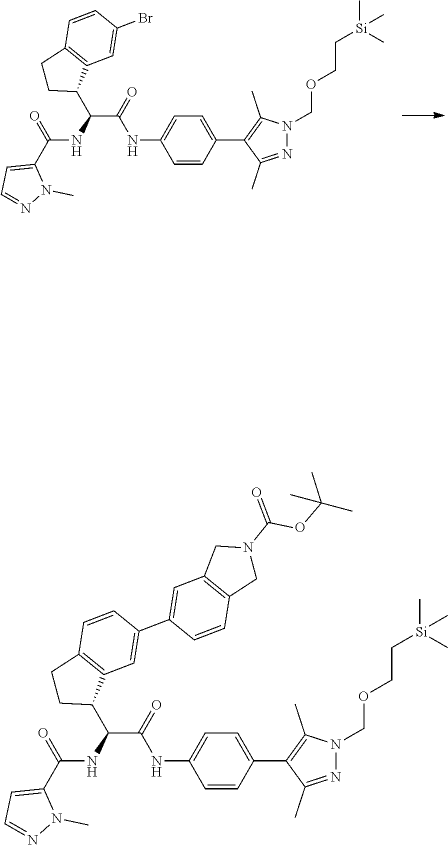 [SMILES: Cc1nn(COCC[Si](C)(C)C)c(C)c1-c1ccc(NC(=O)[C@@H](NC(=O)c2ccnn2C)[C@@H]2CCc3ccc(Br)cc32)cc1 ; Cc1nn(COCC[Si](C)(C)C)c(C)c1-c1ccc(NC(=O)[C@@H](NC(=O)c2ccnn2C)[C@@H]2CCc3ccc(-c4ccc5c(c4)CN(C(=O)OC(C)(C)C)C5)cc32)cc1]

tert-Butyl 5-(4,4,5,5-tetramethyl-1,3,2-dioxaborolan-2-yl)isoindoline-2-carboxylate (78.4 mg, 0.227 mmol) was added to a solution of the compound from Preparation 15 (77.0 mg, 0.114 mmol) in DMF (1.9 mL) in a 5 mL MW vial. A solution of K₂CO₃ (62.8 mg, 0.45 mmol) in H₂O (0.39 mL) was added and the reaction mixture was degassed with argon for 10 min. PdCl₂(dppf).DCM (9.3 mg, 0.011 mmol) was added and the vial was capped and stirred for 1 h at 80° C. The reaction mixture was passed through a PTFE filter and purified by acidic HPLC directly to afford the title compound as an off-white solid. (64 mg, 69% yield); LCMS (METHOD 3) (ES): m/z 816.8 [M+H]⁺, RT=1.05 min.

### Preparation 17

### (2S)-2-(tert-butoxycarbonylamino)-2-[(1R)-6-[4-(morpholinomethyl)phenyl]indan-1-yl]acetic Acid

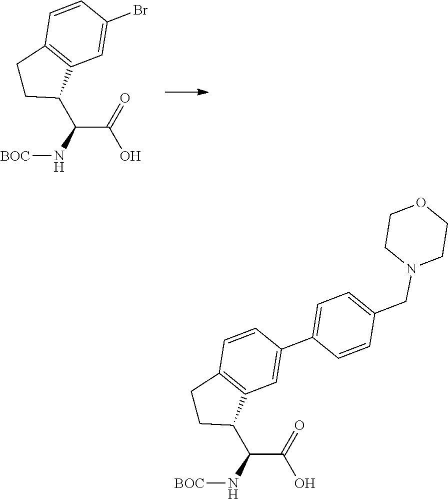 [SMILES: CC(C)(C)OC(=O)N[C@H](C(=O)O)[C@@H]1CCc2ccc(Br)cc21 ; CC(C)(C)OC(=O)N[C@H](C(=O)O)[C@@H]1CCc2ccc(-c3ccc(CN4CCOCC4)cc3)cc21]

K₂CO₃ (31.6 mg, 0.23 mmol) in H₂O (0.10 mL) was added to a solution of the compound from Preparation 13 (28.0 mg, 0.08 mmol) and 4-[[4-(4,4,5,5-tetramethyl-1,3,2-dioxaborolan-2-yl)phenyl]methyl]morpholine hydrochloride (38.5 mg, 0.113 mmol) in DMF (1.0 mL) in a 5 mL MW vial. The reaction mixture was degassed with argon for 10 min. PdCl₂(dppf).DCM (3.10 mg, 0.004 mmol) was added and the reaction mixture was stirred at 80 C for 1 h. The crude material was filtered through a PTFE frit then purified by acidic HPLC to afford title compound as a colourless solid. (21 mg, 60% yield) LCMS (METHOD 3) (ES): m/z 467.5 [M+H]⁺, RT=0.54 min.

### Preparation 18

### tert-butyl N-[(1S)-2-[4-[3,5-dimethyl-1-(2-trimethylsilylethoxymethyl)pyrazol-4-yl]anilino]-1-[(1R)-6-[4-(morpholinomethyl)phenyl]indan-1-yl]-2-oxo-ethyl]carbamate

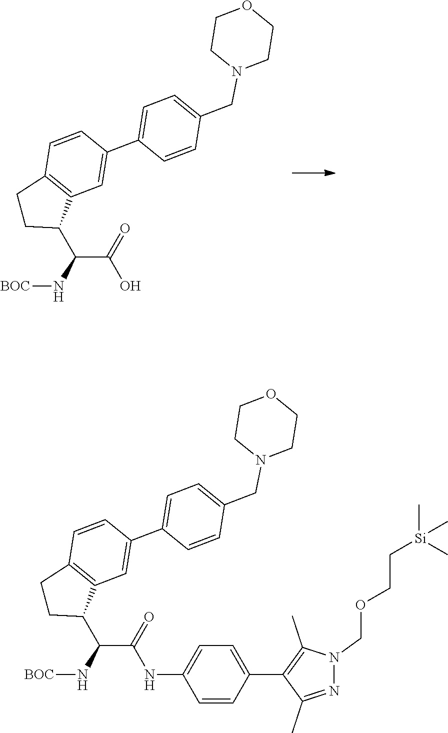 [SMILES: CC(C)(C)OC(=O)N[C@H](C(=O)O)[C@@H]1CCc2ccc(-c3ccc(CN4CCOCC4)cc3)cc21 ; Cc1nn(COCC[Si](C)(C)C)c(C)c1-c1ccc(NC(=O)[C@@H](NC(=O)OC(C)(C)C)[C@@H]2CCc3ccc(-c4ccc(CN5CCOCC5)cc4)cc32)cc1]

DIPEA (29.1 mg, 0.225 mmol) was added to a solution of the compound from Preparation 17 (21.0 mg, 0.045 mmol) and 4-[3,5-dimethyl-1-(2-trimethylsilylethoxymethyl)pyrazol-4-yl]aniline (17.2 mg, 0.054 mmol) in DMF (2 mL) at room temperature. HATU (18.8 mg, 0.05 mmol) was added and the reaction mixture stirred for 1 h. The reaction mixture was purified directly by basic HPLC, to afford title compound as an off-white solid. (33.0 mg, 95% yield) LCMS (METHOD 3) (ES): m/z 766.4 [M+H]⁺, RT=0.91 min.

### Preparation 19

### N-[(1S)-2-[4-[3,5-dimethyl-1-(2-trimethylsilylethoxymethyl)pyrazol-4-yl]anilino]-1-[(1R)-6-[4-(morpholinomethyl)phenyl]indan-1-yl]-2-oxo-ethyl]-2-methyl-pyrazole-3-carboxamide

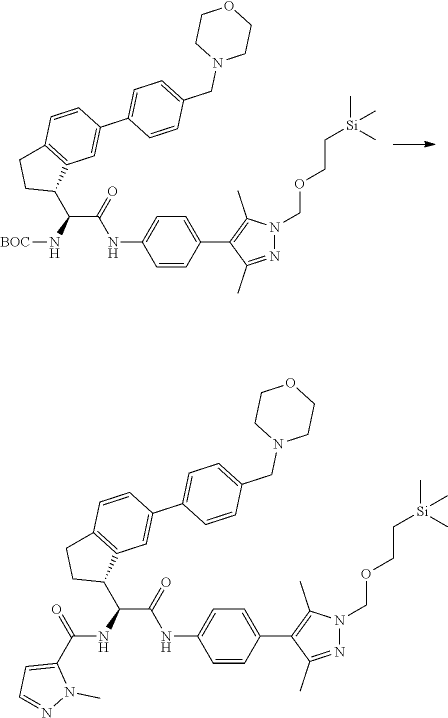 [SMILES: Cc1nn(COCC[Si](C)(C)C)c(C)c1-c1ccc(NC(=O)[C@@H](NC(=O)OC(C)(C)C)[C@@H]2CCc3ccc(-c4ccc(CN5CCOCC5)cc4)cc32)cc1 ; Cc1nn(COCC[Si](C)(C)C)c(C)c1-c1ccc(NC(=O)[C@@H](NC(=O)c2ccnn2C)[C@@H]2CCc3ccc(-c4ccc(CN5CCOCC5)cc4)cc32)cc1]

Hydrogen chloride (4M in dioxan, 5.0 mL) was added to a solution of the compound from Preparation 18 (33 mg, 0.043 mmol) in MeOH (5 mL) at room temperature for 1 h. The reaction mixture was concentrated in vacuo to give crude intermediate (2S)-2-amino-N-[4-[3,5-dimethyl-1-(2-trimethylsilylethoxymethyl)pyrazol-4-yl]phenyl]-2-[(1R)-6-[4-(morpholinomethyl)phenyl]indan-1-yl]acetamide hydrochloride. Assume quantitative yield. LCMS (METHOD 3) (ES): m/z 666.3 [M+H]⁺, RT=0.92 min. DIPEA (0.074 mL, 0.43 mmol) was added to a solution of the crude (2S)-2-amino-N-[4-[3,5-dimethyl-1-(2-trimethylsilylethoxymethyl)pyrazol-4-yl]phenyl]-2-[(1R)-6-[4-(morpholinomethyl)phenyl]indan-1-yl]acetamide hydrochloride (33.6 mg, 0.043 mmol) and 2-methylpyrazole-3-carboxylic acid (6.40 mg, 0.051 mmol) in DMF (2 mL) at room temperature. HATU (19.3 mg, 0.051 mmol) was added and the reaction mixture stirred for 1 h. The reaction mixture was purified directly by basic HPLC, to afford title compound as an off-white solid. (17.3 mg, 52% yield) LCMS (METHOD 3) (ES): m/z 774.4 [M+H]⁺, RT=0.90 min.

### Preparation 20

### tert-butyl N-[(1S)-1-[(1R)-6-bromoindan-1-yl]-2-[4-(3-methylimidazol-4-yl)anilino]-2-oxo-ethyl]carbamate

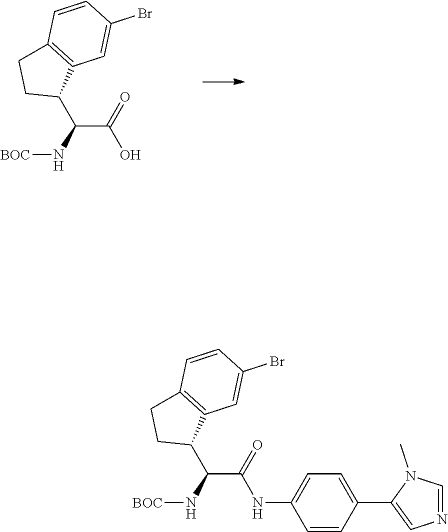 [SMILES: CC(C)(C)OC(=O)N[C@H](C(=O)O)[C@@H]1CCc2ccc(Br)cc21 ; Cn1cncc1-c1ccc(NC(=O)[C@@H](NC(=O)OC(C)(C)C)[C@@H]2CCc3ccc(Br)cc32)cc1]

4-(3-Methylimidazol-4-yl)aniline (48.4 mg, 0.28 mmol) was added to a mixture of the compound from Preparation 13 (86 mg, 0.23 mmol), DIPEA (0.122 mL, 0.699 mmol) and HATU (106.3 mg, 0.28 mmol) in DMF (2 mL) at room temperature. The reaction mixture was stirred for 30 min. The reaction mixture was passed through a PTFE filter and purified by basic HPLC directly to afford the title compound as an off-white solid. (85 mg, 73% yield); LCMS (METHOD 3) (ES): m/z 527.3 [M+H]⁺, RT=0.70 min.

### Preparation 21

### N-[(1S)-1-[(1R)-6-bromoindan-1-yl]-2-[4-(3-methylimidazol-4-yl)anilino]-2-oxo-ethyl]-N,2-dimethyl-pyrazole-3-carboxamide

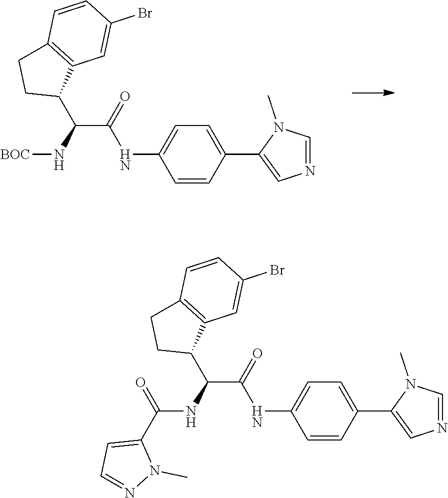 [SMILES: Cn1cncc1-c1ccc(NC(=O)[C@@H](NC(=O)OC(C)(C)C)[C@@H]2CCc3ccc(Br)cc32)cc1 ; Cn1cncc1-c1ccc(NC(=O)[C@@H](NC(=O)c2ccnn2C)[C@@H]2CCc3ccc(Br)cc32)cc1]

Hydrogen chloride (4M in dioxan, 2.0 mL) was added to a solution of the compound from Preparation 20 (85 mg, 0.17 mmol) in MeOH (5 mL) at room temperature for 1 h. The reaction mixture was concentrated in vacuo to give crude intermediate (2S)-2-amino-2-[(1R)-6-bromoindan-1-yl]-N-[4-(3-methylimidazol-4-yl)phenyl]acetamide hydrochloride. Assume quantitative yield. LCMS (METHOD 3) (ES): m/z 427.4 [M+H]⁺, RT=0.38 min. DIPEA (0.09 mL, 0.50 mmol) was added to a solution of the crude (2S)-2-amino-2-[(1R)-6-bromoindan-1-yl]-N-[4-(3-methylimidazol-4-yl)phenyl]acetamide hydrochloride (71.2 mg, 0.17 mmol) and 2-methylpyrazole-3-carboxylic acid (29.6 mg, 0.23 mmol) in DMF (1 mL) at room temperature. HATU (76.4 mg, 0.20 mmol) was added and the reaction mixture stirred for 30 min. The reaction mixture was purified directly by basic HPLC, to afford title compound as an off-white solid. (57.0 mg, 63% yield) LCMS (METHOD 4) (ES): m/z 535.1 [M+H]⁺, RT=0.63 min.

### Preparation 22

### Dimethyl 2-(benzhydrylideneamino)-2-[(1R)-7-bromotetralin-1-yl]propanedioate

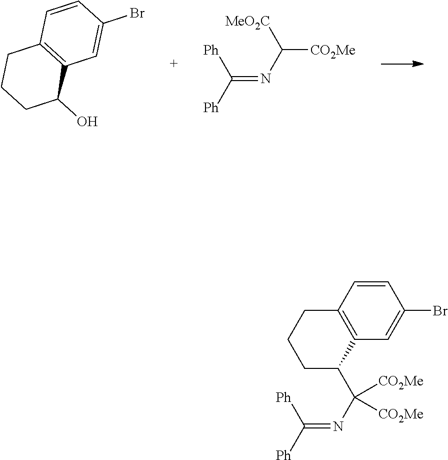

According to the method of Preparation 10 (1S)-7-bromotetralin-1-ol (4.80 g, 21.1 mmol) was reacted to give the title compound (7.90 g, 72%). LCMS (METHOD 4) (ES): m/z 520.3, 522.5 [M+H]⁺, RT=1.05 min.

### Preparation 23

### 2-Amino-2-[(1R)-7-bromotetralin-1-yl]acetic Acid Hydrobromide

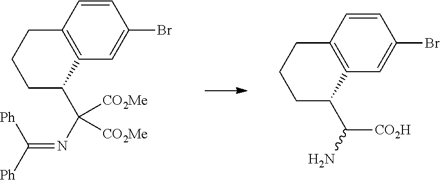 [SMILES: COC(=O)C(N=C(c1ccccc1)c1ccccc1)(C(=O)OC)[C@@H]1CCCc2ccc(Br)cc21 ; NC(C(=O)O)[C@@H]1CCCc2ccc(Br)cc21]

According to the method of Preparation 8 the compound of Preparation 22 (7.9 g, 15 mmol) was reacted to give the title compound (4.9 g, 87%) as a white solid. LCMS (METHOD 3) (ES): m/z 284.1, 286.1 [M+H]⁺, RT=0.40 min.

### Preparation 24

### Nickelous (2S)-2-[(E)-[[2-[(2S)-1-benzylpyrrolidine-2-carbonyl]azanidylphenyl]-phenyl-methylene]amino]-2-[(1R)-7-bromotetralin-1-yl]acetate

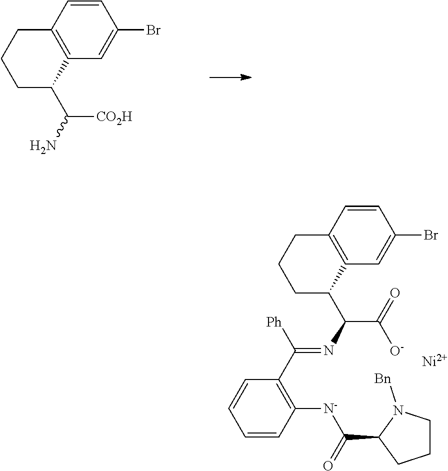

According to the method of Preparation 9 the compound of Preparation 23 (2.0 g, 5.5 mmol) was reacted to give the title compound (2.0 g, 52%) as a red solid. 1H NMR (300 MHz, Chloroform-d) δ 8.39 (dd, J=8.7, 1.1 Hz, 1H), 8.10-7.98 (m, 2H), 7.53-7.43 (m, 1H), 7.38 (tt, J=7.6, 1.3 Hz, 1H), 7.33-7.06 (m, 6H), 7.06-7.01 (m, 1H), 6.98-6.89 (m, 1H), 6.84 (d, J=8.2 Hz, 1H), 6.61 (ddd, J=8.1, 6.8, 1.2 Hz, 1H), 6.53 (dd, J=8.2, 1.8 Hz, 1H), 6.24 (d, J=7.6 Hz, 1H), 4.45 (d, J=2.6 Hz, 1H), 4.40 (d, J=12.6 Hz, 1H), 3.59-3.39 (m, 4H), 3.29-3.04 (m, 2H), 2.97-2.83 (m, 1H), 2.70-2.48 (m, 3H), 2.40-2.24 (m, 1H), 2.16-1.88 (m, 3H), 1.84-1.67 (m, 1H); LCMS (METHOD 3) (ES): m/z 706.3, 708.3 [M+H]⁺, RT=0.93 min.

### Preparation 25

### (2S)-2-[(1R)-7-bromotetralin-1-yl]-2-(tert-butoxycarbonylamino)acetic Acid

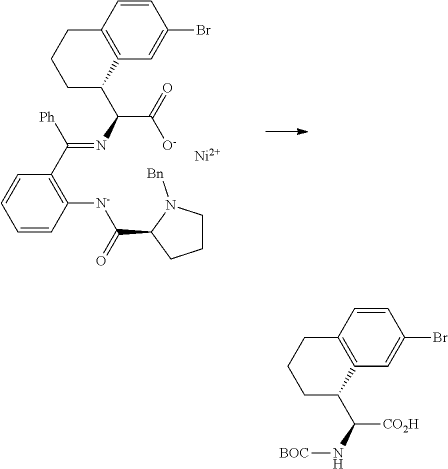

According to the method of Preparation 13 the compound of Preparation 24 (2.0 g, 2.83 mmol) was reacted to give the title compound (620 mg, 57%) as a white solid. ¹H NMR (300 MHz, DMSO-d6) δ 12.7 (br s, 1H), 7.70-7.17 (m, 2H), 7.15-6.81 (m, 2H), 4.63 (br s, 1H), 3.29 (br s, 1H), 2.82-2.55 (m, 2H), 2.01-1.68 (m, 2H), 1.67-1.45 (m, 2H), 1.33 (s, 9H); LCMS (METHOD 3) (ES): m/z 384.4, 386.4 [M+H]⁺, RT=0.78 min.

### Preparation 26

### (2S)-2-(tert-butoxycarbonylamino)-2-[(1R)-7-(1-cyclopropylpyrazol-4-yl)tetralin-1-yl]acetic Acid

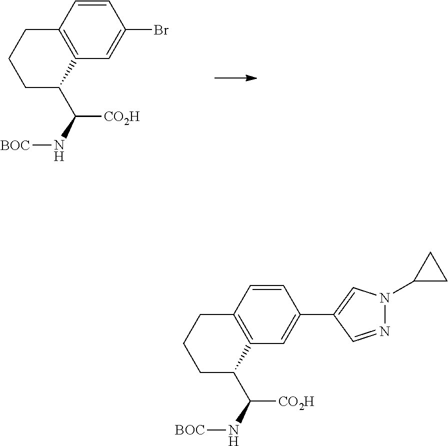 [SMILES: CC(C)(C)OC(=O)N[C@H](C(=O)O)[C@@H]1CCCc2ccc(Br)cc21 ; CC(C)(C)OC(=O)N[C@H](C(=O)O)[C@@H]1CCCc2ccc(-c3cnn(C4CC4)c3)cc21]

According to the method of Preparation 17 the compound of Preparation 25 (23.0 mg, 0.06 mmol) was reacted to give the title compound after basic HPLC as a white solid (13.8 mg, 56%). LCMS (METHOD 3) (ES): m/z 412.4 [M+H]⁺, RT=0.49 min.

### Preparation 27

### tert-butyl N-[(1S)-1-[(1R)-7-(1-cyclopropylpyrazol-4-yl)tetralin-1-yl]-2-[4-[3,5-dimethyl-1-(2-trimethylsilylethoxymethyl)pyrazol-4-yl]anilino]-2-oxo-ethyl]carbamate

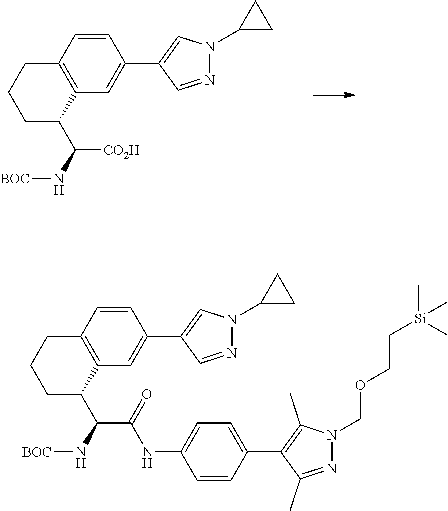 [SMILES: CC(C)(C)OC(=O)N[C@H](C(=O)O)[C@@H]1CCCc2ccc(-c3cnn(C4CC4)c3)cc21 ; Cc1nn(COCC[Si](C)(C)C)c(C)c1-c1ccc(NC(=O)[C@@H](NC(=O)OC(C)(C)C)[C@@H]2CCCc3ccc(-c4cnn(C5CC5)c4)cc32)cc1]

HATU (14.0 mg, 0.037) was added to a solution of the compound from Preparation 26 (13.8 mg, 0.034 mmol) and 4-[3,5-dimethyl-1-(2-trimethylsilylethoxymethyl)pyrazol-4-yl]aniline (11.7 mg, 0.037 mmol) in DMF (1 mL) at 0° C. DIPEA (0.029 mL, 0.168 mmol) was added and the reaction mixture was stirred to, then stirred at room temperature for 30 min. The reaction mixture was diluted with H₂O and extracted with EtOAc (2×10 mL). The combined organic phases were successively washed with H₂O (500 mL) and brine (500 mL) then dried over MgSO₄, filtered and concentrated in vacuo. The obtained crude compound was purified by silica column chromatography (230-400 mesh), eluting with 0-100% EtOAc in heptane, to afford title compound as an off-white solid. (11.3 mg, 47% yield); LCMS (METHOD 3) (ES): m/z 711.8 [M+H]⁺, RT=1.01 min.

### Preparation 28

### (2S)-2-amino-2-[(1R)-7-(1-cyclopropylpyrazol-4-yl)tetralin-1-yl]-N-[4-[3,5-dimethyl-1-(2-trimethylsilylethoxymethyl)pyrazol-4-yl]phenyl]acetamide Hydrochloride

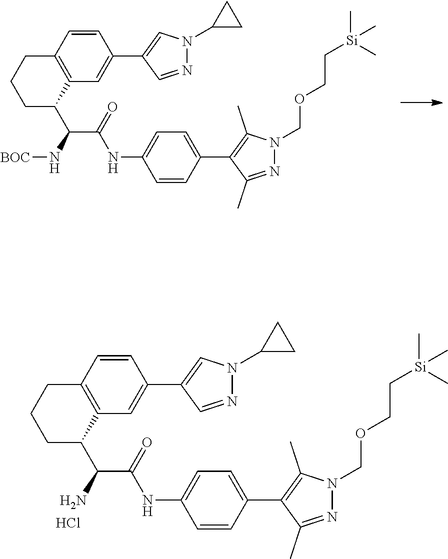 [SMILES: Cc1nn(COCC[Si](C)(C)C)c(C)c1-c1ccc(NC(=O)[C@@H](NC(=O)OC(C)(C)C)[C@@H]2CCCc3ccc(-c4cnn(C5CC5)c4)cc32)cc1 ; Cc1nn(COCC[Si](C)(C)C)c(C)c1-c1ccc(NC(=O)[C@@H](N)[C@@H]2CCCc3ccc(-c4cnn(C5CC5)c4)cc32)cc1 ; Cl]

Hydrogen chloride (4M in dioxan, 2.0 mL) was added to a solution of the compound from Preparation 27 (11.3 mg, 0.016 mmol) in MeOH (2 mL) at room temperature for 1 h. The reaction mixture was concentrated in vacuo to afford crude title compound as a colourless solid. Assume quantitative yield. LCMS (METHOD 3) (ES): m/z 611.6 [M+H]⁺, RT=0.79 min.

### Preparation 29

### N-[(1S)-1-[(1R)-7-(1-cyclopropylpyrazol-4-yl)tetralin-1-yl]-2-[4-[3,5-dimethyl-1-(2-trimethylsilylethoxymethyl)pyrazol-4-yl]anilino]-2-oxo-ethyl]-2-methyl-pyrazole-3-carboxamide

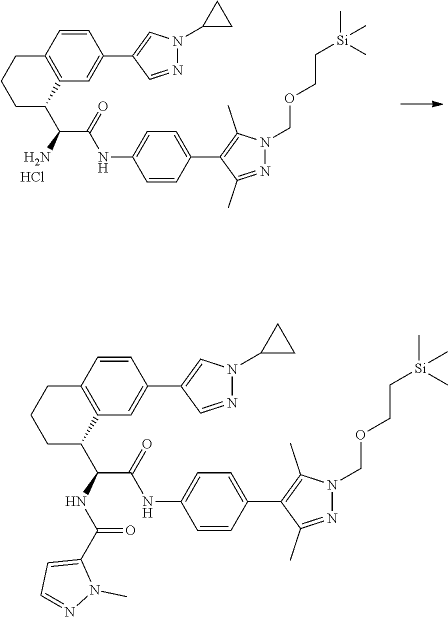 [SMILES: Cc1nn(COCC[Si](C)(C)C)c(C)c1-c1ccc(NC(=O)[C@@H](N)[C@@H]2CCCc3ccc(-c4cnn(C5CC5)c4)cc32)cc1 ; Cl ; Cc1nn(COCC[Si](C)(C)C)c(C)c1-c1ccc(NC(=O)[C@@H](NC(=O)c2ccnn2C)[C@@H]2CCCc3ccc(-c4cnn(C5CC5)c4)cc32)cc1]

HATU (7.2 mg, 0.019 mmol) was added to a solution of the compound from Preparation 28 (9.7 mg, 0.016 mmol) and 2-methylpyrazole-3-carboxylic acid (2.4 mg, 0.019 mmol) in DMF (1 mL) at room temperature. DIPEA (0.011 mL, 0.064 mmol) was added and the reaction mixture was stirred for 1 h. The reaction mixture was purified directly by acidic HPLC to afford title compound as an off-white solid. (11.0 mg, 93% yield); LCMS (METHOD 3) (ES): m/z 719.8 [M+H]⁺, RT=0.93 min.

### Preparation 30

### (2S)-2-(tert-butoxycarbonylamino)-2-[(1R)-7-(1-isopropyl-6-oxo-3-pyridyl)tetralin-1-yl]acetic Acid

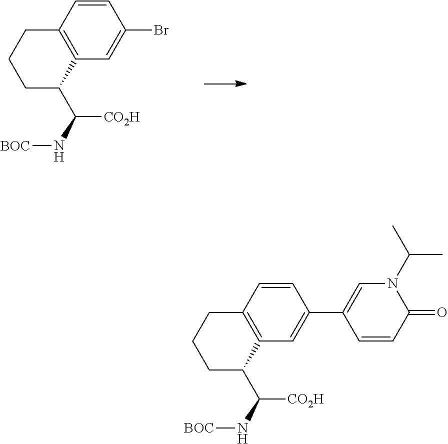 [SMILES: CC(C)(C)OC(=O)N[C@H](C(=O)O)[C@@H]1CCCc2ccc(Br)cc21 ; CC(C)n1cc(-c2ccc3c(c2)[C@H]([C@H](NC(=O)OC(C)(C)C)C(=O)O)CCC3)ccc1=O]

According to the method of Preparation 17 the compound of Preparation 25 (36.0 mg, 0.094 mmol) was reacted to give the title compound after acidic HPLC as a colourless solid (35 mg, 85% yield). LCMS (METHOD 3) (ES): m/z 441.5 [M+H]⁺, RT=0.70 min.

### Preparation 31

### tert-butyl N-[(1S)-2-[4-[3,5-dimethyl-1-(2-trimethylsilylethoxymethyl)pyrazol-4-yl]anilino]-1-[(1R)-7-(1-isopropyl-6-oxo-3-pyridyl)tetralin-1-yl]-2-oxo-ethyl]carbamate

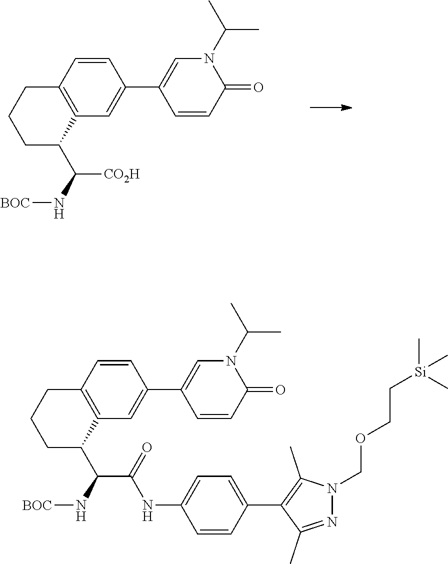 [SMILES: CC(C)n1cc(-c2ccc3c(c2)[C@H]([C@H](NC(=O)OC(C)(C)C)C(=O)O)CCC3)ccc1=O ; Cc1nn(COCC[Si](C)(C)C)c(C)c1-c1ccc(NC(=O)[C@@H](NC(=O)OC(C)(C)C)[C@@H]2CCCc3ccc(-c4ccc(=O)n(C(C)C)c4)cc32)cc1]

4-[3,5-dimethyl-1-(2-trimethylsilylethoxymethyl)pyrazol-4-yl]aniline (32.7 mg, 0.103 mmol) in DMF (0.5 mL) was added to a solution of the compound from Preparation 30 (35.0 mg, 0.079 mmol), HATU (39.2 mg, 0.103 mmol) and DIPEA (0.033 mL, 0.187 mmol) in DMF (1 mL) at room temperature. The reaction mixture was stirred for 40 min. The reaction mixture was purified directly by acidic HPLC to afford title compound as an off-white solid. (35.0 mg, 50% yield); LCMS (METHOD 3) (ES): m/z 740.7 [M+H]⁺, RT=0.99 min.

### Preparation 32

### (2S)-2-amino-N-[4-[3,5-dimethyl-1-(2-trimethylsilylethoxymethyl)pyrazol-4-yl]phenyl]-2-[(1R)-7-(1-isopropyl-6-oxo-3-pyridyl)tetralin-1-yl]acetamide Hydrochloride

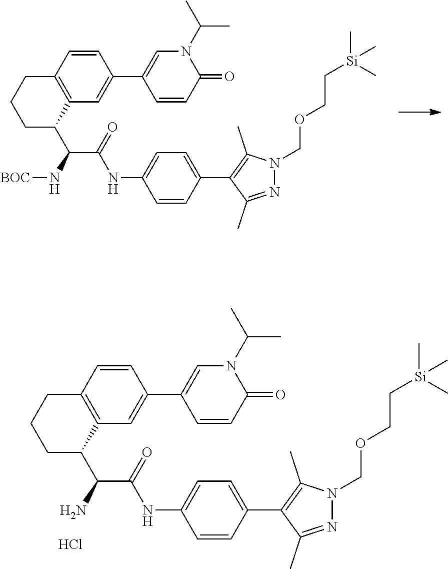 [SMILES: Cc1nn(COCC[Si](C)(C)C)c(C)c1-c1ccc(NC(=O)[C@@H](NC(=O)OC(C)(C)C)[C@@H]2CCCc3ccc(-c4ccc(=O)n(C(C)C)c4)cc32)cc1 ; Cc1nn(COCC[Si](C)(C)C)c(C)c1-c1ccc(NC(=O)[C@@H](N)[C@@H]2CCCc3ccc(-c4ccc(=O)n(C(C)C)c4)cc32)cc1 ; Cl]

According to the method of Preparation 28 the compound of Preparation 31 (35.0 mg, 0.047 mmol) was reacted to afford the title compound as a colourless solid (32 mg, 100% yield). LCMS (METHOD 3) (ES): m/z 640.7 [M+H]⁺, RT=0.75 min.

### Preparation 33

### N-[(1S)-2-[4-[3,5-dimethyl-1-(2-trimethylsilylethoxymethyl)pyrazol-4-yl]anilino]-1-[(1R)-7-(1-isopropyl-6-oxo-3-pyridyl)tetralin-1-yl]-2-oxo-ethyl]-1-fluoro-cyclopropanecarboxamide

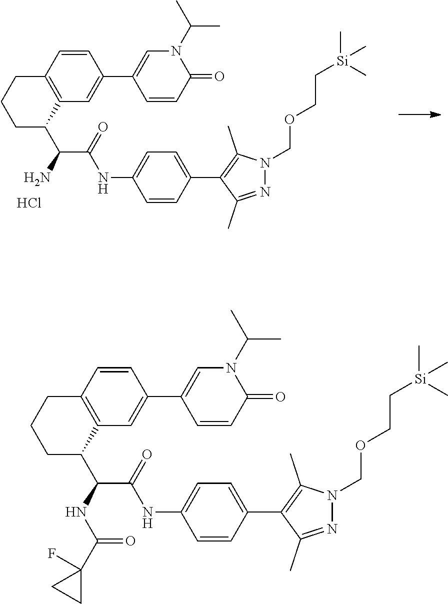 [SMILES: Cc1nn(COCC[Si](C)(C)C)c(C)c1-c1ccc(NC(=O)[C@@H](N)[C@@H]2CCCc3ccc(-c4ccc(=O)n(C(C)C)c4)cc32)cc1 ; Cl ; Cc1nn(COCC[Si](C)(C)C)c(C)c1-c1ccc(NC(=O)[C@@H](NC(=O)C2(F)CC2)[C@@H]2CCCc3ccc(-c4ccc(=O)n(C(C)C)c4)cc32)cc1]

According to the method of Preparation 29 the compound of Preparation 32 (32.0 mg, 0.047 mmol) was reacted to give the title compound after basic HPLC as a colourless solid (24 mg, 70% yield). LCMS (METHOD 3) (ES): m/z 726.7 [M+H]⁺, RT=0.95 min.

### Preparation 34

### (1R,4R)-5-(4-bromo-2-pyridyl)-2-oxa-5-azabicyclo[2.2.1]heptane

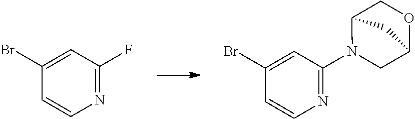 [SMILES: Fc1cc(Br)ccn1 ; Brc1ccnc(N2C[C@H]3C[C@@H]2CO3)c1]

DIPEA (0.38 mL, 2.22 mmol) was added to a solution of 4-bromo-2-fluoro-pyridine (130 mg, 0.74 mmol) and (1R,4R)-2-oxa-5-azabicyclo[2.2.1]heptane hydrochloride (120 mg, 0.89 mmol) in NMP (1 mL). The reaction was stirred at 100° C. for 30 min. The cooled reaction mixture was diluted with H₂O (5 mL) and extracted with Et₂O (3×10 mL). The combined organic phases then dried over MgSO₄, filtered and concentrated in vacuo. The obtained crude compound was purified by silica column chromatography (230-400 mesh), eluting with 0-80% EtOAc in heptane, to afford title compound as a colourless oil. (70 mg, 37% yield); ¹H NMR (300 MHz, Chloroform-d) δ 7.94 (dd, J=5.4, 0.5 Hz, 1H), 6.71 (dd, J=5.4, 1.6 Hz, 1H), 6.51 (dd, J=1.6, 0.5 Hz, 1H), 4.88 (d, J=1.7 Hz, 1H), 4.69 (dt, J=2.0, 1.0 Hz, 1H), 3.96-3.79 (m, 2H), 3.47 (dd, J=9.6, 1.6 Hz, 1H), 3.33 (dd, J=9.7, 1.0 Hz, 1H), 1.95 (dt, J=2.0, 1.3 Hz, 2H).

### Preparation 35

### [2-[(1R,4R)-2-oxa-5-azabicyclo[2.2.1]heptan-5-yl]-4-pyridyl]boronic Acid

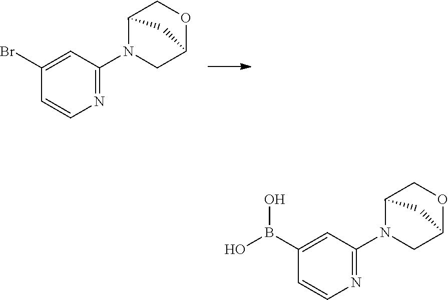 [SMILES: Brc1ccnc(N2C[C@H]3C[C@@H]2CO3)c1 ; OB(O)c1ccnc(N2C[C@H]3C[C@@H]2CO3)c1]

KOAc (127.0 mg, 1.30 mmol) was added to a solution of the compound from Preparation 34 (165 mg, 0.65 mmol) and 4,4,5,5-tetramethyl-2-(4,4,5,5-tetramethyl-1,3,2-dioxaborolan-2-yl)-1,3,2-dioxaborolane (197 mg, 0.77 mmol) in DMF (2 mL). The reaction mixture was purged with argon for 10 min. PdCl2(dppf).DCM (52.8 mg, 0.064 mmol) was added and the reaction mixture was heated at 80° C. for 1 h. The reaction mixture was cooled, passed through a PTFE filter then purified directly by acidic HPLC to afford title compound as an off-white solid. (136 mg, 95% yield); LCMS (METHOD 3) (ES): m/z 221.1 [M+H]⁺, RT=0.21 min.

### Preparation 36

### (2S)-2-(tert-butoxycarbonylamino)-2-[(1R)-7-[2-[(1R,4R)-2-oxa-5-azabicyclo[2.2.1]heptan-5-yl]-4-pyridyl]tetralin-1-yl]acetic Acid

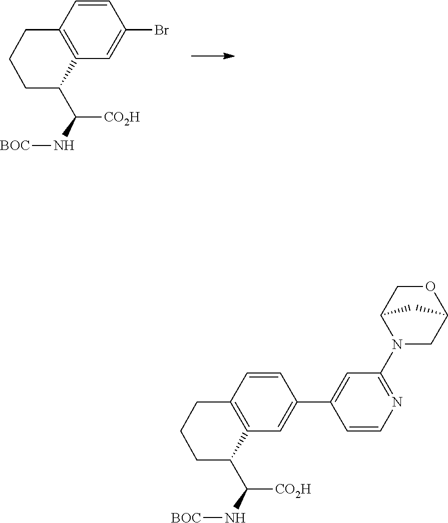 [SMILES: CC(C)(C)OC(=O)N[C@H](C(=O)O)[C@@H]1CCCc2ccc(Br)cc21 ; CC(C)(C)OC(=O)N[C@H](C(=O)O)[C@@H]1CCCc2ccc(-c3ccnc(N4C[C@H]5C[C@@H]4CO5)c3)cc21]

According to the method of Preparation 17 the compound of Preparation 35 (42.9 mg, 0.195 mmol) was reacted to give the title compound after acidic HPLC as a colourless solid (49 mg, 78% yield). LCMS (METHOD 3) (ES): m/z 480.5 [M+H]⁺, RT=0.58 min.

### Preparation 37

### tert-butyl N-[(1S)-2-[4-[3,5-dimethyl-1-(2-trimethylsilylethoxymethyl)pyrazol-4-yl]anilino]-1-[(1R)-7-[2-[(1R,4R)-2-oxa-5-azabicyclo[2.2.1]heptan-5-yl]-4-pyridyl]tetralin-1-yl]-2-oxo-ethyl]carbamate

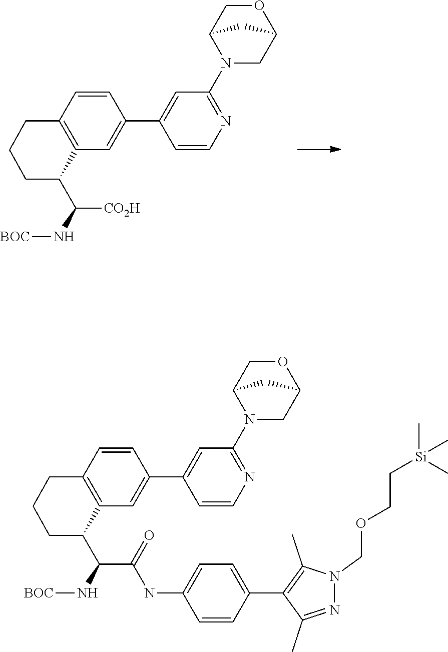 [SMILES: CC(C)(C)OC(=O)N[C@H](C(=O)O)[C@@H]1CCCc2ccc(-c3ccnc(N4C[C@H]5C[C@@H]4CO5)c3)cc21 ; Cc1nn(COCC[Si](C)(C)C)c(C)c1-c1ccc(NC(=O)[C@@H](NC(=O)OC(C)(C)C)[C@@H]2CCCc3ccc(-c4ccnc(N5C[C@H]6C[C@@H]5CO6)c4)cc32)cc1]

According to the method of Preparation 18 the compound of Preparation 36 (42.9 mg, 0.195 mmol) was reacted to give the title compound after acidic HPLC as a colourless solid (21 mg, 54% yield). LCMS (METHOD 3) (ES): m/z 779.6 [M+H]⁺, RT=0.97 min.

### Preparation 38

### (2S)-2-amino-N-[4-[3,5-dimethyl-1-(2-trimethylsilylethoxymethyl)pyrazol-4-yl]phenyl]-2-[(1R)-7-[2-[(1R,4R)-2-oxa-5-azabicyclo[2.2.1]heptan-5-yl]-4-pyridyl]tetralin-1-yl]acetamide

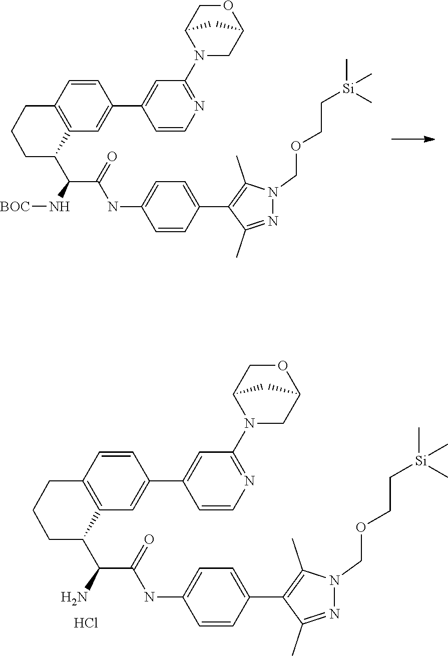 [SMILES: Cc1nn(COCC[Si](C)(C)C)c(C)c1-c1ccc(NC(=O)[C@@H](NC(=O)OC(C)(C)C)[C@@H]2CCCc3ccc(-c4ccnc(N5C[C@H]6C[C@@H]5CO6)c4)cc32)cc1 ; Cc1nn(COCC[Si](C)(C)C)c(C)c1-c1ccc(NC(=O)[C@@H](N)[C@@H]2CCCc3ccc(-c4ccnc(N5C[C@H]6C[C@@H]5CO6)c4)cc32)cc1 ; Cl]

According to the method of Preparation 28, the compound of Preparation 37 (21 mg, 0.027 mmol) was reacted to give the title compound after acidic HPLC as a colourless solid (19.3 mg, assume 100% yield). LCMS (METHOD 3) (ES): m/z 679.5 [M+H]⁺, RT=0.65 min.

### Preparation 39

### N-[(1S)-2-[4-[3,5-dimethyl-1-(2-trimethylsilylethoxymethyl)pyrazol-4-yl]anilino]-1-[(1R)-7-[2-[(1R,4R)-2-oxa-5-azabicyclo[2.2.1]heptan-5-yl]-4-pyridyl]tetralin-1-yl]-2-oxo-ethyl]-1-fluoro-cyclopropanecarboxamide

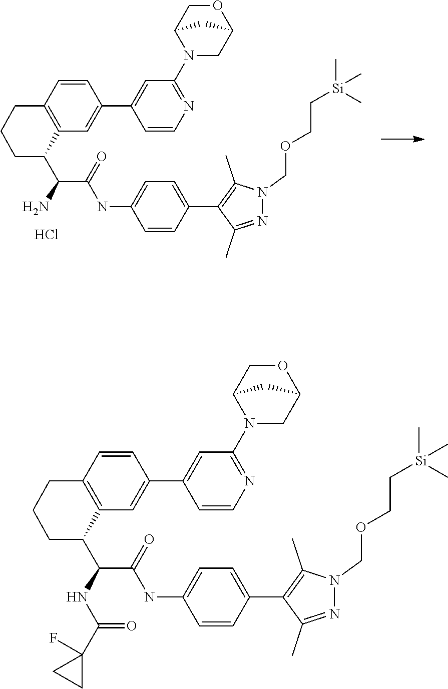 [SMILES: Cc1nn(COCC[Si](C)(C)C)c(C)c1-c1ccc(NC(=O)[C@@H](N)[C@@H]2CCCc3ccc(-c4ccnc(N5C[C@H]6C[C@@H]5CO6)c4)cc32)cc1 ; Cl ; Cc1nn(COCC[Si](C)(C)C)c(C)c1-c1ccc(NC(=O)[C@@H](NC(=O)C2(F)CC2)[C@@H]2CCCc3ccc(-c4ccnc(N5C[C@H]6C[C@@H]5CO6)c4)cc32)cc1]

According to the method of Preparation 29 the compound of Preparation 38 (19.3 mg, 0.027 mmol) was reacted to give the title compound after basic HPLC as a colourless solid (8.0 mg, 39% yield). LCMS (METHOD 3) (ES): m/z 766.9 [M+H]⁺, RT=0.89 min.

### Preparation 40

### tert-butyl N-[(1S)-1-[(1R)-7-bromotetralin-1-yl]-2-[4-[3,5-dimethyl-1-(2-trimethylsilylethoxymethyl)pyrazol-4-yl]anilino]-2-oxo-ethyl]carbamate

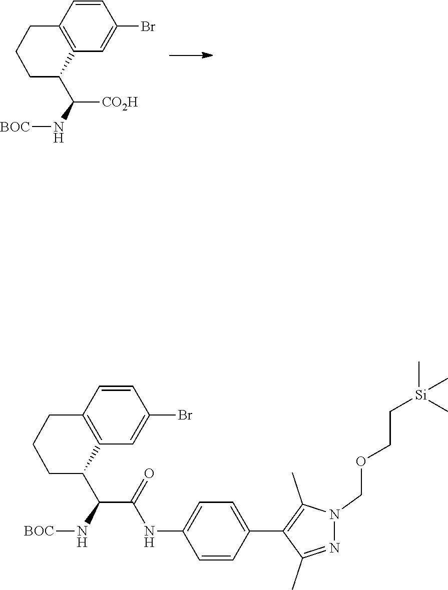 [SMILES: CC(C)(C)OC(=O)N[C@H](C(=O)O)[C@@H]1CCCc2ccc(Br)cc21 ; Cc1nn(COCC[Si](C)(C)C)c(C)c1-c1ccc(NC(=O)[C@@H](NC(=O)OC(C)(C)C)[C@@H]2CCCc3ccc(Br)cc32)cc1]

According to the method of Preparation 14 the compound of Preparation 25 (80.0 mg, 0.21 mmol) was reacted to afford the title compound as a colourless solid (129 mg, 90% yield). LCMS (METHOD 3) (ES): m/z 567.2 [M+H]⁺, RT=0.97 min.

### Preparation 41

### (2S)-2-amino-2-[(1R)-7-bromotetralin-1-yl]-N-[4-[3,5-dimethyl-1-(2-trimethylsilylethoxymethyl)pyrazol-4-yl]phenyl]acetamide Hydrochloride

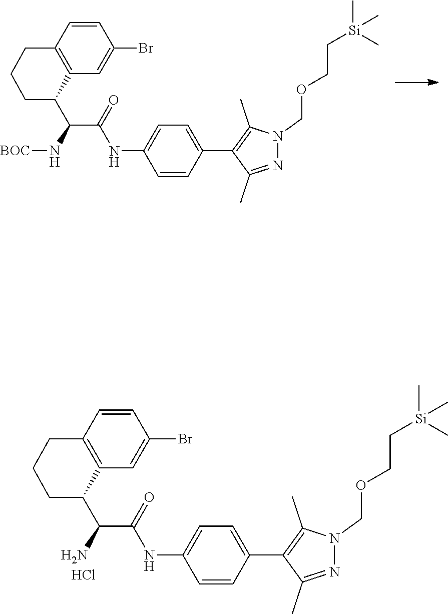 [SMILES: Cc1nn(COCC[Si](C)(C)C)c(C)c1-c1ccc(NC(=O)[C@@H](NC(=O)OC(C)(C)C)[C@@H]2CCCc3ccc(Br)cc32)cc1 ; Cc1nn(COCC[Si](C)(C)C)c(C)c1-c1ccc(NC(=O)[C@@H](N)[C@@H]2CCCc3ccc(Br)cc32)cc1 ; Cl]

According to the method of Preparation 28 the compound of Preparation 40 (129 mg, 0.19 mmol) was reacted to afford the title compound as a colourless solid (117 mg, 100% yield). LCMS (METHOD 3) (ES): m/z 467.1 [M+H]⁺, RT=0.93 min.

### Preparation 42

### N-[(1S)-1-[(1R)-7-bromotetralin-1-yl]-2-[4-[3,5-dimethyl-1-(2-trimethylsilylethoxymethyl)pyrazol-4-yl]anilino]-2-oxo-ethyl]-1-fluoro-cyclopropanecarboxamide

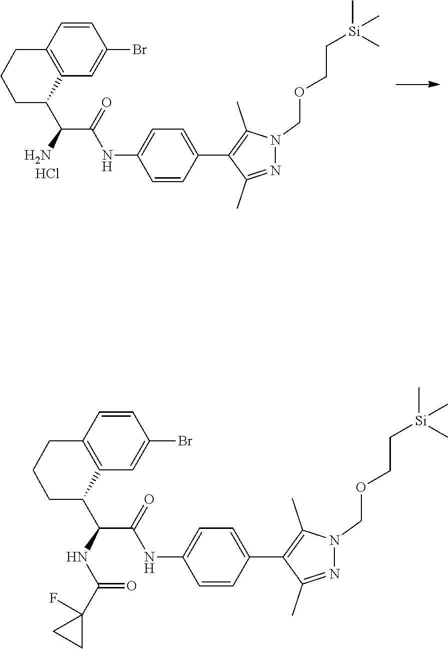 [SMILES: Cc1nn(COCC[Si](C)(C)C)c(C)c1-c1ccc(NC(=O)[C@@H](N)[C@@H]2CCCc3ccc(Br)cc32)cc1 ; Cl ; Cc1nn(COCC[Si](C)(C)C)c(C)c1-c1ccc(NC(=O)[C@@H](NC(=O)C2(F)CC2)[C@@H]2CCCc3ccc(Br)cc32)cc1]

According to the method of Preparation 29 the compound of Preparation 41 (21 mg, 0.027 mmol) was reacted to give the title compound after basic HPLC as a colourless solid (113 mg, 89% yield). LCMS (METHOD 3) (ES): m/z 553.1 [M+H]⁺, RT=0.94 min.

### Preparation 43

### (1S,4S)-5-(4-bromo-2-pyridyl)-2-oxa-5-azabicyclo[2.2.1]heptane

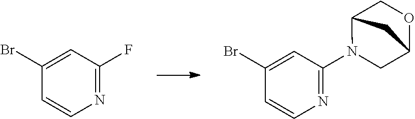 [SMILES: Fc1cc(Br)ccn1 ; Brc1ccnc(N2C[C@@H]3C[C@H]2CO3)c1]

According to the method of Preparation 34, using (1S,4S)-2-oxa-5-azabicyclo[2.2.1]heptane hydrochloride (500 mg, 2.84 mmol) was reacted to give the title compound as a colourless oil (270 mg, 37% yield). ¹H NMR (300 MHz, Chloroform-d) δ 7.93 (dd, J=5.4, 0.5 Hz, 1H), 6.71 (dd, J=5.4, 1.6 Hz, 1H), 6.51 (dd, J=1.6, 0.5 Hz, 1H), 4.88 (p, J=1.4 Hz, 1H), 4.69 (dt, J=2.0, 1.0 Hz, 1H), 3.93-3.81 (m, 2H), 3.46 (dd, J=9.6, 1.5 Hz, 1H), 3.33 (dd, J=9.6, 1.0 Hz, 1H), 2.01-1.89 (m, 2H).

### Preparation 44

### [2-[(1S,4S)-2-oxa-5-azabicyclo[2.2.1]heptan-5-yl]-4-pyridyl]boronic Acid

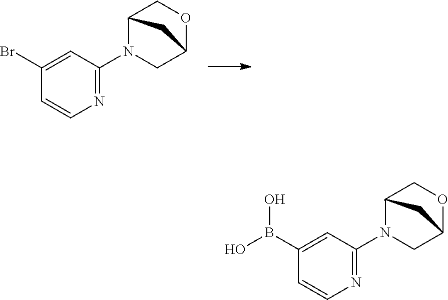 [SMILES: Brc1ccnc(N2C[C@@H]3C[C@H]2CO3)c1 ; OB(O)c1ccnc(N2C[C@@H]3C[C@H]2CO3)c1]

According to the method of Preparation 35 the compound of Preparation 43 (270 mg, 1.06 mmol) was reacted to give the title compound after acidic HPLC as an off-white solid (232 mg, assume quantitative yield). LCMS (METHOD 3) (ES): m/z 221.4 [M+H]⁺, RT=0.22 min.

### Preparation 45

### N-[(1S)-2-[4-[3,5-dimethyl-1-(2-trimethylsilylethoxymethyl)pyrazol-4-yl]anilino]-1-[(1R)-7-[2-[(1S,4S)-2-oxa-5-azabicyclo[2.2.1]heptan-5-yl]-4-pyridyl]tetralin-1-yl]-2-oxo-ethyl]-1-fluoro-cyclopropanecarboxamide

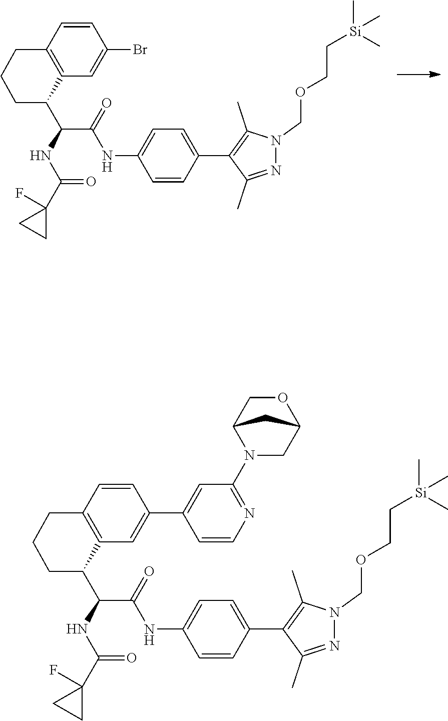 [SMILES: Cc1nn(COCC[Si](C)(C)C)c(C)c1-c1ccc(NC(=O)[C@@H](NC(=O)C2(F)CC2)[C@@H]2CCCc3ccc(Br)cc32)cc1 ; Cc1nn(COCC[Si](C)(C)C)c(C)c1-c1ccc(NC(=O)[C@@H](NC(=O)C2(F)CC2)[C@@H]2CCCc3ccc(-c4ccnc(N5C[C@@H]6C[C@H]5CO6)c4)cc32)cc1]

According to the method of Preparation 17 the compound of Preparation 42 (49 mg, 0.073 mmol) was reacted to give the title compound after basic HPLC as a colourless solid (30 mg, 53% yield). LCMS (METHOD 3) (ES): m/z 765.3 [M+H]⁺, RT=0.89 min.

### Preparation 46

### 4-bromo-2-(1-bromoethyl)-1-chloro-benzene

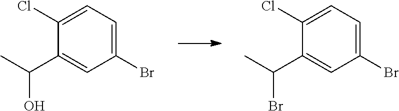

Triphenylphosphane (3.51 g, 13.4 mmol) was dissolved in DCM (20 mL) and added slowly to a solution of 1-(5-bromo-2-chlorophenyl)ethanol (3.00 g, 12.7 mmol) and carbon tetrabromide (4.44 g, 13.4 mmol) in DCM (30 mL) at 0° C. The reaction mixture was stirred at room temperature o/n. The reaction mixture was concentrated in vacuo and slurried in MTBE/heptane (1:1, 100 mL). The precipitated solid was removed via filtration and the filtrate was concentrated in vacuo. The obtained crude compound was purified by silica column chromatography (230-400 mesh), eluting with 0-50% EtOAc in heptane, to afford title compound. (2.93 g, 77% yield); ¹H NMR (300 MHz, Chloroform-d) δ 7.75 (d, J=2.4 Hz, 1H), 7.34 (dd, J=8.5, 2.3 Hz, 1H), 7.22 (d, J=8.5 Hz, 1H), 5.53 (q, J=7.0 Hz, 1H), 2.02 (d, J=6.9 Hz, 3H).

### Preparation 47

### Nickelous 2-[(E)-[[2-[(2S)-1-benzylpyrrolidine-2-carbonyl]azanidylphenyl]-phenyl-methylene]amino]acetate

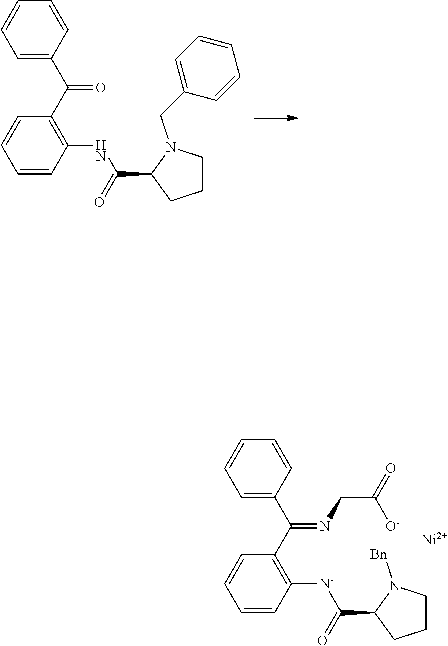

A solution of KOH (13.7 g, 244 mmol) in MeOH (90 mL) was added to a suspension of (2S)—N-(2-benzoylphenyl)-1-benzyl-pyrrolidine-2-carboxamide (13.4 g, 34.9 mmol), 2-aminoacetic acid (13.0 g, 173 mmol) and nickelous nitrate hexahydrate (21.0 g, 72.2 mmol) in MeOH (150 mL) at 40° C. The reaction mixture was stirred at 70° C. for 15 min before NaH (60%, 1.0 g, 25 mmol) was added portionwise. On complete addition the reaction mixture was stirred for 30 min. The reaction mixture was concentrated to low volume and diluted with acetic acid (16 mL) followed by H₂O (200 mL). The partitioned solid was collected by filtration then partitioned between H₂O (100 mL) and DCM (200 mL). The aqueous phase was rewashed with DCM (200 mL) and the combined organic phases were dried over MgSO₄, filtered and concentrated in vacuo to afford title compound as a red solid. (17.3 g, 99% yield); LCMS (METHOD 3) (ES): m/z 498.3 [M+H]⁺, RT=0.69 min.

### Preparation 48

### Nickelous (2S)-2-[(E)-[[2-[(2S)-1-benzylpyrrolidine-2-carbonyl]azanidylphenyl]-phenyl-methylene]amino]-3-(5-bromo-2-chloro-phenyl)butanoate

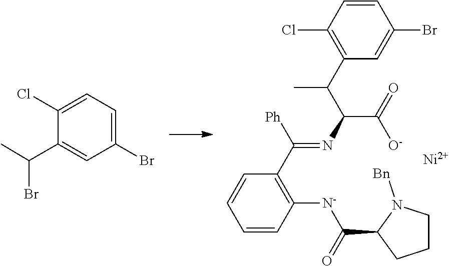

Powdered NaOH (4.57 g, 114 mmol) was added to a solution of the product from Preparation 47 (5.70 g, 11.4 mmol) in DMF (20 mL) at 0° C. and stirred for 5 min. A solution of the product from Preparation 46 (5.12 g, 17.1 mmol) in DMF (20 mL) was added at 0° C., and the reaction mixture was stirred for 30 min. The reaction mixture was quenched with aq. citric acid (0.5 M, 100 mL). The precipitated solid was collected by filtration. The obtained crude compound was purified by silica column chromatography (230-400 mesh), eluting with 20-100% EtOAc in heptane, to afford 2 distinct products. Title compound Diastereomer 1 (1.84 g, 22% yield); ¹H NMR (300 MHz, Chloroform-d) δ 8.41 (dd, J=8.7, 1.0 Hz, 1H), 8.02-7.89 (m, 2H), 7.66 (dd, J=1.9, 0.7 Hz, 1H), 7.64-7.45 (m, 5H), 7.33-7.20 (m, 4H), 7.20-7.03 (m, 2H), 6.76-6.58 (m, 2H), 4.26 (d, J=12.6 Hz, 1H), 4.19-4.06 (m, 1H), 3.60-3.45 (m, 2H), 3.29 (t, J=8.6 Hz, 1H), 2.92 (ddd, J=10.7, 6.2, 4.8 Hz, 1H), 2.29 (dt, J=8.6, 7.1 Hz, 2H), 2.01-1.80 (m, 2H), 1.65-1.48 (m, 1H), 1.08 (d, J=7.3 Hz, 3H). Title compound Diastereomer 2 (1.21 g, 15% yield); ¹H NMR (300 MHz, Chloroform-d) δ 8.25-8.15 (m, 1H), 8.13-8.02 (m, 2H), 7.44-7.27 (m, 4H), 7.20-7.02 (m, 7H), 6.60 (ddd, J=8.2, 6.9, 1.2 Hz, 1H), 6.49 (dd, J=8.2, 1.7 Hz, 1H), 6.10 (ddd, J=7.6, 2.1, 1.1 Hz, 1H), 4.64 (p, J=7.2 Hz, 1H), 4.41 (d, J=12.6 Hz, 1H), 4.18-4.14 (m, 1H), 3.84-3.63 (m, 1H), 3.64-3.42 (m, 3H), 2.95-2.77 (m, 1H), 2.60 (ddd, J=19.9, 13.5, 9.0 Hz, 1H), 2.34-2.19 (m, 1H), 2.13 (td, J=11.1, 6.3 Hz, 1H), 1.71 (d, J=7.2 Hz, 3H).

### Preparation 49

### (2S)-2-amino-3-(5-bromo-2-chloro-phenyl)butanoic Acid

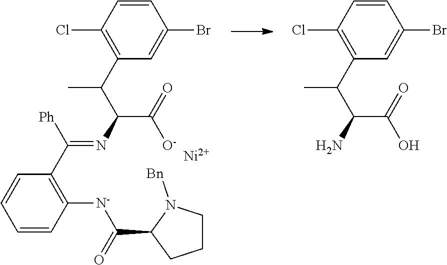

A solution of the product from Preparation 48 (Diastereomer 1, 2.75 g, 3.84 mmol) in MeOH (16 mL) was added to a solution of hydrogen chloride (7.67 mL, 23.0 mmol) in MeOH (8 mL) and the reaction mixture was stirred at 70° C. for 1.5 h. The reaction mixture was concentrated to low volume, diluted with H₂O (30 mL) and re-concentrated in vacuo. Aqueous NH4OH (25%, 20 mL) was added followed by H₂O (20 mL). Stirred for 5 min then reduced under vacuum to half volume. MTBE (5 ml) was added and the solid was collected by filtration to afford title compound. (756 mg, 67% yield); LCMS (METHOD 3) (ES): m/z 294.1 [M+H]⁺, RT=0.40 min.

### Preparation 50

### (2S)-3-(5-bromo-2-chloro-phenyl)-2-(tert-butoxycarbonylamino)butanoic Acid

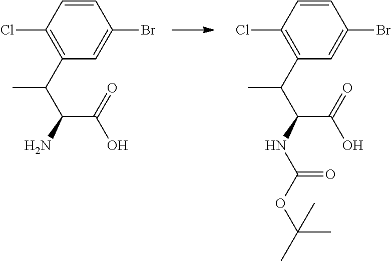 [SMILES: CC(c1cc(Br)ccc1Cl)[C@H](N)C(=O)O ; CC(c1cc(Br)ccc1Cl)[C@H](NC(=O)OC(C)(C)C)C(=O)O]

NaHCO₃ (339 mg, 4.04 mmol) was added to a suspension of the compound from Preparation 49 (787 mg, 2.69 mmol) in H₂O (7 mL) and 1,4-Dioxane (7 mL). To this was added tert-butoxycarbonyl tert-butyl carbonate (705 mg, 3.23 mmol) and the reaction mixture was stirred at room temperature for 72 h. Aqueous NaOH (4 M, 0.67 mL) and tert-butoxycarbonyl tert-butyl carbonate (705 mg, 3.23 mmol) was added and stirred o/n. The reaction mixture was concentrated to low volume and adjusted to pH 1. The aqueous phase was extracted with EtOAc (3×10 mL) and the combined organic phases were dried over Na₂SO₄, filtered and concentrated in vacuo to afford title compound as an off-white solid. (888 mg, 84% yield); LCMS (METHOD 3) (ES): m/z 392.3 [M−H]⁺, RT=0.78 min.

### Preparation 51

### (2S)-2-(tert-butoxycarbonylamino)-3-[2-chloro-5-(1-isopropyl-6-oxo-3-pyridyl)phenyl]butanoic Acid

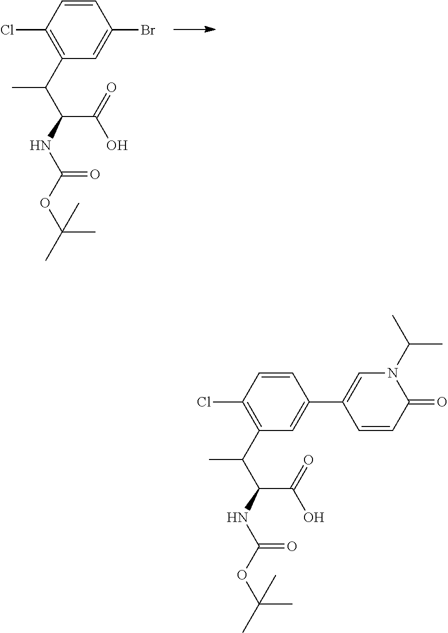 [SMILES: CC(c1cc(Br)ccc1Cl)[C@H](NC(=O)OC(C)(C)C)C(=O)O ; CC(c1cc(-c2ccc(=O)n(C(C)C)c2)ccc1Cl)[C@H](NC(=O)OC(C)(C)C)C(=O)O]

According to the method of Preparation 17 the compound of Preparation 50 (200 mg, 0.51 mmol) was reacted to give the title compound after acidic HPLC as a colourless solid (156 mg, 68% yield). LCMS (METHOD 3) (ES): m/z 448.4 [M+H]⁺, RT=0.69 min.

### Preparation 52

### tert-butyl N-[(1S)-2-[2-chloro-5-(1-isopropyl-6-oxo-3-pyridyl)phenyl]-1-[[4-[3,5-dimethyl-1-(2-trimethylsilylethoxymethyl)pyrazol-4-yl]phenyl]carbamoyl]propyl]carbamate

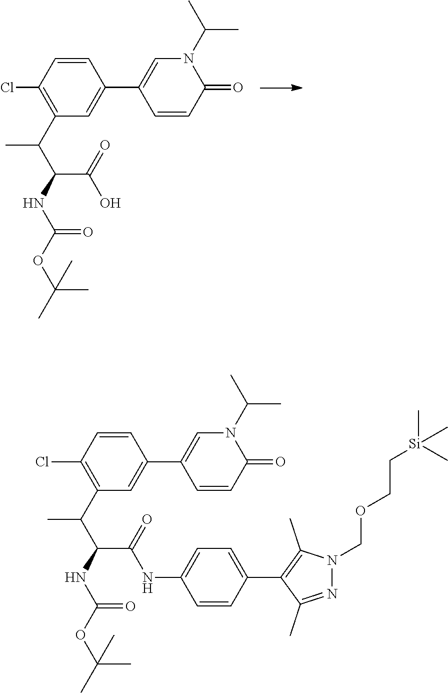 [SMILES: CC(c1cc(-c2ccc(=O)n(C(C)C)c2)ccc1Cl)[C@H](NC(=O)OC(C)(C)C)C(=O)O ; Cc1nn(COCC[Si](C)(C)C)c(C)c1-c1ccc(NC(=O)[C@@H](NC(=O)OC(C)(C)C)C(C)c2cc(-c3ccc(=O)n(C(C)C)c3)ccc2Cl)cc1]

According to the method of Preparation 18 the compound of Preparation 51 (100 mg, 0.223 mmol) was reacted to give the title compound after purification by silica column chromatography (230-400 mesh), eluting with 0-100% EtOAc in heptane to afford the title compound as a colourless oil. (124 mg, 74% yield). LCMS (METHOD 3) (ES): m/z 749.5 [M+H]⁺, RT=0.99 min.

### Preparation 53

### N-[(1S)-2-[2-chloro-5-(1-isopropyl-6-oxo-3-pyridyl)phenyl]-1-[[4-[3,5-dimethyl-1-(2-trimethylsilylethoxymethyl)pyrazol-4-yl]phenyl]carbamoyl]propyl]-1-fluoro-cyclopropanecarboxamide

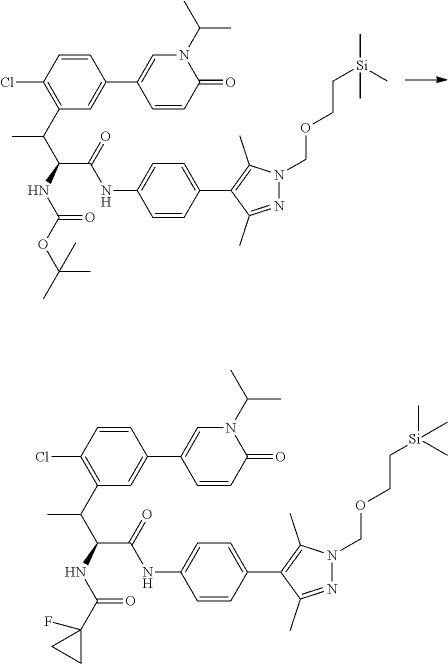 [SMILES: Cc1nn(COCC[Si](C)(C)C)c(C)c1-c1ccc(NC(=O)[C@@H](NC(=O)OC(C)(C)C)C(C)c2cc(-c3ccc(=O)n(C(C)C)c3)ccc2Cl)cc1 ; Cc1nn(COCC[Si](C)(C)C)c(C)c1-c1ccc(NC(=O)[C@@H](NC(=O)C2(F)CC2)C(C)c2cc(-c3ccc(=O)n(C(C)C)c3)ccc2Cl)cc1]

According to the method of Preparation 15 the compound of Preparation 52 (124 mg, 0.166 mmol) was reacted to give the title compound after purification by silica column chromatography (230-400 mesh), eluting with 0-100% MeOH in EtOAc to afford the title compound as a colourless oil. (85.0 mg, 69% yield). LCMS (METHOD 3) (ES): m/z 734.4 [M+H]⁺, RT=0.94 min.

### Preparation 54

### (2S)-2-(1,3-dioxoisoindolin-2-yl)-3-(3-iodophenyl)-N-(8-quinolyl)butanamide

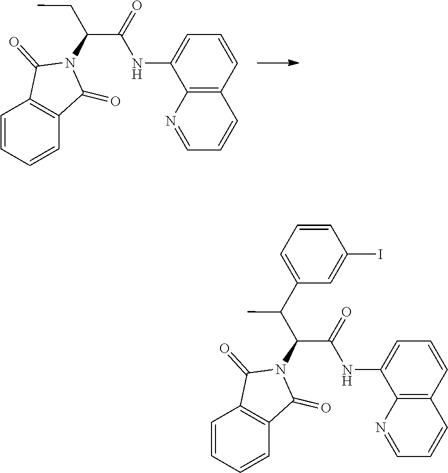 [SMILES: CC[C@@H](C(=O)Nc1cccc2cccnc12)N1C(=O)c2ccccc2C1=O ; CC(c1cccc(I)c1)[C@@H](C(=O)Nc1cccc2cccnc12)N1C(=O)c2ccccc2C1=O]

Ag₂CO₃ (13.7 g, 50.1 mmol) was added to a solution of (2S)-2-(1,3-dioxoisoindolin-2-yl)-N-(8-quinolyl)butanamide (9.0 g, 25.0 mmol) and 1,3-diiodobenzene (8.24 g, 25.0 mmol) in DCE (100 mL) at room temperature. The reaction mixture was degassed under argon for 10 min. Pd(OAc)₂ (112 mg, 0.501 mmol) was added and the reaction mixture was stirred at 70° C. o/n. The reaction mixture was filtered through Celite™ and concentrated under reduced pressure. The obtained crude compound was purified by silica column chromatography (100-200 mesh), eluting with 0-30% EtOAc in hexane, to afford title compound as an off-white solid. (3.8 g, 27% yield); LCMS (METHOD 5) (ES): m/z 562 [M+H]⁺, RT=2.63 min.

### Preparation 55

### tert-butyl N-[(2S)-2-(1,3-dioxoisoindolin-2-yl)-3-(3-iodophenyl)butanoyl]-N-(8-quinolyl)carbamate

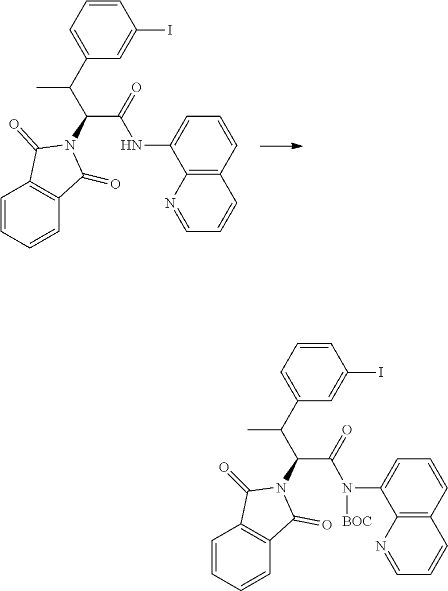 [SMILES: CC(c1cccc(I)c1)[C@@H](C(=O)Nc1cccc2cccnc12)N1C(=O)c2ccccc2C1=O ; CC(c1cccc(I)c1)[C@@H](C(=O)N(C(=O)OC(C)(C)C)c1cccc2cccnc12)N1C(=O)c2ccccc2C1=O]

DMAP (0.83 g, 6.77 mmol) was added to a solution of the compound from Preparation 54 (3.8 g, 6.77 mmol) and tert-butoxycarbonyl tert-butyl carbonate (1.47 g, 6.77 mmol) in MeCN (50 mL) and the reaction mixture was stirred o/n. The reaction mixture was concentrated under reduced pressure to low volume and partitioned between EtOAc (40 mL) and H₂O (10 mL). The organic phase was collected, dried over Na₂SO₄ and concentrated in vacuo. The obtained crude compound was purified by silica column chromatography (100-200 mesh), eluting with 10-30% EtOAc in hexane, to afford title compound as an off-white solid. (3.8 g, 84% yield). LCMS (METHOD 5) (ES): m/z 662 [M+H]⁺, RT=2.65 min.

### Preparation 56

### (2S)-2-(tert-butoxycarbonylamino)-3-(3-iodophenyl)butanoic Acid

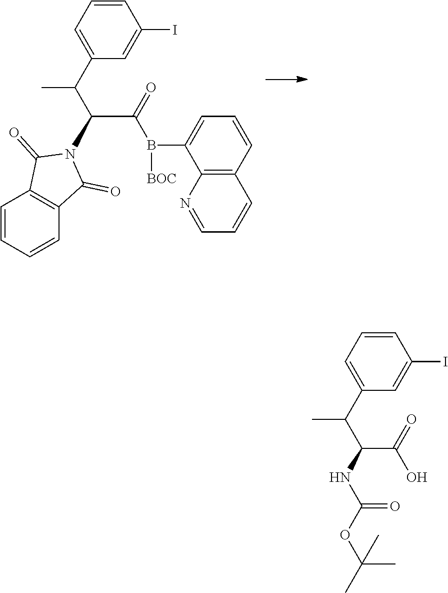 [SMILES: CC(c1cccc(I)c1)[C@@H](C(=O)B(C(=O)OC(C)(C)C)c1cccc2cccnc12)N1C(=O)c2ccccc2C1=O ; CC(c1cccc(I)c1)[C@H](NC(=O)OC(C)(C)C)C(=O)O]

H₂O₂ (30% aq. soln, 1.95 g, 57.5 mmol) was added to a solution of the compound from Preparation 55 (3.8 g, 5.74 mmol) and LiOH.H₂O (1.20 g, 28.7 mmol) in THF/H₂O (3:1, 40 mL) and the resulting mixture was stirred at room temperature o/n. The reaction mixture was diluted with aq. NaOH (0.1M) and washed with DCM (2×50 mL). The aqueous phase was collected and acidified to pH 2 with aq. HCl (6 M). This was then extracted with EtOAc (3×50 mL). The combined extracts were dried over Na₂SO₄, filtered and concentrated in vacuo. The residue was dissolved in Tol (50 mL) and stirred at reflux under Dean-Stark conditions o/n. The reaction mixture was concentrated in vacuo. The residue was dissolved in MeOH (20 mL) and hydrazine monohydrate (430 mg, 8.60 mmol) was added and the resulting reaction mixture was stirred at reflux o/n. To the cooled reaction mixture was added tert-butoxycarbonyl tert-butyl carbonate (1.87 g, 8.60 mmol) and TEA (4.0 mL, 28.7 mmol) and the reaction mixture was stirred at room temperature o/n. The reaction mixture was concentrated under reduced pressure and the residue re-dissolved in aq. NaOH (0.1M) and washed with DCM (2×50 mL). The aqueous phase was collected and acidified to pH 2 with aq. HCl (6 M). This was then extracted with EtOAc (3×50 mL). The combined extracts were dried over Na₂SO₄, filtered and concentrated in vacuo. The obtained crude compound was purified by silica column chromatography (100-200 mesh), eluting with 0-10% MeOH in DCM, to afford title compound. (280 mg, 12% yield); LCMS (METHOD 5) (ES): m/z 406 [M+H]⁺, RT=2.3 min.

### Preparation 57

### tert-butyl N-[(1S)-2-(3-iodophenyl)-1-[[4-(3-methylimidazol-4-yl)phenyl]carbamoyl]propyl]carbamate

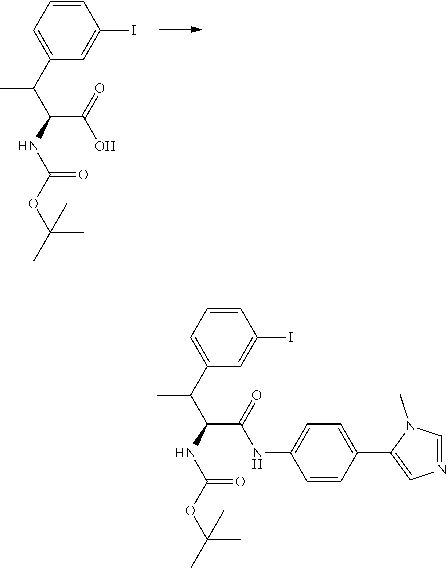 [SMILES: CC(c1cccc(I)c1)[C@H](NC(=O)OC(C)(C)C)C(=O)O ; CC(c1cccc(I)c1)[C@H](NC(=O)OC(C)(C)C)C(=O)Nc1ccc(-c2cncn2C)cc1]

According to the method of Preparation 14 the compound of Preparation 56 (264 mg, 0.65 mmol) was reacted to give the title compound after purification by basic HPLC to afford the title compound as 2 individual diastereomers. Diastereomer 1 (105 mg, 29% yield); ¹H NMR (600 MHz, Chloroform-d) δ 8.83 (s, 1H), 7.62 (t, J=1.8 Hz, 1H), 7.53 (d, J=10.2 Hz, 2H), 7.31-7.21 (m, 3H), 7.05 (s, 1H), 7.00 (t, J=7.8 Hz, 1H), 5.62 (d, J=9.3 Hz, 1H), 4.49 (t, J=8.7 Hz, 1H), 3.61 (s, 3H), 3.25 (s, 1H), 1.43 (s, 9H), 1.38 (d, J=7.1 Hz, 3H), LCMS (METHOD 3) (ES): m/z 562.1 [M+H]⁺, RT=0.67 min. Diastereomer 2 (65 mg, 17.8%) ¹H NMR (600 MHz, Chloroform-d) δ 8.90-8.71 (m, 1H), 7.62 (t, J=1.8 Hz, 1H), 7.59 (d, J=7.9 Hz, 1H), 7.51 (d, J=8.6 Hz, 3H), 7.30-7.22 (m, 3H), 7.08-7.01 (m, 2H), 5.37-5.27 (m, 1H), 4.50 (s, 1H), 3.60 (s, 3H), 3.36 (p, J=7.2 Hz, 1H), 1.41 (s, 9H), 1.36 (d, J=7.1 Hz, 3H). LCMS (METHOD 3) (ES): m/z 562.1 [M+H]⁺, RT=0.69 min.

### Preparation 58

### tert-butyl N-[(1S)-2-[3-(1-isopropyl-6-oxo-3-pyridyl)phenyl]-1-[[4-(3-methylimidazol-4-yl)phenyl]carbamoyl]propyl]carbamate

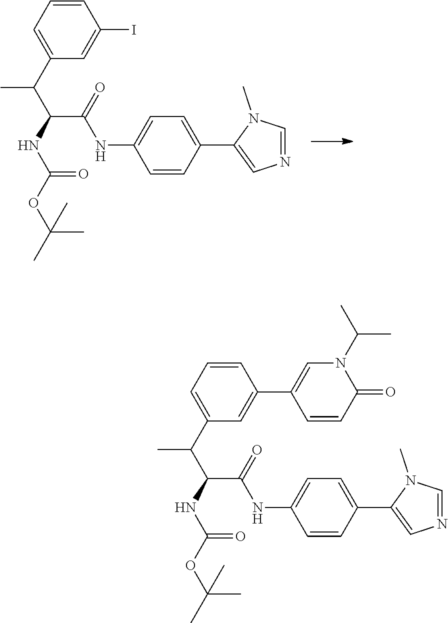 [SMILES: CC(c1cccc(I)c1)[C@H](NC(=O)OC(C)(C)C)C(=O)Nc1ccc(-c2cncn2C)cc1 ; CC(c1cccc(-c2ccc(=O)n(C(C)C)c2)c1)[C@H](NC(=O)OC(C)(C)C)C(=O)Nc1ccc(-c2cncn2C)cc1]

According to the method of Preparation 16 the compound of Preparation 57 (105 mg, 0.187 mmol) was reacted to give the title compound after basic HPLC as a colourless solid (81 mg, 76% yield). LCMS (METHOD 4) (ES): m/z 570.3 [M+H]⁺, RT=0.65 min.

### Preparation 59

### methyl (2S)-2-amino-3-(3-hydroxyphenyl)propanoate Hydrochloride

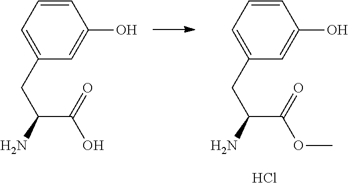

Thionyl chloride (29.8 mL, 414 mmol) was added slowly to a solution of (S)-2-amino-3-(3-hydroxyphenyl) propanoic acid (25.0 g, 138 mmol) in MeOH (300 mL) at 0° C. The resulting reaction mixture was slowly warmed to and stirred at 90° C. for 6 h. The reaction mixture was concentrated under reduced pressure. The obtained crude product was slurried in Et₂O (500 mL), filtered and dried under vacuum to afford title compound as an off-white solid. (31 g, 99% yield); LCMS (METHOD 6) (ES): m/z 196 [M+H]⁺, RT=1.79 min.

### Preparation 60

### methyl (2S)-2-(tert-butoxycarbonylamino)-3-(3-hydroxyphenyl)propanoate

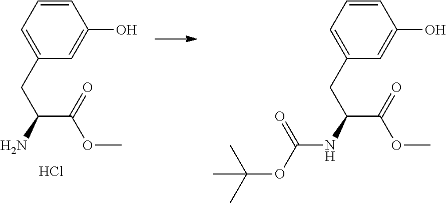 [SMILES: COC(=O)[C@@H](N)Cc1cccc(O)c1 ; Cl ; COC(=O)[C@H](Cc1cccc(O)c1)NC(=O)OC(C)(C)C]

Tert-butoxycarbonyl tert-butyl carbonate (32.1 g, 147 mmol) was added to a solution of the product from Preparation 59 (31 g, 134 mmol) and NaHCO₃ (33.7 g, 401 mmol) in THF/H₂O (350/100 mL) slowly at 0° C. On complete addition the reaction mixture was stirred at room temperature o/n. The reaction mixture was diluted with H₂O (300 mL) and extracted with EtOAc (2×400 mL). The combined organic layers were washed successively with H₂O (300 mL) then aq. brine solution (400 mL), dried over Na₂SO₄, filtered and concentrated in vacuo to afford title compound as colourless oil. (44 g, 93% yield). LCMS (METHOD 5) (ES): m/z 296.1 [M+H]⁺, RT=1.74 min.

### Preparation 61

### methyl (2S)-2-(tert-butoxycarbonylamino)-3-(2-chloro-5-hydroxy-phenyl)propanoate

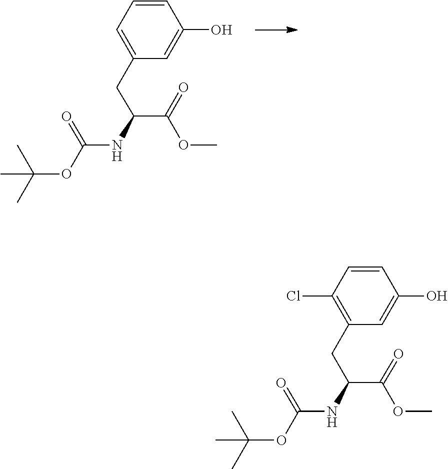 [SMILES: COC(=O)[C@H](Cc1cccc(O)c1)NC(=O)OC(C)(C)C ; COC(=O)[C@H](Cc1cc(O)ccc1Cl)NC(=O)OC(C)(C)C]

NCS (19.91 g, 149 mmol) was added portionwise to a solution of the compound from Preparation 60 (44 g, 149 mmol) in THF (450 mL) at 0° C. On complete addition the reaction mixture was stirred at 90° C. o/n. The reaction mixture was diluted with ice cold H₂O (1 L) and extracted with EtOAc (2×1 L). The combined organic layers were washed successively with H₂O (1 L) then aq. brine solution (1 L), dried over Na₂SO₄, filtered and concentrated in vacuo. The obtained crude compound was purified by silica column chromatography (230-400 mesh), eluting with 15% EtOAc in hexane, to afford title compound as an off-white solid. (20 g, 49% yield); LCMS (METHOD 5) (ES): m/z 328 [M+H]⁺, RT=1.89 min.

### Preparation 62

### (2S)-2-(tert-butoxycarbonylamino)-3-(2-chloro-5-hydroxy-phenyl)propanoic Acid

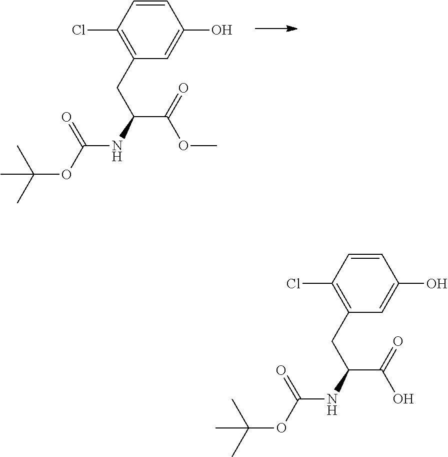 [SMILES: COC(=O)[C@H](Cc1cc(O)ccc1Cl)NC(=O)OC(C)(C)C ; CC(C)(C)OC(=O)N[C@@H](Cc1cc(O)ccc1Cl)C(=O)O]

LiOH.H₂O (3.81 g, 90.9 mmol) was added to a solution of the product from Preparation 61 (10 g, 30.3 mmol) in THF/H₂O (1:1, 200 mL) portionwise at 0° C. The reaction mixture was stirred at room temperature o/n. The reaction mixture was cooled to 0° C., and acidified to pH 6 with 20% aq. citric acid then extracted with EtOAc (2×250 mL). The combined organic layers were washed successively with H₂O (250 mL) then aq. brine solution (250 mL), dried over Na₂SO₄, filtered and concentrated in vacuo to afford title compound as an off-white solid. (8.0 g, 83% yield). LCMS (METHOD 5) (ES): m/z 316.1 [M+H]⁺, RT=1.67 min.

### Preparation 63

### tert-butyl N-[(1S)-1-[(2-chloro-5-hydroxy-phenyl)methyl]-2-[4-(3-methylimidazol-4-yl)anilino]-2-oxo-ethyl]carbamate

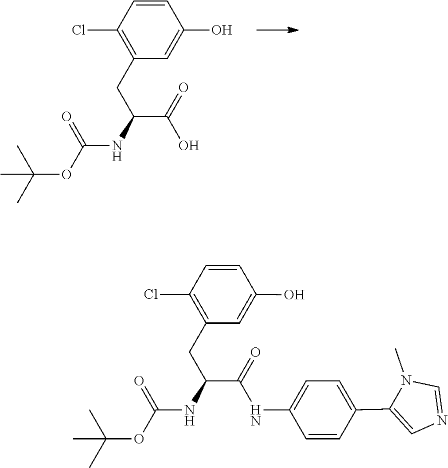 [SMILES: CC(C)(C)OC(=O)N[C@@H](Cc1cc(O)ccc1Cl)C(=O)O ; Cn1cncc1-c1ccc(NC(=O)[C@H](Cc2cc(O)ccc2Cl)NC(=O)OC(C)(C)C)cc1]

T₃P (50% soln in EtOAc, 12.9 mL, 20.3 mmol) was added dropwise to a solution of the compound from Preparation 62 (3.2 g, 10.1 mmol), 4-(3-methylimidazol-4-yl)aniline (1.76 g, 10.1 mmol) and DIPEA (7.4 mL, 40.5 mmol) in THF (30 mL) at 0° C. On complete addition the resulting solution was stirred at 80° C. o/n. The reaction mixture was diluted with ice cold H₂O (100 mL) and extracted with EtOAc (2×100 mL). The combined organic layers were washed successively with H₂O (100 mL) then aq. brine solution (100 mL), dried over Na₂SO₄, filtered and concentrated in vacuo. The obtained crude compound was purified by silica column chromatography (230-400 mesh), eluting with 5% MeOH in DCM, to afford title compound as an off-white solid. (2.6 g, 54% yield); LCMS (METHOD 5) (ES): m/z 471 [M+H]⁺, RT=1.49 min.

### Preparation 64

### (2S)-2-amino-3-(2-chloro-5-hydroxy-phenyl)-N-[4-(3-methylimidazol-4-yl)phenyl]propenamide Hydrochloride

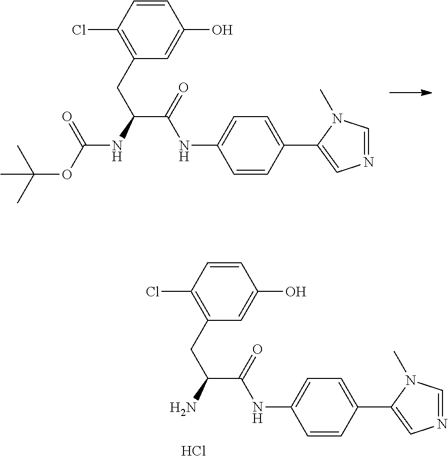 [SMILES: Cn1cncc1-c1ccc(NC(=O)[C@H](Cc2cc(O)ccc2Cl)NC(=O)OC(C)(C)C)cc1 ; Cn1cncc1-c1ccc(NC(=O)[C@@H](N)Cc2cc(O)ccc2Cl)cc1 ; Cl]

According to the method of Preparation 28 the compound of Preparation 63 (2.9 g, 0.195 mmol) was reacted to give the title compound as an off-white solid (2.8 g, assume quantitative yield). LCMS (METHOD 3) (ES): m/z 371.1 [M+H]⁺, RT=0.96 min.

### Preparation 65

### N-[(1S)-1-[(2-chloro-5-hydroxy-phenyl)methyl]-2-[4-(3-methylimidazol-4-yl)anilino]-2-oxo-ethyl]-2-methyl-pyrazole-3-carboxamide

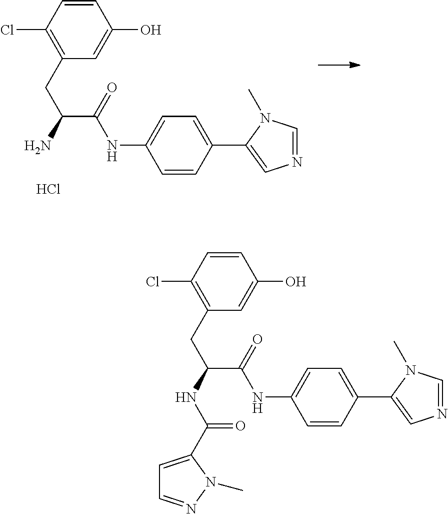 [SMILES: Cn1cncc1-c1ccc(NC(=O)[C@@H](N)Cc2cc(O)ccc2Cl)cc1 ; Cl ; Cn1cncc1-c1ccc(NC(=O)[C@H](Cc2cc(O)ccc2Cl)NC(=O)c2ccnn2C)cc1]

According to the method of Preparation 63 the compound of Preparation 64 (500 mg, 1.23 mmol) was reacted to give the title compound as an off-white solid (200 mg, 34% yield). LCMS (METHOD 3) (ES): m/z 479.3 [M+H]⁺, RT=1.33 min.

### Preparation 66

### [4-chloro-3-[(2S)-3-[4-(3-methylimidazol-4-yl)anilino]-2-[(2-methylpyrazole-3-carbonyl)amino]-3-oxo-propyl]phenyl] trifluoromethanesulfonate

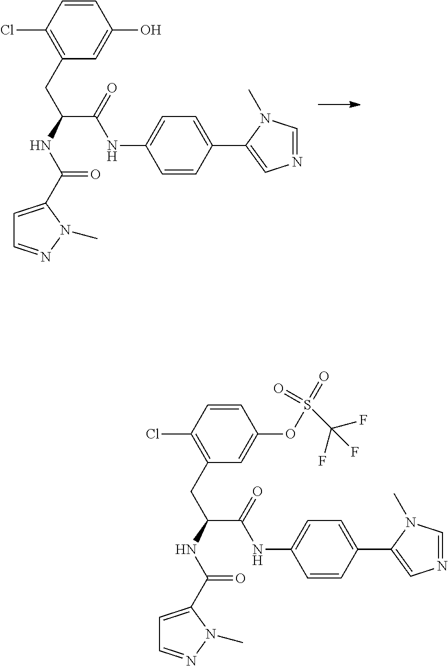 [SMILES: Cn1cncc1-c1ccc(NC(=O)[C@H](Cc2cc(O)ccc2Cl)NC(=O)c2ccnn2C)cc1 ; Cn1cncc1-c1ccc(NC(=O)[C@H](Cc2cc(OS(=O)(=O)C(F)(F)F)ccc2Cl)NC(=O)c2ccnn2C)cc1]

N-Phenylbis(trifluoromethanesulfonimide) (163 mg, 0.46 mmol) was added to a solution of the compound from Preparation 65 (200 mg, 0.42 mmol) and TEA (0.18 ml, 1.25 mmol) portionwise at 0° C. On complete addition the resulting solution was stirred at room temperature o/n. The reaction mixture was diluted with ice cold H₂O (50 mL) and extracted with DCM (2×50 mL). The combined organic layers were dried over Na₂SO₄, filtered and concentrated in vacuo to afford title compound as a viscous oil. (0.22 g); LCMS (METHOD 5) (ES): m/z 611.2 [M+H]⁺, RT=1.70 min.

### Preparation 67

### Diethyl 2-acetamido-2-[(5-bromo-2-chloro-phenyl)methyl]propanedioate

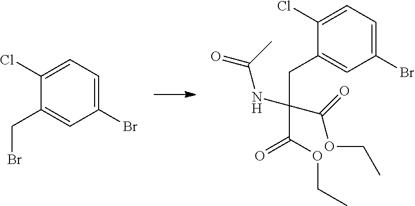 [SMILES: Clc1ccc(Br)cc1CBr ; CCOC(=O)C(Cc1cc(Br)ccc1Cl)(NC(C)=O)C(=O)OCC]

Diethyl acetamidomalonate (22.9 g, 105.6 mmol) was added to a solution of 4-bromo-2-(bromomethyl)-1-chlorobenzene (30.0 g, 105.6 mmol) and cesium carbonate (102.9 g, 316.9 mmol) in DMF (300 mL) slowly at 0° C. On complete addition the reaction mixture was stirred to room temperature o/n. The reaction mixture was quenched with H₂O (500 mL) and extracted with EtOAc (2×500 mL). The combined organic phases were successively washed with H₂O (500 mL) and brine (500 mL) then dried over Na₂SO₄, filtered and concentrated in vacuo. The obtained crude compound was purified by silica column chromatography (100-200 mesh), eluting with 10% EtOAc in hexane, to afford title compound as an off-white solid. (27.5 g, 61% yield); ¹H NMR (300 MHz, DMSO): δ ppm 8.28 (s, 1H), 7.51-7.47 (dd, J=11.2, 3.2 Hz, 1H), 7.41-7.38 (d, J=11.2 Hz, 1H), 7.17-7.16 (d, J=2.8 Hz, 1H), 4.19-4.12 (m, 4H), 3.59 (s, 2H), 1.91 (s, 3H) 1.20-1.16 (t, J=10 Hz, 6H); LCMS (Method 5) (ES): m/z 420 [M+H]⁺; 92%; RT=2.43 min;

### Preparation 68

### 2-acetamido-3-(5-bromo-2-chloro-phenyl)propanoic Acid

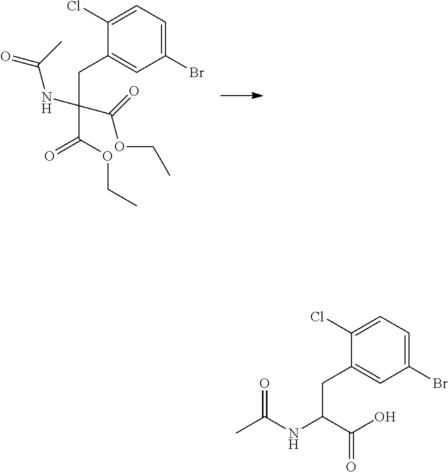 [SMILES: CCOC(=O)C(Cc1cc(Br)ccc1Cl)(NC(C)=O)C(=O)OCC ; CC(=O)NC(Cc1cc(Br)ccc1Cl)C(=O)O]

LiOH.H₂O (16.5 g, 392.8 mmol) was added slowly to a solution of the compound from Preparation 67 in THF:H₂O (260 mL, 1:1) at 0° C. On complete addition the reaction mixture was warmed to 80° C. and stirred o/n. The cooled reaction mixture was acidified to pH3 with sat. aq. KHSO₄ solution then extracted with EtOAc (2×500 mL). The combined organic phases were successively washed with H₂O (500 mL) and brine (500 mL) then dried over Na₂SO₄, filtered and concentrated in vacuo, to afford title compound as an off-white solid. (40.0 g, 95% yield); ¹H NMR (300 MHz, DMSO-d₆): δ ppm 12.80 (br s, 1H), 8.26-8.24 (d, J=8.44 Hz, 1H), 7.54-7.37 (m, 3H), 4.53-4.45 (m, 1H), 3.26-3.19 (dd, J=13.5, 4.95 Hz, 1H), 2.91-2.83 (dd, J=13.8, 9.9 Hz, 1H), 1.77 (s, 3H); LCMS (Method 5) (ES): m/z 321 [M+H]⁺; 84%; RT=1.56 min.

### Preparation 69

### (2S)-2-amino-3-(5-bromo-2-chlorophenyl)propanoic Acid

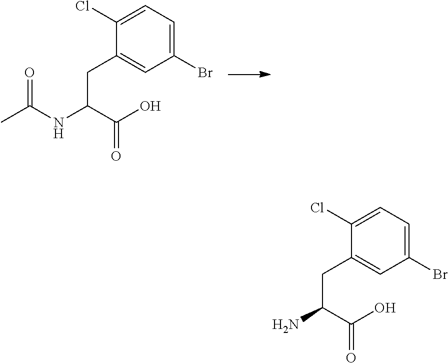 [SMILES: CC(=O)NC(Cc1cc(Br)ccc1Cl)C(=O)O ; N[C@@H](Cc1cc(Br)ccc1Cl)C(=O)O]

NaOH (5% aq. Solution, ˜800 mL) was added to a stirring suspension of the compound from Preparation 68 (60.0 g, 187.5 mmol) in H₂O (350 mL) until a homogenous solution was obtained. The solution was neutralized to pH8 with 5N HCl (aq, ˜500 mL). Acylase I (Aspergillus melleus, 20 g) was added and the reaction mixture was stirred at 37° C. for 72 h. The obtained precipitate was collected by filtration, washed with H₂O (350 mL) and dried to afford title compound as an off-white solid. (20.0 g, 38% yield)

¹H NMR (300 MHz, D₂O): δ ppm 7.56 (s, 1H), 7.53-7.50 (d, J=8.7 Hz, 1H), 7.42-7.34 (d, J=8.7 Hz, 1H), 4.07-4.03 (t, J=7.2 Hz, 1H), 3.47-3.39 (m, 1H) 3.2-3.13 (m, 1H); LCMS (Method 5) (ESI): m/z 276 [M−H]⁻; 97%; RT=1.16 min; Chiral HPLC: Column: CHIRALPAK AD-3 (4.6×150 mm) 3 μm; Co-solvent: Methanol, Total Flow: 3 g/min; % of Co-solvent: 15, ABPR: 1500 psi, Temperature: 30° C., RT: 3.08 (99.54%).

### Preparation 70

### (2S)-3-(5-bromo-2-chlorophenyl)-2-(tert-butoxycarbonylamino)propanoic Acid

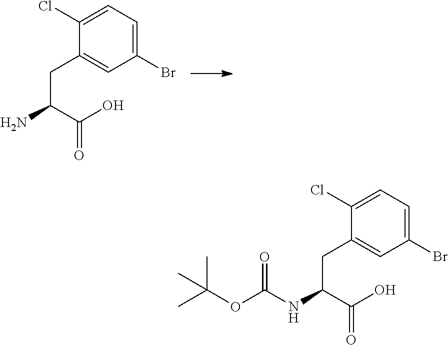 [SMILES: N[C@@H](Cc1cc(Br)ccc1Cl)C(=O)O ; CC(C)(C)OC(=O)N[C@@H](Cc1cc(Br)ccc1Cl)C(=O)O]

NaHCO₃ (12.0 g, 143.9 mmol) was added to a solution of the compound from Preparation 69 (20.0 g, 71.9 mmol) in MeCN:H₂O (150 mL, 1:1) and cooled to 0° C. Tert-butoxycarbonyl tert-butyl carbonate (18.8 g, 86.3 mmol) was added slowly, then allowed to warm to room temperature with stirring o/n. The reaction mixture was acidified with sat. citric acid solution to pH3, then extracted with EtOAc (2×400 mL). The combined organic phases were successively washed with H₂O (400 mL) and brine (400 mL) then dried over Na₂SO₄, filtered and concentrated in vacuo, to afford title compound as an off-white solid. (20.0 g, 73% yield); ¹H NMR (400 MHz, DMSO-d₆): δ ppm 12.76 (br s, 1H), 7.55-7.55 (d, J=2.4 Hz, 1H),

7.46-7.14 (m, 3H), 7.16-7.14 (d, J=8.8 Hz, 1H), 4.24-4.18 (m, 1H), 3.25-3.21 (m, 1H), 2.88-2.81 (m, 1H), 1.30 (s, 9H); LCMS (Method 5) (ES): m/z 376 [M−H]⁻, 99%; RT=2.39 min; Chiral HPLC: Column: CHIRALPAK AD-3 (4.6×150 mm) 3 μm; Co-solvent: Methanol, Total Flow: 3 g/min; % of Co-solvent: 15, ABPR: 1500 psi, Temperature: 30° C., RT: 1.38 (99.82%).

### Preparation 71

### tert-butyl N-[(1S)-1-[(5-bromo-2-chloro-phenyl)methyl]-2-[4-[3,5-dimethyl-1-(2-trimethylsilylethoxymethyl)pyrazol-4-yl]anilino]-2-oxo-ethyl]carbamate

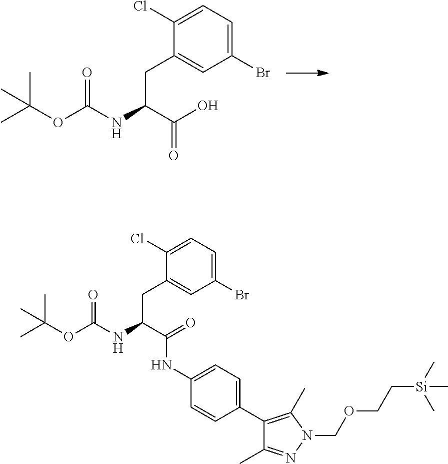 [SMILES: CC(C)(C)OC(=O)N[C@@H](Cc1cc(Br)ccc1Cl)C(=O)O ; Cc1nn(COCC[Si](C)(C)C)c(C)c1-c1ccc(NC(=O)[C@H](Cc2cc(Br)ccc2Cl)NC(=O)OC(C)(C)C)cc1]

DIPEA (0.14 mL, 0.79 mmol) was added to a solution of the compound from Preparation 70 (200 mg, 0.53 mmol) and 4-[3,5-dimethyl-1-(2-trimethylsilylethoxymethyl)pyrazol-4-yl]aniline (218 mg, 0.69 mmol) in DMF (2 mL) at room temperature. HATU (301 mg, 0.79 mmol) was added and the reaction mixture stirred for 30 min. The reaction mixture was purified directly by basic HPLC, to afford title compound as an off-white solid. (312 mg, 87% yield) LCMS (METHOD 3) (ES): m/z 678.4 [M+H]⁺, RT=1.04 min.

### Preparation 72

### N-[(1S)-1-[(5-bromo-2-chloro-phenyl)methyl]-2-[4-[3,5-dimethyl-1-(2-trimethylsilylethoxymethyl)pyrazol-4-yl]anilino]-2-oxo-ethyl]-2-methyl-pyrazole-3-carboxamide

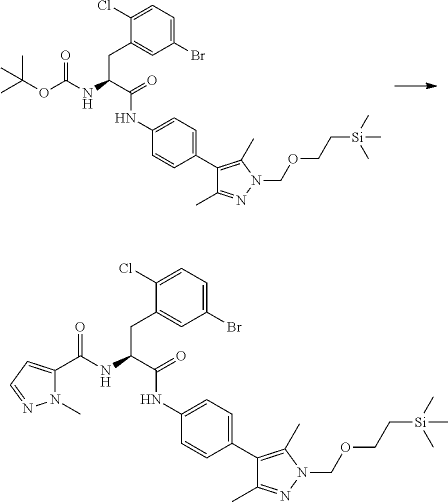 [SMILES: Cc1nn(COCC[Si](C)(C)C)c(C)c1-c1ccc(NC(=O)[C@H](Cc2cc(Br)ccc2Cl)NC(=O)OC(C)(C)C)cc1 ; Cc1nn(COCC[Si](C)(C)C)c(C)c1-c1ccc(NC(=O)[C@H](Cc2cc(Br)ccc2Cl)NC(=O)c2ccnn2C)cc1]

Hydrogen chloride (4M in dioxan, 8 mL) was added to a solution of the compound from Preparation 71 (312 mg, 0.046 mmol) in MeOH (4 mL) at room temperature for 1 h. The reaction mixture was concentrated in vacuo to give crude intermediate (2S)-2-amino-3-(5-bromo-2-chloro-phenyl)-N-[4-[3,5-dimethyl-1-(2-trimethylsilylethoxymethyl)pyrazol-4-yl]phenyl]propenamide hydrochloride. Assume quantitative yield. LCMS (METHOD 3) (ES): m/z 578.3 [M+H]⁺, RT=0.79 min. DIPEA (0.8 mL, 5.3 mmol) was added to a solution of the crude (2S)-2-amino-3-(5-bromo-2-chloro-phenyl)-N-[4-[3,5-dimethyl-1-(2-trimethylsilylethoxymethyl)pyrazol-4-yl]phenyl]propenamide hydrochloride (265 mg, 0.53 mmol) and 2-methylpyrazole-3-carboxylic acid (86.7 mg, 0.79 mmol) in DMF (5 mL) at room temperature. HATU (261 mg, 0.79 mmol) was added and the reaction mixture stirred for 30 min. The reaction mixture was purified directly by basic HPLC, to afford title compound as an off-white solid. (241 mg, 76% yield) LCMS (METHOD 3) (ES): m/z 687.5 [M+H]⁺, RT=0.96 min.

### Preparation 73

### N-[(1S)-1-[[2-chloro-5-[3-(1-hydroxy-1-methyl-ethyl)phenyl]phenyl]methyl]-2-[4-[3,5-dimethyl-1-(2-trimethylsilylethoxymethyl)pyrazol-4-yl]anilino]-2-oxo-ethyl]-2-methyl-pyrazole-3-carboxamide

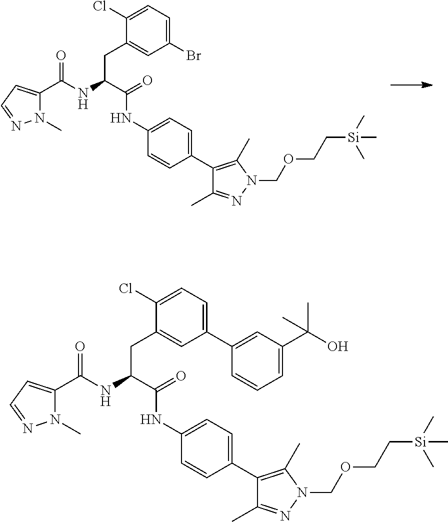 [SMILES: Cc1nn(COCC[Si](C)(C)C)c(C)c1-c1ccc(NC(=O)[C@H](Cc2cc(Br)ccc2Cl)NC(=O)c2ccnn2C)cc1 ; Cc1nn(COCC[Si](C)(C)C)c(C)c1-c1ccc(NC(=O)[C@H](Cc2cc(-c3cccc(C(C)(C)O)c3)ccc2Cl)NC(=O)c2ccnn2C)cc1]

2-[3-(4,4,5,5-tetramethyl-1,3,2-dioxaborolan-2-yl)phenyl]propan-2-ol (30.6 mg, 0.117 mmol) was added to a solution of the compound from Preparation 72 (40.0 mg, 0.058 mmol) in DMF (0.4 mL) in a 5 mL MW vial. A solution of K₂CO₃ (24.2 mg, 0.003 mmol) in H₂O (0.08 mL) was added and the reaction mixture was degassed with argon for 10 min. PdCl₂(dppf).DCM (2.37 mg, 0.003 mmol) was added and the vial was capped and stirred for 30 min at 80° C. The reaction mixture was passed through a PTFE filter and purified by acidic HPLC directly to afford the title compound as an off-white solid. (33 mg, 76% yield)

LCMS (METHOD 3) (ES): m/z 741.6 [M+H]⁺, RT=0.95 min.

### Preparation 74

### N-[(1S)-1-[[5-[3-(1-azido-1-methyl-ethyl)phenyl]-2-chloro-phenyl]methyl]-2-[4-(3,5-dimethyl-1H-pyrazol-4-yl)anilino]-2-oxo-ethyl]-2-methyl-pyrazole-3-carboxamide

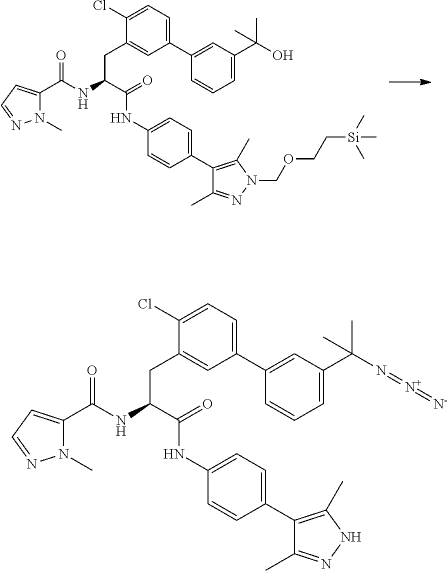 [SMILES: Cc1nn(COCC[Si](C)(C)C)c(C)c1-c1ccc(NC(=O)[C@H](Cc2cc(-c3cccc(C(C)(C)O)c3)ccc2Cl)NC(=O)c2ccnn2C)cc1 ; Cc1n[nH]c(C)c1-c1ccc(NC(=O)[C@H](Cc2cc(-c3cccc(C(C)(C)N=[N+]=[N-])c3)ccc2Cl)NC(=O)c2ccnn2C)cc1]

TFA (0.03 mL) was added to a suspension of the compound from Preparation 73 (35.0 mg, 0.057 mmol) and sodium azide (8.2 mg, 0.13 mmol) in chloroform (0.5 mL) at room temperature. The reaction mixture was stirred o/n. The reaction mixture was concentrated in vacuo then redissolved in DMF (0.5 mL) and purified by acidic HPLC to afford the title compound as an off-white solid. (10.0 mg, 27%), LCMS (METHOD 3) (ES): m/z 636.5 [M+H]⁺, RT=0.85 min.

### Preparation 75

### N-[(1S)-1-[[2-chloro-5-[3-[(dimethylamino)methyl]phenyl]phenyl]methyl]-2-[4-[3,5-dimethyl-1-(2-trimethylsilylethoxymethyl)pyrazol-4-yl]anilino]-2-oxo-ethyl]-2-methyl-pyrazole-3-carboxamide

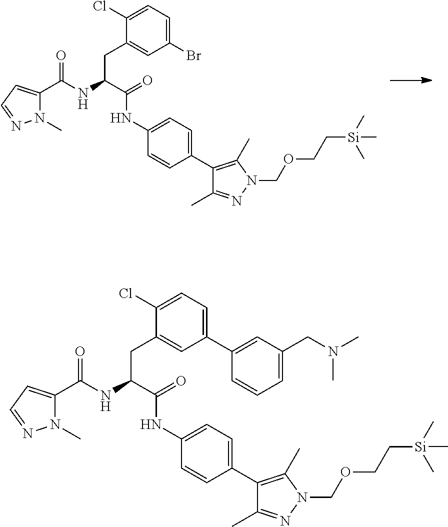 [SMILES: Cc1nn(COCC[Si](C)(C)C)c(C)c1-c1ccc(NC(=O)[C@H](Cc2cc(Br)ccc2Cl)NC(=O)c2ccnn2C)cc1 ; Cc1nn(COCC[Si](C)(C)C)c(C)c1-c1ccc(NC(=O)[C@H](Cc2cc(-c3cccc(CN(C)C)c3)ccc2Cl)NC(=O)c2ccnn2C)cc1]

K₂CO₃ (48.3 mg, 0.35 mmol) in H₂O (0.16 mL) was added to a solution of the compound from Preparation 72 (80.0 mg, 0.117 mmol) and N,N-dimethyl-1-[4-(4,4,5,5-tetramethyl-1,3,2-dioxaborolan-2-yl)phenyl]methanamine hydrochloride (69.4 mg, 0.233 mmol) in DMF (0.8 mL) in a 5 mL MW vial. The reaction mixture was degassed with argon for 10 min. PdCl₂(dppf).DCM (4.74 mg, 0.006 mmol) was added and the reaction run and worked up as described in Preparation 73 to afford title compound as a colourless solid. (76 mg, 86% yield) LCMS (METHOD 3) (ES): m/z 740.8 [M+H]⁺, RT=0.77 min.

### Preparation 76

### tert-butyl N-[[3-[4-chloro-3-[(2S)-3-[4-[3,5-dimethyl-1-(2-trimethylsilylethoxymethyl)pyrazol-4-yl]anilino]-2-[(2-methylpyrazole-3-carbonyl)amino]-3-oxo-propyl]phenyl]phenyl]methyl]carbamate

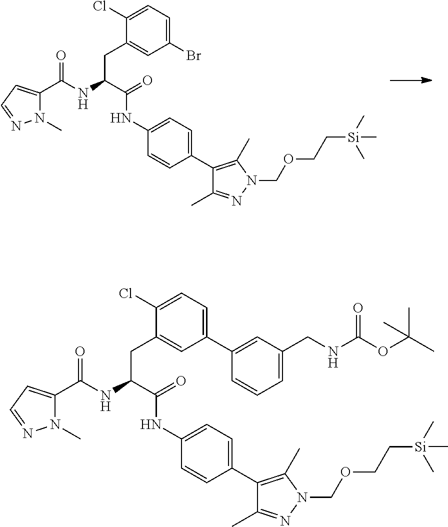 [SMILES: Cc1nn(COCC[Si](C)(C)C)c(C)c1-c1ccc(NC(=O)[C@H](Cc2cc(Br)ccc2Cl)NC(=O)c2ccnn2C)cc1 ; Cc1nn(COCC[Si](C)(C)C)c(C)c1-c1ccc(NC(=O)[C@H](Cc2cc(-c3cccc(CNC(=O)OC(C)(C)C)c3)ccc2Cl)NC(=O)c2ccnn2C)cc1]

K₂CO₃ (30.2 mg, 0.22 mmol) in H₂O (0.1 mL) was added to a solution of the compound from Preparation 72 (50.0 mg, 0.073 mmol) and tert-butyl N-[[4-(4,4,5,5-tetramethyl-1,3,2-dioxaborolan-2-yl)phenyl]methyl]carbamate (48.6 mg, 0.146 mmol) in DMF (0.8 mL) in a 5 mL MW vial. The reaction mixture was degassed with argon for 10 min. PdCl₂(dppf).DCM (2.94 mg, 0.0036 mmol) was added and the reaction run and worked up as described in Preparation 73 to afford title compound as a colourless solid. (56 mg, 94% yield) LCMS (METHOD 3) (ES): m/z 740.8 [M+H]⁺, RT=1.01 min.

### Preparation 77

### (3-bromophenyl)-phenyl-methanol

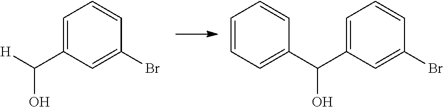 [SMILES: OCc1cccc(Br)c1 ; OC(c1ccccc1)c1cccc(Br)c1]

Phenylmagnesium chloride (2M in THF, 40.5 mL) was added slowly to a solution of 3-bromobenzaldehyde (10.0 g, 54.1 mmol) in THF (200 mL) at −78° C. On complete addition the reaction was slowly warmed to room temperature and stirred for 2 h. The reaction mixture was cooled to 0° C. and quenched with sat. aq. NH4Cl solution (500 mL) and extracted with EtOAc (2×250 mL). The combined organic phases were successively washed with H₂O (500 mL) and brine (500 mL) then dried over Na₂SO₄, filtered and concentrated in vacuo. The obtained crude compound was purified by silica column chromatography (100-200 mesh), eluting with 20-30% EtOAc in hexane, to afford title compound as a colourless liquid. (10.0 g, 70% yield); LCMS (Method 5) (ES): m/z 262 [M+H]⁺, RT=2.5 min.

### Preparation 78

### 1-bromo-3-[bromo(phenyl)methyl]benzene

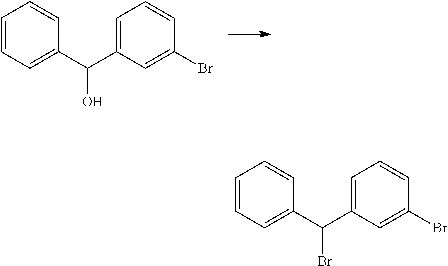 [SMILES: OC(c1ccccc1)c1cccc(Br)c1 ; Brc1cccc(C(Br)c2ccccc2)c1]

Phosphorous tribromide (5.42 mL, 57.0 mmol) was added dropwise to a solution of the compound from Preparation 77 (10.0 g, 38.0 mmol) in Et₂O (200 mL) at 0° C. On complete addition the reaction was slowly warmed to room temperature and stirred for 6 h. The reaction mixture was cooled to 0° C. and quenched with ice cold H₂O (500 mL) and extracted with EtOAc (2×250 mL). The combined organic phases were washed with brine (250 mL) then dried over Na₂SO₄, filtered and concentrated in vacuo to afford title compound as a colourless liquid. (8.0 g, 64% yield); 1H NMR (400 MHz, CDCl₃) δ 7.62-7.59 (m, 1H), 7.29-7.17 (m, 8H), 6.20 (s, 1H).

### Preparation 79

### ethyl 2-(benzhydrylideneamino)-3-(3-bromophenyl)-3-phenyl-propanoate

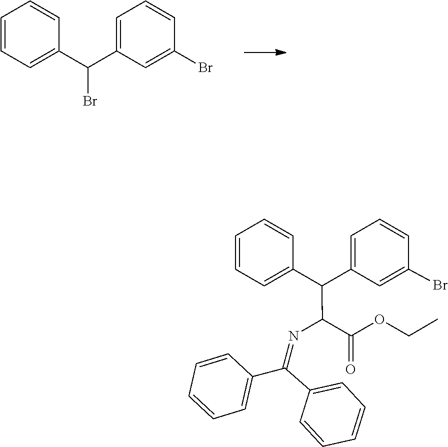 [SMILES: Brc1cccc(C(Br)c2ccccc2)c1 ; CCOC(=O)C(N=C(c1ccccc1)c1ccccc1)C(c1ccccc1)c1cccc(Br)c1]

TBAB (7.9 g, 24.5 mmol) was added to a solution of the compound from Preparation 78 (8.0 g, 24.5 mmol) and ethyl 2-(benzhydrylideneamino)acetate (6.55 g, 24.5 mmol) in DCM (200 mL). The reaction mixture was cooled to 0° C. whereupon 50% NaOH aq. Solution (30 mL) was added. The reaction was stirred at room temperature o/n. The reaction mixture was quenched with ice cold H₂O (200 mL) and extracted with DCM (2×200 mL). The combined organic phases were washed with brine (200 mL) then dried over Na₂SO₄, filtered and concentrated in vacuo to afford title compound (mixture of diastereomers) as a brown viscous liquid. (5.0 g, 39% yield); LCMS (Method 5) (ES): m/z 511 [M+H]⁺, RT=3.22 min.

### Preparation 80

### ethyl 2-amino-3-(3-bromophenyl)-3-phenyl-propanoate

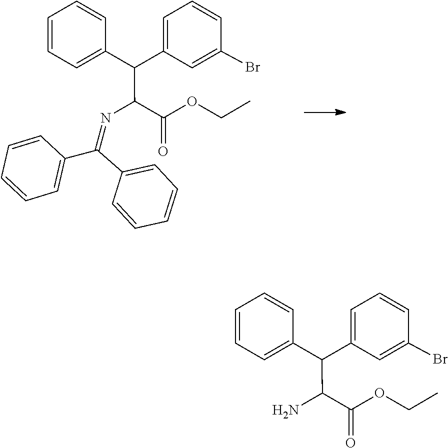 [SMILES: CCOC(=O)C(N=C(c1ccccc1)c1ccccc1)C(c1ccccc1)c1cccc(Br)c1 ; CCOC(=O)C(N)C(c1ccccc1)c1cccc(Br)c1]

Hydrogen chloride (aq. 6N, 100 mL) was added to a solution of the compound from Preparation 79 (20.0 g, 39.0 mmol) in DCM (100 mL) at 0° C. The reaction mixture was stirred at room temperature o/n. The aqueous layer was separated, basified with saturated aq. NaHCO₃ solution (50 mL) and extracted with EtOAc (3×300 mL). The combined organic phases were washed with brine (300 mL) then dried over Na₂SO₄, filtered and concentrated in vacuo to afford the crude title compound (mixture of diastereomers) as a colourless liquid. (13.4 g, assume quantitative yield); LCMS (Method 5) (ES): m/z 347 [M+H]⁺, RT=1.52 min.

### Preparation 81

### ethyl 3-(3-bromophenyl)-2-(tert-butoxycarbonylamino)-3-phenyl-propanoate

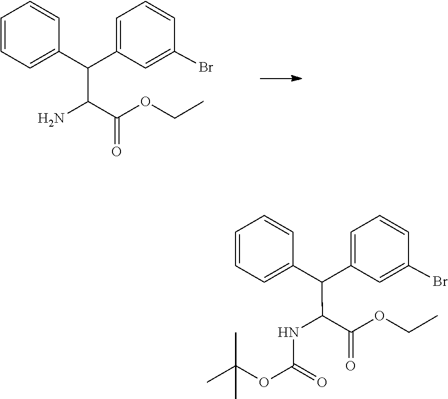 [SMILES: CCOC(=O)C(N)C(c1ccccc1)c1cccc(Br)c1 ; CCOC(=O)C(NC(=O)OC(C)(C)C)C(c1ccccc1)c1cccc(Br)c1]

tert-butoxycarbonyl tert-butyl carbonate (12.2 g, 56.0 mmol) was added to a stirring mixture of the compound from Preparation 80 (13.0 g, 37.4 mmol) in THF (150 mL) and saturated aq. NaHCO₃ (50 mL) portionwise at 0° C. The reaction mixture was stirred to room temperature o/n. The reaction mixture was quenched with ice cold H₂O (100 mL) and extracted with EtOAc (2×250 mL). The combined organic phases were washed with brine (200 mL) then dried over Na₂SO₄, filtered and concentrated in vacuo to give crude product. The obtained crude compound was purified by silica column chromatography (100-200 mesh), eluting with 10-30% EtOAc in hexane, to afford title compound (mixture of diastereomers) as a colourless liquid. (15.0 g, 89% yield); LCMS (Method 5) (ES): m/z 447 [M+H]⁺, RT=2.40 min.

### Preparation 82

### 3-(3-bromophenyl)-2-(tert-butoxycarbonylamino)-3-phenyl-propanoic Acid

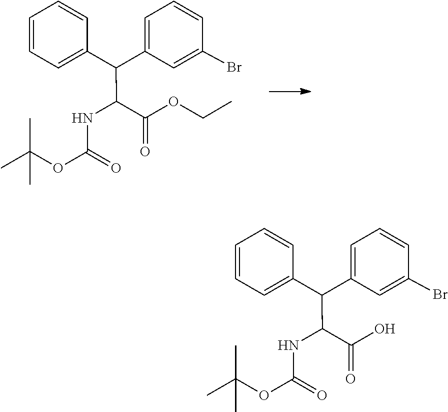 [SMILES: CCOC(=O)C(NC(=O)OC(C)(C)C)C(c1ccccc1)c1cccc(Br)c1 ; CC(C)(C)OC(=O)NC(C(=O)O)C(c1ccccc1)c1cccc(Br)c1]

LiOH.H₂O (7.02 g, 167.4 mmol) was added to a solution of the compound from Preparation 81 (15.0 g, 33.5 mmol) in THF (100 mL) and H₂O (20 mL) at room temperature. The reaction mixture was stirred o/n. The reaction mixture was concentrated in vacuo to low volume and acidified with 1N HCl aq. solution. The precipitate was collected by filtration, washed with H₂O (50 mL) then dried under vacuum to afford title compound (mixture of diastereomers) as an off-white solid. (12.0 g, 85% yield); LCMS (Method 5) (ES): m/z 420 [M+H]⁺, RT=2.50 min.

### Preparation 83

### 2-(tert-butoxycarbonylamino)-3-[3-(1-isopropyl-6-oxo-3-pyridyl)phenyl]-3-phenyl-propanoic Acid

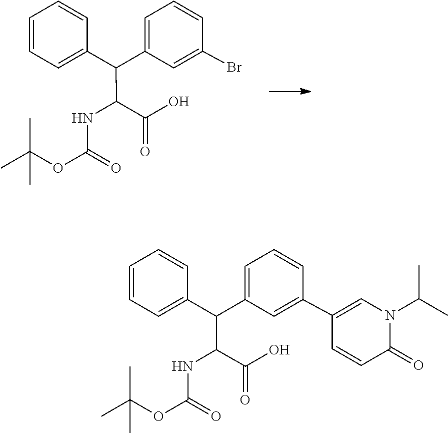 [SMILES: CC(C)(C)OC(=O)NC(C(=O)O)C(c1ccccc1)c1cccc(Br)c1 ; CC(C)n1cc(-c2cccc(C(c3ccccc3)C(NC(=O)OC(C)(C)C)C(=O)O)c2)ccc1=O]

1-isopropyl-5-(4,4,5,5-tetramethyl-1,3,2-dioxaborolan-2-yl)pyridin-2-one (11.3 g, 42.9 mmol) was added to a solution of the compound from Preparation 82 (12.0 g, 28.6 mmol) in 1,4-Dioxane (100 mL). A solution of Na₂CO₃ (9.07 g, 85.7 mmol) in H₂O (20 mL) was added and the reaction mixture was degassed with argon for 10 min. PdCl₂(dppf).DCM (2.32 g, 2.85 mmol) was added and the reaction mixture was stirred at 100° C. o/n. The reaction mixture was filtered through Celite pad and washed with EtOAc and H₂O. The filtrate was collected, aqueous layer separated and acidified to pH2 with hydrogen chloride (4N aq. solution), and extracted with EtOAc (2×250 mL). The combined organic phases were washed with brine (100 mL) then dried over Na₂SO₄, filtered and concentrated in vacuo to afford the title compound (mixture of diastereomers) as a brown solid. (10.0 g, 73% yield); LCMS (Method 5) (ES): m/z 476.6 [M+H]⁺, RT=2.3 min.

### Preparation 84

### tert-butyl N-[1-[(4-iodophenyl)carbamoyl]-2-[3-(1-isopropyl-6-oxo-3-pyridyl)phenyl]-2-phenyl-ethyl]carbamate

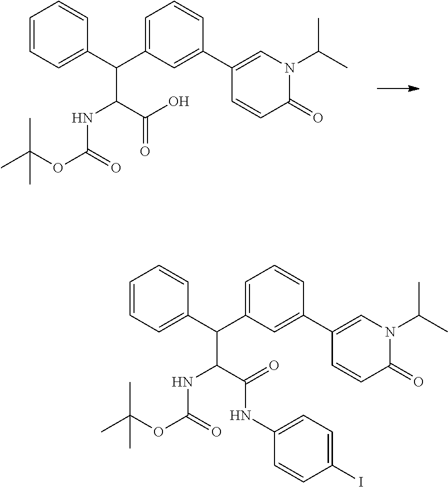

T₃P (16.6 g, 52.5 mmol, 50% in EtOAc) was added to a solution of the compound from Preparation 83 (10.0 g, 21.0 mmol), 4-iodoaniline (4.5 g, 21.0 mmol) and pyridine (4.1 g, 52.5 mmol) in DMF (30 mL) at 0° C. The reaction mixture was stirred o/n. The reaction mixture was quenched with ice cold H₂O (50 mL) with stirring. The solid was collected by filtration and dried under vacuum to afford title compound (mixture of diastereomers) as a brown solid. (9.0 g, 63% yield); LCMS (Method 5) (ES): m/z 677 [M+H]⁺, RT=2.7 min.

### Preparation 85

### 2-amino-N-(4-iodophenyl)-3-[3-(1-isopropyl-6-oxo-3-pyridyl)phenyl]-3-phenyl-propanamide

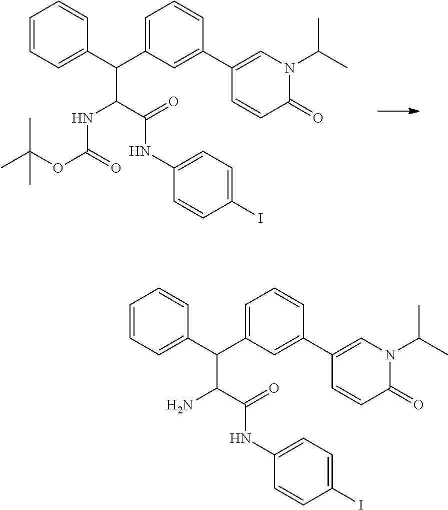 [SMILES: CC(C)n1cc(-c2cccc(C(c3ccccc3)C(NC(=O)OC(C)(C)C)C(=O)Nc3ccc(I)cc3)c2)ccc1=O ; CC(C)n1cc(-c2cccc(C(c3ccccc3)C(N)C(=O)Nc3ccc(I)cc3)c2)ccc1=O]

Hydrogen chloride (4N in 1,4-Dioxane, 20 mL) was added to a solution of the compound from Preparation 84 (9.0 g, 13.3 mmol) in 1,4-Dioxane (20 mL) at 0° C. The reaction mixture was stirred at room temperature o/n. The reaction mixture was concentrated in vacuo. The crude product was partitioned between saturated aq. NaHCO₃ solution (50 mL) and EtOAc (100 mL). The aqueous phase was further extracted with EtOAc (2×100 mL). The combined organic phases were washed with brine (100 mL) then dried over Na₂SO₄, filtered and concentrated in vacuo to afford the crude title compound (mixture of diastereomers) as a brown solid. (5.0 g, 65% yield); LCMS (Method 5) (ES): m/z 577 [M+H]⁺, RT=3.61 and 3.72 min.

### Preparation 86

### N-[1-[(4-iodophenyl)carbamoyl]-2-[3-(1-isopropyl-6-oxo-3-pyridyl)phenyl]-2-phenyl-ethyl]-2-methyl-pyrazole-3-carboxamide

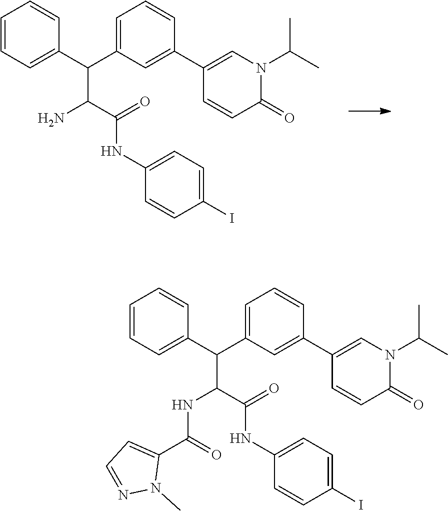 [SMILES: CC(C)n1cc(-c2cccc(C(c3ccccc3)C(N)C(=O)Nc3ccc(I)cc3)c2)ccc1=O ; CC(C)n1cc(-c2cccc(C(c3ccccc3)C(NC(=O)c3ccnn3C)C(=O)Nc3ccc(I)cc3)c2)ccc1=O]

HATU (237 mg, 0.624 mmol) in DMF (1 mL) was added to a mixture of the compound from Preparation 85 (250 mg, 0.41 mmol) and 2-methylpyrazole-3-carboxylic acid (52.0 mg, 0.41 mmol) and DIPEA (160 mg, 1.25 mmol) in DMF (5 mL) at 0° C. The reaction mixture was stirred at room temperature o/n. The reaction mixture was quenched with ice cold H₂O (5 mL) with stirring. The solid was collected by filtration and dried under vacuum to afford title compound (mixture of diastereomers) as a brown solid. (250 mg); LCMS (Method 5) (ES): m/z 685 [M+H]⁺, RT=2.0 and 2.1 min.

### Preparation 87

### Cyclopropyl N-[1-[(4-iodophenyl)carbamoyl]-2-[3-(1-isopropyl-6-oxo-3-pyridyl)phenyl]-2-phenyl-ethyl]carbamate

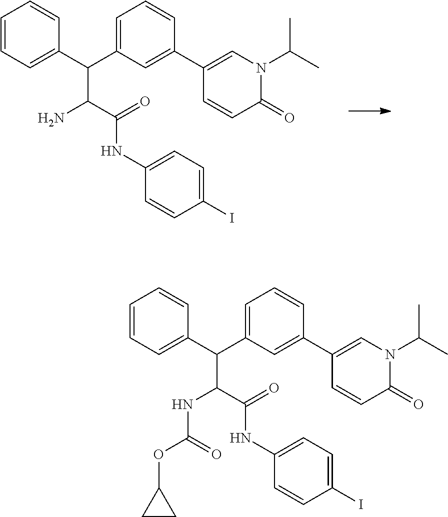 [SMILES: CC(C)n1cc(-c2cccc(C(c3ccccc3)C(N)C(=O)Nc3ccc(I)cc3)c2)ccc1=O ; CC(C)n1cc(-c2cccc(C(c3ccccc3)C(NC(=O)OC3CC3)C(=O)Nc3ccc(I)cc3)c2)ccc1=O]

(4-nitrophenyl) carbonochloridate (694 mg, 3.44 mmol) was added to a solution of cyclopropanol (200 mg, 3.44 mmol) and TEA (1.49 mL, 10.3 mmol) in DCM (2 mL) at 0° C. The reaction mixture was stirred to room temperature over 2 h. The reaction mixture was concentrated in vacuo to afford crude cyclopropyl (4-nitrophenyl) carbonate (770 mg) that was used directly without purification. The crude cyclopropyl (4-nitrophenyl) carbonate (200 mg, 0.89 mmol) was added to a solution of the compound from Preparation 85 (660 mg, 1.07 mmol) and DIPEA (0.5 mL, 2.69 mmol) in THF (5 mL) at 0° C. The reaction mixture was stirred at room temperature for 36 h. The reaction mixture was quenched with ice cold H₂O (5 mL) and extracted with EtOAc (3×10 mL). The combined organic phases were dried over Na₂SO₄, filtered and concentrated in vacuo to afford crude compound. The obtained crude compound was purified by silica column chromatography (100-200 mesh), eluting with 100% EtOAc to 5% MeOH/DCM, to afford title compound (mixture of diastereomers) as a colourless liquid. (400 Mg, 56% yield); LCMS (Method 5) (ES): m/z 661 [M+H]⁺, RT=2.10 and 2.17 min.

### Preparation 88

### (3-Bromophenyl)(cyclopropyl)methanol

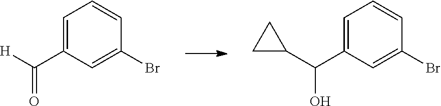 [SMILES: O=Cc1cccc(Br)c1 ; OC(c1cccc(Br)c1)C1CC1]

To a stirred solution of 3-bromobenzaldehyde (60 g, 324 mmol) in THF (300 mL) was added cyclopropyl magnesium bromide (843 ml, 421 mmol) at −5° C. The resulting reaction mixture was stirred at room temperature for 1 hour then quenched with saturated aq. ammonium chloride solution and extracted with EtOAc (2×200 mL). Th combined organic layers were washed with water (2×200 mL), brine (200 mL), dried over anhydrous Na₂SO₄ and concentrated under reduced pressure. The crude compound was purified by silica gel (100-200 mesh) column chromatography (5%-10% EtOAc in Hexane as eluent) to afford the title compound (38 g, 51%) as an oil. 1H NMR (400 MHz, CHLOROFORM-d) δ 7.60 (t, J=1.74 Hz, 1H) 7.40-7.44 (m, 1H), 7.35 (d, J=7.74 Hz, 1H), 7.19-7.25 (m, 1H), 3.98 (dd, J=8.34, 3.00 Hz, 1H), 1.96 (d, J=3.05 Hz, 1H), 1.10-1.24 (m, 1H), 0.53-0.72 (m, 2H), 0.44-0.51 (m, 1H), 0.35-0.43 (m, 1H); LCMS (METHOD 5) (ESI): m/z 228 [M+H⁺]; RT=1.9 min; (ACQUITY UPLC BEH C18 column, 0.1% FA in water with MeCN).

### Preparation 89

### (3-Bromophenyl)(cyclopropyl)methanone

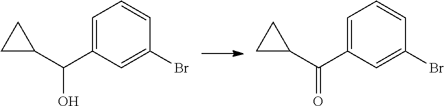 [SMILES: OC(c1cccc(Br)c1)C1CC1 ; O=C(c1cccc(Br)c1)C1CC1]

To a stirred solution of the alcohol of Preparation 88 (38 g, 168 mmol) in DCM (300 mL) was added Dess-Martin Periodinane (179 g, 422 mmol) at room temperature. The resulting reaction mixture was stirred at room temperature for 2 hours then diluted with saturated aq. sodium bicarbonate solution (250 mL) and 10% aq. sodium thiosulphate solution (250 mL). The aqueous layer was extracted with DCM (200 mL), the organic layer was washed with water (2×200 mL), brine (200 mL), dried over anhydrous Na₂SO₄ and concentrated under reduced pressure. The crude compound was purified by silica gel (100-200 mesh) column chromatography (2%-3% EtOAc in Hexane as an eluent) to afford the title compound (33 g, 87%) as an oil. 1H NMR (400 MHz, CHLOROFORM-d) δ 8.14 (t, J=1.74 Hz, 1H), 7.93 (dt, J=7.74, 1.31 Hz, 1H), 7.67-7.71 (m, 1H), 7.36 (t, J=7.85 Hz, 1H), 2.62 (tt, J=7.82, 4.50 Hz, 1H), 1.26 (quin, J=3.84 Hz, 2H), 1.08 (dq, J=7.44, 3.59 Hz, 2H); LCMS (METHOD 5) (ESI): m/z: 225 [M+H⁺]; RT=2.13 min; (ACQUITY UPLC BEH C18 column, 0.1% FA in water with MeCN).

### Preparation 90

### (Z)-1-Bromo-3-(1-cyclopropyl-2-methoxyvinyl) benzene

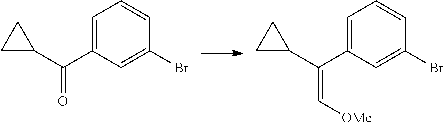 [SMILES: O=C(c1cccc(Br)c1)C1CC1 ; CO/C=C(\c1cccc(Br)c1)C1CC1]

To a stirred solution of (methoxymethyl)triphenylphosphonium chloride (126.5 g, 370 mmol) in THF (300 mL) were added tBuOK (36.5 g, 325.5 mmol) and DMSO (25 g, 320.5 mmol) at 0° C. The reaction mixture was stirred at 0° C. for 30 min then the ketone of Preparation 89 was added. The reaction was stirred for 16 hours at room temperature then diluted with of water (300 mL), extracted with EtOAc (2×300 mL). The combined organic layers were washed with water (2×200 mL) and brine (200 mL), dried over anhydrous Na₂SO₄ and concentrated under reduced pressure. The crude product was purified by silica gel (100-200 mesh) column chromatography (1%-2% EtOAc in Hexane as an eluent) to afford the title compound (30 g, 80%) as a viscous oil. 1H NMR (400 MHz, CHLOROFORM-d) δ 7.92, 7.44 (t, J=1.71 Hz, 1H), 7.70 (d, J=7.83 Hz, 1H), 7.28-7.36 (m, 1H), 7.09-7.25 (m, 1H), 6.27, 6.19 (d, J=0.98 Hz, J=1.47 Hz, 1H), 3.70, 3.71 (s, 3H), 1.59-1.68, 1.45-1.53 (m, 1H), 0.69-0.87 (m, 2H), 0.36-0.49 (m, 2H); LCMS (METHOD 5) (ESI): m/z 254 [M+H⁺]; RT=2.46 min; (ACQUITY UPLC BEH C18 column, 0.1% FA in water with MeCN).

### Preparation 91

### 2-(3-Bromophenyl)-2-cyclopropylacetaldehyde

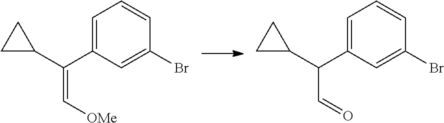 [SMILES: CO/C=C(\c1cccc(Br)c1)C1CC1 ; O=CC(c1cccc(Br)c1)C1CC1]

To a stirred solution of the compound of Preparation 90 (30 g, 118.5 mmol) in THF (150 mL) was added 5M HCl (90 ml) at room temperature. The resulting reaction mixture was stirred at room temperature for 48 hours then diluted with hexane (300 mL) The layers were separated and the aq. layer was extracted with hexane (300 mL). The combined organic layers were washed with water (2×200 mL) and brine (200 mL), dried over anhydrous Na₂SO₄ and concentrated under reduced pressure. The crude product was purified by silica gel (100-200 mesh) column chromatography (1% EtOAc in hexane as an eluent) to afford the title compound (25 g, yield 88%) as an oil. 1H NMR (400 MHz, CHLOROFORM-d) δ 9.74 (d, J=2.40 Hz, 1H), 7.42-7.47 (m, 2H), 7.23-7.28 (m, 1H), 7.16-7.20 (m, 1H), 2.76 (dd, J=9.81, 2.29 Hz, 1H), 1.23-1.32 (m, 1H), 0.74-0.81 (m, 1H), 0.65 (dddd, J=9.13, 8.12, 5.69, 4.85 Hz, 1H), 0.41 (dq, J=10.25, 4.80 Hz, 1H), 0.19-0.27 (m, 1H).

### Preparation 92

### 5-((3-Bromophenyl)(cyclopropyl)methyl)imidazolidine-2,4-dione

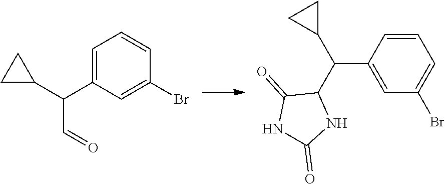 [SMILES: O=CC(c1cccc(Br)c1)C1CC1 ; O=C1NC(=O)C(C(c2cccc(Br)c2)C2CC2)N1]

To a stirred solution of the aldehyde of Preparation 91 (25 g, 105 mmol) in MeOH (500 mL) and water (140 mL) was added KCN (10.3 g, 157.5 mmol) and (NH₄)₂CO₃ (30.3 g, 315 mmol) at room temperature. The resulting reaction mixture was stirred at 65° C. for 16 hours in an autoclave. After cooling to room temperature, the reaction was concentrated under reduced pressure, water (250 mL) was added and the mixture was extracted with EtOAc (2×250 mL). The combined organic layers were washed with water (2×200 mL) and brine (200 mL), dried over anhydrous Na₂SO₄ and concentrated under reduced pressure to afford the title compound (14 g, 43%) as a mixture of diastereomers as an off white solid. 1H NMR (400 MHz, DMSO-d6) δ 10.38 (s, 1H), 8.36 (s, 1H), 7.40-7.45 (m, 2H), 7.18-7.30 (m, 2H), 4.35 (d, J=2.45 Hz, 1H), 2.20-2.27 (m, 1H), 1.33-1.45 (m, 1H), 0.53-0.63 (m, 1H), 0.42-0.51 (m, 1H), 0.36 (dq, J=9.11, 4.63 Hz, 1H), 0.05-0.14 (m, 1H); LCMS (METHOD 5) (ESI): m/z: 310 [M+H⁺]; RT=2.08+2.12 min (3;1 ratio); (ACQUITY UPLC BEH C18 column, 0.05% FA in water with MeCN).

### Preparations 93A-D

### Diastereomers 1 to 4 of 3-(3-bromophenyl)-2-((tert-butoxycarbonyl)amino)-3-cyclopropylpropanoic Acid

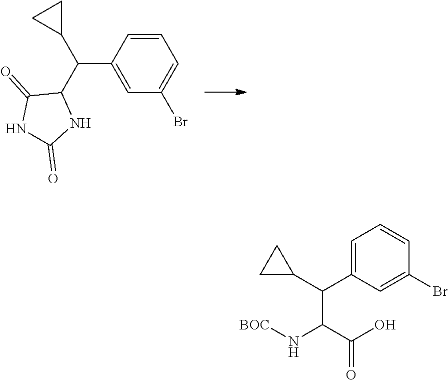 [SMILES: O=C1NC(=O)C(C(c2cccc(Br)c2)C2CC2)N1 ; CC(C)(C)OC(=O)NC(C(=O)O)C(c1cccc(Br)c1)C1CC1]

The dione of Preparation 92 (14 g, 45.3 mmol) was taken up in aq. NaOH (21 g in 140 mL of H₂O, 525 mmol). The resulting reaction mixture was stirred at 120° C. for 16 hours then cooled to 0° C., diluted with 1,4-dioxane (150 mL) and (Boc)₂O (96.1 g, 419 mmol) was added. The resulting reaction mixture was stirred at room temperature for 6 hours. then cooled to 0° C. and acidified to pH 3 with 5M HCl. The reaction mixture was extracted with EtOAc (3×150 mL), the combined organic layers were washed with water (200 mL) and brine (200 mL), dried over anhydrous Na₂SO₄, filtered and concentrated under reduced pressure. The crude product was purified by silica gel (100-200 mesh) column chromatography (2% to 3% of EtOAc in hexane as an eluent) to give the title compound (13 g, 74%) as a mixture of diastereomers.

The isomers were separated by chiral SFC. First preparative SFC Conditions: Column/dimensions: Chiralpak IG (30×250 mm), 5 u; % CO₂: 85%; % Co solvent: 15% (0.5 DEA in EtOH); Total flow: 90.0 g/min; Back pressure: 100 bar; UV: 214 nm. Second preparative SFC Condition: Column/dimensions: Chiralpak IG (30×250 mm), 5 u; % CO₂: 75%; % Co solvent: 25.0% (0.5% isopropylamine in IPA); Total flow: 90.0 g/min; Back pressure: 120.0 bar; UV: 214 nm;

Diastereomer 1 (Prep. 130A): 1H NMR (400 MHz, DMSO-d6) δ 12.60 (s, 1H), 7.41-7.43 (m, 1H), 7.33-7.37 (m, 1H), 7.19-7.28 (m, 2H), 6.02-6.16 (m, 1H), 4.27 (t, J=8.40 Hz, 1H), 2.24 (dd, J=9.72, 8.40 Hz, 1H), 1.2-1.26 (m, 1H), 1.30 (s, 9H), 0.51-0.62 (m, 1H), 0.34-0.41 (m, 1H), 0.24-0.31 (m, 1H), −0.06-−0.01 (m, 1H); LCMS (METHOD 5) (ESI): m/z: 383 [M−H]; RT=2.92 min; (ACQUITY UPLC BEH C18 column, 0.05% FA in water with MeCN). Chiral HPLC: Column: CHIRALPAK IG (4.6×250)mm, 5 u, Co-Solvent: 0.5% DEA in EtOH (15%), Column Temp.: 30° C., Flow: 3 ml/min, RT: 3.46 (99%).

Diastereomer 2 (Prep. 130B): 1H NMR (400 MHz, DMSO-d6) δ 12.60 (s, 1H), 7.42-7.45 (m, 1H), 7.32-7.36 (m, 1H), 7.17-7.27 (m, 2H), 6.28-6.46 (m, 1H), 4.24-4.30 (m, 1H), 2.38 (dd, J=10.37, 5.36 Hz, 1H), 1.33 (s, 9H), 0.50-0.57 (m, 1H), 0.31-0.41 (m, 2H), −0.16-−0.08 (m, 1H); LCMS (METHOD 5) (ESI): m/z: 383 [M−H]; RT=2.91 min; (ACQUITY UPLC BEH C18 column, 0.05% FA in water with MeCN); Chiral HPLC: Column: CHIRALPAK IG (4.6×250)mm, 5 u, Co-Solvent: 0.5% Isopropyl amine in IPA (20%), Column Temp.: 30° C., Flow: 3 ml/min, RT: 4.1 (99%).

Diastereomer 3 (Prep. 130C): 1H NMR (400 MHz, DMSO-d6) δ 12.60 (s, 1H), 7.41-7.43 (m, 1H), 7.33-7.37 (m, 1H), 7.19-7.28 (m, 2H), 6.02-6.16 (m, 1H), 4.27 (t, J=8.40 Hz, 1H), 2.24 (dd, J=9.72, 8.40 Hz, 1H), 1.2-1.26 (m, 1H), 1.30 (s, 9H), 0.51-0.62 (m, 1H), 0.34-0.41 (m, 1H), 0.24-0.31 (m, 1H), −0.06-−0.01 (m, 1H); LCMS (METHOD 5) (ESI): m/z: 383 [M−H]; RT=2.89 min; (ACQUITY UPLC BEH C18 column, 0.05% FA in water with MeCN); Chiral HPLC: Column: CHIRALPAK IG (4.6×250)mm, 5 u, Co-Solvent: 0.5% DEA in Ethanol (15%), Column Temp.: 30° C., Flow: 3 ml/min, RT: 5.07 (99%).

Diastereomer 4 (Prep. 130D): 1H NMR (400 MHz, DMSO-d6) δ 12.60 (s, 1H), 7.42-7.45 (m, 1H), 7.32-7.36 (m, 1H), 7.17-7.27 (m, 2H), 6.28-6.46 (m, 1H), 4.24-4.30 (m, 1H), 2.38 (dd, J=10.37, 5.36 Hz, 1H), 1.33 (s, 9H), 0.50-0.57 (m, 1H), 0.31-0.41 (m, 2H), −0.16-−0.08 (m, 1H); LCMS (METHOD 5) (ESI): m/z: 383 [M−H]; RT=2.95 min; (ACQUITY UPLC BEH C18 column, 0.05% FA in water with MeCN); Chiral HPLC: Column: CHIRALPAK IG (4.6×250)mm, 5 u, Co-Solvent: 0.5% isopropylamine in IPA (20%), Column Temp.: 30° C., Flow: 3 ml/min, RT: 7.32 (99%).

### Example 1

### N-[(1S)-2-[4-(3,5-dimethyl-1H-pyrazol-4-yl)anilino]-1-[(1R)-6-isoindolin-5-ylindan-1-yl]-2-oxo-ethyl]-2-methyl-pyrazole-3-carboxamide

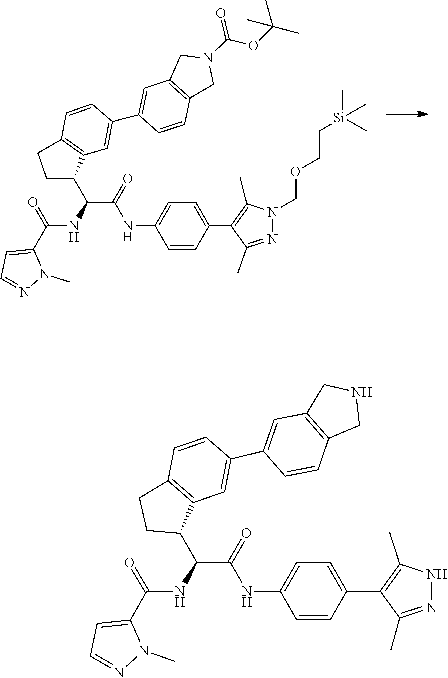 [SMILES: Cc1nn(COCC[Si](C)(C)C)c(C)c1-c1ccc(NC(=O)[C@@H](NC(=O)c2ccnn2C)[C@@H]2CCc3ccc(-c4ccc5c(c4)CN(C(=O)OC(C)(C)C)C5)cc32)cc1 ; Cc1n[nH]c(C)c1-c1ccc(NC(=O)[C@@H](NC(=O)c2ccnn2C)[C@@H]2CCc3ccc(-c4ccc5c(c4)CNC5)cc32)cc1]

TFA (3 mL) was added to a solution of the compound from Preparation 16 (20.0 mg, 0.025 mmol) in DCM (2 mL) at room temperature. The reaction mixture was stirred for 1.5 h. The reaction mixture was concentrated in vacuo, re-dissolved in MeOH (1.0 mL) and purified directly by basic HPLC to afford the title compound as a colourless solid. (8.0 mg, 55% yield); LCMS (METHOD 3) (ES): m/z 588.2 [M+H]⁺, RT=0.57 min. ¹H NMR (600 MHz, DMSO-d6) δ 10.24 (s, 1H), 8.79 (d, J=8.5 Hz, 1H), 7.69 (d, J=8.4 Hz, 2H), 7.46 (d, J=2.1 Hz, 1H), 7.40 (td, J=9.4, 7.8, 4.5 Hz, 1H), 7.36-7.29 (m, 2H), 7.23 (t, J=7.6 Hz, 2H), 7.16 (s, 1H), 7.13-7.07 (m, 2H), 7.04 (d, J=2.2 Hz, 1H), 4.89 (t, J=8.8 Hz, 1H), 4.52 (s, 1H), 4.00 (d, J=4.1 Hz, 3H), 3.97-3.86 (m, 2H), 3.83 (td, J=8.5, 5.1 Hz, 1H), 3.02 (dt, J=15.7, 7.8 Hz, 1H), 2.86 (ddd, J=16.0, 8.7, 5.2 Hz, 1H), 2.29-2.20 (m, 1H), 2.18 (s, 6H), 2.12-2.04 (m, 1H), 1.99 (dt, J=21.6, 7.0 Hz, 1H), 1.24 (d, J=5.4 Hz, 3H).

### Example 2

### N-[(1S)-2-[4-(3,5-dimethyl-1H-pyrazol-4-yl)anilino]-1-[(1R)-6-[4-(morpholinomethyl)phenyl]indan-1-yl]-2-oxo-ethyl]-2-methyl-pyrazole-3-carboxamide

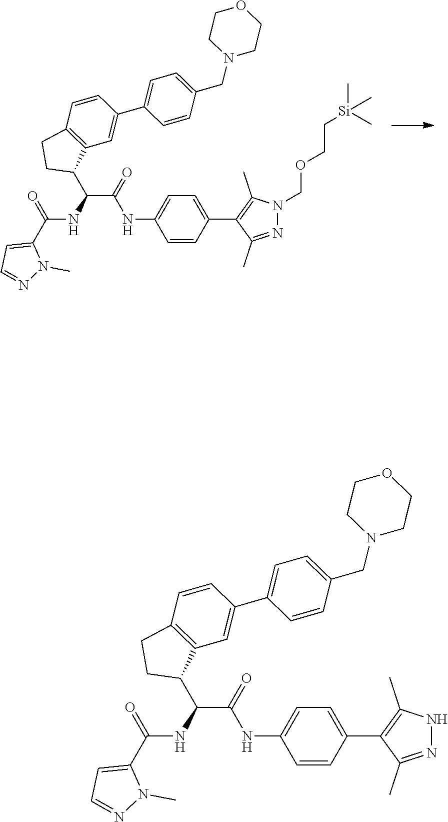 [SMILES: Cc1nn(COCC[Si](C)(C)C)c(C)c1-c1ccc(NC(=O)[C@@H](NC(=O)c2ccnn2C)[C@@H]2CCc3ccc(-c4ccc(CN5CCOCC5)cc4)cc32)cc1 ; Cc1n[nH]c(C)c1-c1ccc(NC(=O)[C@@H](NC(=O)c2ccnn2C)[C@@H]2CCc3ccc(-c4ccc(CN5CCOCC5)cc4)cc32)cc1]

TFA (2 mL) was added to a solution of the compound from Preparation 19 (17.0 mg, 0.002 mmol) in DCM (2 mL) at room temperature. The reaction mixture was stirred for 30 min. The reaction mixture was concentrated in vacuo, re-dissolved in MeOH (0.5 mL) and purified directly by acidic HPLC to afford impure product as a yellow solid (17.0 mg). 2M acetic acid in MeOH was added to a DMSO solution of the impure product and passed through 1 g SCX cartridge eluting with MeOH, then with 2N NH₃ in MeOH. The relevant fractions were combined and concentrated in vacuo to afford title compound as a colourless solid. (10.0 mg, 71% yield); LCMS (METHOD 3) (ES): m/z 644.3 [M+H]⁺, RT=0.68 min. ¹H NMR (300 MHz, DMSO-d6) δ 12.23 (bs, 1H), 10.18 (s, 1H), 8.77 (d, J=8.4 Hz, 1H), 7.74-7.60 (m, 2H), 7.46 (d, J=2.0 Hz, 1H), 7.43-7.29 (m, 3H), 7.26-7.13 (m, 7H), 7.03 (d, J=2.1 Hz, 1H), 4.88 (t, J=8.8 Hz, 1H), 4.00 (s, 3H), 3.81 (p, J=8.0 Hz, 1H), 3.54 (t, J=4.5 Hz, 4H), 3.43 (s, 2H), 3.03 (dt, J=16.0, 7.9 Hz, 1H), 2.95-2.78 (m, 1H), 2.35 (s, 4H), 2.19 (s, 6H).

### Example 3

### N-[(1S)-1-[(1R)-6-(1-isopropyl-6-oxo-3-pyridyl)indan-1-yl]-2-[4-(3-methylimidazol-4-yl)anilino]-2-oxo-ethyl]-2-methyl-pyrazole-3-carboxamide

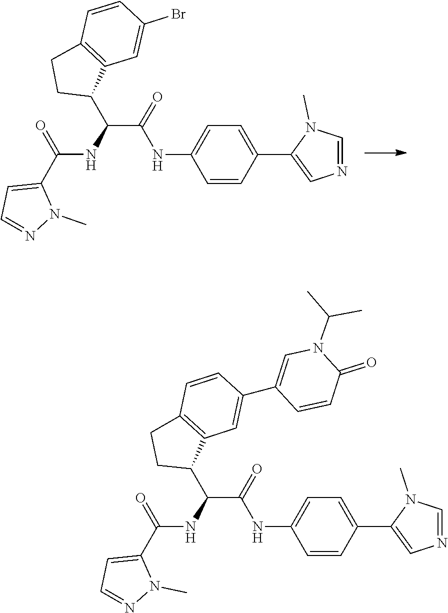 [SMILES: Cn1cncc1-c1ccc(NC(=O)[C@@H](NC(=O)c2ccnn2C)[C@@H]2CCc3ccc(Br)cc32)cc1 ; CC(C)n1cc(-c2ccc3c(c2)[C@H]([C@H](NC(=O)c2ccnn2C)C(=O)Nc2ccc(-c4cncn4C)cc2)CC3)ccc1=O]

1-Isopropyl-5-(4,4,5,5-tetramethyl-1,3,2-dioxaborolan-2-yl)pyridin-2-one (9.87 mg, 0.034 mmol) was added to a solution of the compound from Preparation 21 (10.0 mg, 0.019 mmol) in DMF (0.1 mL) in a 5 mL MW vial. A solution of K₂CO₃ (7.77 mg, 0.056 mmol) in H₂O (0.02 mL) was added and the reaction mixture was degassed with argon for 10 min. PdCl₂(dppf).DCM (0.74 mg, 0.001 mmol) was added and the vial was capped and stirred for 30 min at 80° C. The reaction mixture was passed through a PTFE filter and purified by acidic HPLC directly to afford the title compound as an off-white solid. (4.1 mg, 36% yield); LCMS (METHOD 3) (ES): m/z 590.6 [M+H]⁺, RT=0.56 min. ¹H NMR (300 MHz, DMSO-d6) δ 10.23 (s, 1H), 8.75 (d, J=8.5 Hz, 1H), 7.73-7.57 (m, 4H), 7.46-7.42 (m, 2H), 7.42-7.33 (m, 2H), 7.32-7.24 (m, 2H), 7.21 (s, 1H), 6.98 (dd, J=6.2, 1.5 Hz, 2H), 6.26 (d, J=9.4 Hz, 1H), 5.12-4.88 (m, 1H), 3.98 (s, 3H), 3.81 (d, J=39.3 Hz, 1H), 3.64 (s, 3H), 3.00 (dt, J=15.6, 7.7 Hz, 1H), 2.93-2.76 (m, 1H), 2.30-1.96 (m, 2H), 1.35 (d, J=6.8 Hz, 1H), 1.25 (d, J=6.8 Hz, 3H), 1.20 (d, J=6.8 Hz, 3H).

### Example 26

### N-[(1S)-1-[(1R)-7-(1-cyclopropylpyrazol-4-yl)tetralin-1-yl]-2-[4-(3,5-dimethyl-1H-pyrazol-4-yl)anilino]-2-oxo-ethyl]-2-methyl-pyrazole-3-carboxamide

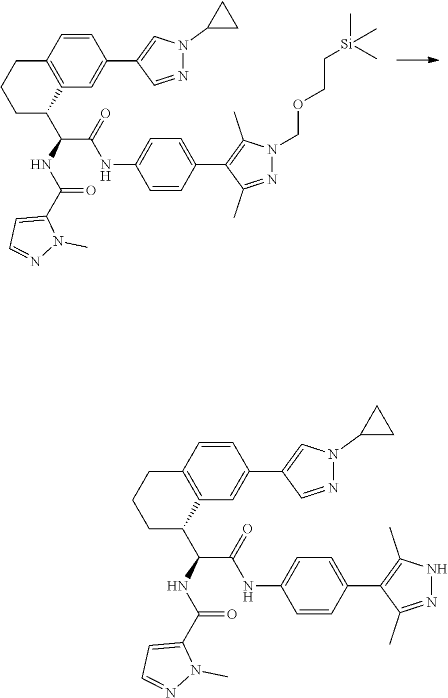 [SMILES: Cc1nn(COCC[Si](C)(C)C)c(C)c1-c1ccc(NC(=O)[C@@H](NC(=O)c2ccnn2C)[C@@H]2CCCc3ccc(-c4cnn(C5CC5)c4)cc32)cc1 ; Cc1n[nH]c(C)c1-c1ccc(NC(=O)[C@@H](NC(=O)c2ccnn2C)[C@@H]2CCCc3ccc(-c4cnn(C5CC5)c4)cc32)cc1]

According to the method of Example 1 the compound of Preparation 29 (11.0 mg, 0.015 mmol) was reacted to give the title compound as a colourless solid. (2.4 mg, 27% yield); ¹H NMR (300 MHz, DMSO-d6) δ 10.00 (s, 1H), 8.61 (d, J=9.0 Hz, 1H), 7.77 (d, J=0.7 Hz, 1H), 7.54 (d, J=8.5 Hz, 2H), 7.49-7.41 (m, 2H), 7.28 (s, 1H), 7.26-7.21 (m, 1H), 7.15 (s, 1H), 7.12 (s, 1H), 7.05 (d, J=7.9 Hz, 1H), 6.99 (d, J=2.1 Hz, 1H), 5.13 (t, J=8.9 Hz, 1H), 3.96 (s, 3H), 3.60 (ddd, J=11.5, 7.0, 4.4 Hz, 2H), 3.44 (s, 3H), 2.14 (s, 6H), 1.98 (s, 2H), 1.68 (s, 2H), 0.97-0.84 (m, 4H); LCMS (METHOD 1) (ES): m/z 589.305 [M+H]⁺, RT=2.17 min.

### Example 27

### N-[(1S)-2-[4-(3,5-dimethyl-1H-pyrazol-4-yl)anilino]-1-[(1R)-7-(1-isopropyl-6-oxo-3-pyridyl)tetralin-1-yl]-2-oxo-ethyl]-1-fluoro-cyclopropanecarboxamide

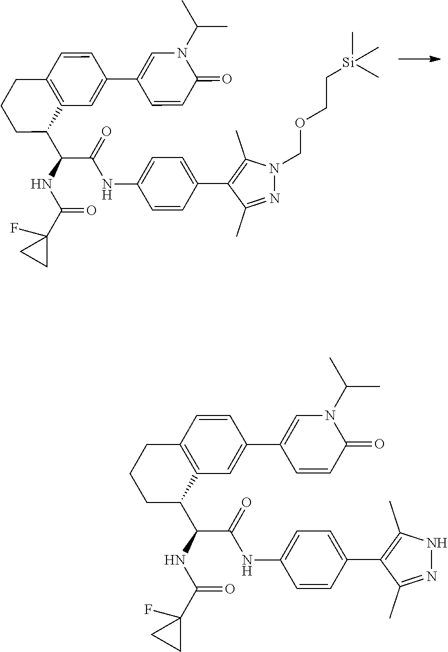 [SMILES: Cc1nn(COCC[Si](C)(C)C)c(C)c1-c1ccc(NC(=O)[C@@H](NC(=O)C2(F)CC2)[C@@H]2CCCc3ccc(-c4ccc(=O)n(C(C)C)c4)cc32)cc1 ; Cc1n[nH]c(C)c1-c1ccc(NC(=O)[C@@H](NC(=O)C2(F)CC2)[C@@H]2CCCc3ccc(-c4ccc(=O)n(C(C)C)c4)cc32)cc1]

According to the method of Example 1 the compound of Preparation 33 (24.0 mg, 0.033 mmol) was reacted to give the title compound after basic HPLC as a colourless solid. (13.7 mg, 69% yield); LCMS (METHOD 3) (ES): m/z 596.6 [M+H]⁺, RT=0.72 min; ¹H NMR (600 MHz, DMSO-d6) δ 12.18 (s, 1H), 10.00 (s, 1H), 8.18 (dd, J=9.4, 1.9 Hz, 1H), 7.71 (d, J=2.6 Hz, 1H), 7.55-7.41 (m, 2H), 7.36 (dd, J=9.4, 2.6 Hz, 1H), 7.30 (dd, J=7.9, 2.0 Hz, 1H), 7.27 (d, J=2.0 Hz, 1H), 7.14 (t, J=8.5 Hz, 3H), 6.28 (d, J=9.4 Hz, 1H), 5.15 (t, J=8.8 Hz, 1H), 5.03 (hept, J=6.8 Hz, 1H), 3.47 (dt, J=8.3, 5.7 Hz, 1H), 2.84 (dt, J=17.1, 6.1 Hz, 1H), 2.73 (dt, J=16.9, 6.9 Hz, 1H), 2.13 (s, 6H), 1.93 (ddt, J=12.8, 9.6, 4.9 Hz, 2H), 1.69 (tt, J=12.7, 6.0 Hz, 2H), 1.41-1.31 (m, 2H), 1.30 (d, J=6.8 Hz, 3H), 1.24 (d, J=6.8 Hz, 3H), 1.22-1.15 (m, 1H), 1.14-1.05 (m, 1H).

### Example 28

### N-[(1S)-2-[4-(3,5-dimethyl-1H-pyrazol-4-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]-1-fluoro-cyclopropanecarboxamide

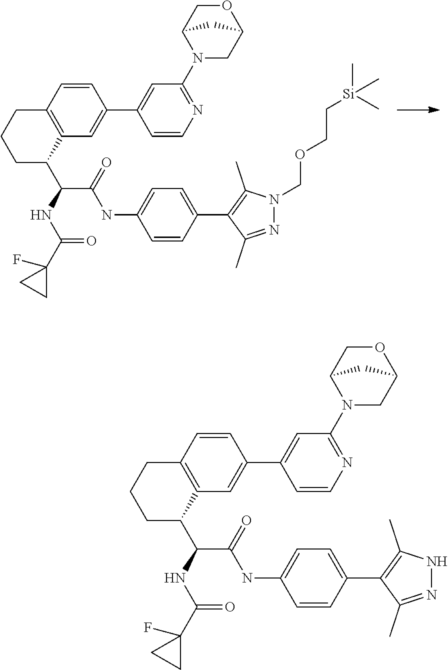 [SMILES: Cc1nn(COCC[Si](C)(C)C)c(C)c1-c1ccc(NC(=O)[C@@H](NC(=O)C2(F)CC2)[C@@H]2CCCc3ccc(-c4ccnc(N5C[C@H]6C[C@@H]5CO6)c4)cc32)cc1 ; Cc1n[nH]c(C)c1-c1ccc(NC(=O)[C@@H](NC(=O)C2(F)CC2)[C@@H]2CCCc3ccc(-c4ccnc(N5C[C@H]6C[C@@H]5CO6)c4)cc32)cc1]

According to the method of Example 1 the compound of Preparation 39 (24.0 mg, 0.033 mmol) was reacted to give the title compound after basic HPLC as a colourless solid. (4.5 mg, 67% yield); LCMS (METHOD 3) (ES): m/z 636.5 [M+H]⁺, RT=0.62 min; ¹H NMR (600 MHz, DMSO-d6) δ 12.21 (s, 1H), 9.99 (s, 1H), 8.17 (dd, J=9.4, 1.9 Hz, 1H), 7.93 (d, J=5.3 Hz, 1H), 7.51-7.40 (m, 4H), 7.19 (d, J=7.8 Hz, 1H), 7.17-7.11 (m, 2H), 6.62-6.50 (m, 2H), 5.17 (t, J=8.8 Hz, 1H), 4.83 (s, 1H), 4.68-4.59 (m, 1H), 3.75-3.72 (m, 1H), 3.60 (d, J=7.3 Hz, 1H), 3.53-3.47 (m, 1H), 3.44-3.38 (m, 1H), 3.22 (d, J=10.0 Hz, 1H), 2.92-2.84 (m, 1H), 2.80-2.72 (m, 1H), 2.13 (s, 6H), 2.00-1.90 (m, 2H), 1.87-1.77 (m, 2H), 1.76-1.64 (m, 2H), 1.38-1.04 (m, 4H).

### Example 29

### N-[(1S)-2-[4-(3,5-dimethyl-1H-pyrazol-4-yl)anilino]-1-[(1R)-7-[2-[(1S,4S)-2-oxa-5-azabicyclo[2.2.1]heptan-5-yl]-4-pyridyl]tetralin-1-yl]-2-oxo-ethyl]-1-fluoro-cyclopropanecarboxamide

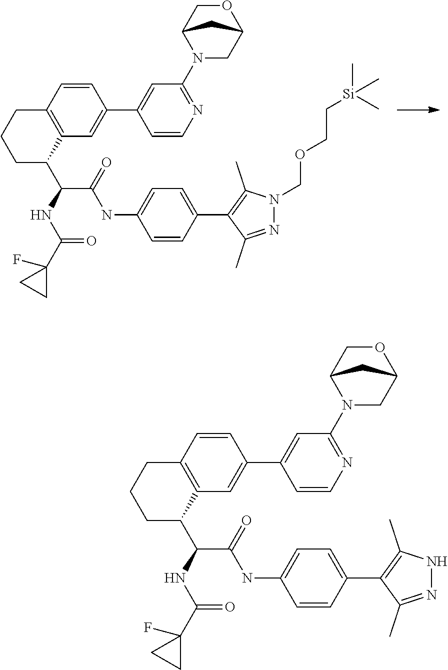 [SMILES: Cc1nn(COCC[Si](C)(C)C)c(C)c1-c1ccc(NC(=O)[C@@H](NC(=O)C2(F)CC2)[C@@H]2CCCc3ccc(-c4ccnc(N5C[C@@H]6C[C@H]5CO6)c4)cc32)cc1 ; Cc1n[nH]c(C)c1-c1ccc(NC(=O)[C@@H](NC(=O)C2(F)CC2)[C@@H]2CCCc3ccc(-c4ccnc(N5C[C@@H]6C[C@H]5CO6)c4)cc32)cc1]

According to the method of Example 1 the compound of Preparation 45 (30.0 mg, 0.039 mmol) was reacted to give the title compound after basic HPLC as a colourless solid. (21.0 mg, 84% yield); LCMS (METHOD 3) (ES): m/z 635.5 [M+H]⁺, RT=0.67 min; ¹H NMR (300 MHz, DMSO-d6) δ 9.97 (s, 1H), 8.20 (d, J=9.2 Hz, 1H), 7.94 (d, J=5.3 Hz, 1H), 7.45 (dt, J=6.8, 4.0 Hz, 4H), 7.19 (d, J=7.9 Hz, 1H), 7.11 (d, J=8.2 Hz, 2H), 6.59 (d, J=5.3 Hz, 1H), 6.52 (s, 1H), 5.12 (t, J=8.8 Hz, 1H), 4.81 (s, 1H), 4.61 (s, 1H), 3.74 (d, J=7.3 Hz, 1H), 3.60 (d, J=7.3 Hz, 1H), 3.19 (d, J=9.9 Hz, 2H), 3.00-2.64 (m, 2H), 2.13 (s, 6H), 2.04-1.87 (m, 2H), 1.79-1.60 (m, 2H), 1.39-1.05 (m, 4H).

### Example 51

### N-[(1S)-2-[2-chloro-5-(1-isopropyl-6-oxo-3-pyridyl)phenyl]-1-[[4-(3,5-dimethyl-1H-pyrazol-4-yl)phenyl]carbamoyl]propyl]-1-fluoro-cyclopropanecarboxamide

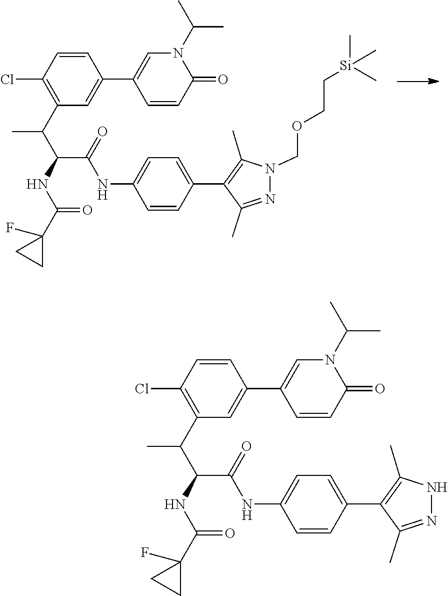 [SMILES: Cc1nn(COCC[Si](C)(C)C)c(C)c1-c1ccc(NC(=O)[C@@H](NC(=O)C2(F)CC2)C(C)c2cc(-c3ccc(=O)n(C(C)C)c3)ccc2Cl)cc1 ; Cc1n[nH]c(C)c1-c1ccc(NC(=O)[C@@H](NC(=O)C2(F)CC2)C(C)c2cc(-c3ccc(=O)n(C(C)C)c3)ccc2Cl)cc1]

According to the method of Example 1 the compound of Preparation 53 (85.0 mg, 0.116 mmol) was reacted to give the title compound after basic HPLC as a colourless solid. (49.0 mg, 70% yield); LCMS (METHOD 3) (ES): m/z 604.4 [M+H]⁺, RT=0.70 min; ¹H NMR (600 MHz, DMSO-d6) δ 12.19 (s, 1H), 10.08 (s, 1H), 8.76-8.61 (m, 1H), 7.90 (s, 2H), 7.66 (d, J=2.2 Hz, 1H), 7.51-7.35 (m, 4H), 7.22-7.05 (m, 2H), 6.50 (dd, J=9.0, 1.0 Hz, 1H), 5.08 (h, J=6.8 Hz, 1H), 5.01 (t, J=9.3 Hz, 1H), 4.03 (dq, J=9.8, 7.0 Hz, 1H), 2.13 (s, 6H), 1.35 (dd, J=13.8, 6.9 Hz, 8H), 1.29 (d, J=6.9 Hz, 3H), 1.21 (qdd, J=9.6, 7.4, 4.0 Hz, 2H).

### Example 56

### N-[(1S)-2-[3-(1-isopropyl-6-oxo-3-pyridyl)phenyl]-1-[[4-(3-methylimidazol-4-yl)phenyl]carbamoyl]propyl]-2-methyl-pyrazole-3-carboxamide

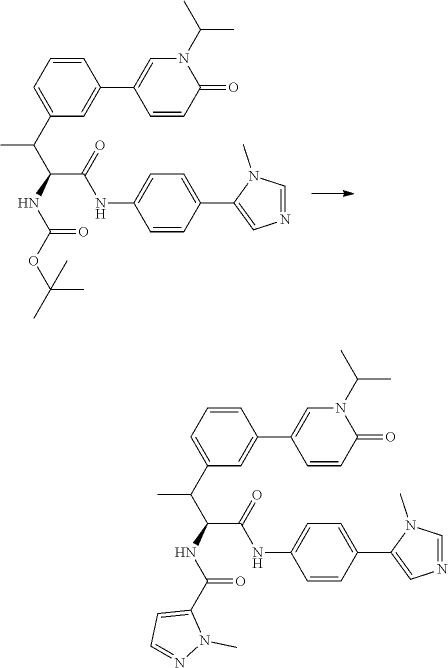 [SMILES: CC(c1cccc(-c2ccc(=O)n(C(C)C)c2)c1)[C@H](NC(=O)OC(C)(C)C)C(=O)Nc1ccc(-c2cncn2C)cc1 ; CC(c1cccc(-c2ccc(=O)n(C(C)C)c2)c1)[C@H](NC(=O)c1ccnn1C)C(=O)Nc1ccc(-c2cncn2C)cc1]

According to the method of Preparation 15 the compound of Preparation 58 (75.0 mg, 0.132 mmol) was reacted to give the title compound after basic HPLC as a colourless solid. (18.7 mg, 24% yield); LCMS (METHOD 3) (ES): m/z 578.5 [M+H]⁺, RT=0.51 min; ¹H NMR (600 MHz, DMSO-d6) δ 10.13 (s, 1H), 8.80 (d, J=8.6 Hz, 1H), 7.85 (d, J=2.6 Hz, 1H), 7.76 (dd, J=9.4, 2.6 Hz, 1H), 7.64 (d, J=1.1 Hz, 1H), 7.52-7.45 (m, 4H), 7.37 (dt, J=7.7, 1.5 Hz, 1H), 7.35-7.29 (m, 3H), 7.25 (dt, J=7.6, 1.4 Hz, 1H), 7.10 (d, J=2.1 Hz, 1H), 6.95 (d, J=1.1 Hz, 1H), 6.48 (d, J=9.3 Hz, 1H), 5.09 (hept, J=6.9 Hz, 1H), 4.94 (t, J=9.0 Hz, 1H), 4.03 (s, 3H), 3.61 (s, 3H), 3.46 (dq, J=9.5, 7.1 Hz, 1H), 1.45-1.28 (m, 9H).

### Example 58

### N-[(1S)-1-[[2-chloro-5-[3-(1-hydroxy-1-methyl-ethyl)phenyl]phenyl]methyl]-2-[4-(3,5-dimethyl-1H-pyrazol-4-yl)anilino]-2-oxo-ethyl]-2-methyl-pyrazole-3-carboxamide

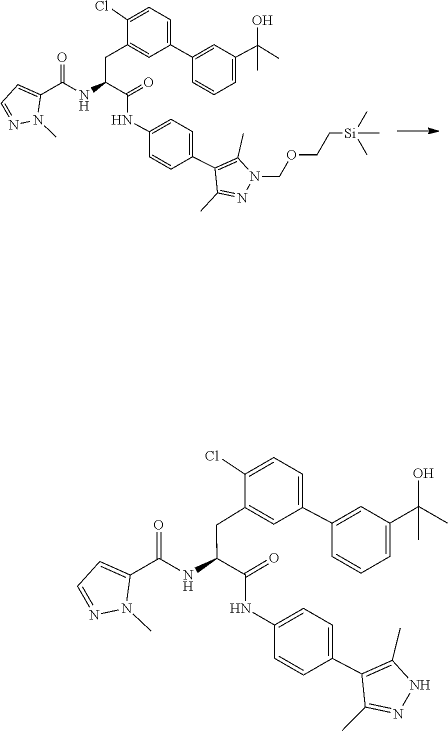 [SMILES: Cc1nn(COCC[Si](C)(C)C)c(C)c1-c1ccc(NC(=O)[C@H](Cc2cc(-c3cccc(C(C)(C)O)c3)ccc2Cl)NC(=O)c2ccnn2C)cc1 ; Cc1n[nH]c(C)c1-c1ccc(NC(=O)[C@H](Cc2cc(-c3cccc(C(C)(C)O)c3)ccc2Cl)NC(=O)c2ccnn2C)cc1]

TBAF (0.223 mL, 1.0 M sol. in THF) was added to a solution of the compound from Preparation 73 (33.0 mg, 0.045 mmol) in THF (1.0 mL) at room temperature. The reaction mixture was stirred at 80° C. o/n. The crude reaction mixture was concentrated in vacuo, dissolved in DMF (1.0 mL) and purified by acidic HPLC (10-100% MeCN in 0.1% HCOOH/H₂O) to afford the title compound as a colourless solid (13.0 mg, 47% yield).

¹H NMR (300 MHz, DMSO-d6) δ 12.25 (s, 1H), 10.18 (s, 1H), 8.86 (d, J=8.3 Hz, 1H), 7.76 (d, J=1.9 Hz, 1H), 7.69-7.60 (m, 3H), 7.54-7.50 (m, 2H), 7.47 (ddt, J=6.5, 4.8, 2.2 Hz, 1H), 7.41 (d, J=2.0 Hz, 1H), 7.37-7.31 (m, 2H), 7.25-7.17 (m, 2H), 6.99 (d, J=2.1 Hz, 1H), 5.04 (d, J=3.1 Hz, 2H), 3.94 (s, 3H), 2.17 (s, 6H), 1.45 (d, J=1.3 Hz, 6H); LCMS (METHOD 1) (ES): m/z 611.254 [M+H]⁺, RT=2.25 min.

### Example 59

### N-[(1S)-1-[[5-[3-(1-amino-1-methyl-ethyl)phenyl]-2-chloro-phenyl]methyl]-2-[4-(3,5-dimethyl-1H-pyrazol-4-yl)anilino]-2-oxo-ethyl]-2-methyl-pyrazole-3-carboxamide

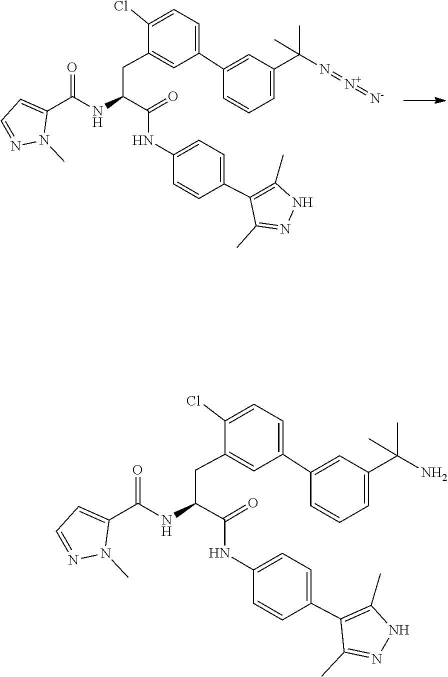 [SMILES: Cc1n[nH]c(C)c1-c1ccc(NC(=O)[C@H](Cc2cc(-c3cccc(C(C)(C)N=[N+]=[N-])c3)ccc2Cl)NC(=O)c2ccnn2C)cc1 ; Cc1n[nH]c(C)c1-c1ccc(NC(=O)[C@H](Cc2cc(-c3cccc(C(C)(C)N)c3)ccc2Cl)NC(=O)c2ccnn2C)cc1]

Platinum/C (2.0 mg) was added to a solution of the compound from Preparation 74 (10.0 mg, 0.016 mmol) in EtOAc (0.3 mL) and the reaction mixture was shaken under an atmosphere of hydrogen o/n. The reaction mixture was concentrated in vacuo then redissolved in MeOH (0.5 mL) and purified by acidic HPLC to afford the title compound as an off-white solid. (2.0 mg, 20% yield); ¹H NMR (300 MHz, DMSO-d6) δ 10.3 (s, 1H), 9.01 (d, J=8.4 Hz, 1H), 8.38 (s, 1H), 7.87-7.73 (m, 2H), 7.66 (d, J=8.5 Hz, 2H), 7.58-7.46 (m, 3H), 7.44-7.33 (m, 3H), 7.21 (d, J=8.6 Hz, 2H), 7.00 (d, J=2.1 Hz, 1H), 5.02 (s, 1H), 3.93 (s, 4H), 2.17 (s, 6H), 1.45 (d, J=1.3 Hz, 6H), LCMS (METHOD 1) (ES): m/z 610.269 [M+H]⁺, RT=1.95 min.

### Example 60

### N-[(1S)-1-[[2-chloro-5-[3-[(dimethylamino)methyl]phenyl]phenyl]methyl]-2-[4-(3,5-dimethyl-1H-pyrazol-4-yl)anilino]-2-oxo-ethyl]-2-methyl-pyrazole-3-carboxamide

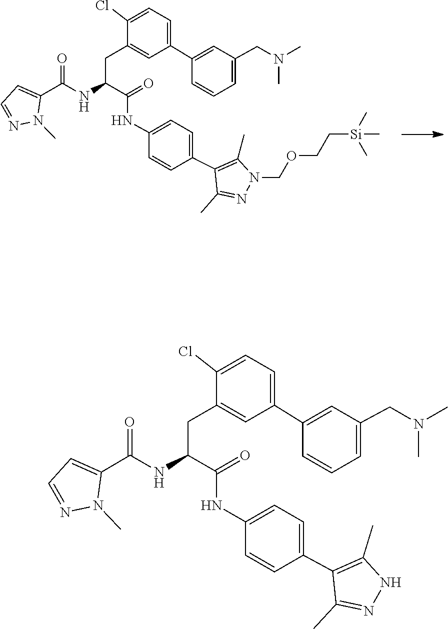 [SMILES: Cc1nn(COCC[Si](C)(C)C)c(C)c1-c1ccc(NC(=O)[C@H](Cc2cc(-c3cccc(CN(C)C)c3)ccc2Cl)NC(=O)c2ccnn2C)cc1 ; Cc1n[nH]c(C)c1-c1ccc(NC(=O)[C@H](Cc2cc(-c3cccc(CN(C)C)c3)ccc2Cl)NC(=O)c2ccnn2C)cc1]

TFA (0.5 mL) was added to a solution of the compound from Preparation 75 (74.0 mg, 0.10 mmol) in DCM (0.5 mL) at room temperature. The reaction mixture was stirred for 30 min. The reaction mixture was concentrated in vacuo, re-dissolved in MeOH (0.5 mL) and purified directly by acidic HPLC to afford title compound. (7.4 mg, 12% yield); LCMS (METHOD 3) (ES): m/z 610.6 [M+H]⁺, RT=0.55 min. ¹H NMR (300 MHz, DMSO-d6) δ 10.21 (s, 1H), 8.89 (d, J=8.3 Hz, 1H), 8.20 (s, 1H), 7.76 (t, J=1.3 Hz, 1H), 7.72-7.60 (m, 2H), 7.58-7.28 (m, 7H), 7.28-7.20 (m, 2H), 6.99 (d, J=2.1 Hz, 1H), 5.03 (td, J=8.8, 5.7 Hz, 2H), 3.94 (s, 3H), 3.63 (s, 2H), 3.50-3.21 (m, 2H), 2.30 (s, 6H), 2.18 (s, 6H).

### Example 61

### N-[(1S)-1-[[5-[3-(aminomethyl)phenyl]-2-chloro-phenyl]methyl]-2-[4-(3,5-dimethyl-1H-pyrazol-4-yl)anilino]-2-oxo-ethyl]-2-methyl-pyrazole-3-carboxamide

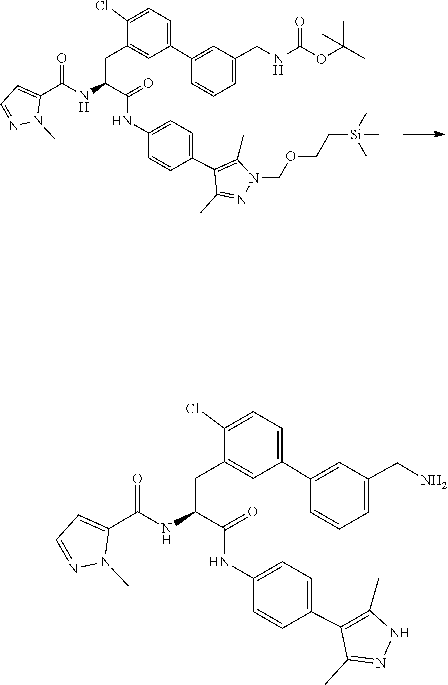 [SMILES: Cc1nn(COCC[Si](C)(C)C)c(C)c1-c1ccc(NC(=O)[C@H](Cc2cc(-c3cccc(CNC(=O)OC(C)(C)C)c3)ccc2Cl)NC(=O)c2ccnn2C)cc1 ; Cc1n[nH]c(C)c1-c1ccc(NC(=O)[C@H](Cc2cc(-c3cccc(CN)c3)ccc2Cl)NC(=O)c2ccnn2C)cc1]

TFA (0.5 mL) was added to a solution of the compound from Preparation 76 (54.0 mg, 0.066 mmol) in DCM (0.5 mL) at room temperature. The reaction mixture was stirred for 30 min. The reaction mixture was concentrated in vacuo, re-dissolved in MeOH (0.5 mL) and purified directly by acidic HPLC to afford title compound. (14.7 mg, 38% yield); LCMS (METHOD 3) (ES): m/z 610.6 [M+H]⁺, RT=0.52 min. ¹H NMR (300 MHz, DMSO-d6) δ 10.41 (s, 1H), 9.11 (d, J=8.3 Hz, 1H), 8.45 (s, 1H), 7.82 (s, 1H), 7.77-7.61 (m, 3H), 7.58-7.34 (m, 7H), 7.22 (d, J=8.2 Hz, 2H), 7.03 (d, J=2.0 Hz, 1H), 5.02 (td, J=8.8, 5.5 Hz, 2H), 3.93 (s, 3H), 3.52-3.17 (m, 2H), 2.17 (s, 6H).

### Example 230

### N-[1-[[3-(1-isopropyl-6-oxo-3-pyridyl)phenyl]-phenyl-methyl]-2-[4-(3-methylimidazol-4-yl)anilino]-2-oxo-ethyl]-2-methyl-pyrazole-3-carboxamide

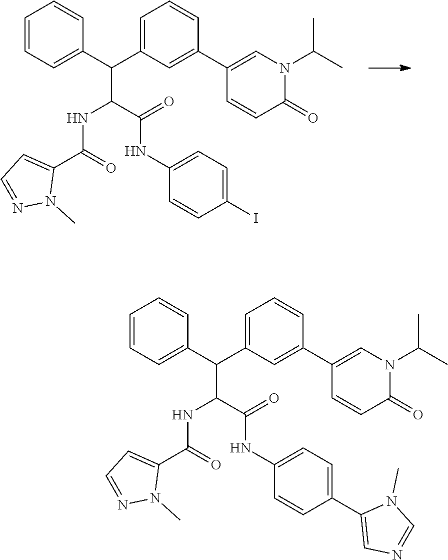 [SMILES: CC(C)n1cc(-c2cccc(C(c3ccccc3)C(NC(=O)c3ccnn3C)C(=O)Nc3ccc(I)cc3)c2)ccc1=O ; CC(C)n1cc(-c2cccc(C(c3ccccc3)C(NC(=O)c3ccnn3C)C(=O)Nc3ccc(-c4cncn4C)cc3)c2)ccc1=O]

1-isopropyl-5-(4,4,5,5-tetramethyl-1,3,2-dioxaborolan-2-yl)pyridin-2-one (30.0 mg, 0.14 mmol) was added to a solution of the compound from Preparation 86 (100 mg, 0.14 mmol) in 1,4-Dioxane (2 mL). A solution of Na₂CO₃ (46.0 mg, 0.43 mmol) in H₂O (20 mL) was added and the reaction mixture was degassed with argon for 10 min. PdCl₂(dppf).DCM (11.0 mg, 0.014 mmol) was added and the reaction mixture was stirred at 100° C. o/n. The reaction mixture was filtered through Celite pad and washed with 10% MeOH/DCM (10 mL) and H₂O (5 mL). The filtrate was collected and extracted with 10% MeOH/DCM (2×10 mL). The combined organic phases were washed with brine (100 mL) then dried over Na₂SO₄, filtered and concentrated in vacuo to afford compound (mixture of diastereomers) as a brown solid. The individual diastereomers were separated first by prep HPLC (10 mM aq. solution ammonium bicarbonate/acetonitrile; column: X-BRIDGE C18 (19*250) 5 u, 25 mL/min), then secondly by SFC (Chiracel OD-H (30×250 mm), 5p; 80% CO₂, 20% co-solvent (MeOH); Total flow rate: 60 g/min; UV: 214 nm.), to afford the title compound as a colourless solid (7.5 mg, 8% yield). ¹H NMR (400 MHz, DMSO-d6): δ ppm 10.48 (br s, 1H), 8.95-8.89 (m, 1H), 7.83-7.80 (m, 2H), 7.64-7.62 (d, J=6.8 Hz, 2H), 7.49-7.46 (m, 4H) 7.38-7.25 (m, 8H), 7.16-7.12 (m, 1H), 6.93 (s, 1H), 6.80 (d, J=1.32 Hz, 1H), 6.49-6.46 (d, J=8.99 Hz, 1H), 5.70-5.67 (m, 1H), 5.08-5.02 (m, 1H), 4.70-4.67 (d, J=11.62 Hz, 1H), 3.91 (s, 3H), 3.59 (s, 3H), 1.34-1.30 (m, 6H). LCMS (ESI): m/z 639 [M+H+]; 98%; RT=1.76 min.

### Example 231

### Cyclopropyl N-[1-[[3-(1-isopropyl-6-oxo-3-pyridyl)phenyl]-phenyl-methyl]-2-[4-(3-methylimidazol-4-yl)anilino]-2-oxo-ethyl]carbamate

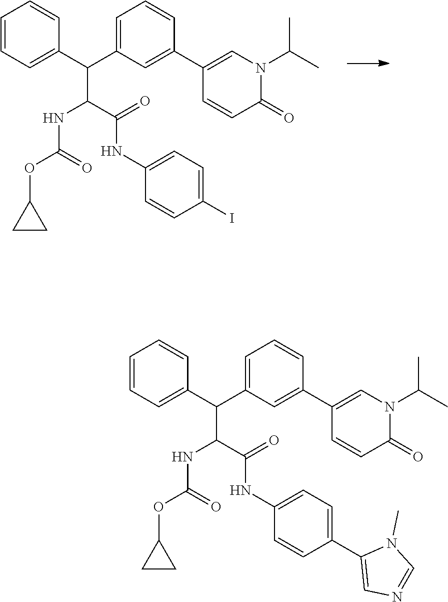 [SMILES: CC(C)n1cc(-c2cccc(C(c3ccccc3)C(NC(=O)OC3CC3)C(=O)Nc3ccc(I)cc3)c2)ccc1=O ; CC(C)n1cc(-c2cccc(C(c3ccccc3)C(NC(=O)OC3CC3)C(=O)Nc3ccc(-c4cncn4C)cc3)c2)ccc1=O]

According to the method of Example 230, the compound from Preparation 87 (350 mg, 0.53 mmol) was reacted to give the title compound (12.0 mg, 4%). Prep HPLC conditions: 0.1% TFA/H₂O/MeCN; column: X-BRIDGE C18 (19*250) 5 u; 25 mL/min. SFC conditions: Chiralpak IC (250×30 mm), 5 u; 0.2% TFA/MeOH; 40.0 mL/min; UV 265 nm. ¹H NMR (400 MHz, DMSO-d6): δ ppm 10.30 (br s, 1H), 7.94 (br s, 1H), 7.78-7.70 (m, 3H), 7.56 (br s, 1H), 7.47-7.40 (m, 4H), 7.35-7.30 (m, 5H), 7.24-7.18 (m, 3H), 7.11 (br s, 1H), 6.47-6.44 (d, J=10 Hz, 1H), 5.22-5.18 (m, 1H), 5.06-5.02 (m, 1H), 4.41 (br d, J=11.62 Hz, 1H), 3.88-3.85 (m, 1H), 3.63 (s, 3H), 1.33-1.29 (m, 6H), 0.56-0.39 (m, 4H). LCMS (ESI): m/z 615 [M+H+]; 99%; RT=1.82 min.

Examples of the general formula (A) are tabled below

| | | | | | | (A) |
|---|---|---|---|---|---|---|
| 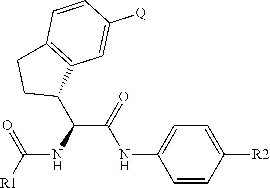 | | | | | | |
| Example | | | | | Mass | |
| number | R1 | Q | R2 | Name | ion | RT* |
| 4^{a} | 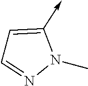 | 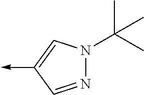 | 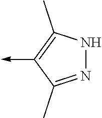 | N-[(1S)-1-[(1R)-6-(1-tert- butylpyrazol-4-yl)indan-1-yl]-2- [4-(3,5-dimethyl-1H-pyrazol-4- yl)anilino]-2-oxo-ethyl]-2- methyl-pyrazole-3-carboxamide | 591.321 | 2.23 |
| 5^{a} | 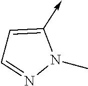 | 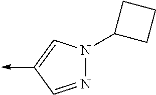 | 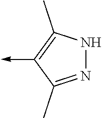 | N-[(1S)-1-[(1R)-6-(1- cyclobutylpyrazol-4-yl)indan-1- yl]-2-[4-(3,5-dimethyl-1H- pyrazol-4-yl)anilino]-2-oxo- ethyl]-2-methyl-pyrazole-3- carboxamide | 589.305 | 2.20 |
| 6^{a} | 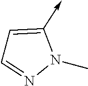 | 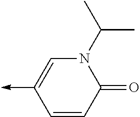 | 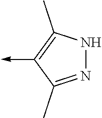 | N-[(1S)-2-[4-(3,5-dimethyl-1H- pyrazol-4-yl)anilino]-1-[(1R)-6- (1-isopropyl-6-oxo-3- pyridyl)indan-1-yl]-2-oxo-ethyl]- 1-fluoro- cyclopropanecarboxamide | 582.29 | 2.19 |
| 7^{a} | 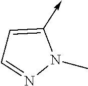 | 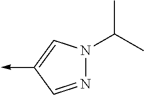 | 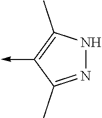 | N-[(1S)-2-[4-(3,5-dimethyl-1H- pyrazol-4-yl)anilino]-1-[(1R)-6- (1-isopropyl-6-oxo-3- pyridyl)indan-1-yl]-2-oxo-ethyl]- 1-fluoro- cyclopropanecarboxamide | 577.305 | 2.16 |
| 8^{a} | 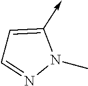 | 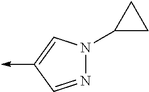 | 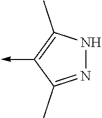 | N-[(1S)-1-[(1R)-6-(1- cyclopropylpyrazol-4-yl)indan-1- yl]-2-[4-(3,5-dimethyl-1H- pyrazol-4-yl)anilino]-2-oxo- ethyl]-2-methyl-pyrazole-3- carboxamide | 575.29 | 2.13 |
| 9^{a} | 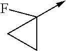 | 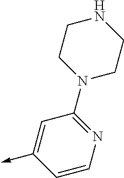 | 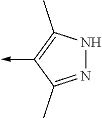 | N-[(1S)-2-[4-(3,5-dimethyl-1H- pyrazol-4-yl)anilino]-2-oxo-1- [(1R)-6-(2-piperazin-1-yl-4- pyridyl)indan-1-yl]ethyl]-1- fluoro-cyclopropanecarboxamide | 608.316 | 1.92 |
| 10^{a} | 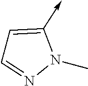 | 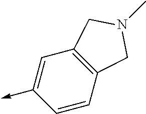 | 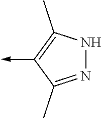 | N-[(1S)-2-[4-(3,5-dimethyl-1H- pyrazol-4-yl)anilino]-1-[(1R)-6- (2-methylisoindolin-5-yl)indan-1- yl]-2-oxo-ethyl]-2-methyl- pyrazole-3-carboxamide | 600.31 | 1.91 |
| 11^{a} | 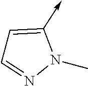 | 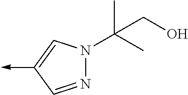 | 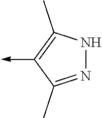 | N-[(1S)-2-[4-(3,5-dimethyl-1H- pyrazol-4-yl)anilino]-1-[(1R)-6- [1-(2-hydroxy-1,1-dimethyl- ethyl]pyrazol-4-yl]indan-1-yl]-2- oxo-ethyl]-2-methyl-pyrazole-3- carboxamide | 607.315 | 2.06 |
| 12^{a} | 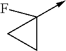 | 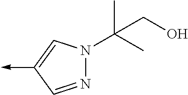 | 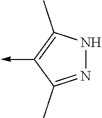 | N-[(1S)-2-[4-(3,5-dimethyl-1H- pyrazol-4-yl)anilino]-1-[(1R)-6- [1-(2-hydroxy-1,1-dimethyl- ethyl)pyrazol-4-yl]indan-1-yl]-2- oxo-ethyl]-1-fluoro- cyclopropanecarboxamide | 585.30 | 2.16 |
| 13^{a} | 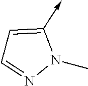 | 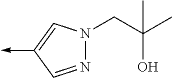 | 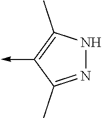 | N-[(1S)-2-[4-(3,5-dimethyl-1H- pyrazol-4-yl)anilino]-1-[(1R)-6- [1-(2-hydroxy-2-methyl- propyl)pyrazol-4-yl]indan-1-yl]- 2-oxo-ethyl]-2-methyl-pyrazole- 3-carboxamide | 607.315 | 2.04 |
| 14^{a} | 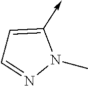 | 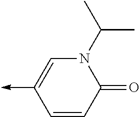 | 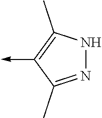 | N-[(1S)-2-[4-(3,5-dimethyl-1H- pyrazol-4-yl)anilino]-1-[(1R)-6- (2-isopropyl-4-pyridyl)indan-1- yl]-2-oxo-ethyl]-2-methyl- pyrazole-3-carboxamide | 588.311 | 1.96 |
| 15^{a} | 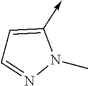 | 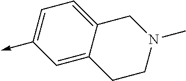 | 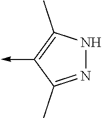 | N-[(1S)-2-[4-(3,5-dimethyl-1H- pyrazol-4-yl)anilino]-1-[(1R)-6- (2-methyl-3,4-dihydro-1H- isoquinolin-6-yl)indan-1-yl]-2- oxo-ethyl]-2-methyl-pyrazole-3- carboxamide | 614.326 | 1.93 |
| 16^{a} | 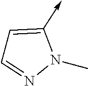 | 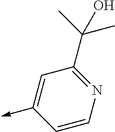 | 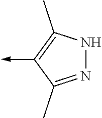 | N-[(1S)-2-[4-(3,5-dimethyl-1H- pyrazol-4-yl)anilino]-1-[(1R)-6- [2-(1-hydroxy-1-methyl-ethyl)- 4-pyridyl]indan-1-yl]-2-oxo- ethyl]-2-methyl-pyrazole-3- carboxamide | 604.305 | 1.90 |
| 17^{a} | 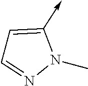 | 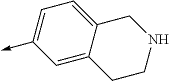 | 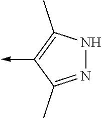 | N-[(1S)-2-[4-(3,5-dimethyl-1H- pyrazol-4-yl)anilino]-2-oxo-1- [(1R)-6-(1,2,3,4- tetrahydroisoquinolin-6-yl)indan- 1-yl]ethyl]-2-methyl-pyrazole-3- carboxamide | 600.31 | 1.92 |
| 18^{a} | 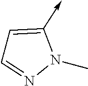 | 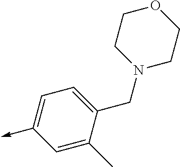 | 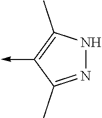 | N-[(1S)-2-[4-(3,5-dimethyl-1H- pyrazol-4-yl)anilino]-1-[(1R)-6- [3-methyl-4- (morpholinomethyl)phenyl]indan- 1-yl]-2-oxo-ethyl]-2-methyl- pyrazole-3-carboxamide | 658.353 | 1.96 |
| 19^{a} | 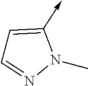 | 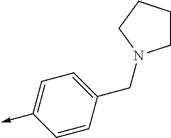 | 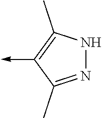 | N-[(1S)-2-[4-(3,5-dimethyl-1H- pyrazol-4-yl)anilino]-2-oxo-1- [(1R)-6-[4-(pyrrolidin-1- ylmethyl)phenyl]indan-1- yl]ethyl]-2-methyl-pyrazole-3- carboxamide | 628.342 | 1.96 |
| 20^{a} | 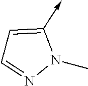 | 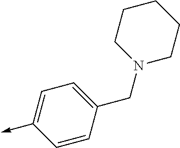 | 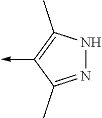 | N-[(1S)-2-[4-(3,5-dimethyl-1H- pyrazol-4-yl)anilino]-2-oxo-1- [(1R)-6-[4-(1- piperidylmethyl)phenyl]indan-1- yl]ethyl]-2-methyl-pyrazole-3- carboxamide | 642.357 | 1.98 |
| 21^{a} | 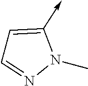 | 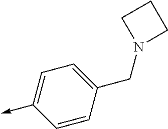 | 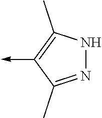 | N-[(1S)-1-[(1R)-6-[4-(azetidin- 1-ylmethyl)phenyl]indan-1-yl]-2- [4-(3,5-dimethyl-1H-pyrazole-4- yl)anilino]-2-oxo-ethyl]-2- methyl-pyrazole-3-carboxamide | 614.327 | 1.94 |
| 22^{a} | 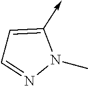 | 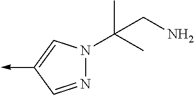 | 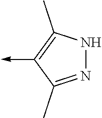 | N-[(1S)-1-[(1R)-6-[1-(2-amino- 1,1-dimethyl-ethyl)pyrazol-4- yl]indan-1-yl]-2-[4-(3,5- dimethyl-1H-pyrazol-4- yl)anilino]-2-oxo-ethyl]-2- methyl-pyrazole-3-carboxamide | 606.333 | 1.89 |
| 23^{a} | 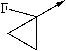 | 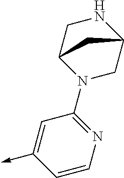 | 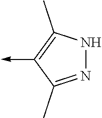 | N-[(1S)-1-[(1R)-6-[2-[(1S,4S)- 2,5-diazabicyclo[2.2.1]heptan-2- yl]-4-pyridyl]indan-1-yl]-2-[4- (3,5-dimethyl-1H-pyrazol-4- yl)anilino]-2-oxo-ethyl]-1-fluoro- cyclopropanecarboxamide | 620.317 | 1.84 |
| 24^{a} | 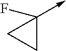 | 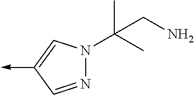 | 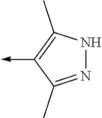 | N-[(1S)-1-[(1R)-6-[1-(2-amino- 1,1-dimethyl-ethyl)pyrazol-4- yl]indan-1-yl]-2-[4-(3,5- dimethyl-1H-pyrazol-4- yl)anilino]-2-oxo-ethyl]-1-fluoro- cyclopropanecarboxamide | 584.317 | 1.95 |
| 25^{b} | 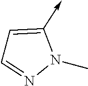 | 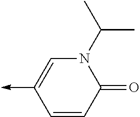 | 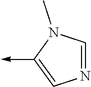 | N-[(1S)-1-[6-(1-isopropyl-6-oxo- 3-pyridyl)indan-1-yl]-2-[4-(3- methylimidazol-4-yl)anilino]-2- oxo-ethyl]-2-methyl-pyrazole-3- carboxamide | 590.289 | 1.92 |

*RT are XEV Metode 1 CM-ES+ unless stated
^{a}prepared according to the method of Example 1
^{b}prepared according to the method of Example 3

Examples of the general formula (B) are tabled below

| | | | | | | (B) |
|---|---|---|---|---|---|---|
| 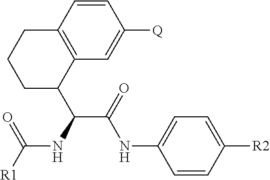 | | | | | | |
| Example | | | | | Mass | |
| number | R1 | Q | R2 | Name | ion | RT* |
| 30^{c} | 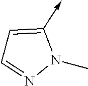 | 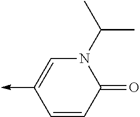 | 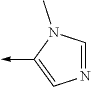 | N-[(1S)-1-[(1R)-7-(1- isopropyl-6-oxo-3- pyridyl)tetralin-1-yl]-2-[4-(3- methylimidazol-4-yl)anilino]- 2-oxo-ethyl]-2-methyl- pyrazole-3-carboxamide | 604.304 | 1.95 |
| 32^{d} | 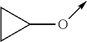 | 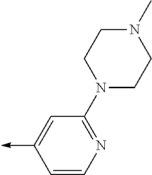 | 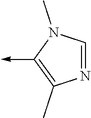 | cyclopropyl N-[(1S)-2-[4-(3,5- dimethylimidazol-4- yl)anilino]-1-[(1R)-7-[2-(4- methylpiperazin-1-yl)-4- pyridyl]tetralin-1-yl]-2-oxo- ethyl]carbamate | 634.351 | 1.81 |
| 34^{a} | 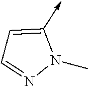 | 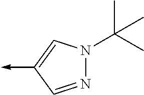 | 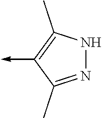 | N-[(1S)-1-[(1R)-7-(1-tert- butylpyrazol-4-yl)tetralin-1- yl]-2-[4-(3,5-dimethyl-1H- pyrazol-4-yl)anilino]-2-oxo- ethyl]-2-methyl-pyrazole-3- carboxamide | 605.336 | 2.26 |
| 35^{a} | 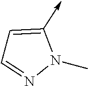 | 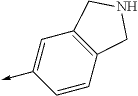 | 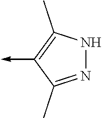 | N-[(1S)-2-[4-(3,5-dimethyl- 1H-pyrazol-4-yl)anilino]-1- [(1R)-7-isoindolin-5-yltetralin- 1-yl]-2-oxo-ethyl]-2-methyl- pyrazole-3-carboxamide | 600.31 | 1.94 |
| 36^{a} | 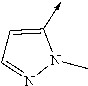 | 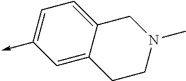 | 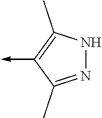 | N-[(1S)-2-[4-(3,5-dimethyl- 1H-pyrazol-4-yl)anilino]-1- [(1R)-7-(2-methyl-3,4- dihydro-1H-isoquinolin-6- yl)tetralin-1-yl]-2-oxo-ethyl]- 2-methyl-pyrazole-3- carboxamide | 628.341 | 1.97 |
| 37^{a} | 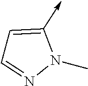 | 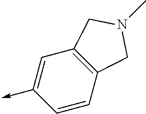 | 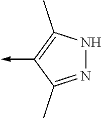 | N-[(1S)-2-[4-(3,5-dimethyl- 1H-pyrazol-4-yl)anilino]-1- [(1R)-7-(2-methylisoindolin-5- yl)tetralin-1-yl]-2-oxo-ethyl]- 2-methyl-pyrazole-3- carboxamide | 614.325 | 1.95 |
| 38^{a} | 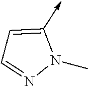 | 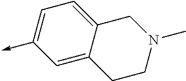 | 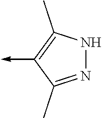 | N-[(1S)-2-[4-(3,5-dimethyl- 1H-pyrazol-4-yl)anilino]-2- oxo-1-[(1R)-7-(1,2,3,4- tetrahydroisoquinolin-6- yl)tetralin-1-yl]ethyl]-2- methyl-pyrazole-3- carboxamide | 614.325 | 1.96 |
| 39^{a} | 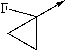 | 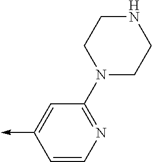 | 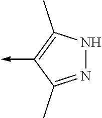 | N-[(1S)-2-[4-(3,5-dimethyl- 1H-pyrazol-4-yl)anilino]-2- oxo-1-[(1R)-7-(2-piperazin-1- yl-4-pyridyl)tetralin-1- yl]ethyl]-1-fluoro- cyclopropanecarboxamide | 622.333 | 1.95 |
| 40^{e} | 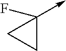 | 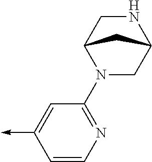 | 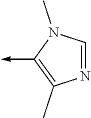 | N-[(1S)-1-[(1R)-7-[2- [(1S,4S)-2,5- diazabicyclo[2.2.1]heptan-2- yl]-4-pyridyl]tetralin-1-yl]-2- [4-(3,5-dimethylimidazol-4- yl)anilino]-2-oxo-ethyl]-1- fluoro-cyclopropanecarboxamide | 634.333 | 1.74 |
| 41^{b} | 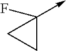 | 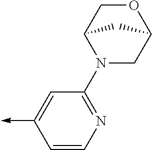 | 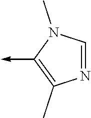 | N-[(1S)-1-[(1R)-7-[2- [(1S,4S)-2,5- diazabicyclo[2.2.1]heptan-2- yl]-4-pyridyl]tetralin-1-yl]-2- [4-(3,5-dimethylimidazol-4- yl)anilino]-2-oxo-ethyl]-1- fluoro-cyclopropanecarboxamide | 635.318 | 1.84 |
| 42^{e} | 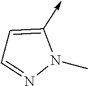 | 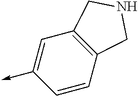 | 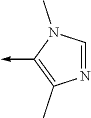 | N-[(1S)-2-[4-(3,5- dimethylimidazol-4- yl)anilino]-1-[(1R)-7- isoindolin-5-yltetralin-1-yl]-2- oxo-ethyl]-2-methyl-pyrazole- 3-carboxamide | 600.312 | 1.79 |
| 43^{e} | 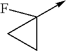 | 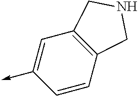 | 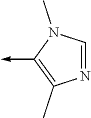 | N-[(1S)-2-[4-(3,5- dimethylimidazol-4- yl)anilino]-1-[(1R)-7- isoindolin-5-yltetralin-1-yl]-2- oxo-ethyl]-1-fluoro- cyclopropanecarboxamide | 578.296 | 1.84 |
| 44^{c} | 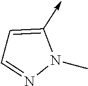 | 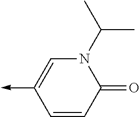 | 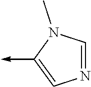 | N-[(1S)-1-[(1R)-7-(1- isopropyl-6-oxo-3- pyridyl)tetralin-1-yl]-2-[4-(3- methylimidazol-4-yl)anilino]- 2-oxo-ethyl]-2-methyl- pyrazole-3-carboxamide | 604.304 | 1.96 |
| 45^{b} | 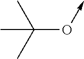 | 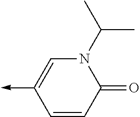 | 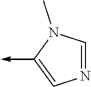 | tert-butyl N-[(1R)-1-[(1S)-7- (1-isopropyl-6-oxo-3- pyridyl)tetralin-1-yl]-2-[4-(3- methylimidazol-4-yl)anilino]- 2-oxo-ethyl]carbamate | 596.325 | 2.12 |
| 48^{c} | 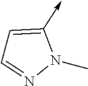 | 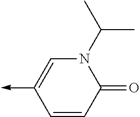 | 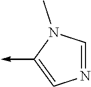 | N-[(1R)-1-[(1S)-7-(1- isopropyl-6-oxo-3- pyridyl)tetralin-1-yl]-2-[4-(3- methylimidazol-4-yl)anilino]- 2-oxo-ethyl]-2-methyl- pyrazole-3-carboxamide | 604.304 | 1.95 |
| 50^{f} | 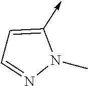 | 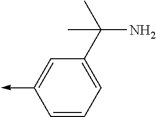 | 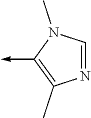 | N-[(1S)-1-[(1R)-7-[3-(1- amino-1-methyl- ethyl)phenyl]tetralin-1-yl]-2- [4-(3,5-dimethylimidazol-4- yl)anilino]-2-oxo-ethyl]-2- methyl-pyrazole-3- carboxamide | 616.34 | 1.81 |

*RT are XEV Metode 1 CM-ES+ unless stated
^{a}prepared according to the method of Example 1
^{b}prepared according to the method of Example 3
^{c}prepared according to the method of Preparation 29
^{d}prepared according to the method of Preparation 87
^{e}prepared according to the method of Example 3, followed by Boc deprotection
^{f}prepared according to the method of Example 59

Examples of the general formula (C) are tabled below

| | | | | | | (C) |
|---|---|---|---|---|---|---|
| 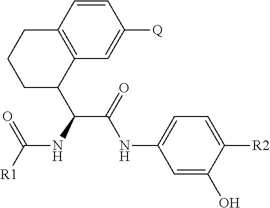 | | | | | | |
| Example | | | | | Mass | |
| number | R1 | Q | R2 | Name | ion | RT* |
| 31^{d} | 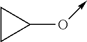 | 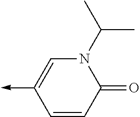 | 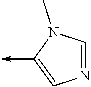 | cyclopropyl N-[(1S)-2-[3- hydroxy-4-(3-methylimidazol- 4-yl)anilino]-1-[(1R)-7-(1- isopropyl-6-oxo-3- pyridyl)tetralin-1-yl]-2-oxo- ethyl]carbamate | 596.288 | 1.99 |
| 33^{a} | 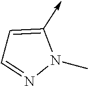 | 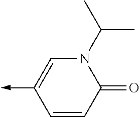 | 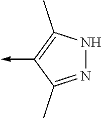 | N-[(1S)-2-[4-(3,5-dimethyl- 1H-pyrazol-4-yl)-3-hydroxy- anilino]-1-[(1R)-7-(1- isopropyl-6-oxo-3- pyridyl)tetralin-1-yl]-2-oxo- ethyl]-2-methyl-pyrazole-3- carboxamide | 634.315 | 2.05 |
| 46^{a} | 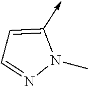 | 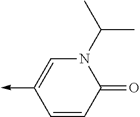 | 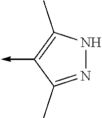 | N-[(1S)-2-[4-(3,5-dimethyl- 1H-pyrazol-4-yl)-3-hydroxy- anilino]-1-[(1R)-7-(1- isopropyl-6-oxo-3- pyridyl)tetralin-1-yl]-2-oxo- ethyl]-2-methyl-pyrazole-3- carboxamide | 634.314 | 2.06 |
| 47^{a} | 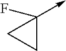 | 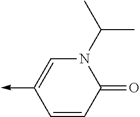 | 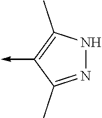 | N-[(1S)-2-[4-(3,5-dimethyl- 1H-pyrazol-4-yl)-3-hydroxy- anilino]-1-[(1R)-7-(1- isopropyl-6-oxo-3- pyridyl)tetralin-1-yl]-2-oxo- ethyl]-1-fluoro- cyclopropanecarboxamide | 612.299 | 2.15 |
| 49^{c} | 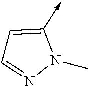 | 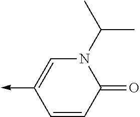 | 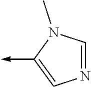 | N-[(1S)-2-[3-hydroxy-4-(3- methylimidazol-4-yl)anilino]- 1-[(1R)-7-(1-isopropyl-6-oxo- 3-pyridyl)tetralin-1-yl]-2-oxo- ethyl]-2-methyl-pyrazole-3- carboxamide | 620.299 | 1.93 |

*RT are XEV Metode 1 CM-ES+ unless stated
^{a}prepared according to the method of Example 1
^{c}prepared according to the method of Preparation 29
^{d}prepared according to the method of Preparation 87

Examples of the general formula (D) are tabled below

| | | | | | | (D) |
|---|---|---|---|---|---|---|
| 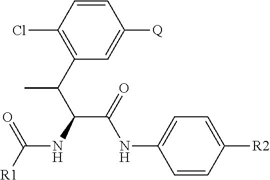 | | | | | | |
| Example | | | | | Mass | |
| number | R1 | Q | R2 | Name | ion | RT* |
| 52^{c} | 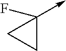 | 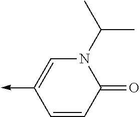 | 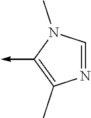 | N-[(1S)-2-[2-chloro-5-(1-isopropyl- 6-oxo-3-pyridyl)phenyl]-1-[[4-(3,5- dimethylimidazol-4- yl)phenyl]carbamoyl]propyl]-1- fluoro-cyclopropanecarboxamide | 604.248 | 2.04 |
| 53^{d} | 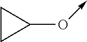 | 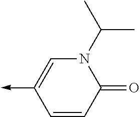 | 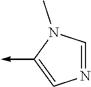 | cyclopropyl N-[(1S)-2-[2-chloro-5- (1-isopropyl-6-oxo-3- pyridyl)phenyl]-1-[[4-(3- methylimidazol-4- yl)phenyl]carbamoyl]propyl] carbamate | 588.238 | 2.02 |
| 54^{c} | 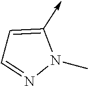 | 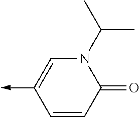 | 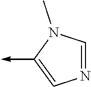 | N-[(1S)-2-[6-chloro-3-(1-isopropyl- 6-oxo-3-pyridyl)cyclohexa-2,4-dien- 1-yl]-1-[[4-(3-methylimidazol-4- yl)phenyl]carbamoyl]propyl]-2- methyl-pyrazole-3-carboxamide | 612.25 | 1.97 |
| 55^{c} | 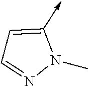 | 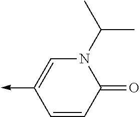 | 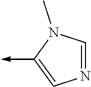 | N-[2-[2-chloro-5-(1-isopropyl-6-oxo- 3-pyridyl)phenyl]-1-[[4-(3- methylimidazol-4- yl)phenyl]carbamoyl]propyl]-2- methyl-pyrazole-3-carboxamide | 612.25 | 1.97 |

*RT are XEV Metode 1 CM-ES+ unless stated
^{c}prepared according to the method of Preparation 29
^{d}prepared according to the method of Preparation 87

Examples of the general formula (E) are tabled below

| | | | | | | (E) |
|---|---|---|---|---|---|---|
| 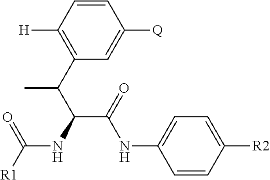 | | | | | | |
| Example | | | | | Mass | |
| number | R1 | Q | R2 | Name | ion | RT* |
| 57^{c} | 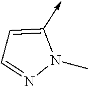 | 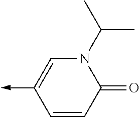 | 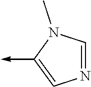 | N-[(1S)-2-[3-(1-isopropyl-6-oxo-3- pyridyl)phenyl]-1-[[4-(3- methylimidazol-4- yl)phenyl]carbamoyl]propyl]-2- methyl-pyrazole-3-carboxamide (Diasteromer 2) | 578.288 | 1.89 |

*RT are XEV Metode 1 CM-ES+ unless stated
^{c}prepared according to the method of Preparation 29

Examples of the general formula (F) are tabled below

| | |
|---|---|
| | (F) |
| 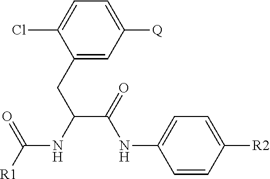 | |

| Example | | | | | Mass | |
|---|---|---|---|---|---|---|
| number | R1 | Q | R2 | Name | ion | RT* |
| 62^{b} | 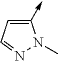 | 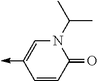 | 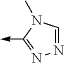 | N-[1-[[2-chloro-5-(1-isopropyl-6-oxo-3- pyridyl)phenyl]methyl]-2-[4-(4-methyl- 1,2,4-triazol-3-yl)anilino]-2-oxo-ethyl]-2- methyl-pyrazole-3-carboxamide | 599.228 | 1.99 |
| 63^{c} | 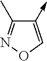 | 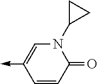 | 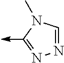 | N-[1-[[2-chloro-5-(1-cyclopropyl-6-oxo- 3-pyridyl)phenyl]methyl]-2-[4-(4-methyl- 1,2,4-triazol-3-yl)anilino]-2-oxo-ethyl]-3- methyl-isoxazole-4-carboxamide | 598.198 | 1.97 |
| 64^{b} | 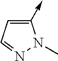 | 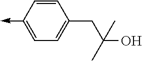 | 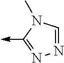 | N-[1-[[2-chloro-5-[2-(2-hydroxy-2- methyl-propyl)-4-pyridyl]phenyl]methyl]- 2-[4-(4-methyl-1,2,4-triazol-3- yl)anilino]-2-oxo-ethyl]-2-methyl- pyrazole-3-carboxamide | 613-245 | 1.81 |
| 65^{c} | 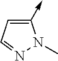 | 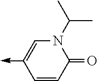 | 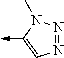 | N-[1-[[2-chloro-5-(1-isopropyl-6-oxo-3- pyridyl)phenyl]methyl]-2-[4-(3- methyltriazol-4-yl)anilino]-2-oxo-ethyl]- 2-methyl-pyrazole-3-carboxamide | 599.23 | 2.10 |
| 66^{c} | 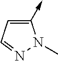 | 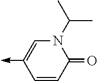 | 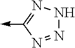 | N-[1-[[2-chloro-5-(1-isopropyl-6-oxo-3- pyridyl)phenyl]methyl]-2-oxo-2-[4-(2H- tetrazol-5-yl)anilino]ethyl]-2-methyl- pyrazole-3-carboxamide | 586.209 | 2.06 |
| 67^{g} | 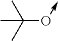 | 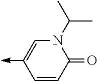 | 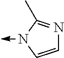 | tert-butyl N-[1-[[2-chloro-5-(1-isopropyl- 6-oxo-3-pyridyl)phenyl]methyl]-2-[4-(2- methylimidazol-1-yl)anilino]-2-oxo- ethyl]carbamate | 590.254 | 2.06 |
| 68^{c} | 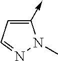 | 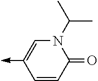 | 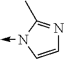 | N-[1-[[2-chloro-5-(1-isopropyl-6-oxo-3- pyridyl)phenyl]methyl]-2-[4-(2- methylimidazol-1-yl)anilino]-2-oxo- ethyl]-2-methyl-pyrazole-3-carboxamide | 598.234 | 1.92 |
| 72^{b} | 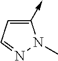 | 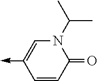 | 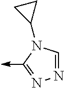 | N-[1-[[2-chloro-5-(1-isopropyl-6-oxo-3- pyridyl)phenyl]methyl]-2-[4-(4- cyclopropyl-1,2,4-triazol-3-yl)anilino]-2- oxo-ethyl]-2-methyl-pyrazole-3- carboxamide | 625.247 | 2.05 |
| 73^{b} | 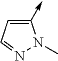 | 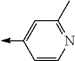 | 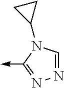 | N-[1-[[2-chloro-5-(2-methyl-4- pyridyl)phenyl]methyl]-2-[4-(4- cyclopropyl-1,2,4-triazol-3-yl)anilino]-2- oxo-ethyl]-2-methyl-pyrazole-3- carboxamide | 581.218 | 1.85 |
| 74^{b} | 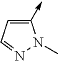 | 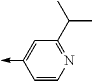 | 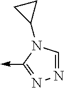 | N-[1-[[2-chloro-5-(2-isopropyl-4- pyridyl)phenyl]methyl]-2-[4-(4- cyclopropyl-1,2,4-triazol-3-yl)anilino]-2- oxo-ethyl]-2-methyl-pyrazole-3- carboxamide | 609.25 | 1.96 |
| 75^{b} | 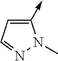 | 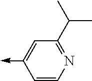 | 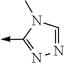 | N-[1-[[2-chloro-5-(2-isopropyl-4- pyridyl)phenyl]methyl]-2-[4-(4-methyl- 1,2,4-triazol-3-yl)anilino]-2-oxo-ethyl]-2- methyl-pyrazole-3-carboxamide | 583.233 | 1.89 |
| 76^{g} | 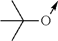 | 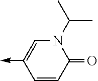 | 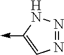 | tert-butyl N-[1-[[2-chloro-5-(1-isopropyl- 6-oxo-3-pyridyl)phenyl]methyl]-2-oxo-2- [4-(1H-triazol-5- yl)anilino]ethyl]carbamate | 577.233 | 2.25 |
| 77^{c} | 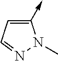 | 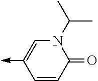 | 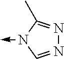 | N-[1-[[2-chloro-5-(1-isopropyl-6-oxo-3- pyridyl)phenyl]methyl]-2-[4-(3-methyl- 1,2,4-triazol-4-yl)anilino]-2-oxo-ethyl]-2- methyl-pyrazole-3-carboxamide | 599.224 | 2.01 |
| 78^{c} | 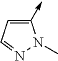 | 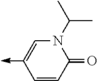 | 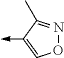 | N-[1-[[2-chloro-5-(1-isopropyl-6-oxo-3- pyridyl)phenyl]methyl]-2-[4-(3- methylisoxazol-4-yl)anilino]-2-oxo-ethyl]- 2-methyl-pyrazole-3-carboxamide | 599.219 | 2.25 |
| 79^{c} | 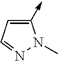 | 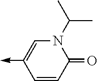 | 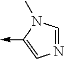 | N-[1-[[2-chloro-5-(1-isopropyl-6-oxo-3- pyridyl)phenyl]methyl]-2-[4-(3- methylimidazol-4-yl)anilino]-2-oxo- ethyl]-2-methyl-pyrazole-3-carboxamide | 598.233 | 1.92 |
| 80^{c} | 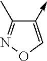 | 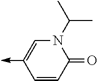 | 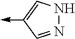 | N-[1-[[2-chloro-5-(1-isopropyl-6-oxo-3- pyridyl)phenyl]methyl]-2-oxo-2-[4-(1H- pyrazol-4-yl)anilino]ethyl]-3-methyl- isoxazole-4-carboxamide | 585.202 | 2.11 |
| 81^{c} | 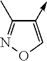 | 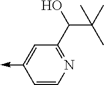 | 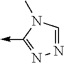 | N-[(1S)-1-[[2-chloro-5-[2-(1-hydroxy- 2,2-dimethyl-propyl)-4- pyridyl]phenyl]methyl]-2-[4-(4-methyl- 1,2,4-triazol-3-yl)anilino]-2-oxo-ethyl]-3- methyl-isoxazole-4-carboxamide | 628.245 | 1.99 |
| 82^{c} | 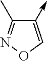 | 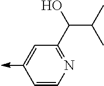 | 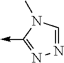 | N-[(1S)-1-[[2-chloro-5-[2-(1-hydroxy-2- methyl-propyl)-4-pyridyl]phenyl]methyl]- 2-[4-(4-methyl-1,2,4-triazol-3- yl)anilino]-2-oxo-ethyl]-3-methyl- isoxazole-4-carboxamide | 614.229 | 1.94 |
| 83^{c} | 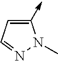 | 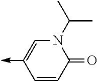 | 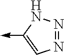 | N-[1-[[2-chloro-5-(1-isopropyl-6-oxo-3- pyridyl)phenyl]methyl]-2-oxo-2-[4-(1H- triazol-5-yl)anilino]ethyl]-2-methyl- pyrazole-3-carboxamide | 585.214 | 2.07 |
| 84^{h} | 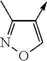 | 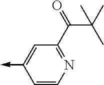 | 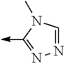 | N-[(1S)-1-[[2-chloro-5-[2-(2,2- dimethylpropanoyl)-4- pyridyl]phenyl]methyl]-2-[4-(4-methyl- 1,2,4-triazol-3-yl)anilino]-2-oxo-ethyl]-3- methyl-isoxazole-4-carboxamide | 626.229 | 2.40 |
| 85^{h} | 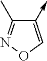 | 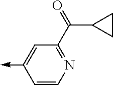 | 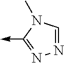 | N-[(1S)-1-[[2-chloro-5-[2- (cyclopropanecarbonyl)-4- pyridyl]phenyl]methyl]-2-[4-(4-methyl- 1,2,4-triazol-3-yl)anilino]-2-oxo-ethyl]-3- methyl-isoxazole-4-carboxamide | 610.198 | 2.20 |
| 86^{g} | 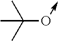 | 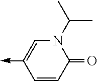 | 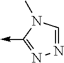 | tert-butyl N-[(1S)-1-[[2-chloro-5-(1- isopropyl-6-oxo-3- pyridyl)phenyl]methyl]-2-[4-(4-methyl- 1,2,4-triazol-3-yl)anilino]-2-oxo- ethyl]carbamate | 591.246 | 2.15 |
| 87ⁱ | 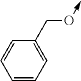 | 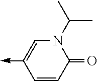 | 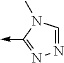 | benzyl N-[(1S)-1-[[2-chloro-5-(1- isopropyl-6-oxo-3- pyridyl)phenyl]methyl]-2-[4-(4-methyl- 1,2,4-triazol-3-yl)anilino]-2-oxo- ethyl]carbamate | 625.233 | 2.18 |
| 88^{c} | 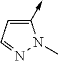 | 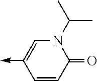 | 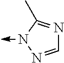 | N-[1-[[2-chloro-5-(1-isopropyl-6-oxo-3- pyridyl)phenyl]methyl]-2-[4-(5-methyl- 1,2,4-triazol-1-yl)anilino]-2-oxo-ethyl]-2- methyl-pyrazole-3-carboxamide | 599.228 | 2.07 |
| 89^{c} | 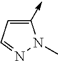 | 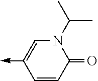 | 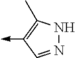 | N-[1-[[2-chloro-5-(1-isopropyl-6-oxo-3- pyridyl)phenyl]methyl]-2-[4-(5-methyl- 1H-pyrazol-4-yl)anilino]-2-oxo-ethyl]-2- methyl-pyrazole-3-carboxamide | 598.236 | 2.10 |
| 90^{g} | 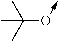 | 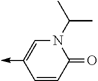 | 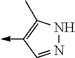 | tert-butyl N-[1-[[2-chloro-5-(1-isopropyl- 6-oxo-3-pyridyl)phenyl]methyl]-2-[4-(5- methyl-1H-pyrazol-4-yl)anilino]-2-oxo- ethyl]carbamate | 590.253 | 2.28 |
| 91^{c} | 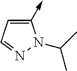 | 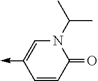 | 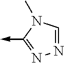 | N-[(1S)-1-[[2-chloro-5-(1-isopropyl-6- oxo-3-pyridyl)phenyl]methyl]-2-[4-(4- methyl-1,2,4-triazol-3-yl)anilino]-2-oxo- ethyl]-2-isopropyl-pyrazole-3- carboxamide | 627.262 | 2.09 |
| 92^{c} | 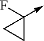 | 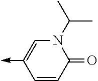 | 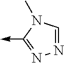 | N-[(1S)-1-[[2-chloro-5-(1-isopropyl-6- oxo-3-pyridyl)phenyl]methyl]-2-[4-(4- methyl-1,2,4-triazol-3-yl)anilino]-2-oxo- ethyl]-1-fluoro-cyclopropanecarboxamide | 577.216 | 2.05 |
| 93^{c} | 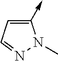 | 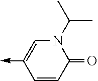 | 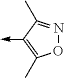 | N-[1-[[2-chloro-5-(1-isopropyl-6-oxo-3- pyridyl)phenyl]methyl]-2-[4-(3,5- dimethylisoxazol-4-yl)anilino]-2-oxo- ethyl]-2-methyl-pyrazole-3-carboxamide | 613.233 | 2.28 |
| 94^{a} | 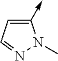 | 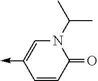 | 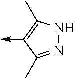 | N-[1-[[2-chloro-5-(1-isopropyl-6-oxo-3- pyridyl)phenyl]methyl]-2-[4-(3,5- dimethyl-1H-pyrazol-4-yl)anilino]-2-oxo- ethyl]-2-methyl-pyrazole-3-carboxamide | 612.249 | 2.11 |
| 95^{c} | 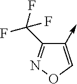 | 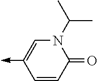 | 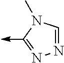 | N-[1-[[2-chloro-5-(1-isopropyl-6-oxo-3- pyridyl)phenyl]methyl]-2-[4-(4-methyl- 1,2,4-triazol-3-yl)anilino]-2-oxo-ethyl]-3- (trifluoromethyl)isoxazole-4-carboxamide | 654.185 | 2.14 |
| 96^{c} | 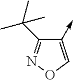 | 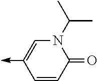 | 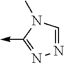 | 3-tert-butyl-N-[1-[[2-chloro-5-(1- isopropyl-6-oxo-3- pyridyl)phenyl]methyl]-2-[4-(4-methyl- 1,2,4-triazol-3-yl)anilino]-2-oxo- ethyl]isoxazole-4-carboxamide | 642.259 | 2.17 |
| 97^{c} | 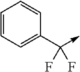 | 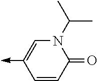 | 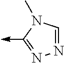 | 3-[2-chloro-5-(1-isopropyl-6-oxo-3- pyridyl)phenyl]-2-[(2,2-difluoro-2- phenyl-acetyl)amino]-N-[4-(4-methyl- 1,2,4-triazol-3-yl)phenyl]propanamide | 645.22 | 2.18 |
| 98^{c} | 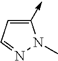 | 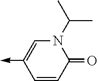 | 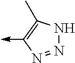 | N-[1-[[2-chloro-5-(1-isopropyl-6-oxo-3- pyridyl)phenyl]methyl]-2-[4-(5-methyl- 1H-triazol-4-yl)anilino]-2-oxo-ethyl]-2- methyl-pyrazole-3-carboxamide | 599.229 | 2.08 |
| 99ⁱ | 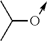 | 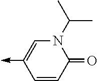 | 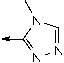 | isopropyl N-[(1S)-1-[[2-chloro-5-(1- isopropyl-6-oxo-3- pyridyl)phenyl]methyl]-2-[4-(4-methyl- 1,2,4-triazol-3-yl)anilino]-2-oxo- ethyl]carbamate | 577.234 | 2.09 |
| 100ⁱ | 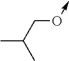 | 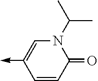 | 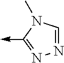 | isobutyl N-[(1S)-1-[[2-chloro-5-(1- isopropyl-6-oxo-3- pyridyl)phenyl]methyl]-2-[4-(4-methyl- 1,2,4-triazol-3-yl)anilino]-2-oxo- ethyl]carbamate | 591.249 | 2.16 |
| 104^{c} | 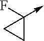 | 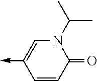 | 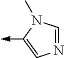 | N-[(1S)-1-[[2-chloro-5-(1-isopropyl-6- oxo-3-pyridyl)phenyl]methyl]-2-[4-(3- methylimidazol-4-yl)anilino]-2-oxo- ethyl]-1-fluoro-cyclopropanecarboxamide | 576.218 | 1.98 |
| 105ⁱ | 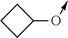 | 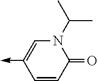 | 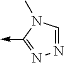 | cyclobutyl N-[1-[[2-chloro-5-(1-isopropyl- 6-oxo-3-pyridyl)phenyl]methyl]-2-[4-(4- methyl-1,2,4-triazol-3-yl)anilino]-2-oxo- ethyl]carbamate | 589.233 | 2.14 |
| 106ⁱ | 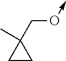 | 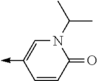 | 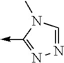 | (1-methylcyclopropyl)methyl N-[1-[[2- chloro-5-(1-isopropyl-6-oxo-3- pyridyl)phenyl]methyl]-2-[4-(4-methyl- 1,2,4-triazol-3-yl)anilino]-2-oxo- ethyl]carbamate | 603.25 | 2.18 |
| 107ⁱ | 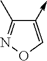 | 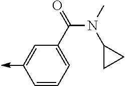 | 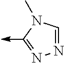 | N-[1-[[2-chloro-5-[3- [cyclopropyl(methyl)carbamoyl]phenyl] phenyl]methyl]-2-[4-(4-methyl-1,2,4- triazol-3-yl)anilino]-2-oxo-ethyl]-3- methyl-isoxazole-4-carboxamide | 638.228 | 2.16 |
| 108^{b} | 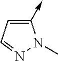 | 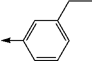 | 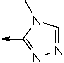 | N-[1-[[2-chloro-5-(3- ethylphenyl)phenyl]methyl]-2-[4-(4- methyl-1,2,4-triazol-3-yl)anilino]-2-oxo- ethyl]-2-methyl-pyrazole-3-carboxamide | 568.223 | 2.37 |
| 109^{b} | 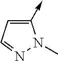 | 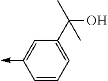 | 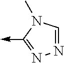 | N-[1-[[2-chloro-5-[3-(1-hydroxy-1- methyl-ethyl)phenyl]phenyl]methyl]-2- [4-(4-methyl-1,2,4-triazol-3-yl)anilino]- 2-oxo-ethyl]-2-methyl-pyrazole-3- carboxamide | 598.234 | 2.13 |
| 110^{b} | 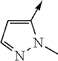 | 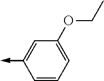 | 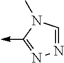 | N-[1-[[2-chloro-5-(3- ethoxyphenyl)phenyl]methyl]-2-[4-(4- methyl-1,2,4-triazol-3-yl)anilino]-2-oxo- ethyl]-2-methyl-pyrazole-3-carboxamide | 584.218 | 2.30 |
| 111^{b} | 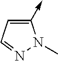 | 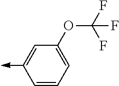 | 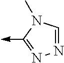 | N-[1-[[2-chloro-5-[3- (trifluoromethoxy)phenyl]phenyl]methyl]- 2-[4-(4-methyl-1,2,4-triazol-3- yl)anilino]-2-oxo-ethyl]-2-methyl- pyrazole-3-carboxamide | 624.174 | 2.37 |
| 112^{b} | 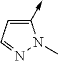 | 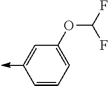 | 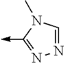 | N-[1-[[2-chloro-5-[3- (difluoromethoxy)phenyl]phenyl]methyl]- 2-[4-(4-methyl-1,2,4-triazol-3- yl)anilino]-2-oxo-ethyl]-2-methyl- pyrazole-3-carboxamide | 606.184 | 2.28 |
| 113ⁱ | 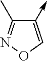 | 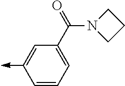 | 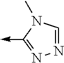 | N-[1-[[5-[3-(azetidine-1- carbonyl)phenyl]-2-chloro- phenyl]methyl]-2-[4-(4-methyl-1,2,4- triazol-3-yl)anilino]-2-oxo-ethyl]-3- methyl-isoxazole-4-carboxamide | 606.184 | 2.09 |
| 114^{c} | 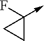 | 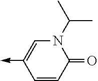 | 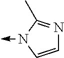 | N-[(1S)-1-[[2-chloro-5-(1-isopropyl-6- oxo-3-pyridyl)phenyl]methyl]-2-[4-(2- methylimidazol-1-yl)anilino]-2-oxo- ethyl]-1-fluoro-cyclopropanecarboxamide | 576.219 | 1.98 |
| 115^{c} | 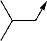 | 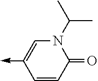 | 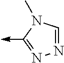 | N-[(1S)-1-[[2-chloro-5-(1-isopropyl-6- oxo-3-pyridyl)phenyl]methyl]-2-[4-(4- methyl-1,2,4-triazol-3-yl)anilino]-2-oxo- ethyl]-3-methyl-butanamide | 574.256 | 2.08 |
| 116^{c} | 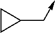 | 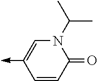 | 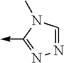 | (2S)-3-[2-chloro-5-(1-isopropyl-6-oxo-3- pyridyl)phenyl]-2-[(2- cyclopropylacetyl)amino]-N-[4-(4- methyl-1,2,4-triazol-3- yl)phenyl]propanamide | 572.239 | 2.05 |
| 117^{c} | 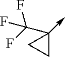 | 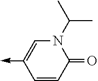 | 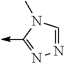 | N-[(1S)-1-[[2-chloro-5-(1-isopropyl-6- oxo-3-pyridyl)phenyl]methyl]-2-[4-(4- methyl-1,2,4-triazol-3-yl)anilino]-2-oxo- ethyl]-1-(trifluoromethyl) cyclopropanecarboxamide | 627.21 | 2.16 |
| 118^{c} | 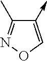 | 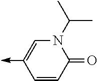 | 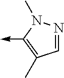 | N-[(1S)-1-[[2-chloro-5-(1-isopropyl-6- oxo-3-pyridyl)phenyl]methyl]-2-[4-(2,4- dimethylpyrazol-3-yl)anilino]-2-oxo- ethyl]-3-methyl-isoxazole-4-carboxamide | 613-233 | 2.27 |
| 119^{c} | 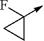 | 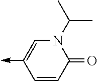 | 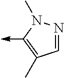 | N-[(1S)-1-[[2-chloro-5-(1-isopropyl-6- oxo-3-pyridyl)phenyl]methyl]-2-[4-(2,4- dimethylpyrazol-3-yl)anilino]-2-oxo- ethyl]-1-fluoro-cyclopropanecarboxamide | 590.234 | 2.32 |
| 120ⁱ | 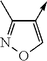 | 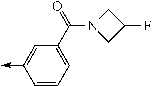 | 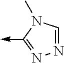 | N-[1-[[2-chloro-5-[3-(3-fluoroazetidine- 1-carbonyl)phenyl]phenyl]methyl]-2-[4- (4-methyl-1,2,4-triazol-3-yl)anilino]-2- oxo-ethyl]-3-methyl-isoxazole-4- carboxamide | 642.203 | 2.11 |
| 121^{j} | 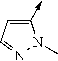 | 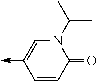 | 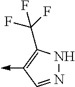 | N-[1-[[2-chloro-5-(1-isopropyl-6-oxo-3- pyridyl)phenyl]methyl]-2-oxo-2-[4-[3- (trifluoromethyl)-1H-pyrazol-4- yl]anilino]ethyl]-2-methyl-pyrazole-3- carboxamide | 652.205 | 2.24 |
| 122^{b} | 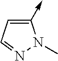 | 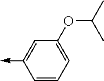 | 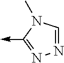 | N-[1-[[2-chloro-5-(3- isopropoxyphenyl)phenyl]methyl]-2-[4- (4-methyl-1,2,4-triazol-3-yl)anilino]-2- oxo-ethyl]-2-methyl-pyrazole-3- carboxamide | 598.234 | 2.36 |
| 123^{c} | 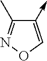 | 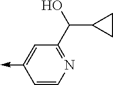 | 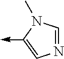 | N-[(1S)-1-[[2-chloro-5-[2- [cyclopropyl(hydroxy)methyl]-4- pyridyl]phenyl]methyl]-2-[4-(3- methylimidazol-4-yl)anilino]-2-oxo- ethyl]-3-methyl-isoxazole-4-carboxamide | 611.217 | 1.85 |
| 124^{c} | 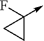 | 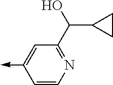 | 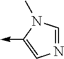 | N-[(1S)-1-[[2-chloro-5-[2- [cyclopropyl(hydroxy)methyl]-4- pyridyl]phenyl]methyl]-2-[4-(3- methylimidazol-4-yl)anilino]-2-oxo- ethyl]-1-fluoro-cyclopropanecarboxamide | 588.218 | 1.88 |
| 125^{c} | 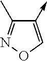 | 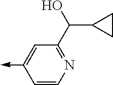 | 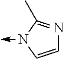 | N-[(1S)-1-[[2-chloro-5-[2- [cyclopropyl(hydroxy)methyl]-4- pyridyl]phenyl]methyl]-2-[4-(2- methylimidazol-1-yl)anilino]-2-oxo- ethyl]-3-methyl-isoxazole-4-carboxamide | 611.217 | 1.85 |
| 126^{c} | 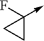 | 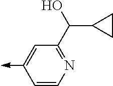 | 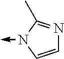 | N-[(1S)-1-[[2-chloro-5-[2- [cyclopropyl(hydroxy)methyl]-4- pyridyl]phenyl]methyl]-2-[4-(2- methylimidazol-1-yl)anilino]-2-oxo- ethyl]-1-fluoro-cyclopropanecarboxamide | 588.219 | 1.87 |
| 127^{c} | 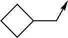 | 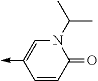 | 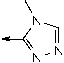 | (2S)-3-[2-chloro-5-(1-isopropyl-6-oxo-3- pyridyl)phenyl]-2-[(2- cyclobutylacetyl)amino]-N-[4-(4-methyl- 1,2,4-triazol-3-yl)phenyl]propanamide | 587.254 | 2.10 |
| 128^{c} | 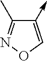 | 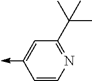 | 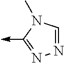 | N-[(1S)-1-[[5-(2-tert-butyl-4-pyridyl)-2- chloro-phenyl]methyl]-2-[4-(4-methyl- 1,2,4-triazol-3-yl)anilino]-2-oxo-ethyl]-3- methyl-isoxazole-4-carboxamide | 598.234 | 2.02 |
| 129^{c} | 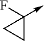 | 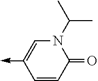 | 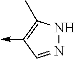 | N-[1-[[2-chloro-5-(1-isopropyl-6-oxo-3- pyridyl)phenyl]methyl]-2-[4-(5-methyl- 1H-pyrazol-4-yl)anilino]-2-oxo-ethyl]-1- fluoro-cyclopropanecarboxamide | 576.218 | 2.18 |
| 130^{k} | 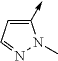 | 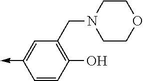 | 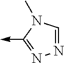 | N-[1-[[2-chloro-5-[4-hydroxy-3- (morpholinomethyl)phenyl]phenyl] methyl]-2-[4-(4-methyl-1,2,4-triazol-3- yl)anilino]-2-oxo-ethyl]-2-methyl- pyrazole-3-carboxamide | 655.257 | 1.84 |
| 131^{d} | 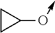 | 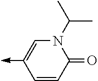 | 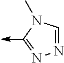 | cyclopropyl N-[(1S)-1-[[2-chloro-5-(1- isopropyl-6-oxo-3- pyridyl)phenyl]methyl]-2-[4-(4-methyl- 1,2,4-triazol-3-yl)anilino]-2-oxo- ethyl]carbamate | 575.217 | 2.05 |
| 136^{j} | 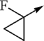 | 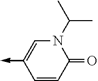 | 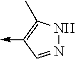 | N-[(1S)-1-[[2-chloro-5-(1-isopropyl-6- oxo-3-pyridyl)phenyl]methyl]-2-[4-(3- methyl-1H-pyrazol-4-yl)anilino]-2-oxo- ethyl]-1-fluoro-cyclopropanecarboxamide | 576.218 | 2.18 |
| 137^{j} | 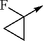 | 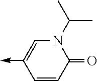 | 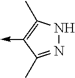 | N-[(1S)-1-[[2-chloro-5-(1-isopropyl-6- oxo-3-pyridyl)phenyl]methyl]-2-[4-(3,5- dimethyl-1H-pyrazol-4-yl)anilino]-2-oxo- ethyl]-1-fluoro-cyclopropanecarboxamide | 590.233 | 2.18 |
| 139^{k} | 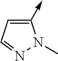 | 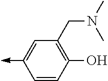 | 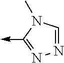 | N-[1-[[2-chloro-5-[3- (dimethylaminomethyl)-4-hydroxy- phenyl]phenyl]methyl]-2-[4-(4-methyl- 1,2,4-triazol-3-yl)anilino]-2-oxo-ethyl]-2- methyl-pyrazole-3-carboxamide | 613.245 | 1.83 |
| 140^{c} | 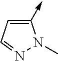 | 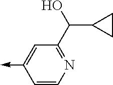 | 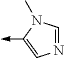 | N-[(1S)-1-[[2-chloro-5-[2- [cyclopropyl(hydroxy)methyl]-4- pyridyl]phenyl]methyl]-2-[4-(3- methylimidazol-4-yl)anilino]-2-oxo- ethyl]-2-methyl-pyrazole-3-carboxamide | 610.234 | 1.83 |
| 141^{c} | 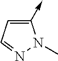 | 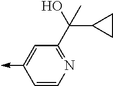 | 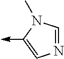 | N-[(1S)-1-[[2-chloro-5-[2-(1-cyclopropyl- 1-hydroxy-ethyl)-4- pyridyl]phenyl]methyl]-2-[4-(3- methylimidazol-4-yl)anilino]-2-oxo- ethyl]-2-methyl-pyrazole-3-carboxamide | 624.249 | 1.87 |
| 142^{c} | 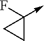 | 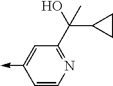 | 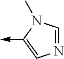 | N-[(1S)-1-[[2-chloro-5-[2-(1-cyclopropyl- 1-hydroxy-ethyl)-4- pyridyl]phenyl]methyl]-2-[4-(3- methylimidazol-4-yl)anilino]-2-oxo- ethyl]-1-fluoro-cyclopropanecarboxamide | 602.234 | 1.93 |
| 143^{b} | 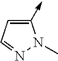 | 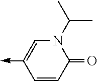 | 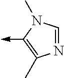 | N-[(1S)-1-[[2-chloro-5-(1-isopropyl-6- oxo-3-pyridyl)phenyl]methyl]-2-[4-(3,5- dimethylimidazol-4-yl)anilino]-2-oxo- ethyl]-2-methyl-pyrazole-3-carboxamide | 612.249 | 1.94 |
| 144^{b} | 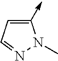 | 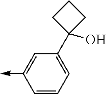 | 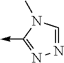 | N-[1-[[2-chloro-5-[3-(1- hydroxycyclobutyl)phenyl]phenyl] methyl]-2-[4-(4-methyl-1,2,4-triazol-3- yl)anilino]-2-oxo-ethyl]-2-methyl- pyrazole-3-carboxamide | 610.234 | 2.17 |
| 145^{b} | 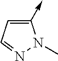 | 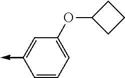 | 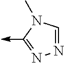 | N-[1-[[2-chloro-5-[3- (cyclobutoxy)phenyl]phenyl]methyl]-2- [4-(4-methyl-1,2,4-triazol-3-yl)anilino]- 2-oxo-ethyl]-2-methyl-pyrazole-3- carboxamide | 610.234 | 2.41 |
| 146^{b} | 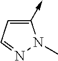 | 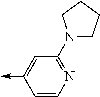 | 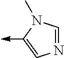 | N-[1-[[2-chloro-5-(2-pyrrolidin-1-yl-4- pyridyl)phenyl]methyl]-2-[4-(3- methylimidazol-4-yl)anilino]-2-oxo- ethyl]-2-methyl-pyrazole-3-carboxamide | 609.244 | 1.78 |
| 147^{c} | 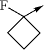 | 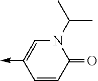 | 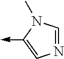 | N-[(1S)-1-[[2-chloro-5-(1-isopropyl-6- oxo-3-pyridyl)phenyl]methyl]-2-[4-(3- methylimidazol-4-yl)anilino]-2-oxo- ethyl]-1-fluoro-cyclobutanecarboxamide | 590.234 | 2.03 |
| 148^{c} | 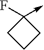 | 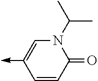 | 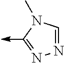 | N-[(1S)-1-[[2-chloro-5-(1-isopropyl-6- oxo-3-pyridyl)phenyl]methyl]-2-[4-(4- methyl-1,2,4-triazol-3-yl)anilino]-2-oxo- ethyl]-1-fluoro-cyclobutanecarboxamide | 591.236 | 2.11 |
| 149^{b} | 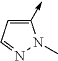 | 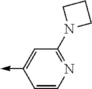 | 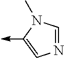 | N-[1-[[5-[2-(azetidin-1-yl)-4-pyridyl]-2- chloro-phenyl]methyl]-2-[4-(3- methylimidazol-4-yl)anilino]-2-oxo- ethyl]-2-methyl-pyrazole-3-carboxamide | 595.234 | 1.76 |
| 150^{b} | 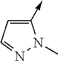 | 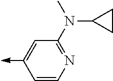 | 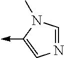 | N-[1-[[2-chloro-5-[2- [cyclopropyl(methyl)amino]-4- pyridyl]phenyl]methyl]-2-[4-(3- methylimidazol-4-yl)anilino]-2-oxo- ethyl]-2-methyl-pyrazole-3-carboxamide | 609.249 | 1.79 |
| 151^{b} | 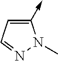 | 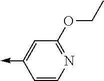 | 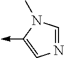 | N-[1-[[2-chloro-5-(2-ethoxy-4- pyridyl)phenyl]methyl]-2-[4-(3- methylimidazol-4-yl)anilino]-2-oxo- ethyl]-2-methyl-pyrazole-3-carboxamide | 584.218 | 2.09 |
| 152^{b} | 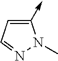 | 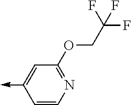 | 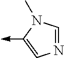 | N-[1-[[2-chloro-5-[2-(2,2,2- trifluoroethoxy)-4- pyridyl]phenyl]methyl]-2-[4-(3- methylimidazol-4-yl)anilino]-2-oxo- ethyl]-2-methyl-pyrazole-3-carboxamide | 638.19 | 2.20 |
| 153^{b} | 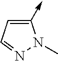 | 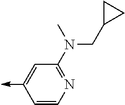 | 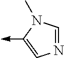 | N-[1-[[2-chloro-5-[2- [cyclopropylmethyl(methyl)amino]-4- pyridyl]phenyl]methyl]-2-[4-(3- methylimidazol-4-yl)anilino]-2-oxo- ethyl]-2-methyl-pyrazole-3-carboxamide | 623.265 | 1.85 |
| 154^{c} | 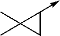 | 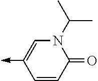 | 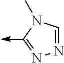 | N-[(1S)-1-[[2-chloro-5-(1-isopropyl-6- oxo-3-pyridyl)phenyl]methyl]-2-[4-(4- methyl-1,2,4-triazol-3-yl)anilino]-2-oxo- ethyl]-2,2-dimethyl- cyclopropanecarboxamide | 587.254 | 2.12 |
| 155ⁱ | 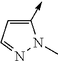 | 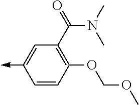 | 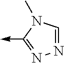 | N-[1-[[2-chloro-5-[3- (dimethylcarbamoyl)-4- (methoxymethoxy)phenyl]phenyl] methyl]-2-[4-(4-methyl-1,2,4-triazol-3- yl)anilino]-2-oxo-ethyl]-2-methyl- pyrazole-3-carboxamide | 671.25 | 2.06 |
| 156ⁱ | 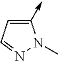 | 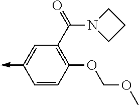 | 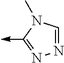 | N-[1-[[5-[3-(azetidine-1-carbonyl)-4- (methoxymethoxy)phenyl]-2-chloro- phenyl]methyl]-2-[4-(4-methyl-1,2,4- triazol-3-yl)anilino]-2-oxo-ethyl]-2- methyl-pyrazole-3-carboxamide | 683.251 | 2.06 |
| 157^{k} | 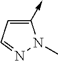 | 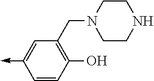 | 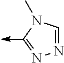 | N-[1-[[2-chloro-5-[4-hydroxy-3- (piperazin-1- ylmethyl)phenyl]phenyl]methyl]-2-[4-(4- methyl-1,2,4-triazol-3-yl)anilino]-2-oxo- ethyl]-2-methyl-pyrazole-3-carboxamide | 654.271 | 1.83 |
| 158^{b} | 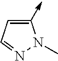 | 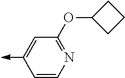 | 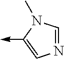 | N-[1-[[2-chloro-5-[2-(cyclobutoxy)-4- pyridyl]phenyl]methyl]-2-[4-(3- methylimidazol-4-yl)anilino]-2-oxo- ethyl]-2-methyl-pyrazole-3-carboxamide | 610.233 | 2.19 |
| 159^{b} | 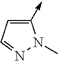 | 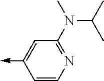 | 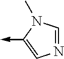 | N-[1-[[2-chloro-5-[2- [isopropyl(methyl)amino]-4- pyridyl]phenyl]methyl]-2-[4-(3- methylimidazol-4-yl)anilino]-2-oxo- ethyl]-2-methyl-pyrazole-3-carboxamide | 611.265 | 1.81 |
| 160^{b} | 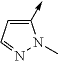 | 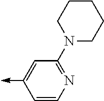 | 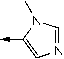 | N-[1-[[2-chloro-5-[2-(1-piperidyl)-4- pyridyl]phenyl]methyl]-2-[4-(3- methylimidazol-4-yl)anilino]-2-oxo- ethyl]-2-methyl-pyrazole-3-carboxamide | 623.264 | 1.85 |
| 161^{b} | 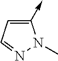 | 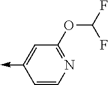 | 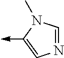 | N-[1-[[2-chloro-5-[2-(difluoromethoxy)- 4-pyridyl]phenyl]methyl]-2-[4-(3- methylimidazol-4-yl)anilino]-2-oxo- ethyl]-2-methyl-pyrazole-3-carboxamide | 606.184 | 2.13 |
| 162^{b} | 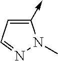 | 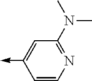 | 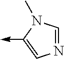 | N-[1-[[2-chloro-5-[2-(dimethylamino)-4- pyridyl]phenyl]methyl]-2-[4-(3- methylimidazol-4-yl)anilino]-2-oxo- ethyl]-2-methyl-pyrazole-3-carboxamide | 583.234 | 1.74 |
| 163^{c} | 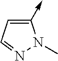 | 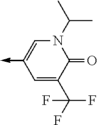 | 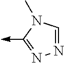 | N-[1-[[2-chloro-5-[1-isopropyl-6-oxo-5- (trifluoromethyl)-3- pyridyl]phenyl]methyl]-2-[4-(4-methyl- 1,2,4-triazol-3-yl)anilino]-2-oxo-ethyl]-2- methyl-pyrazole-3-carboxamide | 667.216 | 2.17 |
| 164^{b} | 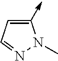 | 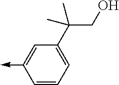 | 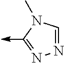 | N-[1-[[2-chloro-5-[3-(2-hydroxy-1,1- dimethyl-ethyl)phenyl]phenyl]methyl]-2- [4-(4-methyl-1,2,4-triazol-3-yl)anilino]- 2-oxo-ethyl]-2-methyl-pyrazole-3- carboxamide | 612.249 | 2.19 |
| 165^{b} | 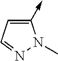 | 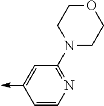 | 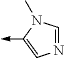 | N-[1-[[2-chloro-5-(2-morpholino-4- pyridyl)phenyl]methyl]-2-[4-(3- methylimidazol-4-yl)anilino]-2-oxo- ethyl]-2-methyl-pyrazole-3-carboxamide | 625.244 | 1.86 |
| 166^{b} | 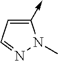 | 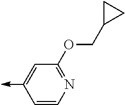 | 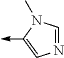 | N-[1-[[2-chloro-5-[2- (cyclopropylmethoxy)-4- pyridyl]phenyl]methyl]-2-[4-(3- methylimidazol-4-yl)anilino]-2-oxo- ethyl]-2-methyl-pyrazole-3-carboxamide | 610.234 | 2.18 |
| 167^{b} | 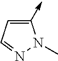 | 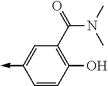 | 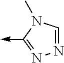 | N-[1-[[2-chloro-5-[3- (dimethylcarbamoyl)-4-hydroxy- phenyl]phenyl]methyl]-2-[4-(4-methyl- 1,2,4-triazol-3-yl)anilino]-2-oxo-ethyl]-2- methyl-pyrazole-3-carboxamide | 627.224 | 1.97 |
| 168^{k} | 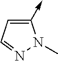 | 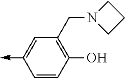 | 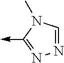 | N-[1-[[5-[3-(azetidin-1-ylmethyl)-4- hydroxy-phenyl]-2-chloro- phenyl]methyl]-2-[4-(4-methyl-1,2,4- triazol-3-yl)anilino]-2-oxo-ethyl]-2- methyl-pyrazole-3-carboxamide | 625.244 | 1.84 |
| 169^{b} | 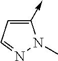 | 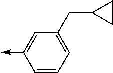 | 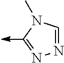 | N-[1-[[2-chloro-5-[3- (cyclopropylmethyl)phenyl]phenyl] methyl]-2-[4-(4-methyl-1,2,4-triazol-3- yl)anilino]-2-oxo-ethyl]-2-methyl- pyrazole-3-carboxamide | 594.239 | 2.43 |
| 170^{b} | 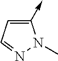 | 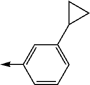 | 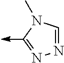 | N-[1-[[2-chloro-5-(3- cyclopropylphenyl)phenyl]methyl]-2-[4- (4-methyl-1,2,4-triazol-3-yl)anilino]-2- oxo-ethyl]-2-methyl-pyrazole-3- carboxamide | 580.223 | 2.36 |
| 171^{c} | 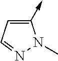 | 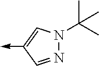 | 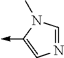 | N-[(1S)-1-[[5-(1-(1-butylpyrazol-4-yl)- 2-chloro-phenyl]methyl]-2-[4-(3- methylimidazol-4-yl)anilino]-2-oxo- ethyl]-2-methyl-pyrazole-3-carboxamide | 585.25 | 2.06 |
| 172ⁱ | 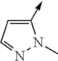 | 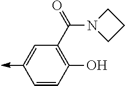 | 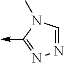 | N-[1-[[5-[3-(azetidine-1-carbonyl)-4- hydroxy-phenyl]-2-chloro- phenyl]methyl]-2-[4-(4-methyl-1,2,4- triazol-3-yl)anilino]-2-oxo-ethyl]-2- methyl-pyrazole-3-carboxamide | 639.225 | 2.12 |
| 173^{k} | 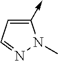 | 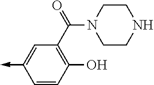 | 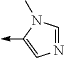 | N-[1-[[2-chloro-5-[4-hydroxy-3- (piperazine-1- carbonyl)phenyl]phenyl]methyl]-2-[4-(4- methyl-1,2,4-triazol-3-yl)anilino]-2-oxo- ethyl]-2-methyl-pyrazole-3-carboxamide | 668.25 | 1.79 |
| 176^{b} | 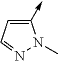 | 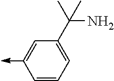 | 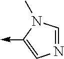 | N-[1-[[5-[3-(1-amino-1-methyl- ethyl)phenyl]-2-chloro-phenyl]methyl]-2- [4-(3-methylimidazol-4-yl)anilino]-2-oxo- ethyl]-2-methyl-pyrazole-3-carboxamide | 596.254 | 1.78 |
| 178^{b} | 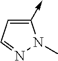 | 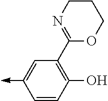 | 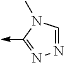 | N-[1-[[2-chloro-5-[3-(5,6-dihydro-4H- 1,3-oxazin-2-yl)-4-hydroxy- phenyl]phenyl]methyl]-2-[4-(4-methyl- 1,2,4-triazol-3-yl)anilino]-2-oxo-ethyl]-2- methyl-pyrazole-3-carboxamide | 639.224 | 1.98 |
| 179^{c} | 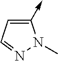 | 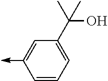 | 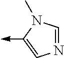 | N-[1-[[2-chloro-5-[3-(1-hydroxy-1- methyl-ethyl)phenyl]phenyl]methyl]-2- [4-(3-methylimidazol-4-yl)anilino]-2-oxo- ethyl]-2-methyl-pyrazole-3-carboxamide | 597.238 | 2.05 |
| 180^{g} | 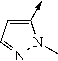 | 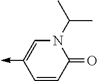 | 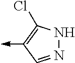 | N-[1-[[2-chloro-5-(1-(l-6-oxo-3- pyridyl)phenyl]methyl]-2-[4-(3-chloro- 1H-pyrazol-4-yl)anilino]-2-oxo-ethyl]-2- methyl-pyrazole-3-carboxamide | 618.179 | 2.18 |
| 181^{b} | 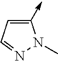 | 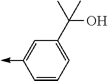 | 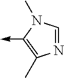 | N-[(1S)-[[2-chloro-5-[3-(1-hydroxy-1- methyl-ethyl)phenyl]phenyl]methyl]-2- [4-(3,5-dimethylimidazol-4-yl)anilino]-2- oxo-ethyl]-2-methyl-pyrazole-3- carboxamide | 611.254 | 2.07 |
| 182^{b} | 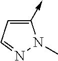 | 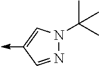 | 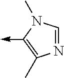 | N-[(1S)-1-[[5-(1-tert-butylpyrazol-4-yl)- 2-chloro-phenyl]methyl]-2-[4-(3,5- dimethylimidazol-4-yl)anilino]-2-oxo- ethyl]-2-methyl-pyrazole-3-carboxamide | 599.265 | 2.08 |
| 183^{b} | 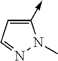 | 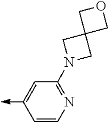 | 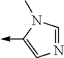 | N-[1-[[2-chloro-5-[2-(6-oxa-2- azaspiro[3.3]heptan-2-yl)-4- pyridyl]phenyl]methyl]-2-[4-(3- methylimidazol-4-yl)anilino]-2-oxo- ethyl]-2-methyl-pyrazole-3-carboxamide | 637.246 | 1.75 |
| 184^{b} | 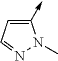 | 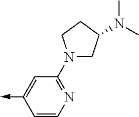 | 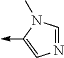 | N-[1-[[2-chloro-5-[2-[(3S)-3- (dimethylamino)pyrrolidin-1-yl]-4- pyridyl]phenyl]methyl]-2-[4-(3- methylimidazol-4-yl)anilino]-2-oxo- ethyl]-2-methyl-pyrazole-3-carboxamide | 652.292 | 1.66 |
| 185^{b} | 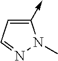 | 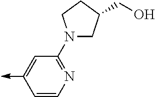 | 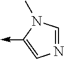 | N-[1-[[2-chloro-5-[2-[(3S)-3- (hydroxymethyl)pyrrolid in-1-yl]-4- pyridyl]phenyl]methyl]-2-[4-(3- methylimidazol-4-yl)anilino]-2-oxo- ethyl]-2-methyl-pyrazole-3-carboxamide | 639.261 | 1.73 |
| 186^{b} | 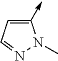 | 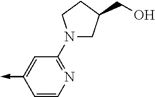 | 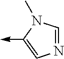 | N-[1-[[2-chloro-5-[2-[(3R)-3- (hydroxymethyl)pyrrolidin-1-yl]-4- pyridyl]phenyl]methyl]-2-[4-(3- methylimidazol-4-yl)anilino]-2-oxo- ethyl]-2-methyl-pyrazole-3-carboxamide | 639.26 | 1.73 |
| 187^{b} | 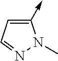 | 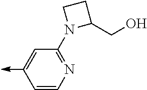 | 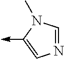 | N-[1-[[2-chloro-5-[2-[2- (hydroxymethyl)azetidin-1-yl]-4- pyridyl]phenyl]methyl]-2-[4-(3- methylimidazol-4-yl)anilino]-2-oxo- ethyl]-2-methyl-pyrazole-3-carboxamide | 625.246 | 1.75 |
| 188^{b} | 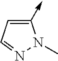 | 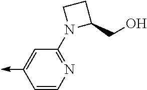 | 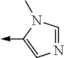 | N-[1-[[2-chloro-5-[2-[(2S)-2- (hydroxymethyl)azetidin-1-yl]-4- pyridyl]phenyl]methyl]-2-[4-(3- methylimidazol-4-yl)anilino]-2-oxo- ethyl]-2-methyl-pyrazole-3-carboxamide | 625.245 | 1.75 |
| 189^{b} | 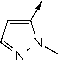 | 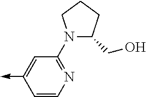 | 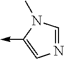 | N-[1-[[2-chloro-5-[2-[(2R)-2- (hydroxymethyl)pyrrolidin-1-yl]-4- pyridyl]phenyl]methyl]-2-[4-(3- methylimidazol-4-yl)anilino]-2-oxo- ethyl]-2-methyl-pyrazole-3-carboxamide | 639.26 | 1.75 |
| 190^{b} | 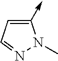 | 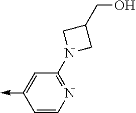 | 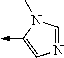 | N-[1-[[2-chloro-5-[2-[3- (hydroxymethyl)azetidin-1-yl]-4- pyridyl]phenyl]methyl]-2-[4-(3- methylimidazol-4-yl)anilino]-2-oxo- ethyl]-2-methyl-pyrazole-3-carboxamide | 625.245 | 1.72 |
| 191^{b} | 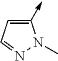 | 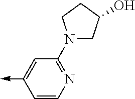 | 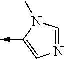 | N-[1-[[2-chloro-5-[2-[(3S)-3- hydroxypyrrolidin-1-yl]-4- pyridyl]phenyl]methyl]-2-[4-(3- methylimidazol-4-yl)anilino]-2-oxo- ethyl]-2-methyl-pyrazole-3-carboxamide | 625.244 | 1.71 |
| 192^{b} | 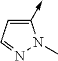 | 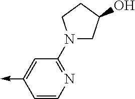 | 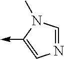 | N-[1-[[2-chloro-5-[2-[(3R)-3- hydroxypyrrolidin-1-yl]-4- pyridyl]phenyl]methyl]-2-[4-(3- methylimidazol-4-yl)anilino]-2-oxo- ethyl]-2-methyl-pyrazole-3-carboxamide | 625.245 | 1.71 |
| 193^{b} | 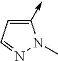 | 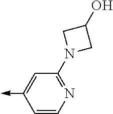 | 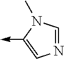 | N-[1-[[2-chloro-5-[2-(3-hydroxyazetidin- 1-yl)-4-pyridyl]phenyl]methyl]-2-[4-(3- methylimidazol-4-yl)anilino]-2-oxo- ethyl]-2-methyl-pyrazole-3-carboxamide | 611.229 | 1.72 |
| 194^{b} | 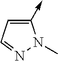 | 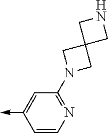 | 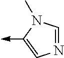 | N-[1-[[2-chloro-5-[2-(2,6- diazaspiro[3.3]heptan-6-yl)-4- pyridyl]phenyl]methyl]-2-[4-(3- methylimidazol-4-yl)anilino]-2-oxo- ethyl]-2-methyl-pyrazole-3-carboxamide | 636.262 | 1.64 |
| 195^{b} | 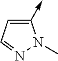 | 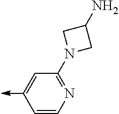 | 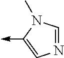 | N-[1-[[5-[2-(3-aminoazetidin-1-yl)-4- pyridyl]-2-chloro-phenyl]methyl]-2-[4-(3- methylimidazol-4-yl)anilino]-2-oxo- ethyl]-2-methyl-pyrazole-3-carboxamide | 610.245 | 1.65 |
| 196^{b} | 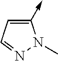 | 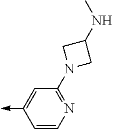 | 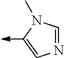 | N-[1-[[2-chloro-5-[2-[3- (methylamino)azetidin-1-yl]-4- pyridyl]phenyl]methyl]-2-[4-(3- methylimidazol-4-yl)anilino]-2-oxo- ethyl]-2-methyl-pyrazole-3-carboxamide | 624.26 | 1.67 |
| 197^{b} | 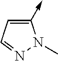 | 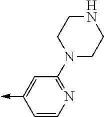 | 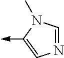 | N-[1-[[2-chloro-5-(2-piperazin-1-yl-4- pyridyl)phenyl]methyl]-2-[4-(3- methylimidazol-4-yl)anilino]-2-oxo- ethyl]-2-methyl-pyrazole-3-carboxamide | 624.261 | 1.74 |
| 198^{b} | 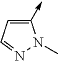 | 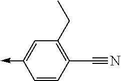 | 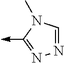 | N-[1-[[2-chloro-5-(4-cyano-3-ethyl- phenyl)phenyl]methyl]-2-[4-(4-methyl- 1,2,4-triazol-3-yl)anilino]-2-oxo-ethyl]-2- methyl-pyrazole-3-carboxamide | 593.218 | 2.29 |
| 199^{b} | 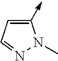 | 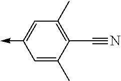 | 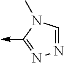 | N-[1-[[2-chloro-5-(4-cyano-3,5-dimethyl- phenyl)phenyl]methyl]-2-[4-(4-methyl- 1,2,4-triazol-3-yl)anilino]-2-oxo-ethyl]-2- methyl-pyrazole-3-carboxamide | 593.219 | 2.29 |
| 200^{a} | 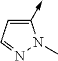 | 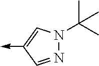 | 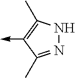 | N-[(1S)-1-[[5-(1-tert-butylpyrazol-4-yl)- 2-chloro-phenyl]methyl]-2-[4-(3,5- dimethyl-1H-pyrazol-4-yl)anilino]-2-oxo- ethyl]-2-methyl-pyrazole-3-carboxamide | 599.265 | 2.28 |
| 201^{d} | 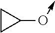 | 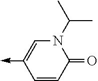 | 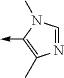 | cyclopropyl N-[(1S)-1-[[2-chloro-5-(1- isopropyl-6-oxo-3- pyridyl)phenyl]methyl]-2-[4-(3,5- dimethylimidazol-4-yl)anilino]-2-oxo- ethyl]carbamate | 588.239 | 2.00 |
| 202^{b} | 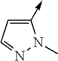 | 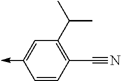 | 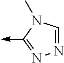 | N-[1-[[2-chloro-5-(4-cyano-3-isopropyl- phenyl)phenyl]methyl]-2-[4-(4-methyl- 1,2,4-triazol-3-yl)anilino]-2-oxo-ethyl]-2- methyl-pyrazole-3-carboxamide | 607.234 | 2.34 |
| 203^{b} | 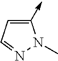 | 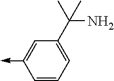 | 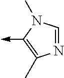 | N-[(1S)-1-[[5-[3-(1-amino-1-methyl- ethyl)phenyl]-2-chloro-phenyl]methyl]-2- [4-(3,5-dimethylimidazol-4-yl)anilino]-2- oxo-ethyl]-2-methyl-pyrazole-3- carboxamide | 610.27 | 1.79 |
| 204^{b} | 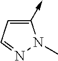 | 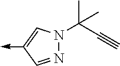 | 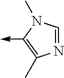 | N-[(1S)-1-[[2-chloro-5-[1-(1-cyano-1- methyl-ethyl)pyrazol-4- yl]phenyl]methyl]-2-[4-(3,5- dimethylimidazol-4-yl)anilino]-2-oxo- ethyl]-2-methyl-pyrazole-3-carboxamide | 610.246 | 2.03 |
| 205^{c} | 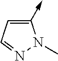 | 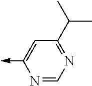 | 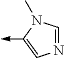 | N-[(1S)-1-[[2-chloro-5-(6- isopropylpyrimidin-4-yl)phenyl]methyl]- 2-[4-(3-methylimidazol-4-yl)anilino]-2- oxo-ethyl]-2-methyl-pyrazole-3- carboxamide | 583.234 | 2.02 |
| 206^{b} | 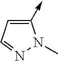 | 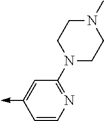 | 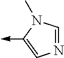 | N-[(1S)-1-[[2-chloro-5-[2-(4- methylpiperazin-1-yl)-4- pyridyl]phenyl]methyl]-2-[4-(3- methylimidazol-4-yl)anilino]-2-oxo- ethyl]-2-methyl-pyrazole-3-carboxamide | 638.276 | 1.74 |
| 207^{e} | 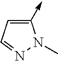 | 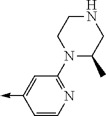 | 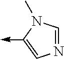 | N-[(1S)-1-[[2-chloro-5-[2-[(2R)-2- methylpiperazin-1-yl]-4- pyridyl]phenyl]methyl]-2-[4-(3- methylimidazol-4-yl)anilino]-2-oxo- ethyl]-2-methyl-pyrazole-3-carboxamide | 638.276 | 1.75 |
| 208^{e} | 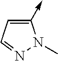 | 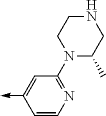 | 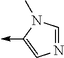 | N-[(1S)-1-[[2-chloro-5-[2-[(2S)-2- methylpiperazin-1-yl]-4- pyridyl]phenyl]methyl]-2-[4-(3- methylimidazol-4-yl)anilino]-2-oxo- ethyl]-2-methyl-pyrazole-3-carboxamide | 638.276 | 1.75 |
| 209^{e} | 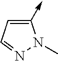 | 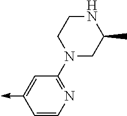 | 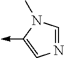 | N-[(1S)-1-[[2-chloro-5-[2-[(3S)-3- methylpiperazin-1-yl]-4- pyridyl]phenyl]methyl]-2-[4-(3- methylimidazol-4-yl)anilino]-2-oxo- ethyl]-2-methyl-pyrazole-3-carboxamide | 638.276 | 1.74 |
| 210^{e} | 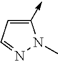 | 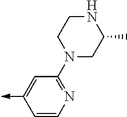 | 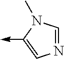 | N-[(1S)-1-[[2-chloro-5-[2-[(3R)-3- methylpiperazin-1-yl]-4- pyridyl]phenyl]methyl]-2-[4-(3- methylimidazol-4-yl)anilino]-2-oxo- ethyl]-2-methyl-pyrazole-3-carboxamide | 638.277 | 1.75 |
| 211^{d} | 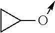 | 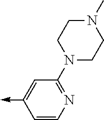 | 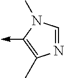 | cyclopropyl N-[(1S)-1-[[2-chloro-5-[2-(4- methylpiperazin-1-yl)-4- pyridyl]phenyl]methyl]-2-[4-(3,5- dimethylimidazol-4-yl)anilino]-2-oxo- ethyl]carbamate | 628.281 | 1.79 |
| 212^{c} | 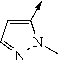 | 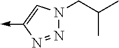 | 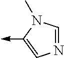 | N-[(1S)-1-[[2-chloro-5-(1-isobutyltriazol- 4-yl)phenyl]methyl]-2-[4-(3- methylimidazol-4-yl)anilino]-2-oxo- ethyl]-2-methyl-pyrazole-3-carboxamide | 586.245 | 2.00 |
| 213^{c} | 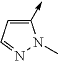 | 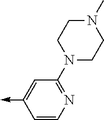 | 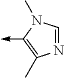 | N-[(1S)-1-[[2-chloro-5-[2-(4- methylpiperazin-1-yl)-4- pyridyl]phenyl]methyl]-2-[4-(3,5- dimethylimidazol-4-yl)anilino]-2-oxo- ethyl]-2-methyl-pyrazole-3-carboxamide | 652.292 | 1.76 |
| 214^{d} | 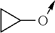 | 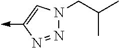 | 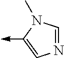 | cyclopropyl N-[(1S)-1-[[2-chloro-5-(1- isobutyltriazol-4-yl)phenyl]methyl]-2-[4- (3-methylimidazol-4-yl)anilino]-2-oxo- ethyl]carbamate | 562.235 | 2.06 |
| 215^{e} | 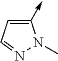 | 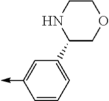 | 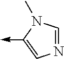 | N-[(1S)-1-[[2-chloro-5-[3-[(3S)- morpholin-3-yl]phenyl]phenyl]methyl]-2- [4-(3-methylimidazol-4-yl)anilino]-2-oxo- ethyl]-2-methyl-pyrazole-3-carboxamide | 624.25 | 1.75 |
| 216^{e} | 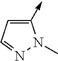 | 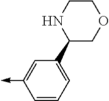 | 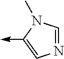 | N-[(1S)-1-[[2-chloro-5-[3-[(3R)- morpholin-3-yl]phenyl]phenyl]methyl]-2- [4-(3-methylimidazol-4-yl)anilino]-2-oxo- ethyl]-2-methyl-pyrazole-3-carboxamide | 624.251 | 1.76 |
| 217^{b} | 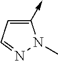 | 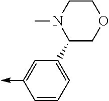 | 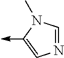 | N-[(1S)-1-[[2-chloro-5-[3-[(3S)-4- methylmorpholin-3- yl]phenyl]phenyl]methyl]-2-[4-(3- methylimidazol-4-yl)anilino]-2-oxo- ethyl]-2-methyl-pyrazole-3-carboxamide | 638.265 | 1.78 |
| 218^{b} | 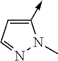 | 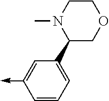 | 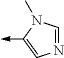 | N-[(1S)-1-[[2-chloro-5-[3-[(3R)-4- methylmorpholin-3- yl]phenyl]phenyl]methyl]-2-[4-(3- methylimidazol-4-yl)anilino]-2-oxo- ethyl]-2-methyl-pyrazole-3-carboxamide | 638.265 | 1.78 |
| 219^{e} | 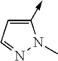 | 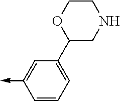 | 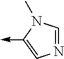 | N-[(1S)-1-[[2-chloro-5-(3-morpholin-2- ylphenyl)phenyl]methyl]-2-[4-(3- methylimidazol-4-yl)anilino]-2-oxo- ethyl]-2-methyl-pyrazole-3-carboxamide hydrochloride | 624.253 | 1.79 |
| 220^{e} | 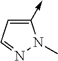 | 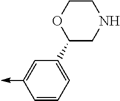 | 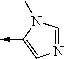 | N-[(1S)-1-[[2-chloro-5-[3-[(2S)- morpholin-2-yl]phenyl]phenyl]methyl]-2- [4-(3-methylimidazol-4-yl)anilino]-2-oxo- ethyl]-2-methyl-pyrazole-3-carboxamide | 624.253 | 1.79 |
| 221^{e} | 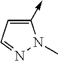 | 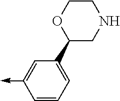 | 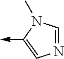 | N-[(1S)-1-[[2-chloro-5-[3-[(2R)- morpholin-2-yl]phenyl]phenyl]methyl]-2- [4-(3-methylimidazol-4-yl)anilino]-2-oxo- ethyl]-2-methyl-pyrazole-3-carboxamide | 624.252 | 1.79 |
| 222^{a} | 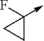 | 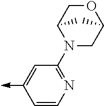 | 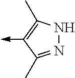 | N-[(1S)-1-[[2-chloro-5-[2-[(1R,4R)-2- oxa-5-azabicyclo[2.2.1]heptan-5-yl]-4- pyridyl]phenyl]methyl]-2-[4-(3,5- dimethyl-1H-pyrazol-4-yl)anilino]-2-oxo- ethyl]-1-fluoro-cyclopropanecarboxamide | 629.247 | 1.97 |
| 223^{a} | 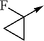 | 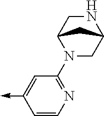 | 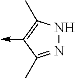 | N-[(1S)-1-[[2-chloro-5-[2-[(1S,4S)-2,5- diazabicyclo[2.2.1]heptan-2-yl]-4- pyridyl]phenyl]methyl]-2-[4-(3,5- dimethyl-1H-pyrazol-4-yl)anilino]-2-oxo- ethyl]-1-fluoro-cyclopropanecarboxamide | 628.262 | 1.87 |
| 224^{c} | 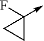 | 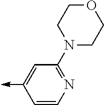 | 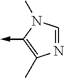 | N-[(1S)-1-[[2-chloro-5-(2-morpholino-4- pyridyl)phenyl]methyl]-2-[4-(3,5- dimethylimidazol-4-yl)anilino]-2-oxo- ethyl]-1-fluoro-cyclopropanecarboxamide | 617.246 | 1.93 |
| 225^{a} | 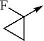 | 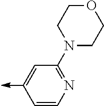 | 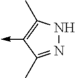 | N-[(1S)-1-[[2-chloro-5-(2-morpholino-4- pyridyl)phenyl]methyl]-2-[4-(3,5- dimethyl-1H-pyrazol-4-yl)anilino]-2-oxo- ethyl]-1-fluoro-cyclopropanecarboxamide | 617.245 | 2.11 |
| 226^{a} | 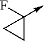 | 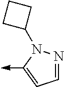 | 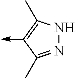 | N-[(1S)-1-[[2-chloro-5-(2- cyclobutylpyrazol-3-yl)phenyl]methyl]-2- [4-(3,5-dimethyl-1H-pyrazol-4- yl)anilino]-2-oxo-ethyl]-1-fluoro- cyclopropanecarboxamide | 575.235 | 2.38 |
| 227^{a} | 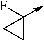 | 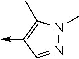 | 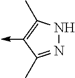 | N-[(1S)-1-[[2-chloro-5-(1,5- dimethylpyrazol-4-yl)phenyl]methyl]-2- [4-(3,5-dimethyl-1H-pyrazol-4- yl)anilino]-2-oxo-ethyl]-1-fluoro- cyclopropanecarboxamide | 549.22 | 2.21 |
| 228^{a} | 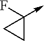 | 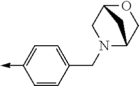 | 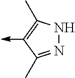 | N-[(1S)-1-[[2-chloro-5-[4-[[(1R,4R)-2- oxa-5-azabicyclo[2.2.1]heptan-5- yl]methyl]phenyl]phenyl]methyl]-2-[4- (3,5-dimethyl-1H-pyrazol-4-yl)anilino]-2- oxo-ethyl]-1-fluoro- cyclopropanecarboxamide | 642.267 | 1.98 |
| 229^{a} | 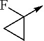 | 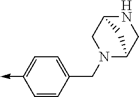 | 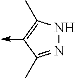 | N-[(1S)-1-[[2-chloro-5-[4-[[(1S,4S)-2,5- diazabicyclo[2.2.1]heptan-2- yl]methyl]phenyl]phenyl]methyl]-2-[4- (3,5-dimethyl-1H-pyrazol-4-yl)anilino]-2- oxo-ethyl]-1-fluoro- cyclopropanecarboxamide | 641.283 | 1.90 |

*RT are XEV Metode 1 CM − ES+ unless stated
^{a}prepared according to the method of Example 1
^{b}prepared according to the method of Example 3
^{c}prepared according to the method of Preparation 29
^{d}prepared according to the method of Preparation 87
^{e}prepared according to the method of Example 3, followed by Boc deprotection
^{g}prepared according to the methodology described in Prepration 14
^{h}prepared according to literature methodology of alcohol oxidation
ⁱprepared according to literature methodology of carbamate formation
^{j}prepared according to the method described in Example 230
^{k}prepared according to the method of Example 3, followed by reductive amination
^{l}prepared according to literature methodology of amide formation

Examples of the general formula (G) are tabled below

(G)

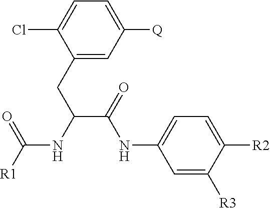

| Example | | | | | | Mass | |
|---|---|---|---|---|---|---|---|
| number | R1 | Q | R2 | R3 | Name | ion | RT* |
| 69^{m} | 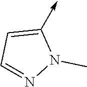 | 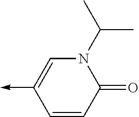 | 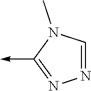 | OH | N-[1-[[2-chloro-5-(1-isopropyl- 6-oxo-3-pyridyl)phenyl]methyl]- 2-[3-hydroxy-4-(4-methyl- 1,2,4-triazol-3-yl)anilino]-2-oxo- ethyl]-2-methyl-pyrazole-3- carboxamide | 615.226 | 1.98 |
| 70^{c} | 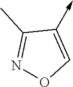 | 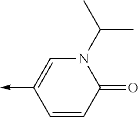 | 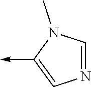 | OCH₃ | N-[(1S)-1-[[2-chloro-5-(1- isopropyl-6-oxo-3- pyridyl)phenyl]methyl]-2-[3- methoxy-4-(2-methylpyrazol-3- yl)anilino]-2-oxo-ethyl]-3- methyl-isoxazole-4-carboxamide | 629.228 | 2.22 |
| 71^{m} | 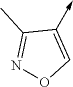 | 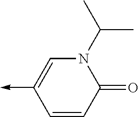 | 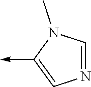 | OH | N-[(1S)-1-[[2-chloro-5-(1- isopropyl-6-oxo-3- pyridyl)phenyl]methyl]-2-[3- hydroxy-4-(2-methylpyrazol-3- yl)anilino]-2-oxo-ethyl]-3- methyl-isoxazole-4-carboxamide | 615.214 | 2.13 |
| 101^{g} | 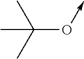 | 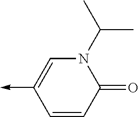 | 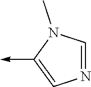 | Cl | tert-butyl N-[1-[[2-chloro-5-(1- isopropyl-6-oxo-3- pyridyl)phenyl]methyl]-2-[3- chloro-4-(3-methylimidazol-4- yl)anilino]-2-oxo- ethyl]carbamate | 624.215 | 2.13 |
| 102^{c} | 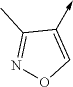 | 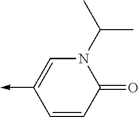 | 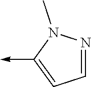 | Cl | N-[1-[[2-chloro-5-(1-isopropyl- 6-oxo-3-pyridyl)phenyl]methyl]- 2-[3-chloro-4-(2-methylpyrazol- 3-yl)anilino]-2-oxo-ethyl]-3- methyl-isoxazole-4-carboxamide | 633.179 | 2.30 |
| 103^{c} | 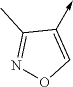 | 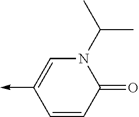 | 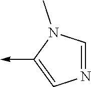 | Cl | N-[1-[[2-chloro-5-(1-isopropyl- 6-oxo-3-pyridyl)phenyl]methyl]- 2-[3-chloro-4-(3- methylimidazol-4-yl)anilino]-2- oxo-ethyl]-3-methyl-isoxazole- 4-carboxamide | 633.179 | 1.99 |
| 132^{m} | 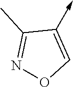 | 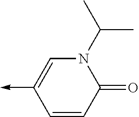 | 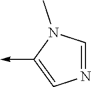 | OH | N-[(1S)-1-[[2-chloro-5-(1- isopropyl-6-oxo-3- pyridyl)phenyl]methyl]-2-[3- hydroxy-4-(3-methylimidazol-4- yl)anilino]-2-oxo-ethyl]-3- methyl-isoxazole-4-carboxamide | 615.212 | 1.93 |
| 133^{m} | 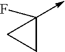 | 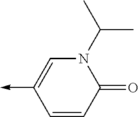 | 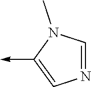 | OH | N-[(1S)-1-[[2-chloro-5-(1- isopropyl-6-oxo-3- pyridyl)phenyl]methyl]-2-[3- hydroxy-4-(3-methylimidazol-4- yl)anilino]-2-oxo-ethyl]-1- fluoro-cyclopropanecarboxamide | 592.213 | 1.95 |
| 134^{j} | 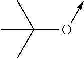 | 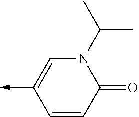 | 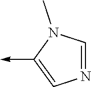 | F | tert-butyl N-[(1S)-1-[[2-chloro- 5-(1-isopropyl-6-oxo-3- pyridyl)phenyl]methyl]-2-[3- fluoro-4-(3-methylimidazol-4- yl)anilino]-2-oxo- ethyl]carbamate | 608.246 | 2.11 |
| 135^{j} | 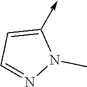 | 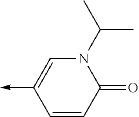 | 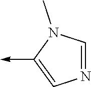 | F | N-[(1S)-1-[[2-chloro-5-(1- isopropyl-6-oxo-3- pyridyl)phenyl]methyl]-2-[3- fluoro-4-(3-methylimidazol-4- yl)anilino]-2-oxo-ethyl]-2- methyl-pyrazole-3-carboxamide | 616.226 | 1.95 |
| 138^{j} | 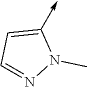 | 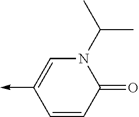 | 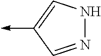 | F | N-[(1S)-1-[[2-chloro-5-(1- isopropyl-6-oxo-3- pyridyl)phenyl]methyl]-2-[3- fluoro-4-(1H-pyrazol-4- yl)anilino]-2-oxo-ethyl]-2- methyl-pyrazole-3-carboxamide | 602.209 | 2.12 |
| 174^{m} | 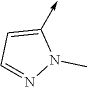 | 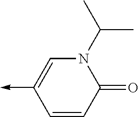 | 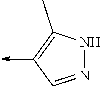 | OH | N-[(1S)-1-[[2-chloro-5-(1- isopropyl-6-oxo-3- pyridyl)phenyl]methyl]-2-[3- hydroxy-4-(3-methyl-1H- pyrazol-4-yl)anilino]-2-oxo- ethyl]-2-methyl-pyrazole-3- carboxamide | 614.229 | 2.03 |
| 175^{m} | 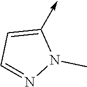 | 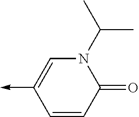 | 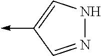 | OH | N-[(1S)-1-[[2-chloro-5-(1- isopropyl-6-oxo-3- pyridyl)phenyl]methyl]-2-[3- hydroxy-4-(1H-pyrazol-4- yl)anilino]-2-oxo-ethyl]-2- methyl-pyrazole-3-carboxamide | 600.213 | 2.02 |
| 177^{m} | 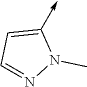 | 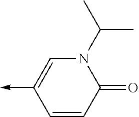 | 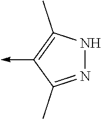 | OH | N-[(1S)-1-[[2-chloro-5-(1- isopropyl-6-oxo-3- pyridyl)phenyl]methyl]-2-[4- (3,5-dimethyl-1H-pyrazol-4-yl)- 3-hydroxy-anilino]-2-oxo-ethyl]- 2-methyl-pyrazole-3- carboxamide | 628.244 | 2.03 |

*RT are XEV Metode 1 CM - ES+ unless stated
^{c}prepared according to the method of Preparation 29
^{g}prepared according to the methodology described in Prepration 14
^{j}prepared according to the method describe in Example 230
^{m}prepared according to literature methodology for phenolmethyl ether deprotection

Examples of the general formula (H) are tabled below

(H)

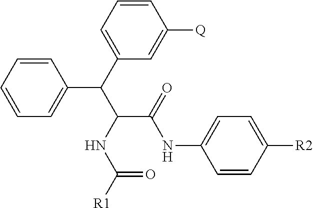

| Example | | | | | Mass | |
|---|---|---|---|---|---|---|
| number | R1 | Q | R2 | Name | ion | RT* |
| 232^{j} | 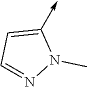 | 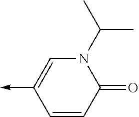 | 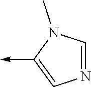 | N-[1-[[3-(1-isopropyl-6-oxo- 3-pyridyl)phenyl]-phenyl- methyl]-2-[4-(3- methylimidazol-4-yl)anilino]- 2-oxo-ethyl]-2-methyl- pyrazole-3-carboxamide (Diastereomer 2) | 640.308 | 1.98 |
| 233^{j} | 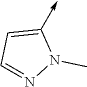 | 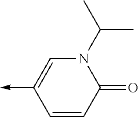 | 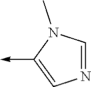 | N-[1-[[3-(1-isopropyl-6-oxo 3-pyridyl)phenyl]-phenyl- methyl]-2-[4-(3- methylimidazol-4-yl)anilino] 2-oxo-ethyl]-2-methyl- pyrazole-3-carboxamide (Diastereomer 3) | 640.304 | 1.96 |
| 234^{j} | 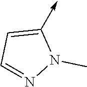 | 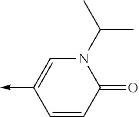 | 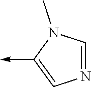 | N-[1-[[3-(1-isopropyl-6-oxo- 3-pyridyl)phenyl]-phenyl- methyl]-2-[4-(3- methylimidazol-4-yl)anilino]- 2-oxo-ethyl]-2-methyl- pyrazole-3-carboxamide (Diastereomer 4) | 640.304 | 1.96 |
| 235^{a} | 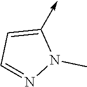 | 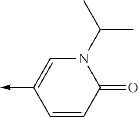 | 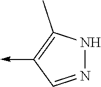 | N-[1-[[3-(1-isopropyl-6-oxo- 3-pyridyl)phenyl]-phenyl- methyl]-2-[4-(3-methyl-1H- pyrazol-4-yl)anilino]-2-oxo- ethyl]-2-methyl-pyrazole-3- carboxamide (Diastereomer 1) | 640.304 | 2.13 |
| 236^{a} | 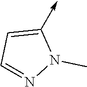 | 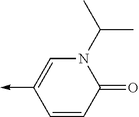 | 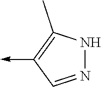 | N-[1-[[3-(1-isopropyl-6-oxo- 3-pyridyl)phenyl]-phenyl- methyl]-2-[4-(3-methyl-1H- pyrazol-4-yl)anilino]-2-oxo- ethyl]-2-methyl-pyrazole-3- carboxamide (Diastereomer 2) | 640.303 | 2.14 |
| 237^{a} | 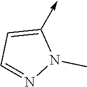 | 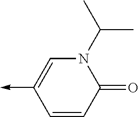 | 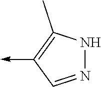 | N-[1-[[3-(1-isopropyl-6-oxo- 3-pyridyl)phenyl]-phenyl- methyl]-2-[4-(3-methyl-1H- pyrazol-4-yl)anilino]-2-oxo- ethyl]-2-methyl-pyrazole-3- carboxamide (Diastereomer 3) | 640.304 | 2.13 |
| 238^{a} | 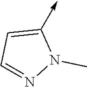 | 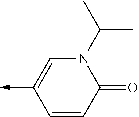 | 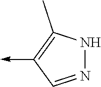 | N-[1-[[3-(1-isopropyl-6-oxo- 3-pyridyl)phenyl]-phenyl- methyl]-2-[4-(3-methyl-1H- pyrazol-4-yl)anilino]-2-oxo- ethyl]-2-methyl-pyrazole-3- carboxamide (Diastereomer 4) | 640.303 | 2.14 |
| 239^{j} | 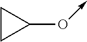 | 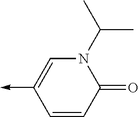 | 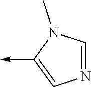 | cyclopropyl N-[1-[[3-(1- isopropyl-6-oxo-3- pyridyl)phenyl]-phenyl- methyl]-2-[4-(3- methylimidazol-4-yl)anilino]- 2-oxo-ethyl]carbamate (Diastereomer 2) | 616.294 | 2.01 |
| 240^{j} | 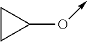 | 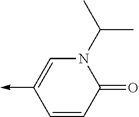 | 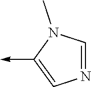 | cyclopropyl N-[1-[[3-(1- isopropyl-6-oxo-3- pyridyl)phenyl]-phenyl- methyl]-2-[4-(3- methylimidazol-4-yl)anilino]- 2-oxo-ethyl]carbamate (Diastereomer 3) | 616.293 | 2.01 |
| 241^{j} | 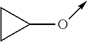 | 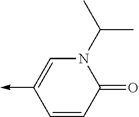 | 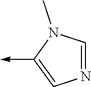 | cyclopropyl N-[1-[[3-(1- isopropyl-6-oxo-3- pyridyl)phenyl]-phenyl- methyl]-2-[4-(3- methylimidazol-4-yl)anilino]- 2-oxo-ethyl]carbamate (Diastereomer 4) | 616.292 | 2.03 |
| 242^{a} | 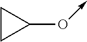 | 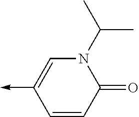 | 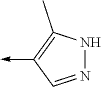 | cyclopropyl N-[1-[[3-(1- isopropyl-6-oxo-3- pyridyl)phenyl]-phenyl- methyl]-2-[4-(3-methyl-1H- pyrazol-4-yl)anilino]-2-oxo- ethyl]carbamate (Diastereomer 1) | 616.293 | 2.20 |
| 243^{a} | 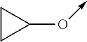 | 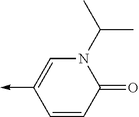 | 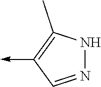 | cyclopropyl N-[1-[[3-(1- isopropyl-6-oxo-3- pyridyl)phenyl]-phenyl- methyl]-2-[4-(3-methyl-1H- pyrazol-4-yl)anilino]-2-oxo- ethyl]carbamate (Diastereomer 2) | 616.293 | 2.20 |
| 244^{a} | 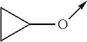 | 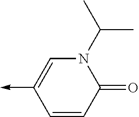 | 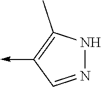 | cyclopropyl N-[1-[[3-(1- isopropyl-6-oxo-3- pyridyl)phenyl]-phenyl- methyl]-2-[4-(3-methyl-1H- pyrazol-4-yl)anilino]-2-oxo- ethyl]carbamate (Diastereomer 3) | 616.293 | 2.20 |
| 245^{a} | 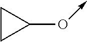 | 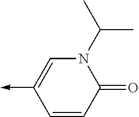 | 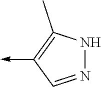 | cyclopropyl N-[1-[[3-(1- isopropyl-6-oxo-3- pyridyl)phenyl]-phenyl- methyl]-2-[4-(3-methyl-1H- pyrazol-4-yl)anilino]-2-oxo- ethyl]carbamate (Diastereomer 4) | 616.293 | 2.20 |

*RT are XEV Metode 1 CM - ES+ unless stated
^{a}prepared according to the method of Example 1
^{j}prepared according to the method described in Example 230

Examples 246-409 were synthesised by methods analogous to those described for the synthesis of Examples 1-245 and Preparations 1-93.

| Example | | | | |
|---|---|---|---|---|
| number | Structure | Name | Mass ion | RT* |
| 246 | 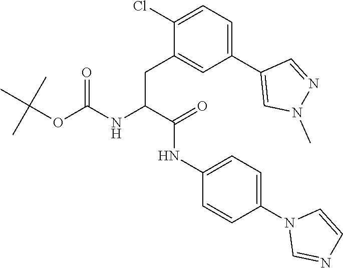 [SMILES: Cn1cc(-c2ccc(Cl)c(CC(NC(=O)OC(C)(C)C)C(=O)Nc3ccc(-n4ccnc4)cc3)c2)cn1] | tert-butyl N-[1-[[2-chloro-5-(1- methylpyrazol-4- yl)phenyl]methyl]-2-(4- imidazol-1-ylanilino)-2-oxo- ethyl]carbamate | 521.211 | 2.04 |
| 247 | 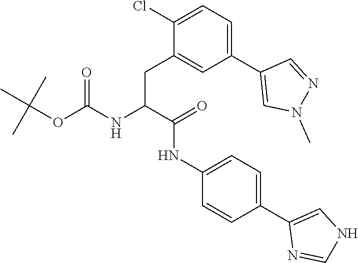 [SMILES: Cn1cc(-c2ccc(Cl)c(CC(NC(=O)OC(C)(C)C)C(=O)Nc3ccc(-c4c[nH]cn4)cc3)c2)cn1] | tert-butyl N-[1-[[2-chloro-5-(1- methylpyrazol-4- yl)phenyl]methyl]-2-[4-(1H- imidazol-4-yl)anilino]-2-oxo- ethyl]carbamate | 521.209 | 2.03 |
| 248 | 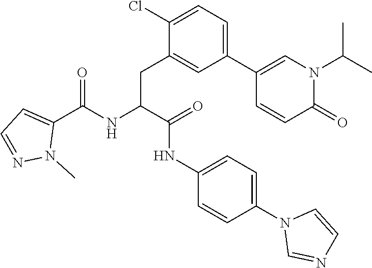 [SMILES: CC(C)n1cc(-c2ccc(Cl)c(CC(NC(=O)c3ccnn3C)C(=O)Nc3ccc(-n4ccnc4)cc3)c2)ccc1=O] | N-[1-[[2-chloro-5-(1-isopropyl- 6-oxo-3- pyridyl)phenyl]methyl]-2-(4- imidazol-1-ylanilino)-2-oxo- ethyl]-2-methyl-pyrazole-3- carboxamide | 584.222 | 1.91 |
| 249 | 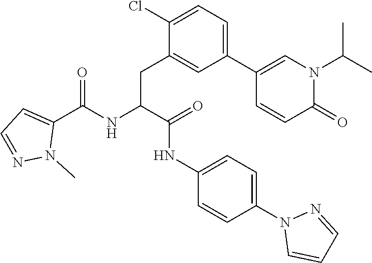 [SMILES: CC(C)n1cc(-c2ccc(Cl)c(CC(NC(=O)c3ccnn3C)C(=O)Nc3ccc(-n4cccn4)cc3)c2)ccc1=O] | N-[1-[[2-chloro-5-(1-isopropyl 6-oxo-3- pyridyl)phenyl]methyl]-2-oxo- 2-(4-pyrazol-1-ylanilino)ethyl] 2-methyl-pyrazole-3- carboxamide | 584.221 | 2.21 |
| 250 | 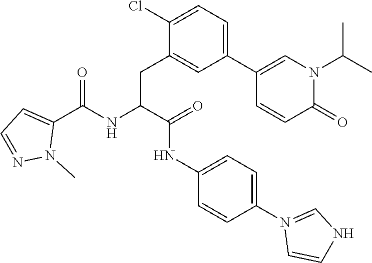 | N-[1-[[2-chloro-5-(1-isopropyl- 6-oxo-3- pyridyl)phenyl]methyl]-2-[4- (1H-imidazol-4-yl)anilino]-2- oxo-ethyl]-2-methyl-pyrazole- 3-carboxamide | 584.222 | 1.90 |
| 251 | 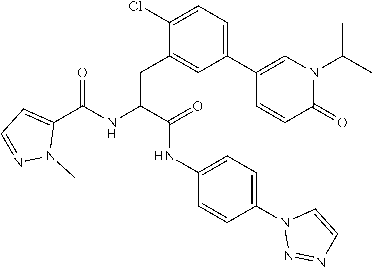 [SMILES: CC(C)n1cc(-c2ccc(Cl)c(CC(NC(=O)c3ccnn3C)C(=O)Nc3ccc(-n4ccnn4)cc3)c2)ccc1=O] | N-[1-[[2-chloro-5-(1-isopropyl- 6-oxo-3- pyridyl)phenyl]methyl]-2-oxo- 2-[4-(1,2,4-triazol-1- yl)anilino]ethyl]-2-methyl- pyrazole-3-carboxamide | 585.217 | 2.08 |
| 252 | 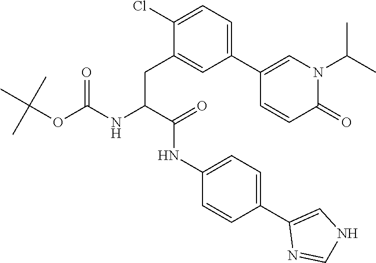 [SMILES: CC(C)n1cc(-c2ccc(Cl)c(CC(NC(=O)OC(C)(C)C)C(=O)Nc3ccc(-c4c[nH]cn4)cc3)c2)ccc1=O] | tert-butyl N-[1-[[2-chloro-5-(1- isopropyl-6-oxo-3- pyridyl)phenyl]methyl]-2-[4- (1H-imidazol-4-yl)anilino]-2- oxo-ethyl]carbamate | 576.24 | 2.03 |
| 253 | 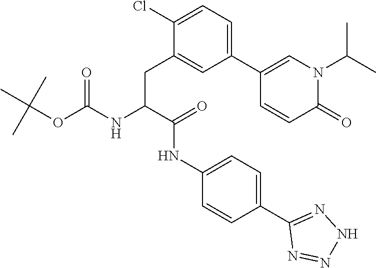 [SMILES: CC(C)n1cc(-c2ccc(Cl)c(CC(NC(=O)OC(C)(C)C)C(=O)Nc3ccc(-c4nn[nH]n4)cc3)c2)ccc1=O] | tert-butyl N-[1-[[2-chloro-5-(1- isopropyl-6-oxo-3- pyridyl)phenyl]methyl]-2-oxo- 2-[4-(2H-tetrazol-5- yl)anilino]ethyl]carbamate | 578.233 | 2.22 |
| 254 | 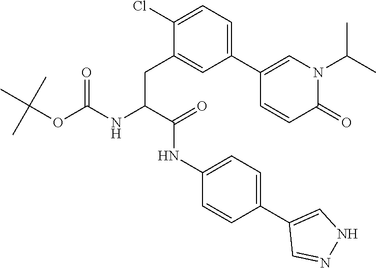 [SMILES: CC(C)n1cc(-c2ccc(Cl)c(CC(NC(=O)OC(C)(C)C)C(=O)Nc3ccc(-c4cn[nH]c4)cc3)c2)ccc1=O] | tert-butyl N-[1-[[2-chloro-5-(1- isopropyl-6-oxo-3- pyridyl)phenyl]methyl]-2-oxo- 2-[4-(1H-pyrazol-4- yl)anilino]ethyl]carbamate | 576.24 | 2.76 |
| 255 | 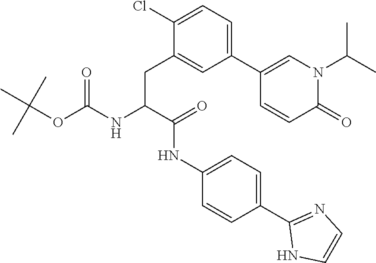 [SMILES: CC(C)n1cc(-c2ccc(Cl)c(CC(NC(=O)OC(C)(C)C)C(=O)Nc3ccc(-c4ncc[nH]4)cc3)c2)ccc1=O] | tert-butyl N-[1-[[2-chloro-5-(1- isopropyl-6-oxo-3- pyndyl)phenyl]methyl]-2-[4- (1H-imidazol-2-yl)anilino]-2- oxo-ethyl]carbamate | 576.241 | 2.04 |
| 256 | 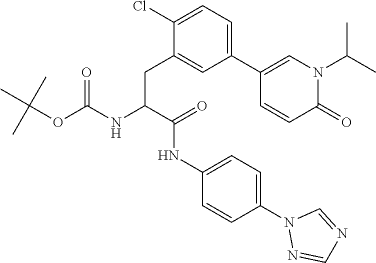 [SMILES: CC(C)n1cc(-c2ccc(Cl)c(CC(NC(=O)OC(C)(C)C)C(=O)Nc3ccc(-n4cncn4)cc3)c2)ccc1=O] | tert-butyl N-[1-[[2-chloro-5-(1- isopropyl-6-oxo-3- pyridyl)phenyl]methyl]-2-oxo- 2-[4-(1,2,4-triazol-1- yl)anilino]ethyl]carbamate | 577.236 | 2.27 |
| 257 | 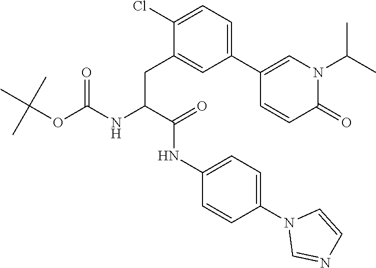 [SMILES: CC(C)n1cc(-c2ccc(Cl)c(CC(NC(=O)OC(C)(C)C)C(=O)Nc3ccc(-n4ccnc4)cc3)c2)ccc1=O] | tert-butyl N-[1-[[2-chloro-5-(1- isopropyl-6-oxo-3- pyridyl)phenyl]methyl]-2-(4- imidazol-1-ylanilino)-2-oxo- ethyl]carbamate | 576.24 | 2.07 |
| 258 | 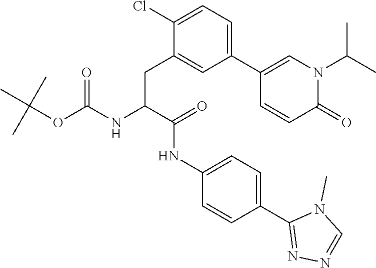 [SMILES: CC(C)n1cc(-c2ccc(Cl)c(CC(NC(=O)OC(C)(C)C)C(=O)Nc3ccc(-c4nncn4C)cc3)c2)ccc1=O] | tert-butyl N-[1-[[2-chloro-5-(1- isopropyl-6-oxo-3- pyridyl)phenyl]methyl]-2-[4- (4-methyl-1,2,4-triazol-3- yl)anilino]-2-oxo- ethyl]carbamate | 591.251 | 2.15 |
| 259 | 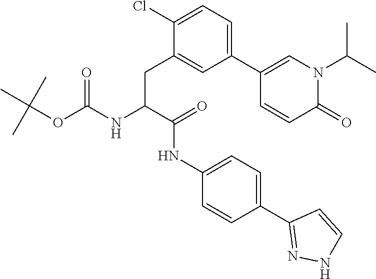 [SMILES: CC(C)n1cc(-c2ccc(Cl)c(CC(NC(=O)OC(C)(C)C)C(=O)Nc3ccc(-c4cc[nH]n4)cc3)c2)ccc1=O] | tert-butyl N-[1-[[2-chloro-5-(1- isopropyl-6-oxo-3- pyridyl)phenyl]methyl]-2-oxo- 2-[4-(1H-pyrazol-3- yl)anilino]ethyl]carbamate | 576.238 | 2.29 |
| 260 | 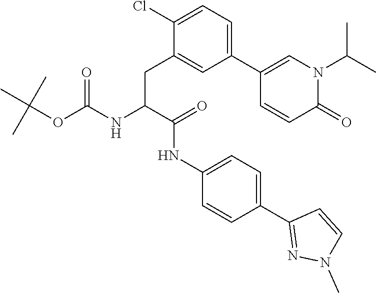 [SMILES: CC(C)n1cc(-c2ccc(Cl)c(CC(NC(=O)OC(C)(C)C)C(=O)Nc3ccc(-c4ccn(C)n4)cc3)c2)ccc1=O] | tert-butyl N-[1-[[2-chloro-5-(1- isopropyl-6-oxo-3- pyridyl)phenyl]methyl]-2-[4- (1-methylpyrazol-3-yl)anilino]- 2-oxo-ethyl]carbamate | 590.255 | 2.37 |
| 261 | 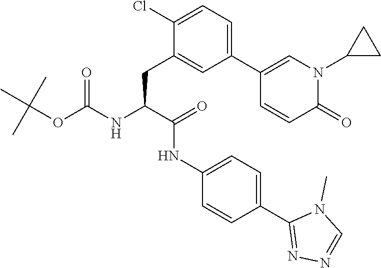 [SMILES: Cn1cnnc1-c1ccc(NC(=O)[C@H](Cc2cc(-c3ccc(=O)n(C4CC4)c3)ccc2Cl)NC(=O)OC(C)(C)C)cc1] | tert-butyl N-[(1S)-1-[[2-chloro- 5-(1-cyclopropyl-6-oxo-3- pyridyl)phenyl]methyl]-2-[4- (4-methyl-1,2,4-triazol-3- yl)anilino]-2-oxo- ethyl]carbamate | 589.234 | 2.09 |
| 262 | 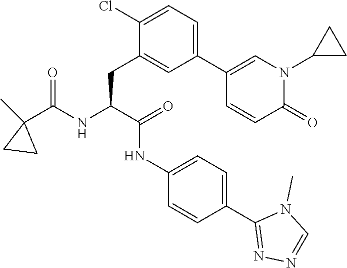 [SMILES: Cn1cnnc1-c1ccc(NC(=O)[C@H](Cc2cc(-c3ccc(=O)n(C4CC4)c3)ccc2Cl)NC(=O)C2(C)CC2)cc1] | N-[(1S)-1-[[2-chloro-5-(1- cyclopropyl-6-oxo-3- pyridyl)phenyl]methyl]-2-[4- (4-methyl-1,2,4-triazol-3- yl)anilino]-2-oxo-ethyl]-1- methyl- cyclopropanecarboxamide | 571.222 | 2.04 |
| 263 | 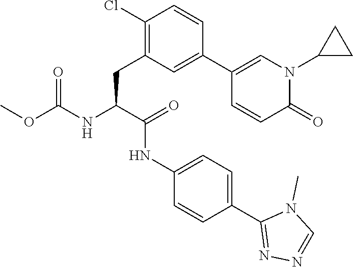 [SMILES: COC(=O)N[C@@H](Cc1cc(-c2ccc(=O)n(C3CC3)c2)ccc1Cl)C(=O)Nc1ccc(-c2nncn2C)cc1] | methyl N-[(1S)-1-[[2-chloro-5- (1-cyclopropyl-6-oxo-3- pyridyl)phenyl]methyl]-2-[4- (4-methyl-1,2,4-triazol-3- yl)anilino]-2-oxo- ethyl]carbamate | 547.186 | 1.96 |
| 264 | 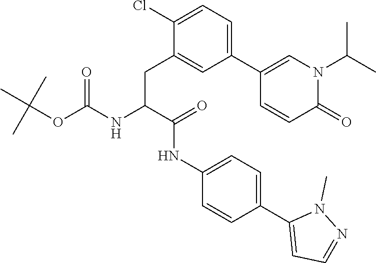 [SMILES: CC(C)n1cc(-c2ccc(Cl)c(CC(NC(=O)OC(C)(C)C)C(=O)Nc3ccc(-c4ccnn4C)cc3)c2)ccc1=O] | tert-butyl N-[1-[[2-chloro-5-(1- isopropyl-6-oxo-3- pyridyl)phenyl]methyl]-2-[4- (2-methylpyrazol-3-yl)anilino]- 2-oxo-ethyl]carbamate | 590.254 | 2.37 |
| 265 | 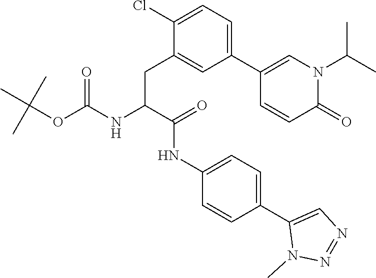 [SMILES: CC(C)n1cc(-c2ccc(Cl)c(CC(NC(=O)OC(C)(C)C)C(=O)Nc3ccc(-c4cnnn4C)cc3)c2)ccc1=O] | tert-butyl N-[1-[[2-chloro-5-(1- isopropyl-6-oxo-3- pyridyl)phenyl]methyl]-2-[4- (3-methyltriazol-4-yl)anilino]- 2-oxo-ethyl]carbamate | 591.249 | 2.29 |
| 266 | 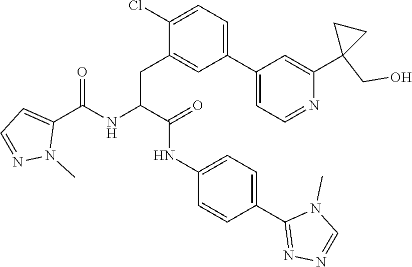 [SMILES: Cn1cnnc1-c1ccc(NC(=O)C(Cc2cc(-c3ccnc(C4(CO)CC4)c3)ccc2Cl)NC(=O)c2ccnn2C)cc1] | N-[1-[[2-chloro-5-[2-[1- (hydroxymethyl)cyclopropyl]- 4-pyridyl]phenyl]methyl]-2-[4- (4-methyl-1,2,4-triazol-3- yl)anilino]-2-oxo-ethyl]-2- methyl-pyrazole-3- carboxamide | 611.23 | 1.83 |
| 267 | 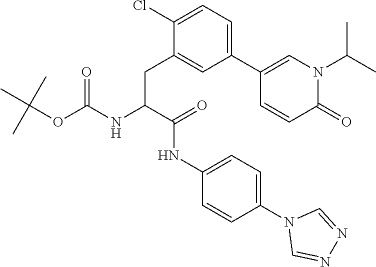 [SMILES: CC(C)n1cc(-c2ccc(Cl)c(CC(NC(=O)OC(C)(C)C)C(=O)Nc3ccc(-n4cnnc4)cc3)c2)ccc1=O] | tert-butyl N-[1-[[2-chloro-5-(1- isopropyl-6-oxo-3- pyridyl)phenyl]methyl]-2-oxo- 2-[4-(1,2,4-triazol-4- yl)anilino]ethyl]carbamate | 577.235 | 2.16 |
| 268 | 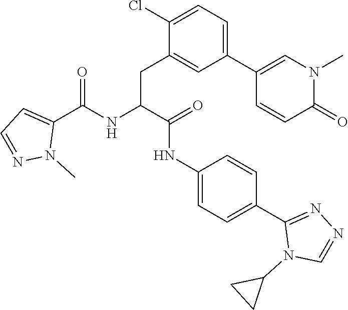 [SMILES: Cn1nccc1C(=O)NC(Cc1cc(-c2ccc(=O)n(C)c2)ccc1Cl)C(=O)Nc1ccc(-c2nncn2C2CC2)cc1] | N-[1-[[2-chloro-5-(1-methyl-6- oxo-3-pyridyl)phenyl]methyl]- 2-[4-(4-cyclopropyl-1,2,4- triazol-3-yl)anilino]-2-oxo- ethyl]-2-methyl-pyrazole-3- carboxamide | 597.212 | 1.96 |
| 269 | 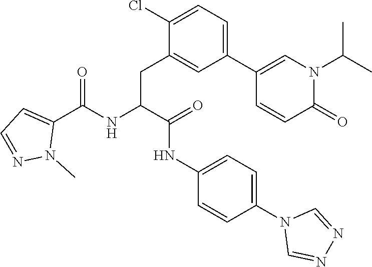 [SMILES: CC(C)n1cc(-c2ccc(Cl)c(CC(NC(=O)c3ccnn3C)C(=O)Nc3ccc(-n4cnnc4)cc3)c2)ccc1=O] | N-[1-[[2-chloro-5-(1-isopropyl- 6-oxo-3- pyridyl)phenyl]methyl]-2-oxo- 2-[4-(1,2,4-triazol-4- yl)anilino]ethyl]-2-methyl- pyrazole-3-carboxamide | 584.213 | 2.00 |
| 270 | 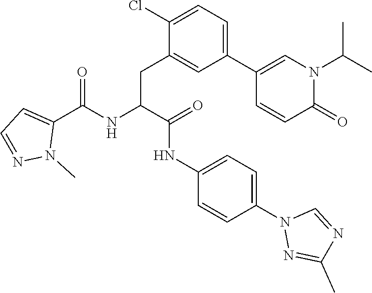 [SMILES: Cc1ncn(-c2ccc(NC(=O)C(Cc3cc(-c4ccc(=O)n(C(C)C)c4)ccc3Cl)NC(=O)c3ccnn3C)cc2)n1] | N-[1-[[2-chloro-5-(1-isopropyl- 6-oxo-3- pyridyl)phenyl]methyl]-2-[4- (3-methyl-1,2,4-triazol-1- yl)anilino]-2-oxo-ethyl]-2- methyl-pyrazole-3- carboxamide | 599.226 | 2.10 |
| 271 | 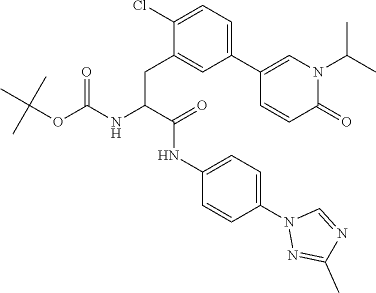 [SMILES: Cc1ncn(-c2ccc(NC(=O)C(Cc3cc(-c4ccc(=O)n(C(C)C)c4)ccc3Cl)NC(=O)OC(C)(C)C)cc2)n1] | tert-butyl N-[1-[[2-chloro-5-(1- isopropyl-6-oxo-3- pyridyl)phenyl]methyl]-2-[4- (3-methyl-1,2,4-triazol-1- yl)anilino]-2-oxo- ethyl]carbamate | 591.252 | 2.28 |
| 272 | 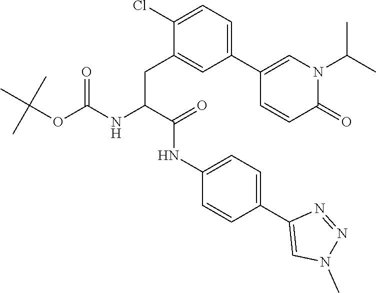 [SMILES: CC(C)n1cc(-c2ccc(Cl)c(CC(NC(=O)OC(C)(C)C)C(=O)Nc3ccc(-c4cn(C)nn4)cc3)c2)ccc1=O] | tert-butyl N-[1-[[2-chloro-5-(1- isopropyl-6-oxo-3- pyridyl)phenyl]methyl]-2-[4- (1-methyltriazol-4-yl)anilino]- 2-oxo-ethyl]carbamate | 591.251 | 2.27 |
| 273 | 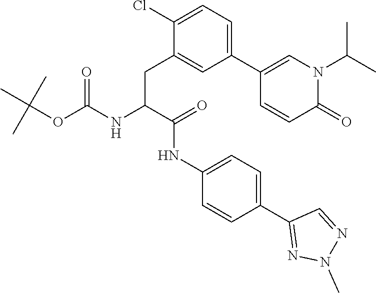 [SMILES: CC(C)n1cc(-c2ccc(Cl)c(CC(NC(=O)OC(C)(C)C)C(=O)Nc3ccc(-c4cnn(C)n4)cc3)c2)ccc1=O] | tert-butyl N-[1-[[2-chloro-5-(1- isopropyl-6-oxo-3- pyridyl)phenyl]methyl]-2-[4- (2-methyltriazol-4-yl)anilino]- 2-oxo-ethyl]carbamate | 591.249 | 2.41 |
| 274 | 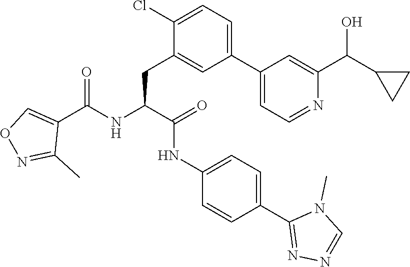 [SMILES: Cc1nocc1C(=O)N[C@@H](Cc1cc(-c2ccnc(C(O)C3CC3)c2)ccc1Cl)C(=O)Nc1ccc(-c2nncn2C)cc1] | N-[(1S)-1-[[2-chloro-5-[2- [cyclopropyl(hydroxy)methyl]- 4-pyridyl]phenyl]methyl]-2-[4- (4-methyl-1,2,4-triazol-3- yl)anilino]-2-oxo-ethyl]-3- methyl-isoxazole-4- carboxamide | 612.215 | 1.90 |
| 275 | 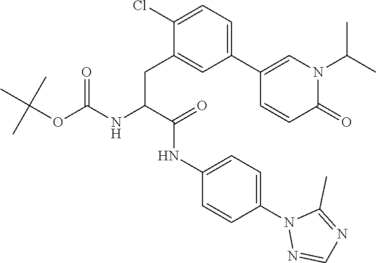 [SMILES: Cc1ncnn1-c1ccc(NC(=O)C(Cc2cc(-c3ccc(=O)n(C(C)C)c3)ccc2Cl)NC(=O)OC(C)(C)C)cc1] | tert-butyl N-[1-[[2-chloro-5-(1- isopropyl-6-oxo-3- pyridyl)phenyl]methyl]-2-[4- (5-methyl-1,2,4-triazol-1- yl)anilino]-2-oxo- ethyl]carbamate | 591.25 | 2.25 |
| 276 | 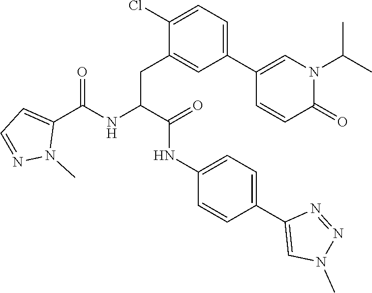 [SMILES: CC(C)n1cc(-c2ccc(Cl)c(CC(NC(=O)c3ccnn3C)C(=O)Nc3ccc(-c4cn(C)nn4)cc3)c2)ccc1=O] | N-[1-[[2-chloro-5-(1-isopropyl- 6-oxo-3- pyridyl)phenyl]methyl]-2-[4- (1-methyltriazol-4-yl)anilino]- 2-oxo-ethyl]-2-methyl- pyrazole-3-carboxamide | 599.238 | 2.09 |
| 277 | 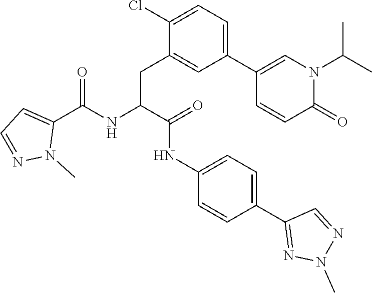 [SMILES: CC(C)n1cc(-c2ccc(Cl)c(CC(NC(=O)c3ccnn3C)C(=O)Nc3ccc(-c4cnn(C)n4)cc3)c2)ccc1=O] | N-[1-[[2-chloro-5-(1-isopropyl- 6-oxo-3- pyridyl)phenyl]methyl]-2-[4- (2-methyltriazol-4-yl)anilino]- 2-oxo-ethyl]-2-methyl- pyrazole-3-carboxamide | 599.229 | 2.21 |
| 278 | 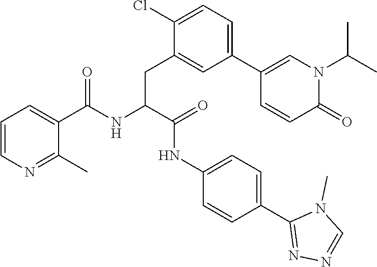 [SMILES: Cc1ncccc1C(=O)NC(Cc1cc(-c2ccc(=O)n(C(C)C)c2)ccc1Cl)C(=O)Nc1ccc(-c2nncn2C)cc1] | N-[1-[[2-chloro-5-(1-isopropyl- 6-oxo-3- pyridyl)phenyl]methyl]-2-[4- (4-methyl-1,2,4-triazol-3- yl)anilino]-2-oxo-ethyl]-2- methyl-pyridine-3-carboxamide | 610.234 | 1.90 |
| 279 | 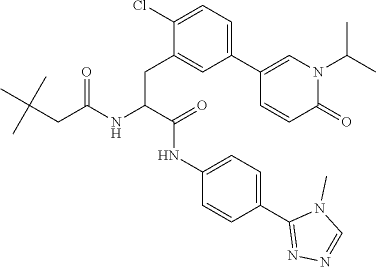 [SMILES: CC(C)n1cc(-c2ccc(Cl)c(CC(NC(=O)CC(C)(C)C)C(=O)Nc3ccc(-c4nncn4C)cc3)c2)ccc1=O] | N-[1-[[2-chloro-5-(1-isopropyl- 6-oxo-3- pyridyl)phenyl]methyl]-2-[4- (4-methyl-1,2,4-triazol-3- yl)anilino]-2-oxo-ethyl]-3,3- dimethyl-butanamide | 589.272 | 2.14 |
| 280 | 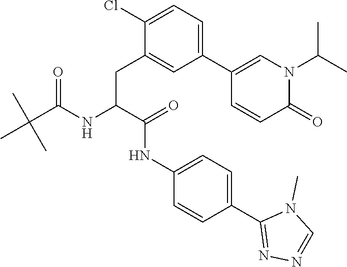 [SMILES: CC(C)n1cc(-c2ccc(Cl)c(CC(NC(=O)C(C)(C)C)C(=O)Nc3ccc(-c4nncn4C)cc3)c2)ccc1=O] | N-[1-[[2-chloro-5-(1-isopropyl- 6-oxo-3- pyridyl)phenyl]methyl]-2-[4- (4-methyl-1,2,4-triazol-3- yl)anilino]-2-oxo-ethyl]-2,2- dimethyl-propanamide | 575.253 | 2.10 |
| 281 | 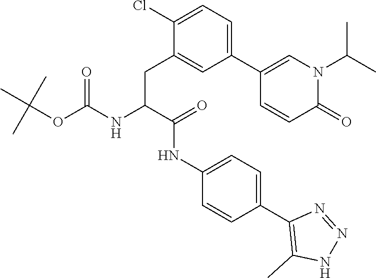 [SMILES: Cc1[nH]nnc1-c1ccc(NC(=O)C(Cc2cc(-c3ccc(=O)n(C(C)C)c3)ccc2Cl)NC(=O)OC(C)(C)C)cc1] | tert-butyl N-[1-[[2-chloro-5-(1- isopropyl-6-oxo-3- pyridyl)phenyl]methyl]-2-[4- (5-methyl-1H-triazol-4- yl)anilino]-2-oxo- ethyl]carbamate | 591.248 | 2.26 |
| 282 | 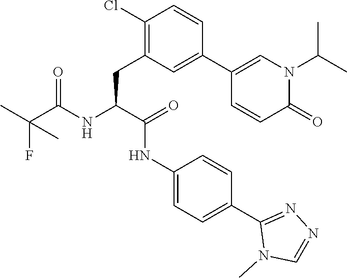 [SMILES: CC(C)n1cc(-c2ccc(Cl)c(C[C@H](NC(=O)C(C)(C)F)C(=O)Nc3ccc(-c4nncn4C)cc3)c2)ccc1=O] | N-[(1S)-1-[[2-chloro-5-(1- isopropyl-6-oxo-3- pyridyl)phenyl]methyl]-2-[4- (4-methyl-1,2,4-triazol-3- yl)anilino]-2-oxo-ethyl]-2- fluoro-2-methyl-propanamide | 579.23 | 2.07 |
| 283 | 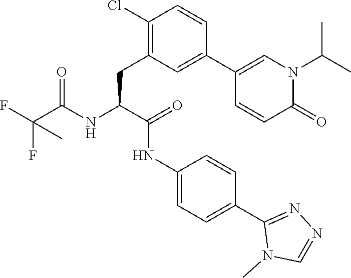 [SMILES: CC(C)n1cc(-c2ccc(Cl)c(C[C@H](NC(=O)C(C)(F)F)C(=O)Nc3ccc(-c4nncn4C)cc3)c2)ccc1=O] | N-[(1S)-1-[[2-chloro-5-(1- isopropyl-6-oxo-3- pyridyl)phenyl]methyl]-2-[4- (4-methyl-1,2,4-triazol-3- yl)anilino]-2-oxo-ethyl]-2,2- difluoro-propanamide | 583.203 | 2.08 |
| 284 | 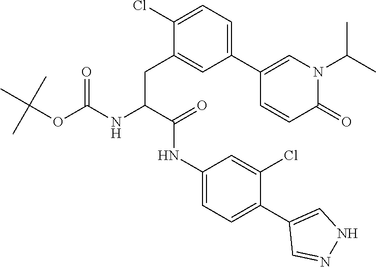 [SMILES: CC(C)n1cc(-c2ccc(Cl)c(CC(NC(=O)OC(C)(C)C)C(=O)Nc3ccc(-c4cn[nH]c4)c(Cl)c3)c2)ccc1=O] | tert-butyl N-[1-[[2-chloro-5-(1- isopropyl-6-oxo-3- pyridyl)phenyl]methyl]-2-[3- chloro-4-(1H-pyrazol-4- yl)anilino]-2-oxo- ethyl]carbamate | 610.199 | 2.35 |
| 285 | 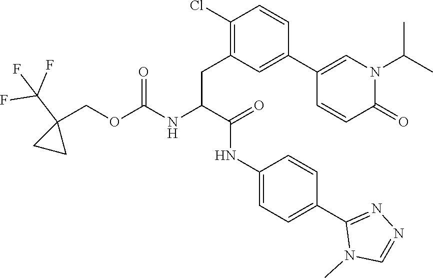 [SMILES: CC(C)n1cc(-c2ccc(Cl)c(CC(NC(=O)OCC3(C(F)(F)F)CC3)C(=O)Nc3ccc(-c4nncn4C)cc3)c2)ccc1=O] | [1- (trifluoromethyl)cyclopropyl] methyl N-[1-[[2-chloro-5-(1- isopropyl-6-oxo-3- pyridyl)phenyl]methyl]-2-[4- (4-methyl-1,2,4-triazol-3- yl)anilino]-2-oxo- ethyl]carbamate | 657.222 | 2.20 |
| 286 | 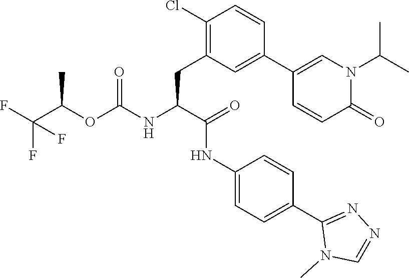 [SMILES: CC(C)n1cc(-c2ccc(Cl)c(C[C@H](NC(=O)O[C@H](C)C(F)(F)F)C(=O)Nc3ccc(-c4nncn4C)cc3)c2)ccc1=O] | [(1R)-2,2,2-trifluoro-1-methyl- ethyl] N-[(1S)-1-[[2-chloro-5- (1-isopropyl-6-oxo-3- pyridyl)phenyl]methyl]-2-[4- (4-methyl-1,2,4-triazol-3- yl)anilino]-2-oxo- ethyl]carbamate | 631.207 | 2.20 |
| 287 | 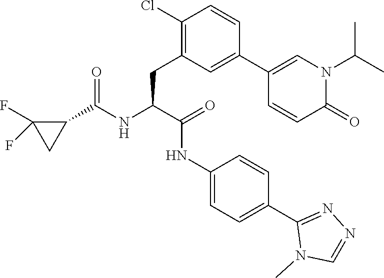 [SMILES: CC(C)n1cc(-c2ccc(Cl)c(C[C@H](NC(=O)[C@@H]3CC3(F)F)C(=O)Nc3ccc(-c4nncn4C)cc3)c2)ccc1=O] | (1S)-N-[(1S)-1-[[2-chloro-5- (1-isopropyl-6-oxo-3- pyridyl)phenyl]methyl]-2-[4- (4-methyl-1,2,4-triazol-3- yl)anilino]-2-oxo-ethyl]-2,2- difluoro- cyclopropanecarboxamide | 595.204 | 2.87 |
| 288 | 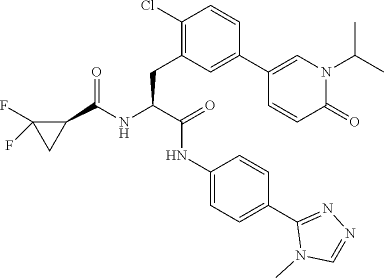 [SMILES: CC(C)n1cc(-c2ccc(Cl)c(C[C@H](NC(=O)[C@H]3CC3(F)F)C(=O)Nc3ccc(-c4nncn4C)cc3)c2)ccc1=O] | (1R)-N-[(1S)-1-[[2-chloro-5- (1-isopropyl-6-oxo-3- pyridyl)phenyl]methyl]-2-[4- (4-methyl-1,2,4-triazol-3- yl)anilino]-2-oxo-ethyl]-2,2- difluoro- cyclopropanecarboxamide | 595.204 | 2.03 |
| 289 | 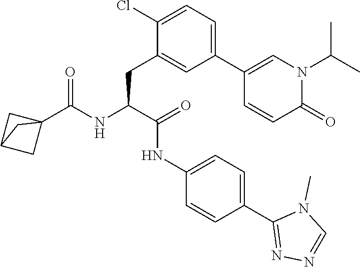 [SMILES: CC(C)n1cc(-c2ccc(Cl)c(C[C@H](NC(=O)C34CC(C3)C4)C(=O)Nc3ccc(-c4nncn4C)cc3)c2)ccc1=O] | N-[(1S)-1-[[2-chloro-5-(1- isopropyl-6-oxo-3- pyridyl)phenyl]methyl]-2-[4- (4-methyl-1,2,4-triazol-3- yl)anilino]-2-oxo- ethyl]bicyclo[1.1.1]pentane-3- carboxamide | 585.235 | 2.07 |
| 290 | 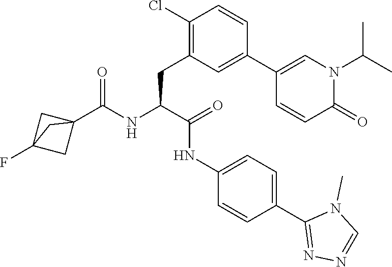 [SMILES: CC(C)n1cc(-c2ccc(Cl)c(C[C@H](NC(=O)C34CC(F)(C3)C4)C(=O)Nc3ccc(-c4nncn4C)cc3)c2)ccc1=O] | N-[(1S)-1-[[2-chloro-5-(1- isopropyl-6-oxo-3- pyridyl)phenyl]methyl]-2-[4- (4-methyl-1,2,4-triazol-3- yl)anilino]-2-oxo-ethyl]-3- fluoro-bicyclo[1.1.1]pentane-1- carboxamide | 603.229 | 2.06 |
| 291 | 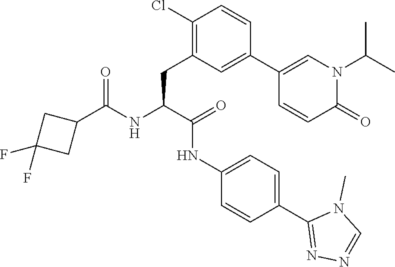 [SMILES: CC(C)n1cc(-c2ccc(Cl)c(C[C@H](NC(=O)C3CC(F)(F)C3)C(=O)Nc3ccc(-c4nncn4C)cc3)c2)ccc1=O] | N-[(1S)-1-[[2-chloro-5-(1- isopropyl-6-oxo-3- pyridyl)phenyl]methyl]-2-[4- (4-methyl-1,2,4-triazol-3- yl)anilino]-2-oxo-ethyl]-3,3- difluoro- cyclobutanecarboxamide | 609.220 | 2.08 |
| 292 | 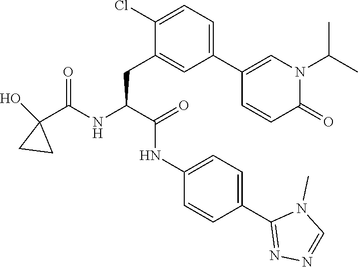 [SMILES: CC(C)n1cc(-c2ccc(Cl)c(C[C@H](NC(=O)C3(O)CC3)C(=O)Nc3ccc(-c4nncn4C)cc3)c2)ccc1=O] | N-[(1S)-1-[[2-chloro-5-(1- isopropyl-6-oxo-3- pyridyl)phenyl]methyl]-2-[4- (4-methyl-1,2,4-triazol-3- yl)anilino]-2-oxo-ethyl]-1- hydroxy- cyclopropanecarboxamide | 575.217 | 1.93 |
| 293 | 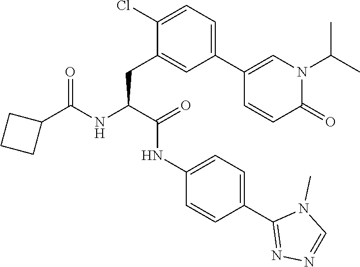 [SMILES: CC(C)n1cc(-c2ccc(Cl)c(C[C@H](NC(=O)C3CCC3)C(=O)Nc3ccc(-c4nncn4C)cc3)c2)ccc1=O] | N-[(1S)-1-[[2-chloro-5-(1- isopropyl-6-oxo-3- pyridyl)phenyl]methyl]-2-[4- (4-methyl-1,2,4-triazol-3- yl)anilino]-2-oxo- ethyl]cyclobutanecarboxamide | 573.239 | 2.05 |
| 294 | 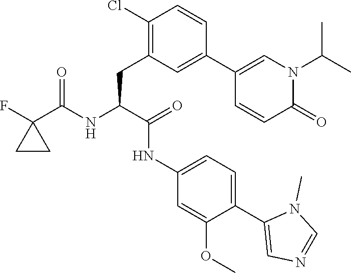 [SMILES: COc1cc(NC(=O)[C@H](Cc2cc(-c3ccc(=O)n(C(C)C)c3)ccc2Cl)NC(=O)C2(F)CC2)ccc1-c1cncn1C] | N-[(1S)-1-[[2-chloro-5-(1- isopropyl-6-oxo-3- pyridyl)phenyl]methyl]-2-[3- methoxy-4-(3-methylimidazol- 4-yl)anilino]-2-oxo-ethyl]-1- fluoro- cyclopropanecarboxamide | 606.228 | 2.00 |
| 295 | 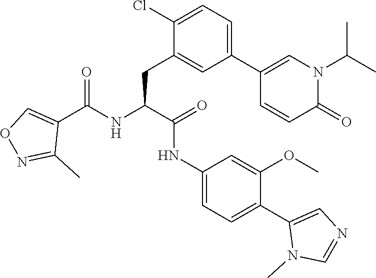 [SMILES: COc1cc(NC(=O)[C@H](Cc2cc(-c3ccc(=O)n(C(C)C)c3)ccc2Cl)NC(=O)c2conc2C)ccc1-c1cncn1C] | N-[(1S)-1-[[2-chloro-5-(1- isopropyl-6-oxo-3- pyridyl)phenyl]methyl]-2-[3- methoxy-4-(3-methylimidazol- 4-yl)anilino]-2-oxo-ethyl]-3- methyl-isoxazole-4- carboxamide | 629.228 | 1.97 |
| 296 | 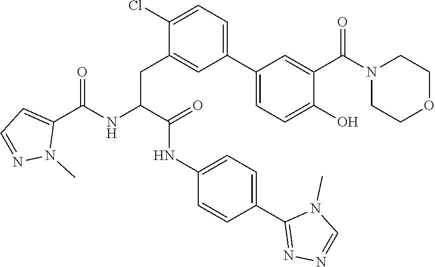 [SMILES: Cn1cnnc1-c1ccc(NC(=O)C(Cc2cc(-c3ccc(O)c(C(=O)N4CCOCC4)c3)ccc2Cl)NC(=O)c2ccnn2C)cc1] | N-[1-[[2-chloro-5-[4-hydroxy- 3-(morpholine-4- carbonyl)phenyl]phenyl]methyl]- 2-[4-(4-methyl-1,2,4-triazol- 3-yl)anilino]-2-oxo-ethyl]-2- methyl-pyrazole-3- carboxamide | 669.234 | 1.96 |
| 297 | 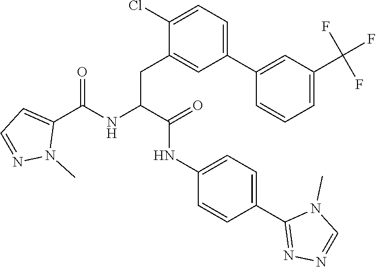 [SMILES: Cn1cnnc1-c1ccc(NC(=O)C(Cc2cc(-c3cccc(C(F)(F)F)c3)ccc2Cl)NC(=O)c2ccnn2C)cc1] | N-[1-[[2-chloro-5-[3- (trifluoromethyl)phenyl]phenyl] methyl]-2-[4-(4-methyl-1,2,4- triazol-3-yl)anilino]-2-oxo- ethyl]-2-methyl-pyrazole-3- carboxamide | 608.179 | 2.34 |
| 298 | 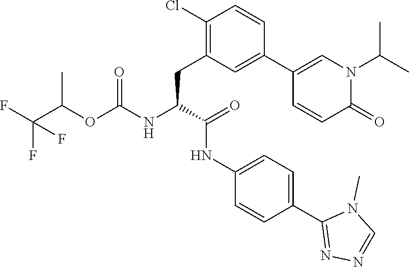 | (2,2,2-trifluoro-1-methyl-ethyl) N-[(1S)-1-[[2-chloro-5-(1- isopropyl-6-oxo-3- pyridyl)phenyl]methyl]-2-[4- (4-methyl-1,2,4-triazol-3- yl)anilino]-2-oxo- ethyl]carbamate | 631.206 | 2.17 |
| 299 | 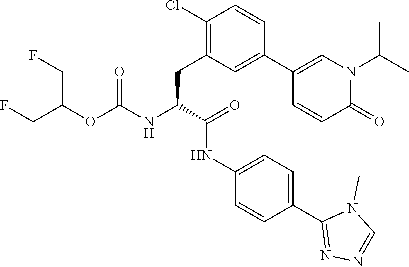 | [2-fluoro-1- (fluoromethyl)ethyl] N-[(1S)-1- [[2-chloro-5-(1-isopropyl-6- oxo-3-pyridyl)phenyl]methyl]- 2-[4-(4-methyl-1,2,4-triazol-3- yl)anilino]-2-oxo- ethyl]carbamate | 613.217 | 2.09 |
| 300 | 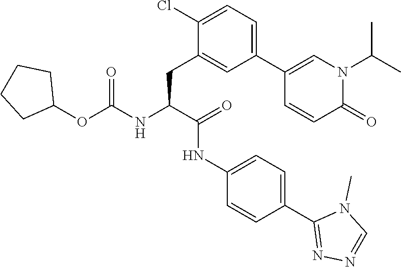 [SMILES: CC(C)n1cc(-c2ccc(Cl)c(C[C@H](NC(=O)OC3CCCC3)C(=O)Nc3ccc(-c4nncn4C)cc3)c2)ccc1=O] | cyclopentyl N-[(1S)-1-[[2- chloro-5-(1-isopropyl-6-oxo-3- pyridyl)phenyl]methyl]-2-[4- (4-methyl-1,2,4-triazol-3- yl)anilino]-2-oxo- ethyl]carbamate | 603.249 | 2.19 |
| 301 | 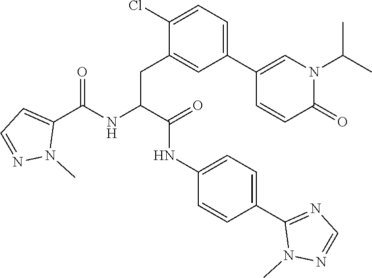 [SMILES: CC(C)n1cc(-c2ccc(Cl)c(CC(NC(=O)c3ccnn3C)C(=O)Nc3ccc(-c4ncnn4C)cc3)c2)ccc1=O] | N-[1-[[2-chloro-5-(1-isopropyl- 6-oxo-3- pyridyl)phenyl]methyl]-2-[4- (2-methyl-1,2,4-triazol-3- yl)anilino]-2-oxo-ethyl]-2- methyl-pyrazole-3- carboxamide | 599.299 | 2.07 |
| 302 | 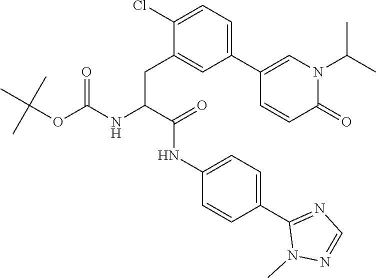 [SMILES: CC(C)n1cc(-c2ccc(Cl)c(CC(NC(=O)OC(C)(C)C)C(=O)Nc3ccc(-c4ncnn4C)cc3)c2)ccc1=O] | tert-butyl N-[1-[[2-chloro-5-(1- isopropyl-6-oxo-3- pyridyl)phenyl]methyl]-2-[4- (2-methyl-1,2,4-triazol-3- yl)anilino]-2-oxo- ethyl]carbamate | 591.249 | 2.25 |
| 303 | 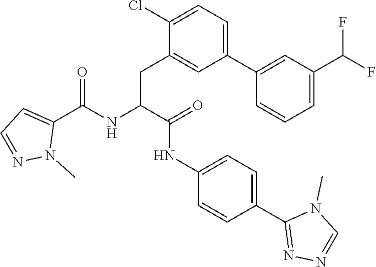 [SMILES: Cn1cnnc1-c1ccc(NC(=O)C(Cc2cc(-c3cccc(C(F)F)c3)ccc2Cl)NC(=O)c2ccnn2C)cc1] | N-[1-[[2-chloro-5-[3- (difluoromethyl)phenyl]phenyl] methyl]-2-[4-(4-methyl-1,2,4- triazol-3-yl)anilino]-2-oxo- ethyl]-2-methyl-pyrazole-3- carboxamide | 590.189 | 2.25 |
| 304 | 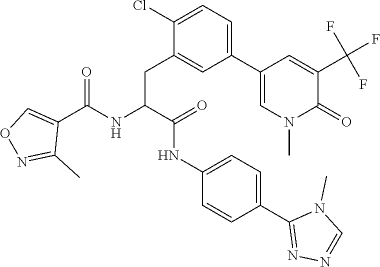 [SMILES: Cc1nocc1C(=O)NC(Cc1cc(-c2cc(C(F)(F)F)c(=O)n(C)c2)ccc1Cl)C(=O)Nc1ccc(-c2nncn2C)cc1] | N-[1-[[2-chloro-5-[1-methyl-6- oxo-5-(trifluoromethyl)-3- pyridyl]phenyl]methyl]-2-[4- (4-methyl-1,2,4-triazol-3- yl)anilino]-2-oxo-ethyl]-3- methyl-isoxazole-4- carboxamide | 640.169 | 2.11 |
| 305 | 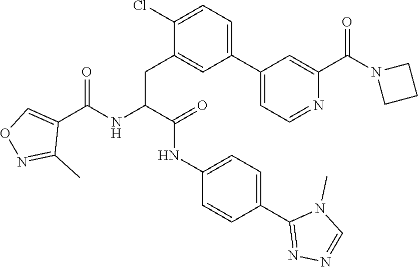 [SMILES: Cc1nocc1C(=O)NC(Cc1cc(-c2ccnc(C(=O)N3CCC3)c2)ccc1Cl)C(=O)Nc1ccc(-c2nncn2C)cc1] | N-[1-[[5-[2-(azetidine-1- carbonyl)-4-pyridyl]-2-chloro- phenyl]methyl]-2-[4-(4- methyl-1,2,4-triazol-3- yl)anilino]-2-oxo-ethyl]-3- methyl-isoxazole-4- carboxamide | 625.208 | 2.05 |
| 306 | 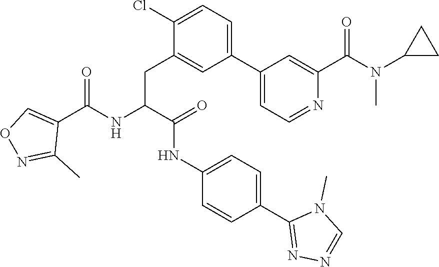 [SMILES: Cc1nocc1C(=O)NC(Cc1cc(-c2ccnc(C(=O)N(C)C3CC3)c2)ccc1Cl)C(=O)Nc1ccc(-c2nncn2C)cc1] | N-[1-[[2-chloro-5-[2- [cyclopropyl(methyl)carbamoyl]- 4-pyridyl]phenyl]methyl]-2- [4-(4-methyl-1,2,4-triazol-3- yl)anilino]-2-oxo-ethyl]-3- methyl-isoxazole-4- carboxamide | 639.223 | 2.02 |
| 307 | 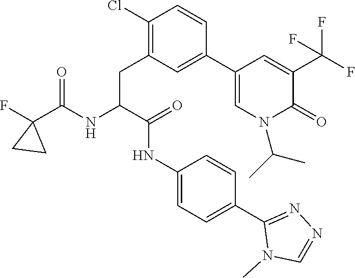 [SMILES: CC(C)n1cc(-c2ccc(Cl)c(CC(NC(=O)C3(F)CC3)C(=O)Nc3ccc(-c4nncn4C)cc3)c2)cc(C(F)(F)F)c1=O] | N-[1-[[2-chloro-5-[1-isopropyl- 6-oxo-5-(trifluoromethyl)-3- pyridyl]phenyl]methyl]-2-[4- (4-methyl-1,2,4-triazol-3- yl)anilino]-2-oxo-ethyl]-1- fluoro- cyclopropanecarboxamide | 645.197 | 2.24 |
| 308 | 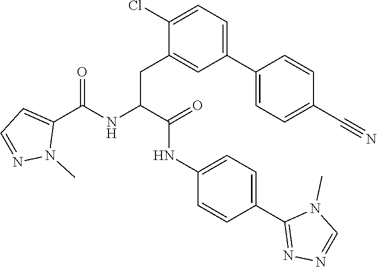 [SMILES: Cn1cnnc1-c1ccc(NC(=O)C(Cc2cc(-c3ccc(C#N)cc3)ccc2Cl)NC(=O)c2ccnn2C)cc1] | N-[1-[[2-chloro-5-(4- cyanophenyl)phenyl]methyl]-2- [4-(4-methyl-1,2,4-triazol-3- yl)anilino]-2-oxo-ethyl]-2- methyl-pyrazole-3- carboxamide | 565.187 | 2.17 |
| 309 | 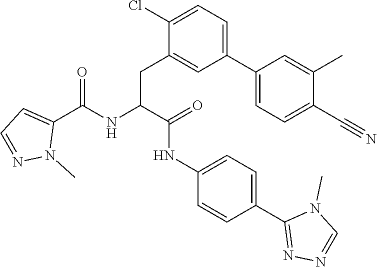 [SMILES: Cc1cc(-c2ccc(Cl)c(CC(NC(=O)c3ccnn3C)C(=O)Nc3ccc(-c4nncn4C)cc3)c2)ccc1C#N] | N-[1-[[2-chloro-5-(4-cyano-3- methyl-phenyl)phenyl]methyl]- 2-[4-(4-methyl-1,2,4-triazol-3- yl)anilino]-2-oxo-ethyl]-2- methyl-pyrazole-3- carboxamide | 579.203 | 2.23 |
| 310 | 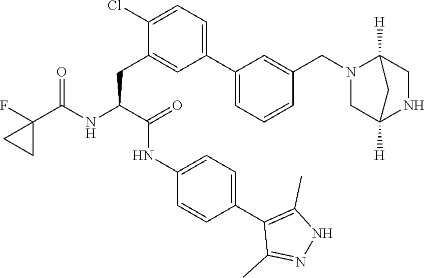 [SMILES: Cc1n[nH]c(C)c1-c1ccc(NC(=O)[C@H](Cc2cc(-c3cccc(CN4C[C@H]5C[C@@H]4CN5)c3)ccc2Cl)NC(=O)C2(F)CC2)cc1] | N-[(1S)-1-[[2-chloro-5-[3- [[(1R,4R)-2,5- diazabicyclo[2.2.1]heptan-2- yl]methyl]phenyl]phenyl]methyl]- 2-[4-(3,5-dimethyl-1H- pyrazol-4-yl)anilino]-2-oxo- ethyl]-1-fluoro- cyclopropanecarboxamide | 641.281 | 1.91 |
| 311 | 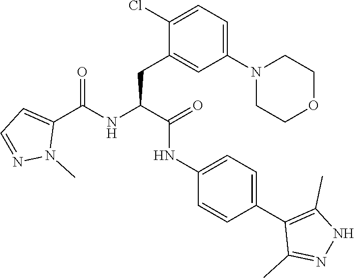 [SMILES: Cc1n[nH]c(C)c1-c1ccc(NC(=O)[C@H](Cc2cc(N3CCOCC3)ccc2Cl)NC(=O)c2ccnn2C)cc1] | N-[(1S)-1-[(2-chloro-5- morpholino-phenyl)methyl]-2- [4-(3,5-dimethyl-1H-pyrazol-4- yl)anilino]-2-oxo-ethyl]-2- methyl-pyrazole-3- carboxamide | 562.232 | 2.14 |
| 312 | 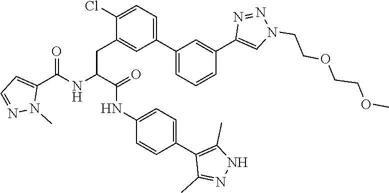 [SMILES: COCCOCCn1cc(-c2cccc(-c3ccc(Cl)c(CC(NC(=O)c4ccnn4C)C(=O)Nc4ccc(-c5c(C)n[nH]c5C)cc4)c3)c2)nn1] | N-[1-[[2-chloro-5-[3-[1-[2-(2- methoxyethoxy)ethyl]triazol-4- yl]phenyl]phenyl]methyl]-2-[4- (3,5-dimethyl-1H-pyrazol-4- yl)anilino]-2-oxo-ethyl]-2- methyl-pyrazole-3- carboxamide | 722.298 | 2.24 |
| 313 | 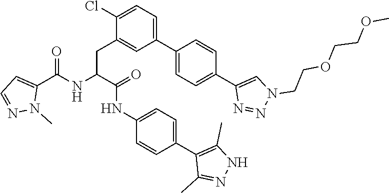 [SMILES: COCCOCCn1cc(-c2ccc(-c3ccc(Cl)c(CC(NC(=O)c4ccnn4C)C(=O)Nc4ccc(-c5c(C)n[nH]c5C)cc4)c3)cc2)nn1] | N-[1-[[2-chloro-5-[4-[1-[2-(2- methoxyethoxy)ethyl]triazol-4- yl]phenyl]phenyl]methyl]-2-[4- (3,5-dimethyl-1H-pyrazol-4- yl)anilino]-2-oxo-ethyl]-2- methyl-pyrazole-3- carboxamide | 722.298 | 2.23 |
| 314 | 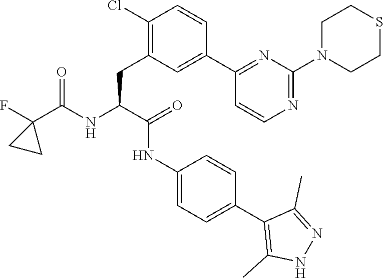 [SMILES: Cc1n[nH]c(C)c1-c1ccc(NC(=O)[C@H](Cc2cc(-c3ccnc(N4CCSCC4)n3)ccc2Cl)NC(=O)C2(F)CC2)cc1] | N-[(1S)-1-[[2-chloro-5-(2- thiomorpholinopyrimidin-4- yl)phenyl]methyl]-2-[4-(3,5- dimethyl-1H-pyrazol-4- yl)anilino]-2-oxo-ethyl]-1- fluoro- cyclopropanecarboxamide | 634.217 | 2.52 |
| 315 | 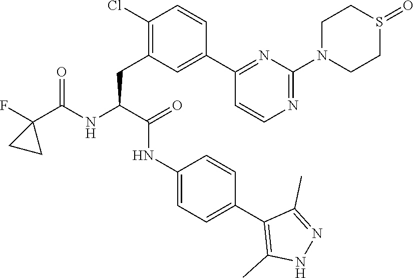 [SMILES: Cc1n[nH]c(C)c1-c1ccc(NC(=O)[C@H](Cc2cc(-c3ccnc(N4CCS(=O)CC4)n3)ccc2Cl)NC(=O)C2(F)CC2)cc1] | N-[(1S)-1-[[2-chloro-5-[2-(1- oxidothiomorpholin-1-ium-4- yl)pyrimidin-4- yl]phenyl]methyl]-2-[4-(3,5- dimethyl-1H-pyrazol-4- yl)anilino]-2-oxo-ethyl]-1- fluoro- cyclopropanecarboxamide | 650.213 | 2.13 |
| 316 | 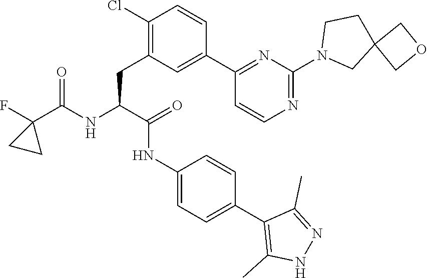 [SMILES: Cc1n[nH]c(C)c1-c1ccc(NC(=O)[C@H](Cc2cc(-c3ccnc(N4CCC5(COC5)C4)n3)ccc2Cl)NC(=O)C2(F)CC2)cc1] | N-[(1S)-1-[[2-chloro-5-[2-(2- oxa-7-azaspiro[3.4]octan-7- yl)pyrimidin-4- yl]phenyl]methyl]-2-[4-(3,5- dimethyl-1H-pyrazol-4- yl)anilino]-2-oxo-ethyl]-1- fluoro- cyclopropanecarboxamide | 644.256 | 2.25 |
| 317 | 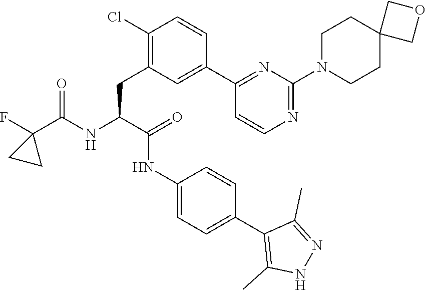 [SMILES: Cc1n[nH]c(C)c1-c1ccc(NC(=O)[C@H](Cc2cc(-c3ccnc(N4CCC5(CC4)COC5)n3)ccc2Cl)NC(=O)C2(F)CC2)cc1] | N-[(1S)-1-[[2-chloro-5-[2-(2- oxa-7-azaspiro[3.5]nonan-7- yl)pyrimidin-4- yl]phenyl]methyl]-2-[4-(3,5- dimethyl-1H-pyrazol-4- yl)anilino]-2-oxo-ethyl]-1- fluoro- cyclopropanecarboxamide | 658.271 | 2.36 |
| 318 | 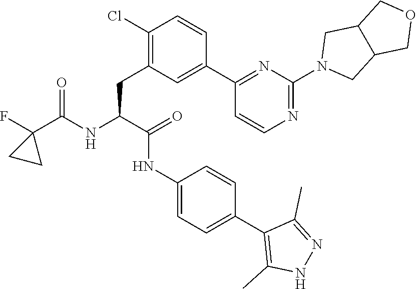 [SMILES: Cc1n[nH]c(C)c1-c1ccc(NC(=O)[C@H](Cc2cc(-c3ccnc(N4CC5COCC5C4)n3)ccc2Cl)NC(=O)C2(F)CC2)cc1] | N-[(1S)-1-[[5-[2- (1,3,3a,4,6,6a- hexahydrofuro[3,4-c]pyrrol-5- yl)pyrimidin-4-yl]-2-chloro- phenyl]methyl]-2-[4-(3,5- dimethyl-1H-pyrazol-4- yl)anilino]-2-oxo-ethyl]-1- fluoro- cyclopropanecarboxamide | 644.256 | 2.27 |
| 319 | 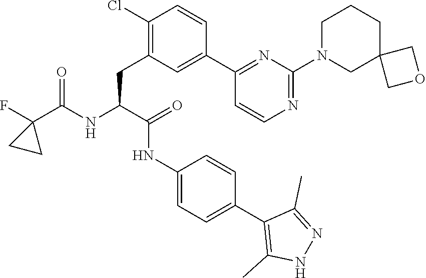 [SMILES: Cc1n[nH]c(C)c1-c1ccc(NC(=O)[C@H](Cc2cc(-c3ccnc(N4CCCC5(COC5)C4)n3)ccc2Cl)NC(=O)C2(F)CC2)cc1] | N-[(1S)-1-[[2-chloro-5-[2-(2- oxa-8-azaspiro[3.5]nonan-8- yl)pyrimidin-4- yl]phenyl]methyl]-2-[4-(3,5- dimethyl-1H-pyrazol-4- yl)anilino]-2-oxo-ethyl]-1- fluoro- cyclopropanecarboxamide | 658.272 | 2.41 |
| 320 | 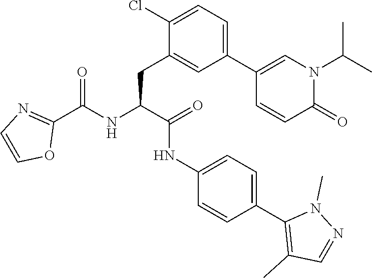 [SMILES: Cc1cnn(C)c1-c1ccc(NC(=O)[C@H](Cc2cc(-c3ccc(=O)n(C(C)C)c3)ccc2Cl)NC(=O)c2ncco2)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo-ethyl]oxazole- 2-carboxamide | 599.218 | 2.26 |
| 321 | 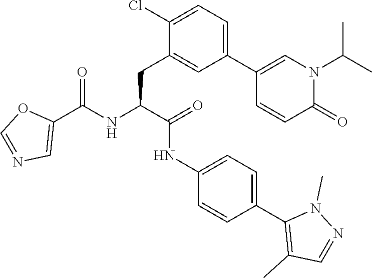 [SMILES: Cc1cnn(C)c1-c1ccc(NC(=O)[C@H](Cc2cc(-c3ccc(=O)n(C(C)C)c3)ccc2Cl)NC(=O)c2cnco2)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo-ethyl]oxazole- 5-carboxamide | 599.217 | 2.19 |
| 322 | 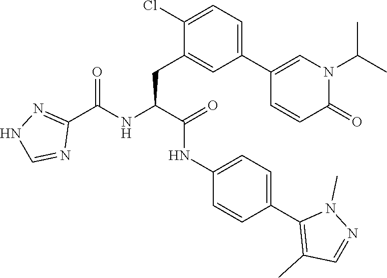 [SMILES: Cc1cnn(C)c1-c1ccc(NC(=O)[C@H](Cc2cc(-c3ccc(=O)n(C(C)C)c3)ccc2Cl)NC(=O)c2nc[nH]n2)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo-ethyl]-1H- 1,2,4-triazole-3-carboxamide | 599.227 | 2.13 |
| 323 | 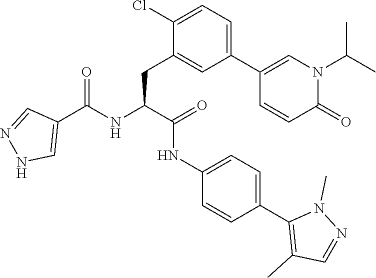 [SMILES: Cc1cnn(C)c1-c1ccc(NC(=O)[C@H](Cc2cc(-c3ccc(=O)n(C(C)C)c3)ccc2Cl)NC(=O)c2cn[nH]c2)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo-ethyl]-1H- pyrazole-4-carboxamide | 598.232 | 2.12 |
| 324 | 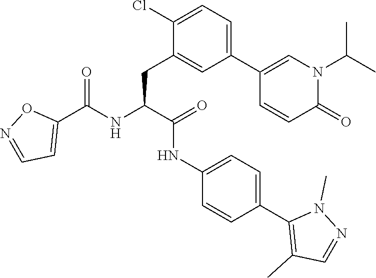 [SMILES: Cc1cnn(C)c1-c1ccc(NC(=O)[C@H](Cc2cc(-c3ccc(=O)n(C(C)C)c3)ccc2Cl)NC(=O)c2ccno2)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo- ethyl]isoxazole-5-carboxamide | 599.218 | 2.27 |
| 325 | 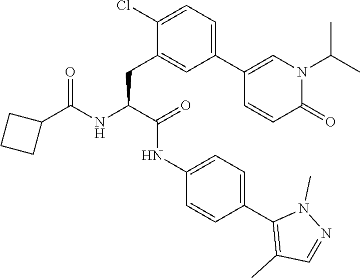 [SMILES: Cc1cnn(C)c1-c1ccc(NC(=O)[C@H](Cc2cc(-c3ccc(=O)n(C(C)C)c3)ccc2Cl)NC(=O)C2CCC2)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo- ethyl]cyclobutanecarboxamide | 586.257 | 2.34 |
| 326 | 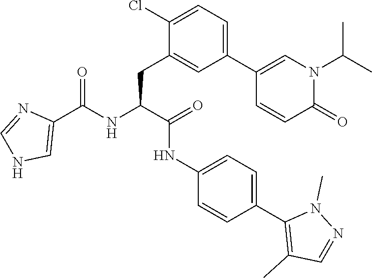 [SMILES: Cc1cnn(C)c1-c1ccc(NC(=O)[C@H](Cc2cc(-c3ccc(=O)n(C(C)C)c3)ccc2Cl)NC(=O)c2c[nH]cn2)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo-ethyl]-1H- imidazole-4-carboxamide | 598.231 | 2.12 |
| 327 | 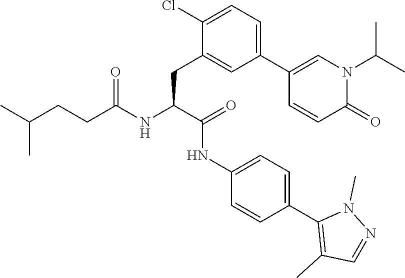 [SMILES: Cc1cnn(C)c1-c1ccc(NC(=O)[C@H](Cc2cc(-c3ccc(=O)n(C(C)C)c3)ccc2Cl)NC(=O)CCC(C)C)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo-ethyl]-4- methyl-pentanamide | 602.290 | 2.43 |
| 328 | 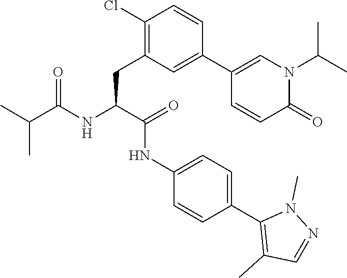 [SMILES: Cc1cnn(C)c1-c1ccc(NC(=O)[C@H](Cc2cc(-c3ccc(=O)n(C(C)C)c3)ccc2Cl)NC(=O)C(C)C)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo-ethyl]-2- methyl-propanamide | 574.259 | 2.31 |
| 329 | 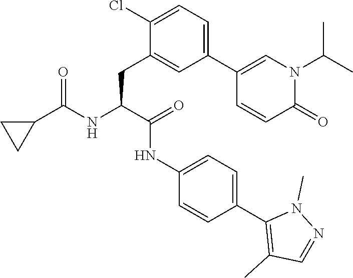 [SMILES: Cc1cnn(C)c1-c1ccc(NC(=O)[C@H](Cc2cc(-c3ccc(=O)n(C(C)C)c3)ccc2Cl)NC(=O)C2CC2)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo- ethyl]cyclopropanecarboxamide | 572.242 | 2.27 |
| 330 | 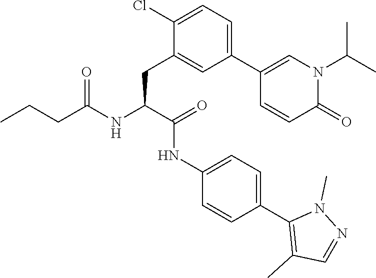 [SMILES: CCCC(=O)N[C@@H](Cc1cc(-c2ccc(=O)n(C(C)C)c2)ccc1Cl)C(=O)Nc1ccc(-c2c(C)cnn2C)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo- ethyl]butanamide | 574.259 | 2.30 |
| 331 | 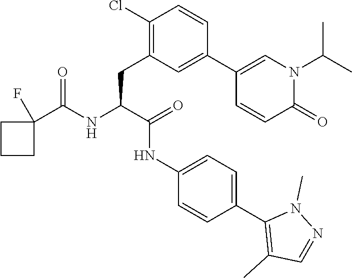 [SMILES: Cc1cnn(C)c1-c1ccc(NC(=O)[C@H](Cc2cc(-c3ccc(=O)n(C(C)C)c3)ccc2Cl)NC(=O)C2(F)CCC2)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo-ethyl]-1- fluoro-cyclobutanecarboxamide | 604.248 | 2.41 |
| 332 | 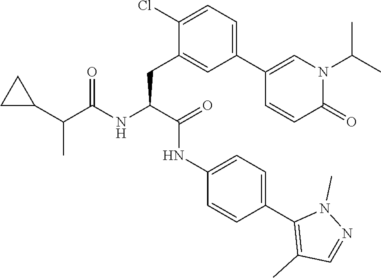 [SMILES: Cc1cnn(C)c1-c1ccc(NC(=O)[C@H](Cc2cc(-c3ccc(=O)n(C(C)C)c3)ccc2Cl)NC(=O)C(C)C2CC2)cc1] | (2S)-3-[2-chloro-5-(1- isopropyl-6-oxo-3- pyridyl)phenyl]-2-(2- cyclopropylpropanoylamino)- [4-(2,4-dimethylpyrazol-3- yl)phenyl]propanamide | 600.273 | 2.39 |
| 333 | 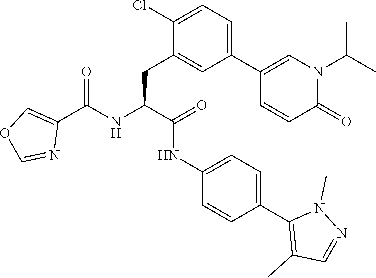 [SMILES: Cc1cnn(C)c1-c1ccc(NC(=O)[C@H](Cc2cc(-c3ccc(=O)n(C(C)C)c3)ccc2Cl)NC(=O)c2cocn2)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo-ethyl]oxazole- 4-carboxamide | 599.216 | 2.26 |
| 334 | 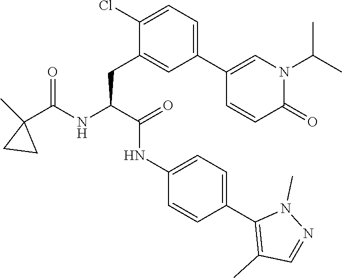 [SMILES: Cc1cnn(C)c1-c1ccc(NC(=O)[C@H](Cc2cc(-c3ccc(=O)n(C(C)C)c3)ccc2Cl)NC(=O)C2(C)CC2)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo-ethyl]-1- methyl- cyclopropanecarboxamide | 586.259 | 2.36 |
| 335 | 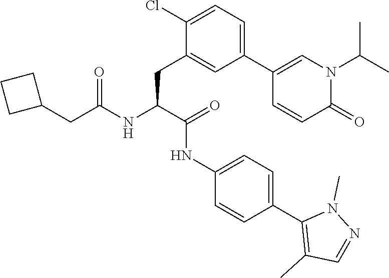 [SMILES: Cc1cnn(C)c1-c1ccc(NC(=O)[C@H](Cc2cc(-c3ccc(=O)n(C(C)C)c3)ccc2Cl)NC(=O)CC2CCC2)cc1] | (2S)-3-[2-chloro-5-(1- isopropyl-6-oxo-3- pyridyl)phenyl]-2-[(2- cyclobutylacetyl)amino]-N-[4- (2,4-dimethylpyrazol-3- yl)phenyl]propanamide | 600.274 | 2.39 |
| 336 | 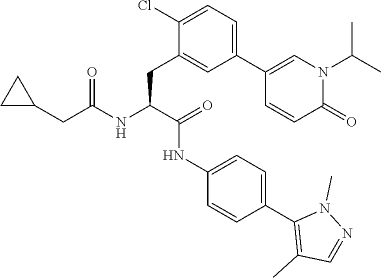 [SMILES: Cc1cnn(C)c1-c1ccc(NC(=O)[C@H](Cc2cc(-c3ccc(=O)n(C(C)C)c3)ccc2Cl)NC(=O)CC2CC2)cc1] | (2S)-3-[2-chloro-5-(1- isopropyl-6-oxo-3- pyridyl)phenyl]-2-[(2- cyclopropylacetyl)amino]-N-[4- (2,4-dimethylpyrazol-3- yl)phenyl]propanamide | 586.259 | 2.32 |
| 337 | 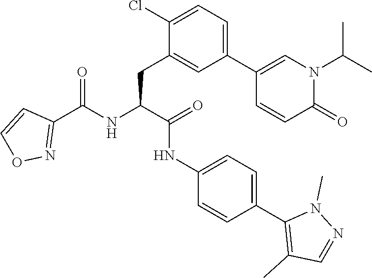 [SMILES: Cc1cnn(C)c1-c1ccc(NC(=O)[C@H](Cc2cc(-c3ccc(=O)n(C(C)C)c3)ccc2Cl)NC(=O)c2ccon2)cc1] | N-[(1S)-1-[[2-chloro-5-(1- isopropyl-6-oxo-3- pyridyl)phenyl]methyl]-2-[4- (2,4-dimethylpyrazol-3- yl)anilino]-2-oxo- ethyl]isoxazole-3-carboxamide | 599.216 | 2.31 |
| 338 | 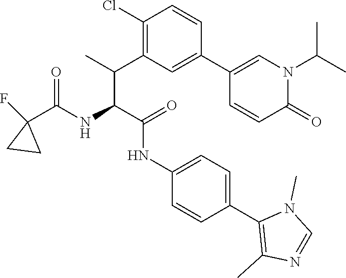 [SMILES: Cc1ncn(C)c1-c1ccc(NC(=O)[C@@H](NC(=O)C2(F)CC2)C(C)c2cc(-c3ccc(=O)n(C(C)C)c3)ccc2Cl)cc1] | Diastereomer 2 of N-[(1S)-2- [2-chloro-5-(1-isopropyl-6-oxo- 3-pyridyl)phenyl]-1-[[4-(3,5- dimethyl-1H-pyrazol-4- yl)phenyl]carbamoyl]propyl]-1- fluoro- cyclopropanecarboxamide | 604.248 | 2.04 |
| 339 | 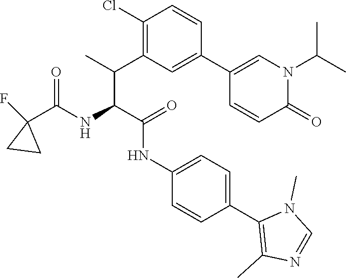 [SMILES: Cc1ncn(C)c1-c1ccc(NC(=O)[C@@H](NC(=O)C2(F)CC2)C(C)c2cc(-c3ccc(=O)n(C(C)C)c3)ccc2Cl)cc1] | Diastereomer 1 of N-[(1S)-2- [2-chloro-5-(1-isopropyl-6-oxo- 3-pyridyl)phenyl]-1-[[4-(3,5- dimethyl-1H-pyrazol-4- yl)phenyl]carbamoyl]propyl]-1- fluoro- cyclopropanecarboxamide | 604.248 | 2.25 |
| 340 | 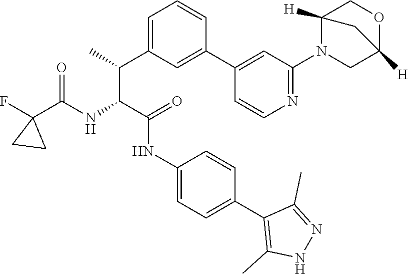 [SMILES: Cc1n[nH]c(C)c1-c1ccc(NC(=O)[C@H](NC(=O)C2(F)CC2)[C@H](C)c2cccc(-c3ccnc(N4C[C@@H]5C[C@H]4CO5)c3)c2)cc1] | N-[(1R,2R)-1-[[4-(3,5- dimethyl-1H-pyrazol-4- yl)phenyl]carbamoyl]-2-[3-[2- [(1S,4S)-2-oxa-5- azabicyclo[2.2.1]heptan-5-yl]- 4-pyridyl]phenyl]propyl]-1- fluoro- cyclopropanecarboxamide | 609.299 | 1.94 |
| 341 | 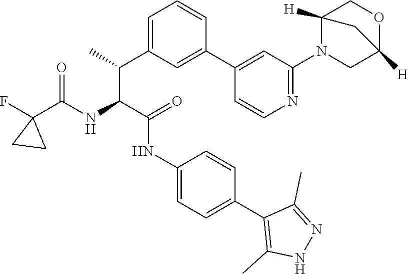 [SMILES: Cc1n[nH]c(C)c1-c1ccc(NC(=O)[C@@H](NC(=O)C2(F)CC2)[C@H](C)c2cccc(-c3ccnc(N4C[C@@H]5C[C@H]4CO5)c3)c2)cc1] | N-[(1S,2R)-1-[[4-(3,5- dimethyl-1H-pyrazol-4- yl)phenyl]carbamoyl]-2-[3-[2- [(1S,4S)-2-oxa-5- azabicyclo[2.2.1]heptan-5-yl]- 4-pyridyl]phenyl]propyl]-1- fluoro- cyclopropanecarboxamide | 609.300 | 1.92 |
| 342 | 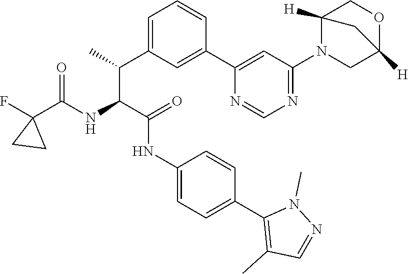 [SMILES: Cc1cnn(C)c1-c1ccc(NC(=O)[C@@H](NC(=O)C2(F)CC2)[C@H](C)c2cccc(-c3cc(N4C[C@@H]5C[C@H]4CO5)ncn3)c2)cc1] | N-[(1S,2R)-1-[[4-(2,4- dimethylpyrazol-3- yl)phenyl]carbamoyl]-2-[3-[6- [(1S,4S)-2-oxa-5- azabicyclo[2.2.1]heptan-5- yl]pyrimidin-4- yl]phenyl]propyl]-1-fluoro- cyclopropanecarboxamide | 610.294 | 2.03 |
| 343 | 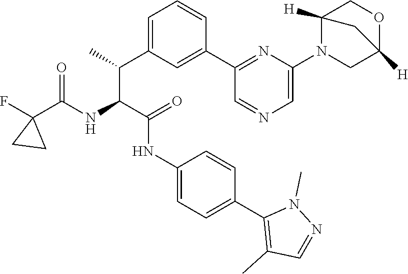 [SMILES: Cc1cnn(C)c1-c1ccc(NC(=O)[C@@H](NC(=O)C2(F)CC2)[C@H](C)c2cccc(-c3cncc(N4C[C@@H]5C[C@H]4CO5)n3)c2)cc1] | N-[(1S,2R)-1-[[4-(2,4- dimethylpyrazol-3- yl)phenyl]carbamoyl]-2-[3-[6- [(1S,4S)-2-oxa-5- azabicyclo[2.2.1]heptan-5- yl]pyrazin-2-yl]phenyl]propyl]- 1-fluoro- cyclopropanecarboxamide | 610.295 | 2.31 |
| 344 | 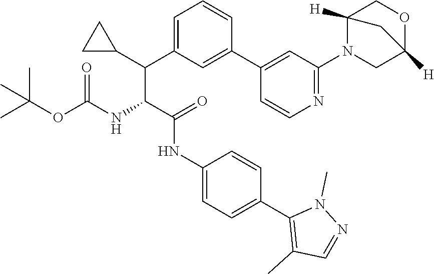 [SMILES: Cc1cnn(C)c1-c1ccc(NC(=O)[C@H](NC(=O)OC(C)(C)C)C(c2cccc(-c3ccnc(N4C[C@@H]5C[C@H]4CO5)c3)c2)C2CC2)cc1] | Diastereomer 2 of tert-butyl N- [1-[(R)-cyclopropyl-[3-[2- [(1S,4S)-2-oxa-5- azabicyclo[2.2.1]heptan-5-yl]- 4-pyridyl]phenyl]methyl]-2-[4- (2,4-dimethylpyrazol-3- yl)anilino]-2-oxo- ethyl]carbamate | 649.351 | 2.14 |
| 345 | 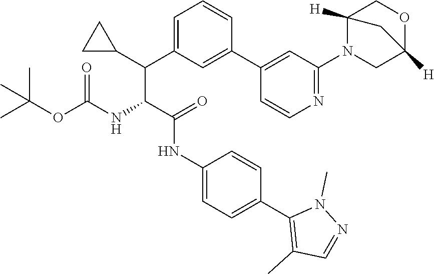 [SMILES: Cc1cnn(C)c1-c1ccc(NC(=O)[C@H](NC(=O)OC(C)(C)C)C(c2cccc(-c3ccnc(N4C[C@@H]5C[C@H]4CO5)c3)c2)C2CC2)cc1] | Diastereomer 1 of tert-butyl N- [1-[(R)-cyclopropyl-[3-[2- [(1S,4S)-2-oxa-5- azabicyclo[2.2.1]heptan-5-yl]- 4-pyridyl]phenyl]methyl]-2-[4- (2,4-dimethylpyrazol-3- yl)anilino]-2-oxo- ethyl]carbamate | 649.35 | 2.15 |
| 346 | 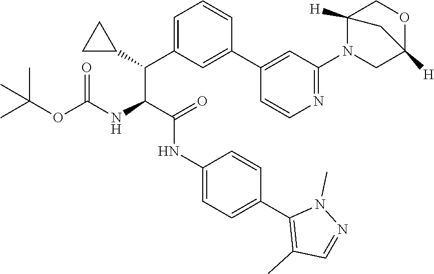 [SMILES: Cc1cnn(C)c1-c1ccc(NC(=O)[C@@H](NC(=O)OC(C)(C)C)[C@@H](c2cccc(-c3ccnc(N4C[C@@H]5C[C@H]4CO5)c3)c2)C2CC2)cc1] | tert-butyl N-[(1S)-1-[(R)- cyclopropyl-[3-[2-[(1S,4S)-2- oxa-5-azabicyclo[2.2.1]heptan- 5-yl]-4-pyridyl]phenyl]methyl]- 2-[4-(2,4-dimethylpyrazol-3- yl)anilino]-2-oxo- ethyl]carbamate | 649.351 | 2.15 |
| 347 | 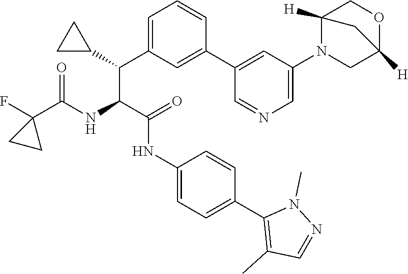 [SMILES: Cc1cnn(C)c1-c1ccc(NC(=O)[C@@H](NC(=O)C2(F)CC2)[C@@H](c2cccc(-c3cncc(N4C[C@@H]5C[C@H]4CO5)c3)c2)C2CC2)cc1] | N-[(1S)-1-[(R)-cyclopropyl-[3- [5-[(1S,4S)-2-oxa-5- azabicyclo[2.2.1]heptan-5-yl]- 3-pyridyl]phenyl]methyl]-2-[4- (2,4-dimethylpyrazol-3- yl)anilino]-2-oxo-ethyl]-1- fluoro- cyclopropanecarboxamide | 635.316 | 2.01 |
| 348 | 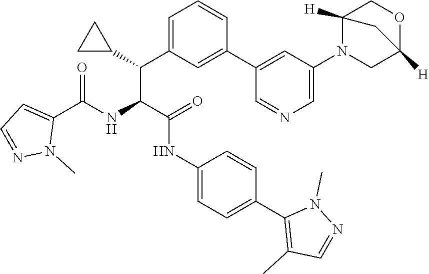 [SMILES: Cc1cnn(C)c1-c1ccc(NC(=O)[C@@H](NC(=O)c2ccnn2C)[C@@H](c2cccc(-c3cncc(N4C[C@@H]5C[C@H]4CO5)c3)c2)C2CC2)cc1] | N-[(1S)-1-[(R)-cyclopropyl-[3- [5-[(1S,4S)-2-oxa-5- azabicyclo[2.2.1]heptan-5-yl]- 3-pyridyl]phenyl]methyl]-2-[4- (2,4-dimethylpyrazol-3- yl)anilino]-2-oxo-ethyl]-2- methyl-pyrazole-3- carboxamide | 657.331 | 2.01 |
| 349 | 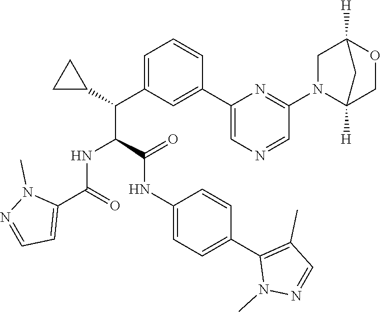 [SMILES: Cc1cnn(C)c1-c1ccc(NC(=O)[C@@H](NC(=O)c2ccnn2C)[C@@H](c2cccc(-c3cncc(N4C[C@@H]5C[C@H]4CO5)n3)c2)C2CC2)cc1] | N-[(1S)-1-[(R)-cyclopropyl-[3- [6-[(1S,4S)-2-oxa-5- azabicyclo[2.2.1]heptan-5- yl]pyrazin-2-yl]phenyl]methyl]- 2-[4-(2,4-dimethylpyrazol-3- yl)anilino]-2-oxo-ethyl]-2- methyl-pyrazole-3- carboxamide | 658.326 | 2.27 |
| 350 | 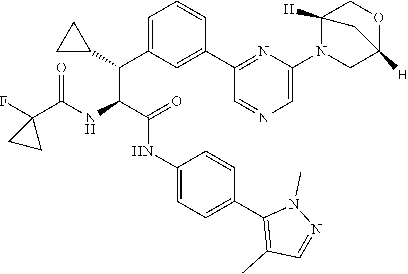 [SMILES: Cc1cnn(C)c1-c1ccc(NC(=O)[C@@H](NC(=O)C2(F)CC2)[C@@H](c2cccc(-c3cncc(N4C[C@@H]5C[C@H]4CO5)n3)c2)C2CC2)cc1] | N-[(1S)-1-[(R)-cyclopropyl-[3- [6-[(1S,4S)-2-oxa-5- azabicyclo[2.2.1]heptan-5- yl]pyrazin-2-yl]phenyl]methyl]- 2-[4-(2,4-dimethylpyrazol-3- yl)anilino]-2-oxo-ethyl]-1- fluoro- cyclopropanecarboxamide | 636.311 | 2.38 |
| 351 | 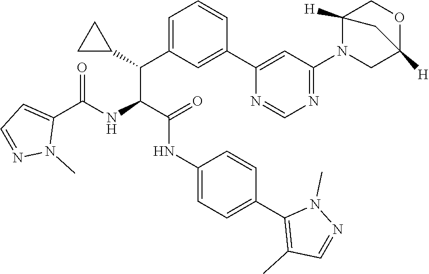 [SMILES: Cc1cnn(C)c1-c1ccc(NC(=O)[C@@H](NC(=O)c2ccnn2C)[C@@H](c2cccc(-c3cc(N4C[C@@H]5C[C@H]4CO5)ncn3)c2)C2CC2)cc1] | N-[(1S)-1-[(R)-cyclopropyl-[3- [6-[(1S,4S)-2-oxa-5- azabicyclo[2.2.1]heptan-5- yl]pyrimidin-4- yl]phenyl]methyl]-2-[4-(2,4- dimethylpyrazol-3-yl)anilino]- 2-oxo-ethyl]-2-methyl- pyrazole-3-carboxamide | 658.326 | 2.02 |
| 352 | 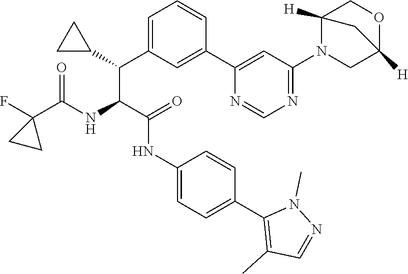 [SMILES: Cc1cnn(C)c1-c1ccc(NC(=O)[C@@H](NC(=O)C2(F)CC2)[C@@H](c2cccc(-c3cc(N4C[C@@H]5C[C@H]4CO5)ncn3)c2)C2CC2)cc1] | N-[(1S)-1-[(R)-cyclopropyl-[3- [6-[(1S,4S)-2-oxa-5- azabicyclo[2.2.1]heptan-5- yl]pyrimidin-4- yl]phenyl]methyl]-2-[4-(2,4- dimethylpyrazol-3-yl)anilino]- 2-oxo-ethyl]-1-fluoro- cyclopropanecarboxamide | 636.311 | 2.10 |
| 353 | 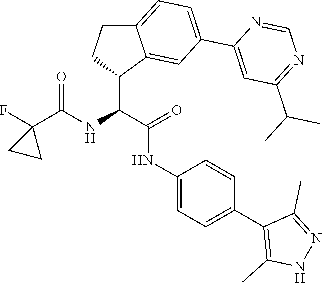 [SMILES: Cc1n[nH]c(C)c1-c1ccc(NC(=O)[C@@H](NC(=O)C2(F)CC2)[C@@H]2CCc3ccc(-c4cc(C(C)C)ncn4)cc32)cc1] | N-[(1S)-2-[4-(3,5-dimethyl- 1H-pyrazol-4-yl)anilino]-1- [(1R)-6-(6-isopropylpyrimidin- 4-yl)indan-1-yl]-2-oxo-ethyl]- 1-fluoro- cyclopropanecarboxamide | 567.291 | 2.32 |
| 354 | 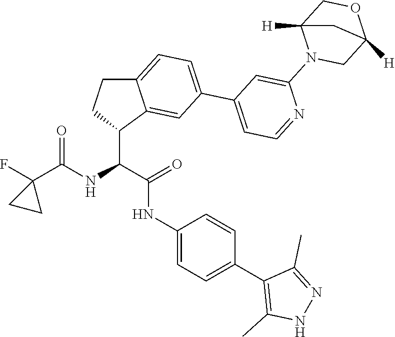 [SMILES: Cc1n[nH]c(C)c1-c1ccc(NC(=O)[C@@H](NC(=O)C2(F)CC2)[C@@H]2CCc3ccc(-c4ccnc(N5C[C@@H]6C[C@H]5CO6)c4)cc32)cc1] | N-[(1S)-2-[4-(3,5-dimethyl- 1H-pyrazol-4-yl)anilino]-1- [(1R)-6-[2-[(1S,4S)-2-oxa-5- azabicyclo[2.2.1]heptan-5-yl]- 4-pyridyl]indan-1-yl]-2-oxo- ethyl]-1-fluoro- cyclopropanecarboxamide | 621.298 | 1.96 |
| 355 | 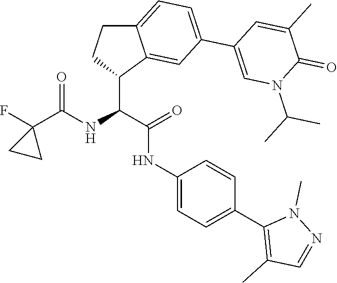 [SMILES: Cc1cnn(C)c1-c1ccc(NC(=O)[C@@H](NC(=O)C2(F)CC2)[C@@H]2CCc3ccc(-c4cc(C)c(=O)n(C(C)C)c4)cc32)cc1] | N-[(1S)-2-[4-(2,4- dimethylpyrazol-3-yl)anilino]- 1-[(1R)-6-(1-isopropyl-5- methyl-6-oxo-3-pyridyl)indan- 1-yl]-2-oxo-ethyl]-1-fluoro- cyclopropanecarboxamide | 596.305 | 2.41 |
| 356 | 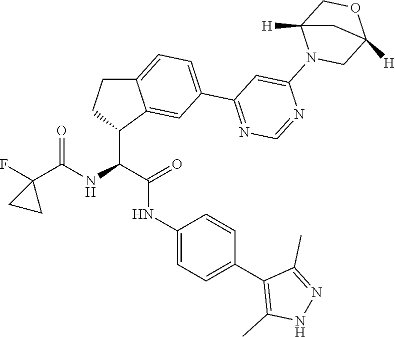 [SMILES: Cc1n[nH]c(C)c1-c1ccc(NC(=O)[C@@H](NC(=O)C2(F)CC2)[C@@H]2CCc3ccc(-c4cc(N5C[C@@H]6C[C@H]5CO6)ncn4)cc32)cc1] | N-[(1S)-2-[4-(3,5-dimethyl- 1H-pyrazol-4-yl)anilino]-1- [(1R)-6-[6-[(1S,4S)-2-oxa-5- azabicyclo[2.2.1]heptan-5- yl]pyrimidin-4-yl]indan-1-yl]-2- oxo-ethyl]-1-fluoro- cyclopropanecarboxamide | 622.295 | 1.92 |
| 357 | 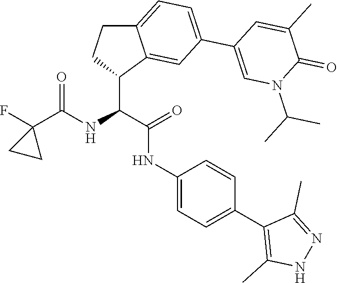 [SMILES: Cc1n[nH]c(C)c1-c1ccc(NC(=O)[C@@H](NC(=O)C2(F)CC2)[C@@H]2CCc3ccc(-c4cc(C)c(=O)n(C(C)C)c4)cc32)cc1] | N-[(1S)-2-[4-(3,5-dimethyl- 1H-pyrazol-4-yl)anilino]-1- [(1R)-6-(1-isopropyl-5-methyl- 6-oxo-3-pyridyl)indan-1-yl]-2- oxo-ethyl]-1-fluoro- cyclopropanecarboxamide | 596.304 | 2.27 |
| 358 | 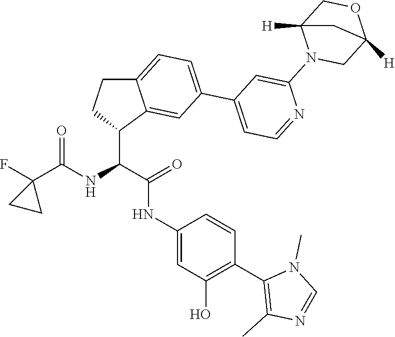 [SMILES: Cc1ncn(C)c1-c1ccc(NC(=O)[C@@H](NC(=O)C2(F)CC2)[C@@H]2CCc3ccc(-c4ccnc(N5C[C@@H]6C[C@H]5CO6)c4)cc32)cc1O] | N-[(1S)-2-[4-(3,5- dimethylimidazol-4-yl)-3- hydroxy-anilino]-1-[(1R)-6-[2- [(1S,4S)-2-oxa-5- azabicyclo[2.2.1]heptan-5-yl]- 4-pyridyl]indan-1-yl]-2-oxo- ethyl]-1-fluoro- cyclopropanecarboxamide | 637.294 | 1.78 |
| 359 | 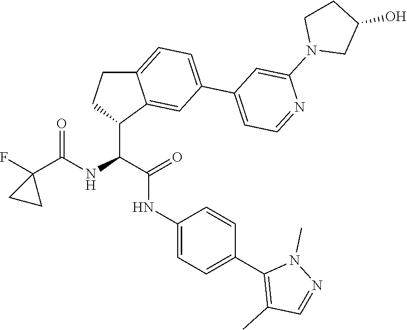 [SMILES: Cc1cnn(C)c1-c1ccc(NC(=O)[C@@H](NC(=O)C2(F)CC2)[C@@H]2CCc3ccc(-c4ccnc(N5CC[C@H](O)C5)c4)cc32)cc1] | N-[(1S)-2-[4-(2,4- dimethylpyrazol-3-yl)anilino]- 1-[(1R)-6-[2-[(3S)-3- hydroxypyrrolidin-1-yl]-4- pyridyl]indan-1-yl]-2-oxo- ethyl]-1-fluoro- cyclopropanecarboxamide | 609.299 | 2.00 |
| 360 | 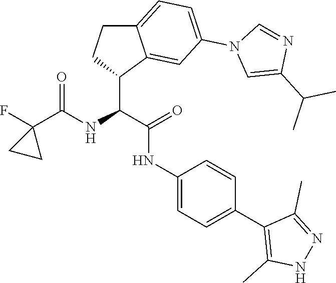 [SMILES: Cc1n[nH]c(C)c1-c1ccc(NC(=O)[C@@H](NC(=O)C2(F)CC2)[C@@H]2CCc3ccc(-n4cnc(C(C)C)c4)cc32)cc1] | N-[(1S)-2-[4-(3,5-dimethyl- 1H-pyrazol-4-yl)anilino]-1- [(1R)-6-(4-isopropylimidazol-1- yl)indan-1-yl]-2-oxo-ethyl]-1- fluoro- cyclopropanecarboxamide | 555.289 | 1.97 |
| 361 | 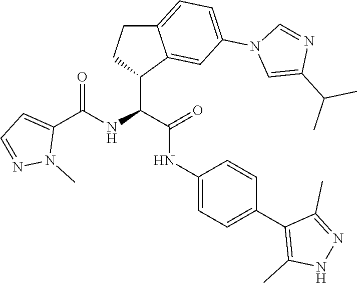 [SMILES: Cc1n[nH]c(C)c1-c1ccc(NC(=O)[C@@H](NC(=O)c2ccnn2C)[C@@H]2CCc3ccc(-n4cnc(C(C)C)c4)cc32)cc1] | N-[(1S)-2-[4-(3,5-dimethyl- 1H-pyrazol-4-yl)anilino]-1- [(1R)-6-(4-isopropylimidazol-1- yl)indan-1-yl]-2-oxo-ethyl]-2- methyl-pyrazole-3- carboxamide | 577.304 | 1.89 |
| 362 | 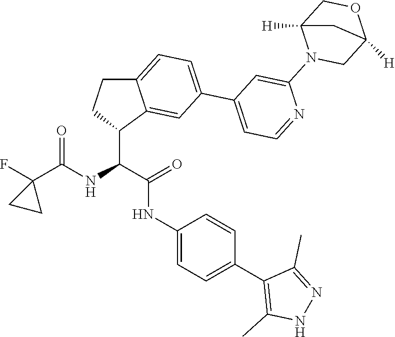 [SMILES: Cc1n[nH]c(C)c1-c1ccc(NC(=O)[C@@H](NC(=O)C2(F)CC2)[C@@H]2CCc3ccc(-c4ccnc(N5C[C@H]6C[C@@H]5CO6)c4)cc32)cc1] | N-[(1S)-2-[4-(3,5-dimethyl- 1H-pyrazol-4-yl)anilino]-1- [(1R)-6-[2-[(1R,4R)-2-oxa-5- azabicyclo[2.2.1]heptan-5-yl]- 4-pyridyl]indan-1-yl]-2-oxo- ethyl]-1-fluoro- cyclopropanecarboxamide | 621.299 | 1.96 |
| 363 | 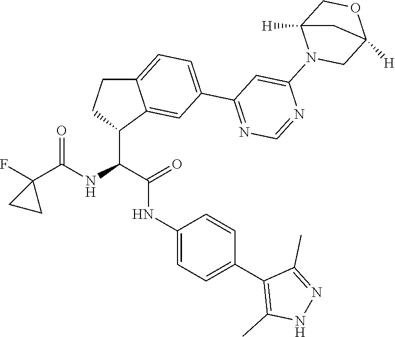 [SMILES: Cc1n[nH]c(C)c1-c1ccc(NC(=O)[C@@H](NC(=O)C2(F)CC2)[C@@H]2CCc3ccc(-c4cc(N5C[C@H]6C[C@@H]5CO6)ncn4)cc32)cc1] | N-[(1S)-2-[4-(3,5-dimethyl- 1H-pyrazol-4-yl)anilino]-1- [(1R)-6-[2-[(1R,4R)-2-oxa-5- azabicyclo[2.2.1]heptan-5- yl]pyrimidin-4-yl]indan-1-yl]-2- oxo-ethyl]-1-fluoro- cyclopropanecarboxamide | 622.295 | 2.19 |
| 364 | 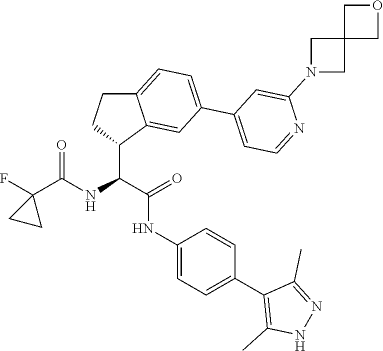 [SMILES: Cc1n[nH]c(C)c1-c1ccc(NC(=O)[C@@H](NC(=O)C2(F)CC2)[C@@H]2CCc3ccc(-c4ccnc(N5CC6(COC6)C5)c4)cc32)cc1] | N-[(1S)-2-[4-(3,5-dimethyl- 1H-pyrazol-4-yl)anilino]-1- [(1R)-6-[2-(6-oxa-2- azaspiro[3.3]heptan-2-yl)-4- pyridyl]indan-1-yl]-2-oxo- ethyl]-1-fluoro- cyclopropanecarboxamide | 621.490 | 0.57 (z) |
| 365 | 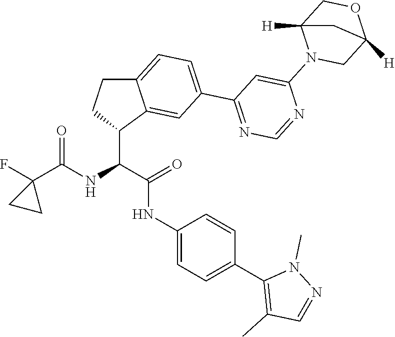 [SMILES: Cc1cnn(C)c1-c1ccc(NC(=O)[C@@H](NC(=O)C2(F)CC2)[C@@H]2CCc3ccc(-c4cc(N5C[C@@H]6C[C@H]5CO6)ncn4)cc32)cc1] | N-[(1S)-2-[4-(2,4- dimethylpyrazol-3-yl)anilino]- 1-[(1R)-6-[6-[(1S,4S)-2-oxa- 5-azabicyclo[2.2.1]heptan-5- yl]pyrimidin-4-yl]indan-1-yl]-2- oxo-ethyl]-1-fluoro- cyclopropanecarboxamide | 621.437 | 2.03 |
| 366 | 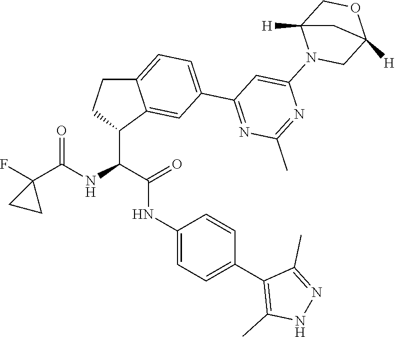 [SMILES: Cc1nc(-c2ccc3c(c2)[C@H]([C@H](NC(=O)C2(F)CC2)C(=O)Nc2ccc(-c4c(C)n[nH]c4C)cc2)CC3)cc(N2C[C@@H]3C[C@H]2CO3)n1] | N-[(1S)-2-[4-(3,5-dimethyl- 1H-pyrazol-4-yl)anilino]-1- [(1R)-6-[2-methyl-6-[(1S,4S)- 2-oxa-5- azabicyclo[2.2.1]heptan-5- yl]pyrimidin-4-yl]indan-1-yl]-2- oxo-ethyl]-1-fluoro- cyclopropanecarboxamide | 636.310 | 1.91 |
| 367 | 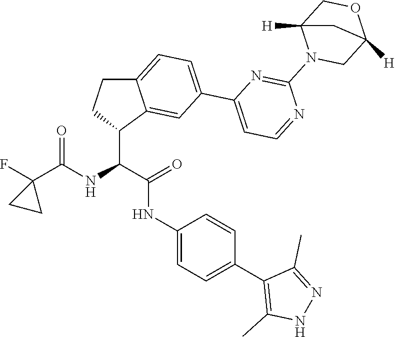 [SMILES: Cc1n[nH]c(C)c1-c1ccc(NC(=O)[C@@H](NC(=O)C2(F)CC2)[C@@H]2CCc3ccc(-c4ccnc(N5C[C@@H]6C[C@H]5CO6)n4)cc32)cc1] | N-[(1S)-2-[4-(3,5-dimethyl- 1H-pyrazol-4-yl)anilino]-1- [(1R)-6-[2-[(1S,4S)-2-oxa-5- azabicyclo[2.2.1]heptan-5- yl]pyrimidin-4-yl]indan-1-yl]-2- oxo-ethyl]-1-fluoro- cyclopropanecarboxamide | 622.296 | 2.19 |
| 368 | 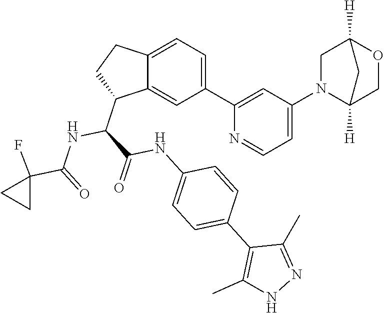 [SMILES: Cc1n[nH]c(C)c1-c1ccc(NC(=O)[C@@H](NC(=O)C2(F)CC2)[C@@H]2CCc3ccc(-c4cc(N5C[C@@H]6C[C@H]5CO6)ccn4)cc32)cc1] | N-[(1S)-2-[4-(3,5-dimethyl- 1H-pyrazol-4-yl)anilino]-1- [(1R)-6-[4-[(1S,4S)-2-oxa-5- azabicyclo[2.2.1]heptan-5-yl]- 2-pyridyl]indan-1-yl]-2-oxo- ethyl]-1-fluoro- cyclopropanecarboxamide | 621.299 | 1.91 |
| 369 | 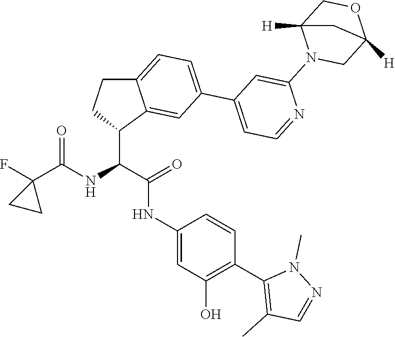 [SMILES: Cc1cnn(C)c1-c1ccc(NC(=O)[C@@H](NC(=O)C2(F)CC2)[C@@H]2CCc3ccc(-c4ccnc(N5C[C@@H]6C[C@H]5CO6)c4)cc32)cc1O] | N-[(1S)-2-[4-(2,4- dimethylpyrazol-3-yl)-3- hydroxy-anilino]-1-[(1R)-6-[2- [(1S,4S)-2-oxa-5- azabicyclo[2.2.1]heptan-5-yl]- 4-pyridyl]indan-1-yl]-2-oxo- ethyl]-1-fluoro- cyclopropanecarboxamide | 637.295 | 1.97 |
| 370 | 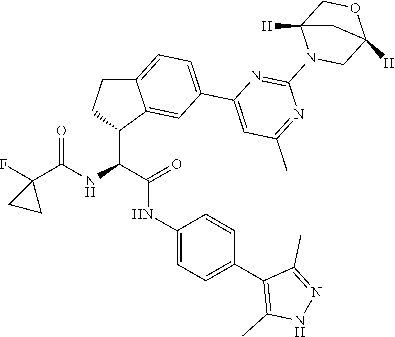 [SMILES: Cc1cc(-c2ccc3c(c2)[C@H]([C@H](NC(=O)C2(F)CC2)C(=O)Nc2ccc(-c4c(C)n[nH]c4C)cc2)CC3)nc(N2C[C@@H]3C[C@H]2CO3)n1] | N-[(1S)-2-[4-(3,5-dimethyl- 1H-pyrazol-4-yl)anilino]-1- [(1R)-6-[6-methyl-2-[(1S,4S)- 2-oxa-5- azabicyclo[2.2.1]heptan-5- yl]pyrimidin-4-yl]indan-1-yl]-2- oxo-ethyl]-1-fluoro- cyclopropanecarboxamide | 636.311 | 2.20 |
| 371 | 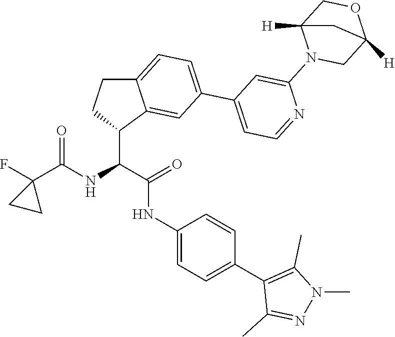 [SMILES: Cc1nn(C)c(C)c1-c1ccc(NC(=O)[C@@H](NC(=O)C2(F)CC2)[C@@H]2CCc3ccc(-c4ccnc(N5C[C@@H]6C[C@H]5CO6)c4)cc32)cc1] | 1-fluoro-N-[(1S)-1-[(1R)-6-[2- [(1S,4S)-2-oxa-5- azabicyclo[2.2.1]heptan-5-yl]- 4-pyridyl]indan-1-yl]-2-oxo-2- [4-(1,3,5-trimethylpyrazol-4- yl)anilino]ethyl] cyclopropanecarboxamide | 635.315 | 2.02 |
| 372 | 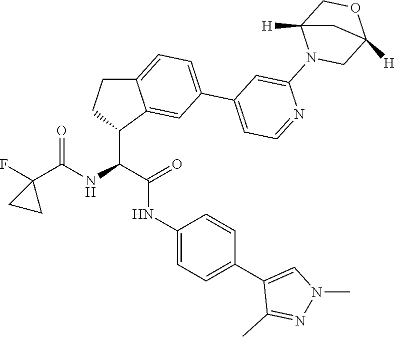 [SMILES: Cc1nn(C)cc1-c1ccc(NC(=O)[C@@H](NC(=O)C2(F)CC2)[C@@H]2CCc3ccc(-c4ccnc(N5C[C@@H]6C[C@H]5CO6)c4)cc32)cc1] | N-[(1S)-2-[4-(1,3- dimethylpyrazol-4-yl)anilino]- 1-[(1R)-6-[2-[(1S,4S)-2-oxa- 5-azabicyclo[2.2.1]heptan-5- yl]-4-pyridyl]indan-1-yl]-2- oxo-ethyl]-1-fluoro- cyclopropanecarboxamide | 621.300 | 1.99 |
| 373 | 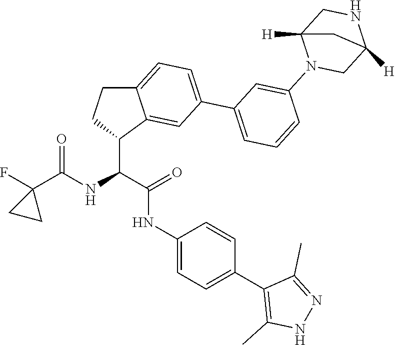 [SMILES: Cc1n[nH]c(C)c1-c1ccc(NC(=O)[C@@H](NC(=O)C2(F)CC2)[C@@H]2CCc3ccc(-c4cccc(N5C[C@@H]6C[C@H]5CN6)c4)cc32)cc1] | N-[(1S)-1-[(1R)-6-[3-[(1S,4S)- 2,5-diazabicyclo[2.2.1]heptan- 2-yl]phenyl]indan-1-yl]-2-[4- (3,5-dimethyl-1H-pyrazol-4- yl)anilino]-2-oxo-ethyl]-1- fluoro- cyclopropanecarboxamide | 619.32 | 2.02 |
| 374 | 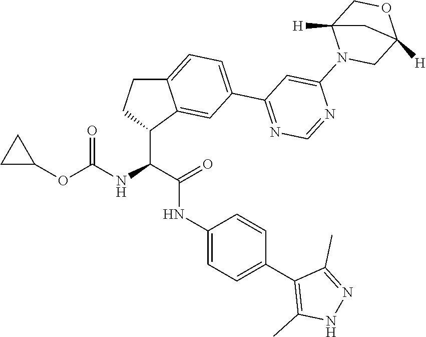 [SMILES: Cc1n[nH]c(C)c1-c1ccc(NC(=O)[C@@H](NC(=O)OC2CC2)[C@@H]2CCc3ccc(-c4cc(N5C[C@@H]6C[C@H]5CO6)ncn4)cc32)cc1] | cyclopropyl N-[(1S)-2-[4-(3,5- dimethyl-1H-pyrazol-4- yl)anilino]-1-[(1R)-6-[6- [(1S,4S)-2-oxa-5- azabicyclo[2.2.1]heptan-5- yl]pyrimidin-4-yl]indan-1-yl]-2- oxo-ethyl]carbamate | 620.299 | 1.90 |
| 375 | 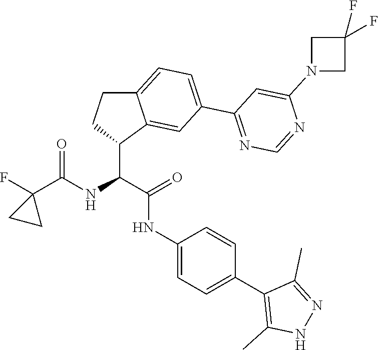 [SMILES: Cc1n[nH]c(C)c1-c1ccc(NC(=O)[C@@H](NC(=O)C2(F)CC2)[C@@H]2CCc3ccc(-c4cc(N5CC(F)(F)C5)ncn4)cc32)cc1] | N-[(1S)-1-[(1R)-6-[6-(3,3- difluoroazetidin-1-yl)pyrimidin- 4-yl]indan-1-yl]-2-[4-(3,5- dimethyl-1H-pyrazol-4- yl)anilino]-2-oxo-ethyl]-1- fluoro- cyclopropanecarboxamide | 616.265 | 2.16 |
| 376 | 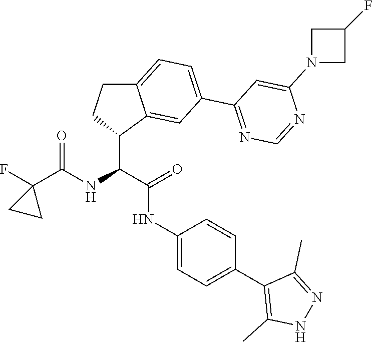 [SMILES: Cc1n[nH]c(C)c1-c1ccc(NC(=O)[C@@H](NC(=O)C2(F)CC2)[C@@H]2CCc3ccc(-c4cc(N5CC(F)C5)ncn4)cc32)cc1] | N-[(1S)-2-[4-(3,5-dimethyl- 1H-pyrazol-4-yl)anilino]-1- [(1R)-6-[6-(3-fluoroazetidin-1- yl)pyrimidin-4-yl]indan-1-yl]-2- oxo-ethyl]-1-fluoro- cyclopropanecarboxamide | 598.275 | 2.00 |
| 377 | 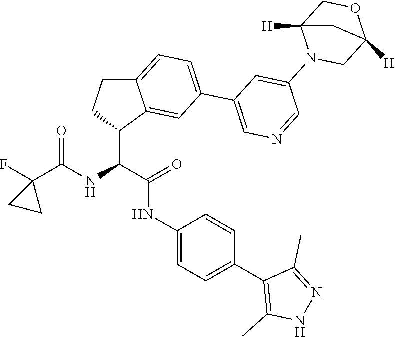 [SMILES: Cc1n[nH]c(C)c1-c1ccc(NC(=O)[C@@H](NC(=O)C2(F)CC2)[C@@H]2CCc3ccc(-c4cncc(N5C[C@@H]6C[C@H]5CO6)c4)cc32)cc1] | N-[(1S)-2-[4-(3,5-dimethyl- 1H-pyrazol-4-yl)anilino]-1- [(1R)-6-[5-[(1S,4S)-2-oxa-5- azabicyclo[2.2.1]heptan-5-yl]- 3-pyridyl]indan-1-yl]-2-oxo- ethyl]-1-fluoro- cyclopropanecarboxamide | 621.300 | 1.96 |
| 378 | 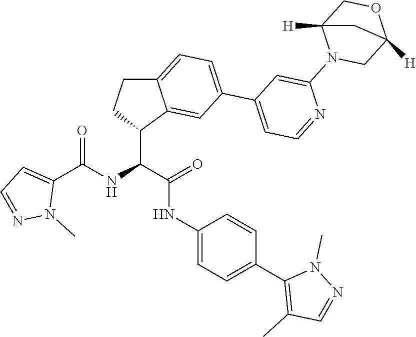 [SMILES: Cc1cnn(C)c1-c1ccc(NC(=O)[C@@H](NC(=O)c2ccnn2C)[C@@H]2CCc3ccc(-c4ccnc(N5C[C@@H]6C[C@H]5CO6)c4)cc32)cc1] | N-[(1S)-2-[4-(2,4- dimethylpyrazol-3-yl)anilino]- 1-[(1R)-6-[2-[(1S,4S)-2-oxa- 5-azabicyclo[2.2.1]heptan-5- yl]-4-pyridyl]indan-1-yl]-2- oxo-ethyl]-2-methyl-pyrazole- 3-carboxamide | 643.315 | 1.97 |
| 379 | 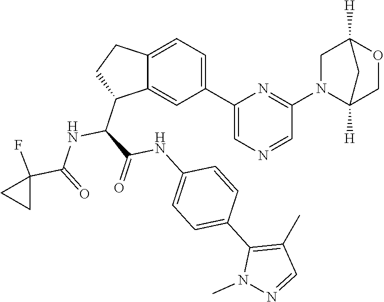 [SMILES: Cc1cnn(C)c1-c1ccc(NC(=O)[C@@H](NC(=O)C2(F)CC2)[C@@H]2CCc3ccc(-c4cncc(N5C[C@@H]6C[C@H]5CO6)n4)cc32)cc1] | N-[(1S)-2-[4-(2,4- dimethylpyrazol-3-yl)anilino]- 1-[(1R)-6-[6-[(1S,4S)-2-oxa- 5-azabicyclo[2.2.1]heptan-5- yl]pyrazin-2-yl]indan-1-yl]-2- oxo-ethyl]-1-fluoro- cyclopropanecarboxamide | 622.295 | 2.35 |
| 380 | 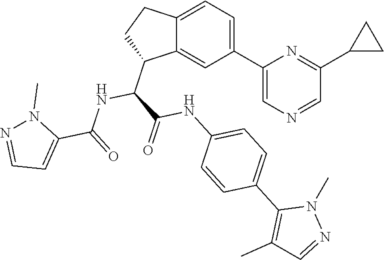 [SMILES: Cc1cnn(C)c1-c1ccc(NC(=O)[C@@H](NC(=O)c2ccnn2C)[C@@H]2CCc3ccc(-c4cncc(C5CC5)n4)cc32)cc1] | N-[(1S)-1-[(1R)-6-(6- cyclopropylpyrazin-2-yl)indan- 1-yl]-2-[4-(2,4- dimethylpyrazol-3-yl)anilino]- 2-oxo-ethyl]-2-methyl- pyrazole-3-carboxamide | 587.29 | 2.42 |
| 381 | 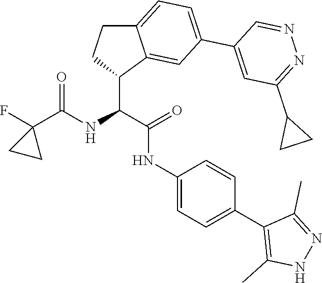 [SMILES: Cc1n[nH]c(C)c1-c1ccc(NC(=O)[C@@H](NC(=O)C2(F)CC2)[C@@H]2CCc3ccc(-c4cnnc(C5CC5)c4)cc32)cc1] | N-[(1S)-1-[(1R)-6-(6- cyclopropylpyridazin-4- yl)indan-1-yl]-2-[4-(3,5- dimethyl-1H-pyrazol-4- yl)anilino]-2-oxo-ethyl]-1- fluoro- cyclopropanecarboxamide | 565.272 | 2.18 |
| 382 | 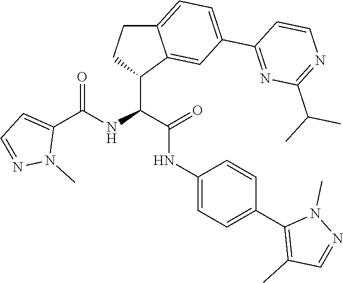 [SMILES: Cc1cnn(C)c1-c1ccc(NC(=O)[C@@H](NC(=O)c2ccnn2C)[C@@H]2CCc3ccc(-c4ccnc(C(C)C)n4)cc32)cc1] | N-[(1S)-2-[4-(2,4- dimethylpyrazol-3-yl)anilino]- 1-[(1R)-6-(2- isopropylpyrimidin-4-yl)indan- 1-yl]-2-oxo-ethyl]-2-methyl- pyrazole-3-carboxamide | 589.306 | 2.41 |
| 383 | 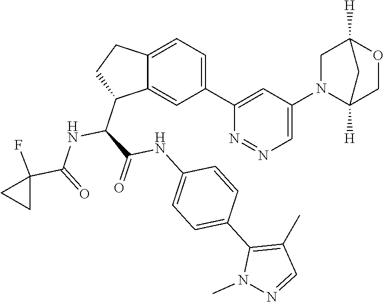 [SMILES: Cc1cnn(C)c1-c1ccc(NC(=O)[C@@H](NC(=O)C2(F)CC2)[C@@H]2CCc3ccc(-c4cc(N5C[C@@H]6C[C@H]5CO6)cnn4)cc32)cc1] | N-[(1S)-2-[4-(2,4- dimethylpyrazol-3-yl)anilino]- 1-[(1R)-6-[5-[(1S,4S)-2-oxa- 5-azabicyclo[2.2.1]heptan-5- yl]pyridazin-3-yl]indan-1-yl]-2- oxo-ethyl]-1-fluoro- cyclopropanecarboxamide | 622.295 | 2.00 |
| 384 | 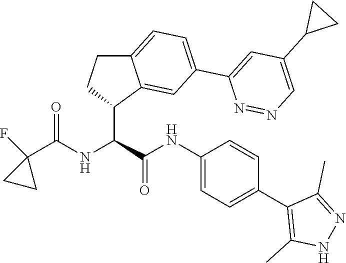 [SMILES: Cc1n[nH]c(C)c1-c1ccc(NC(=O)[C@@H](NC(=O)C2(F)CC2)[C@@H]2CCc3ccc(-c4cc(C5CC5)cnn4)cc32)cc1] | N-[(1S)-1-[(1R)-6-(5- cyclopropylpyridazin-3- yl)indan-1-yl]-2-[4-(3,5- dimethyl-1H-pyrazol-4- yl)anilino]-2-oxo-ethyl]-1- fluoro- cyclopropanecarboxamide | 565.274 | 2.19 |
| 385 | 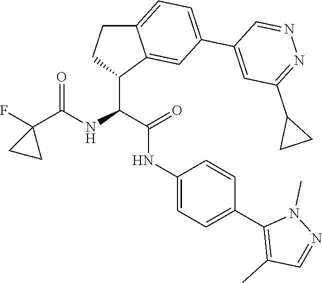 [SMILES: Cc1cnn(C)c1-c1ccc(NC(=O)[C@@H](NC(=O)C2(F)CC2)[C@@H]2CCc3ccc(-c4cnnc(C5CC5)c4)cc32)cc1] | N-[(1S)-1-[(1R)-6-(6- cyclopropylpyridazin-4- yl)indan-1-yl]-2-[4-(2,4- dimethylpyrazol-3-yl)anilino]- 2-oxo-ethyl]-1-fluoro- cyclopropanecarboxamide | 565.273 | 2.33 |
| 386 | 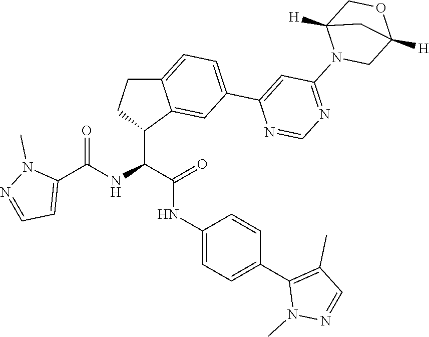 [SMILES: Cc1cnn(C)c1-c1ccc(NC(=O)[C@@H](NC(=O)c2ccnn2C)[C@@H]2CCc3ccc(-c4cc(N5C[C@@H]6C[C@H]5CO6)ncn4)cc32)cc1] | N-[(1S)-2-[4-(2,4- dimethylpyrazol-3-yl)anilino]- 1-[(1R)-6-[6-[(1S,4S)-2-oxa- 5-azabicyclo[2.2.1]heptan-5- yl]pyrimidin-4-yl]indan-1-yl]-2- oxo-ethyl]-2-methyl-pyrazole- 3-carboxamide | 644.31 | 1.97 |
| 387 | 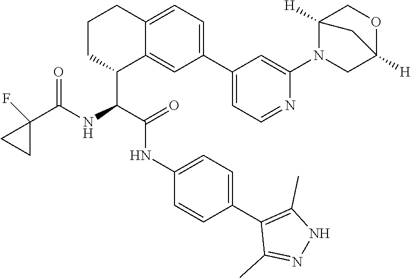 [SMILES: Cc1n[nH]c(C)c1-c1ccc(NC(=O)[C@@H](NC(=O)C2(F)CC2)[C@@H]2CCCc3ccc(-c4ccnc(N5C[C@H]6C[C@@H]5CO6)c4)cc32)cc1] | N-[(1S)-2-[4-(3,5-dimethyl- 1H-pyrazol-4-yl)anilino]-1- [(1R)-7-[2-[(1R,4R)-2-oxa-5- azabicyclo[2.2.1]heptan-5-yl]- 4-pyridyl]tetralin-1-yl]-2-oxo- ethyl]-1-fluoro- cyclopropanecarboxamide635.315 | 635.315 | 1.99 |
| 388 | 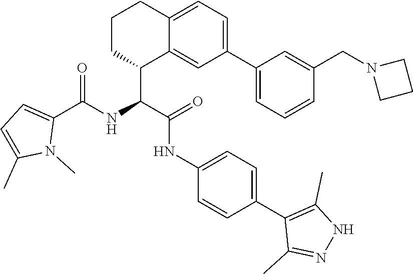 [SMILES: Cc1n[nH]c(C)c1-c1ccc(NC(=O)[C@@H](NC(=O)c2ccc(C)n2C)[C@@H]2CCCc3ccc(-c4cccc(CN5CCC5)c4)cc32)cc1] | N-[(1S)-1-[(1R)-7-[3-(azetidin- 1-ylmethyl)phenyl]tetralin-1- yl]-2-[4-(3,5-dimethyl-1H- pyrazol-4-yl)anilino]-2-oxo- ethyl]-2-methyl-pyrazole-3- carboxamide | 628.342 | 1.97 |
| 389 | 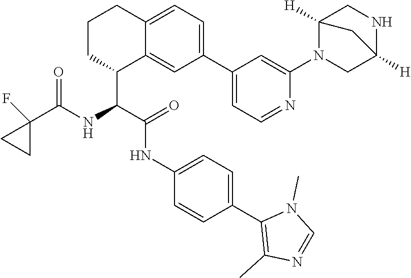 [SMILES: Cc1ncn(C)c1-c1ccc(NC(=O)[C@@H](NC(=O)C2(F)CC2)[C@@H]2CCCc3ccc(-c4ccnc(N5C[C@H]6C[C@@H]5CN6)c4)cc32)cc1] | N-[(1S)-1-[(1R)-7-[2-[(1R,4R)- 2,5-diazabicyclo[2.2.1]heptan- 2-yl]-4-pyridyl]tetralin-1-yl]-2- [4-(3,5-dimethylimidazol-4- yl)anilino]-2-oxo-ethyl]-1- fluoro- cyclopropanecarboxamide | 634.33 | 1.74 |
| 390 | 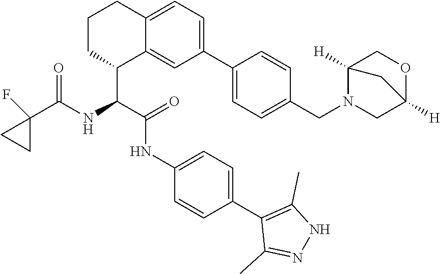 [SMILES: Cc1n[nH]c(C)c1-c1ccc(NC(=O)[C@@H](NC(=O)C2(F)CC2)[C@@H]2CCCc3ccc(-c4ccc(CN5C[C@H]6C[C@@H]5CO6)cc4)cc32)cc1] | N-[(1S)-2-[4-(3,5-dimethyl- 1H-pyrazol-4-yl)anilino]-1- [(1R)-7-[4-[[(1R,4R)-2-oxa-5- azabicyclo[2.2.1]heptan-5- yl]methyl]phenyl]tetralin-1-yl]- 2-oxo-ethyl]-1-fluoro- cyclopropanecarboxamide | 648.333 | 2.03 |
| 391 | 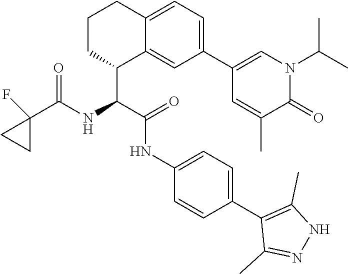 [SMILES: Cc1n[nH]c(C)c1-c1ccc(NC(=O)[C@@H](NC(=O)C2(F)CC2)[C@@H]2CCCc3ccc(-c4cc(C)c(=O)n(C(C)C)c4)cc32)cc1] | N-[(1S)-2-[4-(3,5-dimethyl- 1H-pyrazol-4-yl)anilino]-1- [(1R)-7-(1-isopropyl-5-methyl- 6-oxo-3-pyridyl)tetralin-1-yl]- 2-oxo-ethyl]-1-fluoro- cyclopropanecarboxamide | 610.319 | 2.31 |
| 392 | 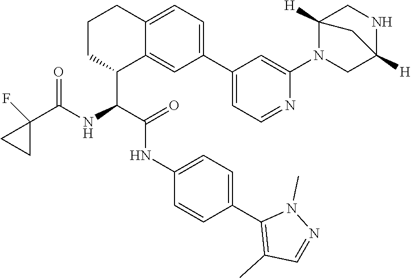 [SMILES: Cc1cnn(C)c1-c1ccc(NC(=O)[C@@H](NC(=O)C2(F)CC2)[C@@H]2CCCc3ccc(-c4ccnc(N5C[C@@H]6C[C@H]5CN6)c4)cc32)cc1] | N-[(1S)-1-[(1R)-7-[2-[(1S,4S)- 2,5-diazabicyclo[2.2.1]heptan- 2-yl]-4-pyridyl]tetralin-1-yl]-2- [4-(2,4-dimethylpyrazol-3- yl)anilino]-2-oxo-ethyl]-1- fluoro- cyclopropanecarboxamide | 634.331 | 1.94 |
| 393 | 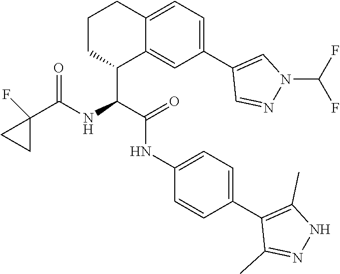 [SMILES: Cc1n[nH]c(C)c1-c1ccc(NC(=O)[C@@H](NC(=O)C2(F)CC2)[C@@H]2CCCc3ccc(-c4cnn(C(F)F)c4)cc32)cc1] | N-[(1S)-1-[(1R)-7-[1- (difluoromethyl)pyrazol-4- yl]tetralin-1-yl]-2-[4-(3,5- dimethyl-1H-pyrazol-4- yl)anilino]-2-oxo-ethyl]-1- fluoro- cyclopropanecarboxamide | 577.214 | 2.34 |
| 394 | 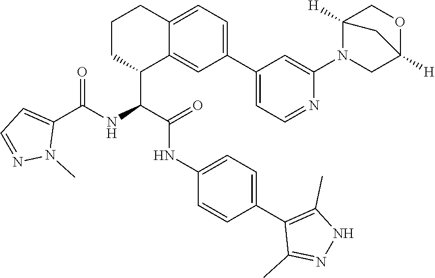 [SMILES: Cc1n[nH]c(C)c1-c1ccc(NC(=O)[C@@H](NC(=O)c2ccnn2C)[C@@H]2CCCc3ccc(-c4ccnc(N5C[C@H]6C[C@@H]5CO6)c4)cc32)cc1] | N-[(1S)-2-[4-(3,5-dimethyl- 1H-pyrazol-4-yl)anilino]-1- [(1R)-7-[2-[(1R,4R)-2-oxa-5- azabicyclo[2.2.1]heptan-5-yl]- 4-pyridyl]tetralin-1-yl]-2-oxo- ethyl]-2-methyl-pyrazole-3- carboxamide | 657.33 | 1.93 |
| 395 | 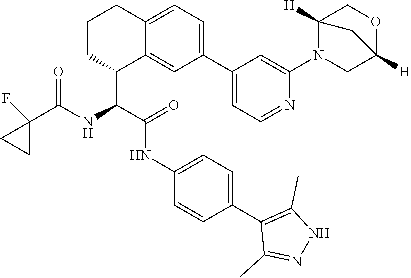 [SMILES: Cc1n[nH]c(C)c1-c1ccc(NC(=O)[C@@H](NC(=O)C2(F)CC2)[C@@H]2CCCc3ccc(-c4ccnc(N5C[C@@H]6C[C@H]5CO6)c4)cc32)cc1] | N-[(1S)-2-[4-(3,5-dimethyl- 1H-pyrazol-4-yl)anilino]-1- [(1R)-7-[2-[(1S,4S)-2-oxa-5- azabicyclo[2.2.1]heptan-5-yl]- 4-pyridyl]tetralin-1-yl]-2-oxo- ethyl]-1-fluoro- cyclopropanecarboxamide | 633.315 | 1.99 |
| 396 | 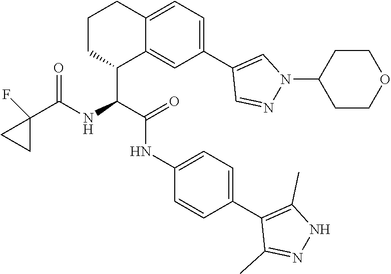 [SMILES: Cc1n[nH]c(C)c1-c1ccc(NC(=O)[C@@H](NC(=O)C2(F)CC2)[C@@H]2CCCc3ccc(-c4cnn(C5CCOCC5)c4)cc32)cc1] | N-[(1S)-2-[4-(3,5-dimethyl- 1H-pyrazol-4-yl)anilino]-2-oxo- 1-[(1R)-7-(1-tetrahydropyran- 4-ylpyrazol-4-yl)tetralin-1- yl]ethyl]-1-fluoro- cyclopropanecarboxamide | 611.315 | 2.24 |
| 397 | 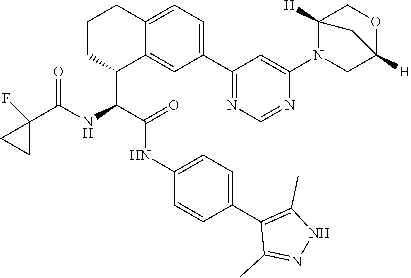 [SMILES: Cc1n[nH]c(C)c1-c1ccc(NC(=O)[C@@H](NC(=O)C2(F)CC2)[C@@H]2CCCc3ccc(-c4cc(N5C[C@@H]6C[C@H]5CO6)ncn4)cc32)cc1] | N-[(1S)-2-[4-(3,5-dimethyl- 1H-pyrazol-4-yl)anilino]-1- [(1R)-7-[6-[(1S,4S)-2-oxa-5- azabicyclo[2.2.1]heptan-5- yl]pyrimidin-4-yl]tetralin-1-yl]- 2-oxo-ethyl]-1-fluoro- cyclopropanecarboxamide | 636.311 | 1.95 |
| 398 | 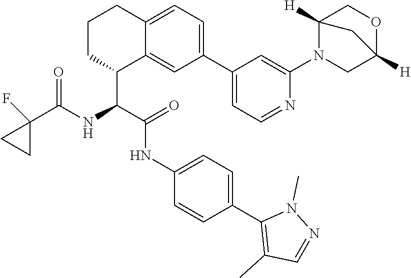 [SMILES: Cc1cnn(C)c1-c1ccc(NC(=O)[C@@H](NC(=O)C2(F)CC2)[C@@H]2CCCc3ccc(-c4ccnc(N5C[C@@H]6C[C@H]5CO6)c4)cc32)cc1] | N-[(1S)-2-[4-(2,4- dimethylpyrazol-3-yl)anilino]- 1-[(1R)-7-[2-[(1S,4S)-2-oxa- 5-azabicyclo[2.2.1]heptan-5- yl]-4-pyridyl]tetralin-1-yl]-2- oxo-ethyl]-1-fluoro- cyclopropanecarboxamide | 635.312 | 2.07 |
| 399 | 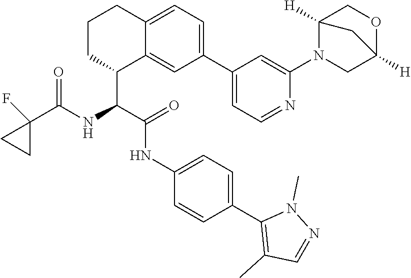 [SMILES: Cc1cnn(C)c1-c1ccc(NC(=O)[C@@H](NC(=O)C2(F)CC2)[C@@H]2CCCc3ccc(-c4ccnc(N5C[C@H]6C[C@@H]5CO6)c4)cc32)cc1] | N-[(1S)-2-[4-(2,4- dimethylpyrazol-3-yl)anilino]- 1-[(1R)-7-[2-[(1R,4R)-2-oxa- 5-azabicyclo[2.2.1]heptan-5- yl]-4-pyridyl]tetralin-1-yl]-2- oxo-ethyl]-1-fluoro- cyclopropanecarboxamide | 635.315 | 2.08 |
| 400 | 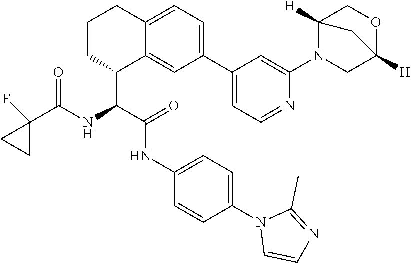 [SMILES: Cc1nccn1-c1ccc(NC(=O)[C@@H](NC(=O)C2(F)CC2)[C@@H]2CCCc3ccc(-c4ccnc(N5C[C@@H]6C[C@H]5CO6)c4)cc32)cc1] | 1-fluoro-N-[(1S)-2-[4-(2- methylimidazol-1-yl)anilino]-1- [(1R)-7-[2-[(1S,4S)-2-oxa-5- azabicyclo[2.2.1]heptan-5-yl]- 4-pyridyl]tetralin-1-yl]-2-oxo- ethyl]cyclopropanecarboxamide | 621.300 | 1.83 |
| 401 | 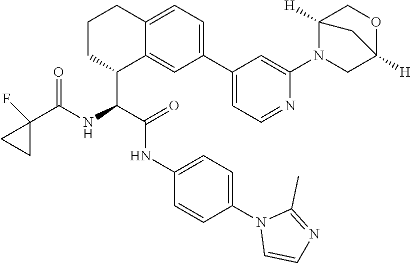 [SMILES: Cc1nccn1-c1ccc(NC(=O)[C@@H](NC(=O)C2(F)CC2)[C@@H]2CCCc3ccc(-c4ccnc(N5C[C@H]6C[C@@H]5CO6)c4)cc32)cc1] | 1-fluoro-N-[(1S)-2-[4-(2- methylimidazol-1-yl)anilino]-1- [(1R)-7-[2-[(1R,4R)-2-oxa-5- azabicyclo[2.2.1]heptan-5-yl]- 4-pyridyl]tetralin-1-yl]-2-oxo- ethyl]cyclopropanecarboxamide | 621.30 | 1.83 |
| 402 | 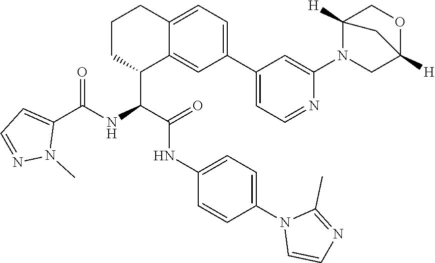 [SMILES: Cc1nccn1-c1ccc(NC(=O)[C@@H](NC(=O)c2ccnn2C)[C@@H]2CCCc3ccc(-c4ccnc(N5C[C@@H]6C[C@H]5CO6)c4)cc32)cc1] | 2-methyl-N-[(1S)-2-[4-(2- methylimidazol-1-yl)anilino]-1- [(1R)-7-[2-[(1S,4S)-2-oxa-5- azabicyclo[2.2.1]heptan-5-yl]- 4-pyridyl]tetralin-1-yl]-2-oxo- ethyl]pyrazole-3-carboxamide | 643.315 | 1.78 |
| 403 | 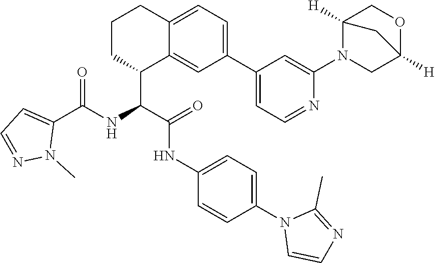 [SMILES: Cc1nccn1-c1ccc(NC(=O)[C@@H](NC(=O)c2ccnn2C)[C@@H]2CCCc3ccc(-c4ccnc(N5C[C@H]6C[C@@H]5CO6)c4)cc32)cc1] | 2-methyl-N-[(1S)-2-[4-(2- methylimidazol-1-yl)anilino]-1- [(1R)-7-[2-[(1R,4R)-2-oxa-5- azabicyclo[2.2.1]heptan-5-yl]- 4-pyridyl]tetralin-1-yl]-2-oxo- ethyl]pyrazole-3-carboxamide | 643.34 | 1.77 |
| 404 | 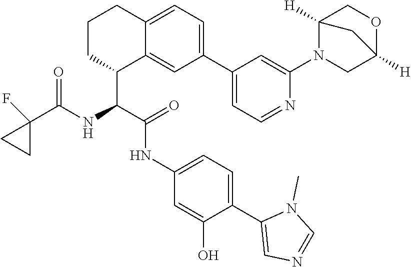 [SMILES: Cn1cncc1-c1ccc(NC(=O)[C@@H](NC(=O)C2(F)CC2)[C@@H]2CCCc3ccc(-c4ccnc(N5C[C@H]6C[C@@H]5CO6)c4)cc32)cc1O] | 1-fluoro-N-[(1S)-2-[3-hydroxy- 4-(3-methylimidazol-4- yl)anilino]-1-[(1R)-7-[2- [(1R,4R)-2-oxa-5- azabicyclo[2.2.1]heptan-5-yl]- 4-pyridyl]tetralin-1-yl]-2-oxo- ethyl]cyclopropanecarboxamide | 637.294 | 1.75 |
| 405 | 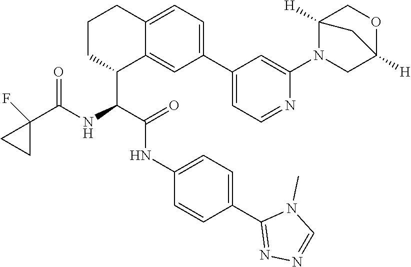 [SMILES: Cn1cnnc1-c1ccc(NC(=O)[C@@H](NC(=O)C2(F)CC2)[C@@H]2CCCc3ccc(-c4ccnc(N5C[C@H]6C[C@@H]5CO6)c4)cc32)cc1] | 1-fluoro-N-[(1S)-2-[4-(4- methyl-1,2,4-triazol-3- yl)anilino]-1-[(1R)-7-[2- [(1R,4R)-2-oxa-5- azabicyclo[2.2.1]heptan-5-yl]- 4-pyridyl]tetralin-1-yl]-2-oxo- ethyl]cyclopropanecarboxamide | 622.294 | 1.90 |
| 406 | 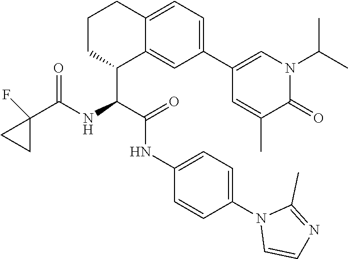 [SMILES: Cc1cc(-c2ccc3c(c2)[C@H]([C@H](NC(=O)C2(F)CC2)C(=O)Nc2ccc(-n4ccnc4C)cc2)CCC3)cn(C(C)C)c1=O] | 1-fluoro-N-[(1S)-1-[(1R)-7-(1- isopropyl-5-methyl-6-oxo-3- pyridyl)tetralin-1-yl]-2-[4-(2- methylimidazol-1-yl)anilino]-2- oxo- ethyl]cyclopropanecarboxamide | 596.304 | 2.07 |
| 407^{#} | 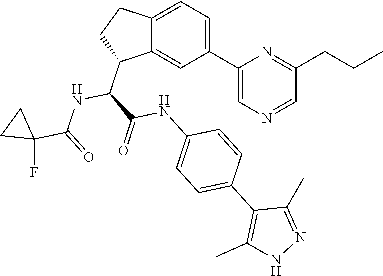 [SMILES: CCCc1cncc(-c2ccc3c(c2)[C@H]([C@H](NC(=O)C2(F)CC2)C(=O)Nc2ccc(-c4c(C)n[nH]c4C)cc2)CC3)n1] | N-[(1S)-2-[4-(3,5-dimethyl- 1H-pyrazol-4-yl)anilino]-1- [(1R)-6-(6-isopropylpyrazin-2- yl)indan-1-yl]-2-oxo-ethyl]-1- fluoro- cyclopropanecarboxamide; N- [(1S)-2-[4-(3,5-dimethyl-1H- pyrazol-4-yl)anilino]-2-oxo-1- [(1R)-6-(6-propylpyrazin-2- yl)indan-1-yl]ethyl]-1-fluoro- cyclopropanecarboxamide | 567.289 | 2.42 |
| | 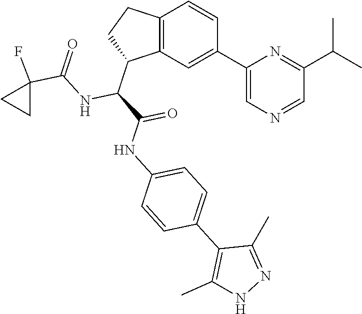 [SMILES: Cc1n[nH]c(C)c1-c1ccc(NC(=O)[C@@H](NC(=O)C2(F)CC2)[C@@H]2CCc3ccc(-c4cncc(C(C)C)n4)cc32)cc1] | | | |
| 408^{#} | 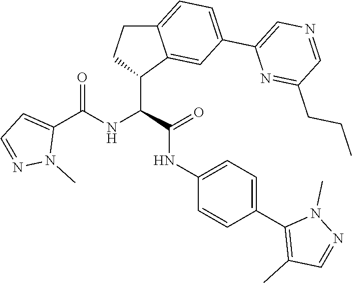 [SMILES: CCCc1cncc(-c2ccc3c(c2)[C@H]([C@H](NC(=O)c2ccnn2C)C(=O)Nc2ccc(-c4c(C)cnn4C)cc2)CC3)n1] | N-[(1S)-2-[4-(2,4- dimethylpyrazol-3-yl)anilino]- 1-[(1R)-6-(6-isopropylpyrazin- 2-yl)indan-1-yl]-2-oxo-ethyl]- 2-methyl-pyrazole-3- carboxamide; N-[(1S)-2-[4- (2,4-dimethylpyrazol-3- yl)anilino]-2-oxo-1-[(1R)-6-(6- propylpyrazin-2-yl)indan-1- yl]ethyl]-2-methyl-pyrazole-3- carboxamide | 589.302 | 2.45 |
| | 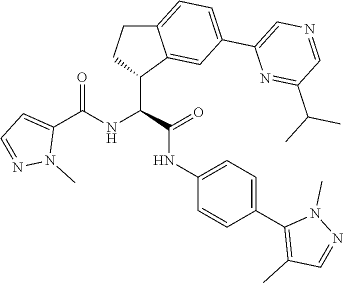 [SMILES: Cc1cnn(C)c1-c1ccc(NC(=O)[C@@H](NC(=O)c2ccnn2C)[C@@H]2CCc3ccc(-c4cncc(C(C)C)n4)cc32)cc1] | | | |
| 409^{#} | 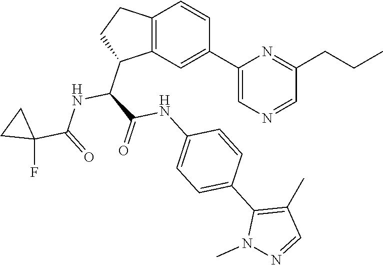 [SMILES: CCCc1cncc(-c2ccc3c(c2)[C@H]([C@H](NC(=O)C2(F)CC2)C(=O)Nc2ccc(-c4c(C)cnn4C)cc2)CC3)n1] | N-[(1S)-2-[4-(2,4- dimethylpyrazol-3-yl)anilino]- 1-[(1R)-6-(6-isopropylpyrazin- 2-yl)indan-1-yl]-2-oxo-ethyl]- 1-fluoro- cyclopropanecarboxamide; N- [(1S)-2-[4-(2,4- dimethylpyrazol-3-yl)anilino]- 2-oxo-1-[(1R)-6-(6- propylpyrazin-2-yl)indan-1- yl]ethyl]-1-fluoro- cyclopropanecarboxamide | 567.29 | 2.58 |
| | 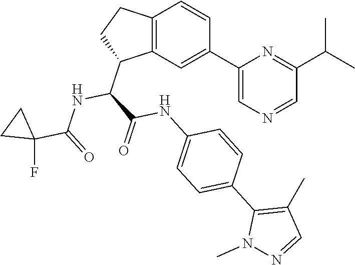 [SMILES: Cc1cnn(C)c1-c1ccc(NC(=O)[C@@H](NC(=O)C2(F)CC2)[C@@H]2CCc3ccc(-c4cncc(C(C)C)n4)cc32)cc1] | | | |

*RT are XEV Metode 1 CM - ES+ unless stated
(z) LCMS Method 3
^{#}inseparable mixture of 2 regioisomers

Example: IL-8 release assay in human epithelial keratinocytes adult (HEKa) Keratinocytes were seeded at 3500 cells/well in 384-well ViewPlates (Perkin Elmer) in Epilife medium (Thermo Fisher) containing human keratinocyte growth supplement (HKGS) without hydrocortisone and incubated in a humid incubator at 37° C., 5% CO2, overnight. The following day growth medium was removed and 25 μl fresh Epilife 10 medium added. 75 nl test compound in 100% DMSO was added into each well reserved

for test compounds, by the use of acoustic pipetting. The remaining wells received an equal volume of DMSO only, as vehicle control, or terfenadine in DMSO, as a positive control for any cytotoxic compounds. Subsequently, another 25 μl Epilife medium was added to each well. Finally, wells containing test compounds and wells prepared to yield 15 maximum stimulation received 25 μl of 9 ng/ml recombinant, human embryonic kidney cell (HEK)-derived human IL-17AA+30 ng/ml human TNF-alpha, in Epilife medium. Wells prepared to define 100% inhibition of IL-17 effects received 25 μl of 30 ng/ml human TNF-alpha alone, in Epilife medium. Final concentrations were 3 ng/ml HEKhuman IL-17AA+10 ng/ml human TNFalpha (maximum stimulation) and 10 ng/ml
20 human TNFalpha alone (100% inhibition, Emax), respectively. Cells were incubated for
68-72 h in the incubator. IL-8 released from the cells was measured by the use of a commercial homogenous time-resolved fluorescence (HTRF) assay (CisBio). 2 μl cell culture supernatant was transferred to a 384-well Proxiplate. 5 μl HTRF reagent was added and the plates incubated sealed in the dark for 3-22 h at room temperature. Time resolved fluorescence was read at 665 vs 620 nm, with excitation at 320 nm, and IL-8 levels calculated as percent of controls. Reduction of the amount of secreted IL-8 indicates decreased IL-17 signaling. Concentration response curves were fitted by the use of a four-parameter logistic equation. Relative IC50 and Emax were reported from 5 curves showing acceptable fit (r2>0.9). Cytotoxicity was measured in the cell-containing

Viewplates following addition of 7 μl PrestoBlue (Thermo Fisher) and incubation for 2.5-3 h at room temperature, by measuring fluorescence at 615 nm (excitation at 535 nm). Fluorescence was directly proportional to the amount of metabolic activity. Reduction of fluorescence signal indicated cytotoxicity.

Compounds of the present invention were tested in the IL-8 release assay in human epithelial keratinocytes. The results are summarized in Table 1.

Compounds having a Relative EC50 of X; wherein X<100 nM; are indicated with *

Compounds having a Relative EC50 of X; wherein 100 nM<X<1000 nM; are indicated with **

Compounds having a Relative EC50 of X; wherein 1000 nM<X; are indicated with ***

TABLE 1

| Example | Rel EC₅₀ L-8 |
|---|---|
| no | release assay |
| 1 | * |
| 2 | * |
| 3 | ** |
| 4 | * |
| 5 | * |
| 6 | ** |
| 7 | * |
| 8 | * |
| 9 | * |
| 10 | * |
| 11 | * |
| 12 | * |
| 13 | * |
| 14 | * |
| 15 | * |
| 16 | * |
| 17 | * |
| 18 | * |
| 19 | ** |
| 20 | ** |
| 21 | * |
| 22 | ** |
| 23 | * |
| 24 | ** |
| 25 | ** |
| 26 | * |
| 27 | * |
| 28 | * |
| 29 | * |
| 30 | * |
| 31 | * |
| 32 | ** |
| 33 | * |
| 34 | * |
| 35 | * |
| 36 | * |
| 37 | * |
| 38 | * |
| 39 | * |
| 40 | * |
| 41 | * |
| 42 | ** |
| 43 | ** |
| 44 | ** |
| 45 | ** |
| 46 | * |
| 47 | * |
| 48 | *** |
| 49 | * |
| 50 | ** |
| 51 | ** |
| 52 | *** |
| 53 | ** |
| 54 | ** |
| 55 | *** |
| 56 | *** |
| 57 | Not tested |
| 58 | * |
| 59 | * |
| 60 | * |
| 61 | ** |
| 62 | *** |
| 63 | *** |
| 64 | *** |
| 65 | *** |
| 66 | *** |
| 67 | ** |
| 68 | *** |
| 69 | ** |
| 70 | *** |
| 71 | * |
| 72 | *** |
| 73 | *** |
| 74 | ** |
| 75 | *** |
| 76 | *** |
| 77 | *** |
| 78 | ** |
| 79 | ** |
| 80 | ** |
| 81 | ** |
| 82 | ** |
| 83 | ** |
| 84 | ** |
| 85 | *** |
| 86 | *** |
| 87 | ** |
| 88 | *** |
| 89 | ** |
| 90 | ** |
| 91 | ** |
| 92 | *** |
| 93 | ** |
| 94 | * |
| 95 | *** |
| 96 | ** |
| 97 | *** |
| 98 | ** |
| 99 | *** |
| 100 | ** |
| 101 | *** |
| 102 | ** |
| 103 | * |
| 104 | ** |
| 105 | ** |
| 106 | *** |
| 107 | ** |
| 108 | ** |
| 109 | ** |
| 110 | ** |
| 111 | ** |
| 112 | ** |
| 113 | *** |
| 114 | ** |
| 115 | *** |
| 116 | *** |
| 117 | *** |
| 118 | * |
| 119 | ** |
| 120 | ** |
| 121 | ** |
| 122 | ** |
| 123 | * |
| 124 | ** |
| 125 | ** |
| 126 | ** |
| 127 | *** |
| 128 | ** |
| 129 | ** |
| 130 | ** |
| 131 | ** |
| 132 | * |
| 133 | ** |
| 134 | ** |
| 135 | ** |
| 136 | ** |
| 137 | * |
| 138 | ** |
| 139 | *** |
| 140 | ** |
| 141 | * |
| 142 | * |
| 143 | ** |
| 144 | ** |
| 145 | ** |
| 146 | ** |
| 147 | ** |
| 148 | *** |
| 149 | ** |
| 150 | * |
| 151 | Not tested |
| 152 | ** |
| 153 | * |
| 154 | *** |
| 155 | *** |
| 156 | *** |
| 157 | ** |
| 158 | ** |
| 159 | * |
| 160 | ** |
| 161 | ** |
| 162 | ** |
| 163 | *** |
| 164 | ** |
| 165 | ** |
| 166 | ** |
| 167 | *** |
| 168 | ** |
| 169 | ** |
| 170 | ** |
| 171 | ** |
| 172 | *** |
| 173 | *** |
| 174 | * |
| 175 | * |
| 176 | * |
| 177 | * |
| 178 | ** |
| 179 | * |
| 180 | ** |
| 181 | * |
| 182 | * |
| 183 | ** |
| 184 | ** |
| 185 | ** |
| 186 | ** |
| 187 | ** |
| 188 | ** |
| 189 | ** |
| 190 | ** |
| 191 | ** |
| 192 | ** |
| 193 | ** |
| 194 | ** |
| 195 | ** |
| 196 | ** |
| 197 | ** |
| 198 | *** |
| 199 | ** |
| 200 | * |
| 201 | ** |
| 202 | ** |
| 203 | ** |
| 204 | ** |
| 205 | ** |
| 206 | ** |
| 207 | * |
| 208 | ** |
| 209 | ** |
| 210 | ** |
| 211 | ** |
| 212 | ** |
| 213 | ** |
| 214 | *** |
| 215 | ** |
| 216 | ** |
| 217 | ** |
| 218 | ** |
| 219 | ** |
| 220 | ** |
| 221 | ** |
| 222 | * |
| 223 | * |
| 224 | * |
| 225 | * |
| 226 | ** |
| 227 | ** |
| 228 | ** |
| 229 | ** |
| 230 | ** |
| 231 | ** |
| 232 | *** |
| 233 | *** |
| 234 | ** |
| 235 | * |
| 236 | * |
| 237 | *** |
| 238 | ** |
| 239 | ** |
| 240 | *** |
| 241 | *** |
| 242 | *** |
| 243 | ** |
| 244 | *** |
| 245 | * |
| 282 | *** |
| 283 | *** |
| 286 | *** |
| 287 | *** |
| 288 | *** |
| 291 | *** |
| 293 | *** |
| 296 | *** |
| 301 | *** |
| 306 | *** |
| 307 | *** |
| 308 | *** |
| 309 | *** |
| 310 | ** |
| 311 | *** |
| 312 | ** |
| 313 | * |
| 314 | * |
| 315 | * |
| 316 | * |
| 317 | * |
| 318 | * |
| 319 | * |
| 321 | *** |
| 323 | ** |
| 324 | ** |
| 325 | *** |
| 327 | ** |
| 328 | *** |
| 329 | ** |
| 330 | ** |
| 331 | * |
| 332 | *** |
| 333 | *** |
| 334 | ** |
| 335 | ** |
| 336 | ** |
| 337 | ** |
| 338 | *** |
| 339 | ** |
| 340 | ** |
| 341 | * |
| 342 | *** |
| 343 | ** |
| 346 | * |
| 347 | * |
| 348 | * |
| 349 | * |
| 350 | * |
| 351 | ** |
| 352 | ** |
| 353 | ** |
| 354 | * |
| 355 | ** |
| 356 | ** |
| 357 | * |
| 358 | * |
| 359 | ** |
| 360 | ** |
| 361 | ** |
| 362 | * |
| 363 | * |
| 364 | * |
| 365 | ** |
| 366 | ** |
| 367 | * |
| 368 | ** |
| 369 | * |
| 370 | * |
| 371 | * |
| 372 | ** |
| 373 | * |
| 374 | ** |
| 375 | ** |
| 376 | ** |
| 377 | * |
| 378 | * |
| 379 | * |
| 380 | ** |
| 381 | ** |
| 382 | ** |
| 383 | *** |
| 384 | ** |
| 385 | ** |
| 386 | ** |
| 387 | * |
| 388 | * |
| 389 | ** |
| 390 | ** |
| 391 | * |
| 392 | ** |
| 393 | ** |
| 394 | * |
| 395 | * |
| 396 | * |
| 397 | * |
| 398 | * |
| 399 | * |
| 400 | ** |
| 401 | ** |
| 402 | * |
| 403 | ** |
| 404 | *** |
| 405 | *** |
| 406 | ** |
| 407 | ** |
| 408 | ** |
| 409 | ** |

The following are embodiments of the invention:

Embodiment 1. A compound according to general formula I,

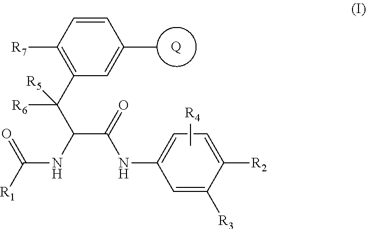

wherein

R₁ is selected from the group consisting of 5- or 6-membered heteroaryl, 9- or 10-membered bicyclic heteroaryl, phenyl, 4-6-membered heterocycloalkyl, (C₁-C₆)alkoxy, (C₃-C₇)cycloalkoxy, (C₃-C₇)cycloalkyl(C₁-C₆)alkyl, (C₃-C₇)cycloalkyl(C₁-C₆)alkoxy, (C₁-C₆)alkyl, phenyl-(C₁-C₄)alkyl, (C₃-C₇)cycloalkyl and —NR_{c}R_{d}, wherein said 5- or 6-membered heteroaryl, 9- or 10-membered bicyclic heteroaryl, phenyl, 4-6-membered heterocycloalkyl, (C₁-C₆)alkoxy, (C₃-C₇)cycloalkoxy, (C₃-C₇)cycloalkyl(C₁-C₆)alkyl, (C₃-C₇)cycloalkyl(C₁-C₆)alkoxy, (C₁-C₆)alkyl, phenyl-(C₁-C₄)alkyl and (C₃-C₇)cycloalkyl is optionally substituted with one or more substituents independently selected from Rₐ;

Rₐ represents deuterium, halogen, hydroxy —NR_{c}R_{d}, (C₁-C₆)alkyl, (C₁-C₆)alkylcarbonyl, (C₃-C₇)cycloalkyl, phenyl, 4-6-membered heterocycloalkyl or 5- or 6-membered heteroaryl, wherein said (C₁-C₆)alkyl, (C₁-C₆)alkylcarbonyl, (C₃-C₇)cycloalkyl, phenyl, 4-6-membered heterocycloalkyl or 5- or 6-membered heteroaryl is optionally substituted with one or more substituents independently selected from deuterium, halogen, hydroxy, (C₁-C₄)alkyl, (C₃-C₇)cycloalkyl, (C₁-C₄)alkoxy, —SO₂—(C₁-C₄)alkyl and —NR_{c}R_{d};

R₂ is 5-membered heteroaryl, wherein said 5-membered heteroaryl is optionally substituted with one or more substituents independently selected from R_{b};

R_{b} represents deuterium, halogen, cyano, hydroxy, —NR_{c}R_{d}, (C₁-C₆)alkyl, (C₁-C₆)alkoxy or (C₃-C₇)cycloalkyl, wherein said (C₁-C₆)alkyl, (C₁-C₆)alkoxy or (C₃-C₇)cycloalkyl is optionally substituted with one or more substituents independently selected from deuterium, halogen, hydroxy, —NR_{c}R_{d} and (C₁-C₄)alkoxy;

R_{c} and R_{d} each independently are selected from the group consisting of hydrogen and (C₁-C₄)alkyl;

R₃ is selected from the group consisting of hydrogen, deuterium, hydroxy, (C₁-C₆)alkoxy and halogen;

or R₂ and R₃ together with the phenyl to which they are attached form a 9- or 10-membered bicyclic heteroaromatic ring system, wherein said 9- or 10-membered bicyclic heteroaromatic ring system is optionally substituted with one or more substituents independently selected from Rₑ;

Rₑ represents deuterium, halogen, (C₁-C₄)alkyl, hydroxy(C₁-C₄)alkyl, (C₃-C₇)cycloalkyl or —NR_{c}R_{d};

R₄ is selected from the group consisting of hydrogen, deuterium and halogen;

R₅ and R₆ each independently are selected from the group consisting of hydrogen, deuterium, (C₁-C₄)alkyl, (C₃-C₇)cycloalkyl and phenyl, with the proviso that at least one of R₅ and R₆ is selected from hydrogen or deuterium;

R₇ is selected from the group consisting of hydrogen and halogen, with the proviso that R₇ is not hydrogen when both R₅ and R₆ are selected from hydrogen or deuterium;

or R₆ and R₇ together with the phenyl to which R₇ is attached form a fused bicyclic ring system selected from the group consisting of tetralin and indane, wherein said tetralin or indane is optionally substituted with one more substituents independently selected from the group consisting of deuterium, halogen and (C₁-C₄)alkyl;

Q represents phenyl, 4-8 membered heterocycloalkyl, 5- or 6-membered heteroaryl, 9- or 10-membered bicyclic heteroaryl, pyridonyl, indolinyl, isoindolinyl, tetrahydroquinolinyl or tetrahydroisoquinolinyl, wherein said pyridonyl, phenyl, 4-8 membered heterocycloalkyl, 5- or 6-membered heteroaryl, 9- or 10-membered bicyclic heteroaryl, indolinyl, isoindolinyl, tetrahydroquinolinyl or tetrahydroisoquinolinyl is optionally substituted with one or more substituents independently selected from R₈;

R₈ represents deuterium, oxo, hydroxy, —S(O)₂R_{f}, —S(O)R_{f}, —NR_{g}Rₕ, —C(O)NR_{g}Rₕ, —C(O)R_{f}, cyano, (C₁-C₆)alkyl, (C₃-C₇)cycloalkyl, 4-8 membered heterocycloalkyl, (C₃-C₇)cycloalkyl(C₁-C₄)alkyl, (C₃-C₇)cycloalkyl(C₁-C₄)alkoxy, (C₁-C₄)alkoxy(C₁-C₄)alkyl, (C₁-C₄)alkoxy, (C₃-C₇)cycloalkoxy, 5- or 6-membered heteroaryl, (5- or 6-membered heteroaryl)-(C₁-C₄)alkyl and (4-8 membered heterocycloalkyl)-(C₁-C₄)alkyl wherein said (C₁-C₆)alkyl, (C₃-C₇)cycloalkyl, 4-8 membered heterocycloalkyl, (C₃-C₇)cycloalkyl(C₁-C₄)alkyl, (C₃-C₇)cycloalkyl(C₁-C₄)alkoxy, (C₁-C₄)alkoxy(C₁-C₄)alkyl, (C₁-C₄)alkoxy, (C₃-C₇)cycloalkoxy, 5- or 6-membered heteroaryl, (5- or 6-membered heteroaryl)-(C₁-C₄)alkyl and (4-8 membered heterocycloalkyl)-(C₁-C₄)alkyl is optionally substituted with one or more substituents independently selected from hydroxy, deuterium, halogen, cyano, oxo, (C₁-C₄)alkyl, (C₃-C₇)cycloalkyl, (C₁-C₄)alkoxy, hydroxy(C₁-C₄)alkyl, (C₁-C₄)alkoxy-(CH₂CH₂O)₀₋₂(C₁-C₄)alkyl, 4-7 membered heterocycloalkyl, —NR_{g}Rₕ, R_{g}RₕN—(C₁-C₄)alkyl and —CO(O)Rᵢ;

R_{f} represents (C₁-C₄)alkyl, or (C₃-C₇)cycloalkyl;

R_{g} and Rₕ each independently represents hydrogen, (C₁-C₄)alkyl, (C₃-C₇)cycloalkyl, (C₃-C₇)cycloalkyl(C₁-C₄)alkyl or hydroxy(C₁-C₄)alkyl;

or R_{g} and Rₕ together with the nitrogen to which they are attached form a 4-8 membered heterocycloalkyl wherein said 4-8 membered heterocycloalkyl is optionally substituted with one or more substituents independently selected from deuterium, halogen, hydroxy, (C₁-C₄)alkyl, hydroxy(C₁-C₄)alkyl, halo(C₁-C₄)alkyl;

Rᵢ represents hydrogen or (C₁-C₄)alkyl;

or pharmaceutically acceptable salts, hydrates, solvates or prodrugs thereof.

Embodiment 2: A compound according to embodiment 1 having the general formula (Ia)

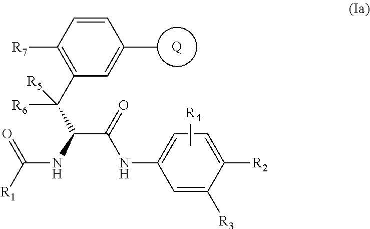

wherein R₁, R₂, R₃, R₄, R₅, R₆, R₇, R₈, Q, Rₐ, R_{b}, R_{c}, R_{d}, Rₑ, R_{f}, R_{g}, Rₕ and Rᵢ and are as indicated in embodiment 1, or pharmaceutically acceptable salts, hydrates, solvates or prodrugs thereof.

Embodiment 3. A compound of general formula (Ib)

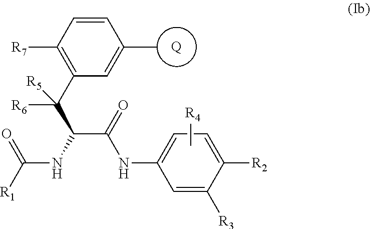

wherein R₁, R₂, R₃, R₄, R₅, R₆, R₇, R₈, Q, Rₐ, R_{b}, R_{c}, R_{d}, Rₑ, R_{f}, Rg, Rₕ and Ri and are as indicated in embodiment 1, or pharmaceutically acceptable salts, hydrates, solvates or prodrugs thereof.

Embodiment 4. A compound according to any of the embodiments above wherein

R₁ is selected from (C₃-C₇)cycloalkyl, (C₃-C₇)cycloalkyl(C₁-C₆)alkyl, (C₃-C₇)cycloalkyl(C₁-C₆)alkoxy and (C₃-C₇)cycloalkoxy wherein said (C₃-C₇)cycloalkyl, (C₃-C₇)cycloalkyl(C₁-C₆)alkyl, (C₃-C₇)cycloalkyl(C₁-C₆)alkoxy and (C₃-C₇)cycloalkoxy is optionally substituted with one or more substituents independently selected from Rₐ;

R₂ is selected from the group consisting of 5-membered heteroaryl, wherein said 5-membered heteroaryl is optionally substituted with one or more substituents independently selected from R_{b};

R₅ and R₆ each independently are selected from the group consisting of hydrogen, deuterium, (C₁-C₄)alkyl, (C₃-C₇)cycloalkyl and phenyl, with the proviso that at least one of R₅ and R₆ is selected from hydrogen or deuterium;

R₇ is selected from the group consisting of hydrogen and halogen, with the proviso that R₇ is not hydrogen when both R₅ and R₆ are selected from hydrogen or deuterium;

or R₆ and R₇ together with the phenyl to which R₇ is attached form a fused bicyclic ring system selected from the group consisting of tetralin and indane, wherein said tetralin or indane is optionally substituted with one more substituents independently selected from the group consisting of deuterium, halogen and (C₁-C₄)alkyl;

Q represents phenyl, 5- or 6-membered heteroaryl, 9- or 10-membered bicyclic heteroaryl, pyridonyl, indolinyl, isoindolinyl, tetrahydroquinolinyl or tetrahydroisoquinolinyl, wherein said pyridonyl, phenyl, 5- or 6-membered heteroaryl, 9- or 10-membered bicyclic heteroaryl, indolinyl, isoindolinyl, tetrahydroquinolinyl or tetrahydroisoquinolinyl is optionally substituted with one or more substituents independently selected from R₈;

and wherein Rₐ, R_{b} and R₈ are as defined above.

and pharmaceutically acceptable salts, hydrates, solvates or prodrugs thereof.

Embodiment 5. A compound according to any of the embodiments above, wherein Q is isoindolinyl, indolinyl, tetrahydroisoquinoline, or tetrahydroquinoline wherein said isoindolinyl, indolinyl, tetrahydroisoquinolinyl, or tetrahydroquinolinyl is optionally substituted with one or more substituents independently selected from R₈.

Embodiment 6. A compound according to any of the embodiments 1-4 wherein Q is phenyl, optionally substituted with one or more substituents independently selected from R₈.

Embodiment 7. A compound according to any of the embodiments 1-4 wherein Q is 5- or 6-membered heteroaryl, optionally substituted with one or more substituents independently selected from R₈.

Embodiment 8. A compound according to any of the embodiments 1-4 wherein Q is pyridine or pyrazole optionally substituted with one or more substituents independently selected from R₈.

Embodiment 9. A compound according to any of the embodiments 1-4 wherein Q is pyridonyl, optionally substituted with one or more substituents independently selected from R₈.

Embodiment 10. A compound according to any of the embodiments 1-9 wherein

R₁ is (C₃-C₇)cycloalkyl, optionally substituted with one or more substituents independently selected from Rₐ.

Embodiment 11. A compound according to any one of the embodiments above, wherein R₂ is selected from pyrazolyl or imidazolyl, wherein said pyrazolyl or imidazolyl is optionally substituted with one or more substituents independently selected from R_{b}.

Embodiment 12. A compound according to any of the embodiments above wherein

R₁ is 5- or 6 membered heteroaryl which is optionally substituted with one or more substituents independently selected from Rₐ;

R₂ is 5-membered heteroaryl which is optionally substituted with one or more substituents independently selected from R_{b};

R₅ and R₆ each independently are selected from the group consisting of hydrogen, deuterium, (C₁-C₄)alkyl, (C₃-C₇)cycloalkyl and phenyl, with the proviso that at least one of R₅ and R₆ is selected from hydrogen or deuterium;

R₇ is selected from the group consisting of hydrogen and halogen, with the proviso that R₇ is not hydrogen when both R₅ and R₆ are selected from hydrogen or deuterium;

or R₆ and R₇ together with the phenyl to which R₇ is attached form a fused bicyclic ring system selected from the group consisting of tetralin and indane, wherein said tetralin or indane is optionally substituted with one more substituents independently selected from the group consisting of deuterium, halogen and (C₁-C₄)alkyl;

Q represents phenyl, 5- or 6-membered heteroaryl, 9- or 10-membered bicyclic heteroaryl, pyridonyl, indolinyl, isoindolinyl, tetrahydroquinolinyl or tetrahydroisoquinolinyl, wherein said pyridonyl, phenyl, 5- or 6-membered heteroaryl, 9- or 10-membered bicyclic heteroaryl, indolinyl, isoindolinyl, tetrahydroquinolinyl or tetrahydroisoquinolinyl is optionally substituted with one or more substituents independently selected from R₈;

and wherein Rₐ, R_{b} and R₈ are as defined above.

and pharmaceutically acceptable salts, hydrates, solvates or prodrugs thereof

Embodiment 13. A Compound according to any of the embodiments 1-3 and 12 above, wherein Q is isoindolinyl, indolinyl, tetrahydroisoquinolinyl, or tetrahydroquinolinyl wherein said isoindolinyl, indolinyl, tetrahydroisoquinolinyl, or tetrahydroquinolinyl is optionally substituted with one or more substituents independently selected from R₈.

Embodiment 14. A compound according to any of the embodiments 1-3 and 12 above wherein Q is phenyl, optionally substituted with one or more substituents independently selected from R₈.

Embodiment 15. A compound according to any of the embodiments 1-3 and 12 above wherein Q is 5- or 6-membered heteroaryl, optionally substituted with one or more substituents independently selected from R₈.

Embodiment 16. A compound according to any of the embodiments 1-3 and 12 above wherein Q is pyridine or pyrazole optionally substituted with one or more substituents independently selected from R₈.

Embodiment 17. A compound according to any of the embodiments 1-3 and 12 above wherein Q is pyridonyl, optionally substituted with one or more substituents independently selected from R₈.

Embodiment 18. A compound according to any one of the embodiments 1-3 and 12-17 above, wherein R₁ is selected from pyrazolyl, imidazolyl, thiazolyl, isoxazolyl and triazolyl, wherein said pyrazolyl, imidazolyl, thiazolyl, isoxazolyl and triazolyl is optionally substituted with one or more substituents independently selected from Rₐ.

Embodiment 19. A compound according to any one of the embodiments 1-3 and 12-18 above, wherein R₂ is selected from pyrazolyl or imidazolyl, wherein said pyrazolyl or imidazolyl is optionally substituted with one or more substituents independently selected from R_{b}.

Embodiment 20. A compound according to any one of the embodiments 1-3 and 12-19 above, wherein R₁ is pyrazolyl which is optionally substituted with one or more substituents independently selected from Rₐ.

Embodiment 21. A compound according to any one of the embodiments 1-3 and 12-20 above, wherein R₁ is pyrazol-3-yl which is optionally substituted with one or more substituents independently selected from Rₐ.

Embodiment 22. A compound of according to embodiments 1-3 and 12-21 wherein R₁ is 2-methyl-pyrazol-3-yl.

Embodiment 23. A compound according to embodiment 1-10 above wherein R₁ is cyclopropyl, optionally substituted with one or more substituents independently selected from Rₐ.

Embodiment 24. A compound according to any of the embodiments embodiment 1-10 and 23 above wherein R₁ is 1-fluoro-cycloalkyl.

Embodiment 25. A compound according to any one of the embodiments above, wherein R₂ is selected from pyrazolyl or imidazolyl, wherein said pyrazolyl or imidazolyl is optionally substituted with one or more substituents independently selected from R_{b}.

Embodiment 26. A compound according to embodiment 25 above, wherein R₂ is selected from pyrazol-3-yl or imidazo-4-yl, wherein said pyrazol-3-yl or imidazo-4-yl is optionally substituted with one or more substituents independently selected from R_{b}.

Embodiment 27. A compound according to embodiment 26 above wherein R₂ is 1H-pyrazolyl, 3-methyl-1H-pyrazol-4-yl, 3,5-dimethyl-1H-pyrazol-4-yl, 2-methyl-pyrazol-3-yl, 3,5-dimethyl-imidazo-4-yl or 3-methyl-imidazo-4-yl.

Embodiment 28. A compounds according to embodiment 27 above wherein R₂ is 3,5-dimethyl-1H-pyrazol-4-yl or 3-methyl-imidazo-4-yl.

Embodiment 29. A compound according to any one of the embodiments above wherein R₃ is hydrogen or hydroxy.

Embodiment 30 A compound according to any one of the embodiments above wherein R₃ is hydroxy.

Embodiment 31. A compound according to any one of the embodiments above wherein R₄ is hydrogen or deuterium.

Embodiment 32. A compound according to any of the embodiments above wherein R₈ is (C₁-C₆)alkyl, (C₃-C₆)cycloalkyl or 4-8 membered heterocycloalkyl wherein said (C₁-C₆)alkyl, (C₃-C₆)cycloalkyl or 4-8 membered heterocycloalkyl is optionally substituted with one or more substituents independently selected from (C₁-C₄)alkyl, deuterium and hydroxy.

Embodiment 33. A compound according to any of the embodiments above wherein R₈ is —NR_{g}Rₕ or (C₁-C₆)alkyl substituted with —NR_{g}Rₕ wherein wherein R_{g} and Rₕ together with the nitrogen to which they are attached form a 4-8 membered heterocycloalkyl wherein said 4-8 membered heterocycloalkyl is optionally substituted with one or more substituents independently selected from deuterium, halogen, hydroxy, (C₁-C₄)alkyl, hydroxy(C₁-C₄)alkyl and halo(C₁-C₄)alkyl.

Embodiment 34. A compound according to embodiment 33 above wherein the 4-8 membered heterocyclic ring formed is pyrrolidinyl, piperidinyl, piperazinyl, azetidinyl, 2,5-diazabicyclo[2.2.1]heptanyl, or 2-oxa-5-aza-[2.2.1]heptanyl and wherein said pyrrolidinyl, piperidinyl, piperazinyl, morpholinyl, azetidinyl, 2,5-diazabicyclo[2.2.1]heptanyl, or 2-oxa-5-aza-[2.2.1]heptanyl is optionally substituted with one or more substituents independently selected from deuterium, halogen, hydroxy, (C₁-C₄)alkyl, hydroxy(C₁-C₄)alkyl, halo(C₁-C₄)alkyl.

Embodiment 35. A compound according to any of the embodiments above wherein Q is unsubstituted or wherein R₈ is deuterium, (C₁-C₆)alkyl,

wherein said (C₁-C₆)alkyl is optionally substituted with one or more substituents independently selected from hydroxy, (C₁-C₄)alkyl, (C₃-C₇)cycloalkyl and —NR_{g}Rₕ wherein R_{g} and Rₕ is independently selected from hydrogen, (C₁-C₄)alkyl, (C₃-C₇)cycloalkyl, (C₃-C₇)cycloalkyl(C₁-C₄)alkyl and hydroxy(C₁-C₄)alkyl

Embodiment 36. A compound according to embodiments 1 or 2

R₁ is selected from (C₃-C₇)cycloalkyl, (C₃-C₇)cycloalkyl(C₁-C₆)alkyl, (C₃-C₇)cycloalkyl(C₁-C₆)alkoxy, (C₃-C₇)cycloalkoxy and 5- or 6 membered heteroaryl wherein said (C₃-C₇)cycloalkyl, (C₃-C₇)cycloalkyl(C₁-C₆)alkyl, (C₃-C₇)cycloalkyl(C₁-C₆)alkoxy, (C₃-C₇)cycloalkoxy and 5- or 6 membered heteroaryl is optionally substituted with one or more substituents independently selected from Rₐ;

R₂ is selected from the group consisting of 5-membered heteroaryl, wherein said 5-membered heteroaryl is optionally substituted with one or more substituents independently selected from R_{b};

R₅ and R₆ each independently are selected from the group consisting of hydrogen, deuterium, (C₁-C₄)alkyl, (C₃-C₇)cycloalkyl and phenyl, with the proviso that at least one of R₅ and R₆ is selected from hydrogen or deuterium;

R₇ is selected from the group consisting of hydrogen and halogen, with the proviso that R₇ is not hydrogen when both R₅ and R₆ are selected from hydrogen or deuterium;

or R₆ and R₇ together with the phenyl to which R₇ is attached form a fused bicyclic ring system selected from the group consisting of tetralin and indane, wherein said tetralin or indane is optionally substituted with one more substituents independently selected from the group consisting of deuterium, halogen and (C₁-C₄)alkyl;

Q represents phenyl, 5- or 6-membered heteroaryl, 9- or 10-membered bicyclic heteroaryl, pyridonyl, indolinyl, isoindolinyl, tetrahydroquinolinyl or tetrahydroisoquinolinyl, wherein said phenyl, 5- or 6-membered heteroaryl, 9- or 10-membered bicyclic heteroaryl, pyridonyl, indolinyl, isoindolinyl, tetrahydroquinolinyl or tetrahydroisoquinolinyl is optionally substituted with one or more substituents independently selected from R₈;

and wherein Rₐ, R_{b} and R₈ are as defined for embodiment 1.

or pharmaceutically acceptable salts, hydrates, solvates or prodrugs thereof

Embodiment 37. A compound according to embodiment 36 wherein

R₁ is selected from (C₃-C₇)cycloalkyl, (C₃-C₇)cycloalkyl(C₁-C₆)alkyl, (C₃-C₇)cycloalkyl(C₁-C₆)alkoxy, (C₃-C₇)cycloalkoxy and 5- or 6 membered heteroaryl wherein said (C₃-C₇)cycloalkyl, (C₃-C₇)cycloalkyl(C₁-C₆)alkyl, (C₃-C₇)cycloalkyl(C₁-C₆)alkoxy, (C₃-C₇)cycloalkoxy and 5- or 6 membered heteroaryl is optionally substituted with one or more substituents independently selected from Rₐ;

R₂ is selected from the group consisting of 5-membered heteroaryl, wherein said 5-membered heteroaryl is optionally substituted with one or more substituents independently selected from R_{b};

R₅ and R₆ each independently are selected from the group consisting of hydrogen, deuterium, (C₁-C₄)alkyl, (C₃-C₇)cycloalkyl and phenyl, with the proviso that at least one of R₅ and R₆ is selected from hydrogen or deuterium;

R₇ is selected from the group consisting of hydrogen and halogen, with the proviso that R₇ is not hydrogen when both R₅ and R₆ are selected from hydrogen or deuterium;

Q represents phenyl, 5- or 6-membered heteroaryl, 9- or 10-membered bicyclic heteroaryl, pyridonyl, indolinyl, isoindolinyl, tetrahydroquinolinyl or tetrahydroisoquinolinyl, wherein said pyridonyl, phenyl, 5- or 6-membered heteroaryl, 9- or 10-membered bicyclic heteroaryl, indolinyl, isoindolinyl, tetrahydroquinolinyl or tetrahydroisoquinolinyl is optionally substituted with one or more substituents independently selected from R₈;

and wherein Rₐ, R_{b} and R₈ are as defined in embodiment 36.

Embodiment 38. A compound according to embodiment 36 above wherein

R₁ is selected from (C₃-C₇)cycloalkyl, (C₃-C₇)cycloalkyl(C₁-C₆)alkyl, (C₃-C₇)cycloalkyl(C₁-C₆)alkoxy, (C₃-C₇)cycloalkoxy and 5- or 6 membered heteroaryl wherein said (C₃-C₇)cycloalkyl, (C₃-C₇)cycloalkyl(C₁-C₆)alkyl, (C₃-C₇)cycloalkyl(C₁-C₆)alkoxy, (C₃-C₇)cycloalkoxy and 5- or 6 membered heteroaryl is optionally substituted with one or more substituents independently selected from Rₐ;

R₂ is selected from the group consisting of 5-membered heteroaryl, wherein said 5-membered heteroaryl is optionally substituted with one or more substituents independently selected from R_{b};

R₆ and R₇ together with the phenyl to which R₇ is attached form a fused bicyclic ring system selected from the group consisting of tetralin and indane, wherein said tetralin or indane is optionally substituted with one more substituents independently selected from the group consisting of deuterium, halogen and (C₁-C₄)alkyl;

Q represents phenyl, 5- or 6-membered heteroaryl, 9- or 10-membered bicyclic heteroaryl, pyridonyl, indolinyl, isoindolinyl, tetrahydroquinolinyl or tetrahydroisoquinolinyl, wherein said phenyl, 5- or 6-membered heteroaryl, 9- or 10-membered bicyclic heteroaryl, pyridonyl, indolinyl, isoindolinyl, tetrahydroquinolinyl or tetrahydroisoquinolinyl is optionally substituted with one or more substituents independently selected from R₈;

and wherein Rₐ, R_{b} and R₈ are as defined in embodiment 36.

Embodiment 39. A compound according to any one of embodiment 37 or 38 wherein

R₁ is (C₃-C₇)cycloalkyl, pyrazolyl or isoxazolyl wherein said (C₃-C₇)cycloalkyl, pyrazolyl or isoxazolyl is optionally substituted with one or more substituents independently selected from Rₐ;

R₂ is pyrazolyl or imidazolyl wherein said pyrazolyl or imidazolyl is optionally substituted with one or more substituents independently selected from R_{b};

Q represents phenyl, 5- or 6-membered heteroaryl, 9- or 10-membered bicyclic heteroaryl, pyridonyl, indolinyl, isoindolinyl, tetrahydroquinolinyl or tetrahydroisoquinolinyl, wherein said phenyl, 5- or 6-membered heteroaryl, 9- or 10-membered bicyclic heteroaryl, pyridonyl, indolinyl, isoindolinyl, tetrahydroquinolinyl or tetrahydroisoquinolinyl is optionally substituted with one or more substituents independently selected from R₈;

wherein and wherein Rₐ, R_{b} and R₈ are as defined in embodiment 36.

Embodiment 40. A compound according to embodiment 39 wherein

R₂ is pyrazol-4-yl, pyrazol-3-yl or imidazo-4-yl wherein said pyrazol-4-yl, pyrazol-3-yl or imidazo-4-yl is optionally independently substituted with one or more substituents independently selected from R_{b};

Q represents phenyl, 5- or 6-membered heteroaryl, 9- or 10-membered bicyclic heteroaryl, pyridonyl, indolinyl, isoindolinyl, tetrahydroquinolinyl or tetrahydroisoquinolinyl, wherein said phenyl, 5- or 6-membered heteroaryl, 9- or 10-membered bicyclic heteroaryl, pyridonyl, indolinyl, isoindolinyl, tetrahydroquinolinyl or tetrahydroisoquinolinyl is optionally substituted with one or more substituents independently selected from R₈;

wherein and wherein Rₐ, R_{b} and R₈ are as defined in embodiment 1.

Embodiment 41. A compound according to any one of embodiments 36-40 wherein

Wherein Q is unsubstituted or R₈ is deuterium, or (C₁-C₆)alkyl wherein said (C₁-C₆)alkyl is optionally substituted with one or more substituents independently selected from hydroxy, (C₁-C₄)alkyl, (C₃-C₇)cycloalkyl and —NR_{g}Rₕ wherein R_{g} and Rₕ is independently selected from (C₁-C₄)alkyl and hydrogen.

Embodiment 42. A compound according to any one of embodiment 36-40 wherein R₈ is —NR_{g}Rₕ, or (C₁-C₆)alkyl substituted with NR_{g}Rₕ wherein R_{g} and Rₕ together with the nitrogen to which they are attached form a 4-8 membered heterocycloalkyl wherein said 4-8 membered heterocycloalkyl is optionally substituted with one or more substituents independently selected from deuterium, halogen, hydroxy, (C₁-C₄)alkyl, hydroxy(C₁-C₄)alkyl and halo(C₁-C₄)alkyl.

Embodiment 43. A compound according to embodiment 42 above wherein the 4-8 membered heterocyclic ring formed is pyrrolidinyl, piperidinyl, piperazinyl, azetidinyl 2,5-diazabicyclo[2.2.1]heptanyl, or 2-oxa-5-aza-[2.2.1]heptanyl and wherein said pyrrolidinyl, piperidinyl, piperazinyl, morpholinyl, azetidinyl, 2,5-diazabicyclo[2.2.1]heptanyl, or 2-oxa-5-aza-[2.2.1]heptanyl is optionally substituted with one or more substituents independently selected from deuterium, halogen, hydroxy, (C₁-C₄)alkyl, hydroxy(C₁-C₄)alkyl, halo(C₁-C₄)alkyl.

Embodiment 44. A compound according to any embodiment above wherein R₁ is a 5 membered heteroaryl comprising one or more nitrogen atoms as the only heteroatom ring member(s); and wherein R₂ is a 5 membered heteroaryl comprising one or more nitrogen atoms as the only heteroatom ring member(s).

Embodiment 45. A compound selected from:
N-[(1S)-2-[4-(3,5-dimethyl-1H-pyrazol-4-yl)anilino]-1-[(1R)-6-isoindolin-5-ylindan-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-6-[4-(morpholinomethyl)phenyl]indan-1-yl]-2-oxo-ethyl]-2-methyl-pyrazole-3-carboxamide;
N-[(1S)-1-[6-(1-isopropyl-6-oxo-3-pyridyl)indan-1-yl]-2-[4-(3-methylimidazol-4-yl)anilino]-2-oxo-ethyl]-2-methyl-pyrazole-3-carboxamide;
N-[(1S)-1-[(1R)-6-(1-tert-butylpyrazol-4-yl)indan-1-yl]-2-[4-(3,5-dimethyl-1H-pyrazol-4-yl)anilino]-2-oxo-ethyl]-2-methyl-pyrazole-3-carboxamide;
N-[(1S)-1-[(1R)-6-(1-cyclobutylpyrazol-4-yl)indan-1-yl]-2-[4-(3,5-dimethyl-1H-pyrazol-4-yl)anilino]-2-oxo-ethyl]-2-methyl-pyrazole-3-carboxamide;
N-[(1S)-2-[4-(3,5-dimethyl-1H-pyrazol-4-yl)anilino]-1-[(1R)-6-(1-isopropyl-6-oxo-3-pyridyl)indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-(1-isopropylpyrazol-4-yl)indan-1-yl]-2-oxo-ethyl]-2-methyl-pyrazole-3-carboxamide;
N-[(1S)-1-[(1R)-6-(1-cyclopropylpyrazol-4-yl)indan-1-yl]-2-[4-(3,5-dimethyl-1H-pyrazol-4-yl)anilino]-2-oxo-ethyl]-2-methyl-pyrazole-3-carboxamide;
N-[(1S)-2-[4-(3,5-dimethyl-1H-pyrazol-4-yl)anilino]-2-oxo-1-[(1R)-6-(2-piperazin-1-yl-4-pyridyl)indan-1-yl]ethyl]-1-fluoro-cyclopropanecarboxamide;
N-[(1S)-2-[4-(3,5-dimethyl-1H-pyrazol-4-yl)anilino]-1-[(1R)-6-(2-methylisoindolin-5-yl)indan-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-6-[1-(2-hydroxy-1,1-dimethyl-ethyl)pyrazol-4-yl]indan-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-6-[1-(2-hydroxy-1,1-dimethyl-ethyl)pyrazol-4-yl]indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-[1-(2-hydroxy-2-methyl-propyl)pyrazol-4-yl]indan-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-6-(2-isopropyl-4-pyridyl)indan-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-6-(2-methyl-3,4-dihydro-1H-isoquinolin-6-yl)indan-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-6-[2-(1-hydroxy-1-methyl-ethyl)-4-pyridyl]indan-1-yl]-2-oxo-ethyl]-2-methyl-pyrazole-3-carboxamide;
N-[(1S)-2-[4-(3,5-dimethyl-1H-pyrazol-4-yl)anilino]-2-oxo-1-[(1R)-6-(1,2,3,4-tetrahydroisoquinolin-6-yl)indan-1-yl]ethyl]-2-methyl-pyrazole-3-carboxamide;
N-[(1S)-2-[4-(3,5-dimethyl-1H-pyrazol-4-yl)anilino]-1-[(1R)-6-[3-methyl-4-(morpholinomethyl)phenyl]indan-1-yl]-2-oxo-ethyl]-2-methyl-pyrazole-3-carboxamide;
N-[(1S)-2-[4-(3,5-dimethyl-1H-pyrazol-4-yl)anilino]-2-oxo-1-[(1R)-6-[4-(pyrrolidin-1-ylmethyl)phenyl]indan-1-yl]ethyl]-2-methyl-pyrazole-3-carboxamide;
N-[(1S)-2-[4-(3,5-dimethyl-1H-pyrazol-4-yl)anilino]-2-oxo-1-[(1R)-6-[4-(1-piperidylmethyl)phenyl]indan-1-yl]ethyl]-2-methyl-pyrazole-3-carboxamide;
N-[(1S)-1-[(1R)-6-[4-(azetidin-1-ylmethyl)phenyl]indan-1-yl]-2-[4-(3,5-dimethyl-1H-pyrazol-4-yl)anilino]-2-oxo-ethyl]-2-methyl-pyrazole-3-carboxamide;
N-[(1S)-1-[(1R)-6-[1-(2-amino-1,1-dimethyl-ethyl)pyrazol-4-yl]indan-1-yl]-2-[4-(3,5-dimethyl-1H-pyrazol-4-yl)anilino]-2-oxo-ethyl]-2-methyl-pyrazole-3-carboxamide;
N-[(1S)-1-[(1R)-6-[2-[(1S,4S)-2,5-diazabicyclo[2.2.1]heptan-2-yl]-4-pyridyl]indan-1-yl]-2-[4-(3,5-dimethyl-1H-pyrazol-4-yl)anilino]-2-oxo-ethyl]-1-fluoro-cyclopropanecarboxamide;
N-[(1S)-1-[(1R)-6-[1-(2-amino-1,1-dimethyl-ethyl)pyrazol-4-yl]indan-1-yl]-2-[4-(3,5-dimethyl-1H-pyrazol-4-yl)anilino]-2-oxo-ethyl]-1-fluoro-cyclopropanecarboxamide;
N-[(1S)-1-[6-(1-isopropyl-6-oxo-3-pyridyl)indan-1-yl]-2-[4-(3-methylimidazol-4-yl)anilino]-2-oxo-ethyl]-2-methyl-pyrazole-3-carboxamide;
N-[(1S)-1-[(1R)-7-(1-cyclopropylpyrazol-4-yl)tetralin-1-yl]-2-[4-(3,5-dimethyl-1H-pyrazol-4-yl)anilino]-2-oxo-ethyl]-2-methyl-pyrazole-3-carboxamide;
N-[(1S)-2-[4-(3,5-dimethyl-1H-pyrazol-4-yl)anilino]-1-[(1R)-7-(1-isopropyl-6-oxo-3-pyridyl)tetralin-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-7-[2-[(1S,4S)-2-oxa-5-azabicyclo[2.2.1]heptan-5-yl]-4-pyridyl]tetralin-1-yl]-2-oxo-ethyl]-1-fluoro-cyclopropanecarboxamide;
N-[(1S)-1-[(1R)-7-(1-isopropyl-6-oxo-3-pyridyl)tetralin-1-yl]-2-[4-(3-methylimidazol-4-yl)anilino]-2-oxo-ethyl]-2-methyl-pyrazole-3-carboxamide;
cyclopropyl N-[(1S)-2-[3-hydroxy-4-(3-methylimidazol-4-yl)anilino]-1-[(1R)-7-(1-isopropyl-6-oxo-3-pyridyl)tetralin-1-yl]-2-oxo-ethyl]carbamate;
cyclopropyl N-[(1S)-2-[4-(3,5-dimethylimidazol-4-yl)anilino]-1-[(1R)-7-[2-(4-methylpiperazin-1-yl)-4-pyridyl]tetralin-1-yl]-2-oxo-ethyl]carbamate;
N-[(1S)-2-[4-(3,5-dimethyl-1H-pyrazol-4-yl)-3-hydroxy-anilino]-1-[(1R)-7-(1-isopropyl-6-oxo-3-pyridyl)tetralin-1-yl]-2-oxo-ethyl]-2-methyl-pyrazole-3-carboxamide;
N-[(1S)-1-[(1R)-7-(1-tert-butylpyrazol-4-yl)tetralin-1-yl]-2-[4-(3,5-dimethyl-1H-pyrazol-4-yl)anilino]-2-oxo-ethyl]-2-methyl-pyrazole-3-carboxamide;
N-[(1S)-2-[4-(3,5-dimethyl-1H-pyrazol-4-yl)anilino]-1-[(1R)-7-isoindolin-5-yltetralin-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-7-(2-methyl-3,4-dihydro-1H-isoquinolin-6-yl)tetralin-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-7-(2-methylisoindolin-5-yl)tetralin-1-yl]-2-oxo-ethyl]-2-methyl-pyrazole-3-carboxamide;
N-[(1S)-2-[4-(3,5-dimethyl-1H-pyrazol-4-yl)anilino]-2-oxo-1-[(1R)-7-(1,2,3,4-tetrahydroisoquinolin-6-yl)tetralin-1-yl]ethyl]-2-methyl-pyrazole-3-carboxamide;
N-[(1S)-2-[4-(3,5-dimethyl-1H-pyrazol-4-yl)anilino]-2-oxo-1-[(1R)-7-(2-piperazin-1-yl-4-pyridyl)tetralin-1-yl]ethyl]-1-fluoro-cyclopropanecarboxamide;
N-[(1S)-1-[(1R)-7-[2-[(1S,4S)-2,5-diazabicyclo[2.2.1]heptan-2-yl]-4-pyridyl]tetralin-1-yl]-2-[4-(3,5-dimethylimidazol-4-yl)anilino]-2-oxo-ethyl]-1-fluoro-cyclopropanecarboxamide;
N-[(1S)-2-[4-(3,5-dimethylimidazol-4-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]-1-fluoro-cyclopropanecarboxamide;
N-[(1S)-2-[4-(3,5-dimethylimidazol-4-yl)anilino]-1-[(1R)-7-isoindolin-5-yltetralin-1-yl]-2-oxo-ethyl]-2-methyl-pyrazole-3-carboxamide;
N-[(1S)-2-[4-(3,5-dimethylimidazol-4-yl)anilino]-1-[(1R)-7-isoindolin-5-yltetralin-1-yl]-2-oxo-ethyl]-1-fluoro-cyclopropanecarboxamide;
N-[(1S)-1-[(1R)-7-(1-isopropyl-6-oxo-3-pyridyl)tetralin-1-yl]-2-[4-(3-methylimidazol-4-yl)anilino]-2-oxo-ethyl]-2-methyl-pyrazole-3-carboxamide;
tert-butyl N-[(1R)-1-[(1S)-7-(1-isopropyl-6-oxo-3-pyridyl)tetralin-1-yl]-2-[4-(3-methylimidazol-4-yl)anilino]-2-oxo-ethyl]carbamate;
N-[(1S)-2-[4-(3,5-dimethyl-1H-pyrazol-4-yl)-3-hydroxy-anilino]-1-[(1R)-7-(1-isopropyl-6-oxo-3-pyridyl)tetralin-1-yl]-2-oxo-ethyl]-2-methyl-pyrazole-3-carboxamide;
N-[(1S)-2-[4-(3,5-dimethyl-1H-pyrazol-4-yl)-3-hydroxy-anilino]-1-[(1R)-7-(1-isopropyl-6-oxo-3-pyridyl)tetralin-1-yl]-2-oxo-ethyl]-1-fluoro-cyclopropanecarboxamide;
N-[(1R)-1-[(1S)-7-(1-isopropyl-6-oxo-3-pyridyl)tetralin-1-yl]-2-[4-(3-methylimidazol-4-yl)anilino]-2-oxo-ethyl]-2-methyl-pyrazole-3-carboxamide;
N-[(1S)-2-[3-hydroxy-4-(3-methylimidazol-4-yl)anilino]-1-[(1R)-7-(1-isopropyl-6-oxo-3-pyridyl)tetralin-1-yl]-2-oxo-ethyl]-2-methyl-pyrazole-3-carboxamide;
N-[(1S)-1-[(1R)-7-[3-(1-amino-1-methyl-ethyl)phenyl]tetralin-1-yl]-2-[4-(3,5-dimethylimidazol-4-yl)anilino]-2-oxo-ethyl]-2-methyl-pyrazole-3-carboxamide;
N-[(1S)-2-[6-chloro-3-(1-isopropyl-6-oxo-3-pyridyl)cyclohexa-2,4-dien-1-yl]-1-[[4-(3,5-dimethyl-1H-pyrazol-4-yl)phenyl]carbamoyl]propyl]-1-fluoro-cyclopropanecarboxamide;
N-[(1S)-2-[2-chloro-5-(1-isopropyl-6-oxo-3-pyridyl)phenyl]-1-[[4-(3,5-dimethylimidazol-4-yl)phenyl]carbamoyl]propyl]-1-fluoro-cyclopropanecarboxamide;
cyclopropyl N-[(1S)-2-[2-chloro-5-(1-isopropyl-6-oxo-3-pyridyl)phenyl]-1-[[4-(3-methylimidazol-4-yl)phenyl]carbamoyl]propyl]carbamate;
N-[(1S)-2-[2-chloro-5-(1-isopropyl-6-oxo-3-pyridyl)phenyl]-1-[[4-(3-methylimidazol-4-yl)phenyl]carbamoyl]propyl]-2-methyl-pyrazole-3-carboxamide;
N-[(1S)-2-[2-chloro-5-(1-isopropyl-6-oxo-3-pyridyl)phenyl]-1-[[4-(3-methylimidazol-4-yl)phenyl]carbamoyl]propyl]-2-methyl-pyrazole-3-carboxamide;
N-[(1S)-2-[3-(1-isopropyl-6-oxo-3-pyridyl)phenyl]-1-[[4-(3-methylimidazol-4-yl)phenyl]carbamoyl]propyl]-2-methyl-pyrazole-3-carboxamide;
N-[(1S)-2-[3-(1-isopropyl-6-oxo-3-pyridyl)phenyl]-1-[[4-(3-methylimidazol-4-yl)phenyl]carbamoyl]propyl]-2-methyl-pyrazole-3-carboxamide;
N-[(1S)-1-[[2-chloro-5-[3-(1-hydroxy-1-methyl-ethyl)phenyl]phenyl]methyl]-2-[4-(3,5-dimethyl-1H-pyrazol-4-yl)anilino]-2-oxo-ethyl]-2-methyl-pyrazole-3-carboxamide;
N-[(1S)-1-[[5-[3-(1-amino-1-methyl-ethyl)phenyl]-2-chloro-phenyl]methyl]-2-[4-(3,5-dimethyl-1H-pyrazol-4-yl)anilino]-2-oxo-ethyl]-2-methyl-pyrazole-3-carboxamide;
N-[(1S)-1-[[2-chloro-5-[3-(dimethylaminomethyl)phenyl]phenyl]methyl]-2-[4-(3,5-dimethyl-1H-pyrazol-4-yl)anilino]-2-oxo-ethyl]-2-methyl-pyrazole-3-carboxamide;
N-[(1S)-1-[[5-[3-(aminomethyl)phenyl]-2-chloro-phenyl]methyl]-2-[4-(3,5-dimethyl-1H-pyrazol-4-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-[4-(4-methyl-1,2,4-triazol-3-yl)anilino]-2-oxo-ethyl]-2-methyl-pyrazole-3-carboxamide;
N-[(1S)-1-[[2-chloro-5-(1-cyclopropyl-6-oxo-3-pyridyl)phenyl]methyl]-2-[4-(4-methyl-1,2,4-triazol-3-yl)anilino]-2-oxo-ethyl]-3-methyl-isoxazole-4-carboxamide;
N-[1-[[2-chloro-5-[2-(2-hydroxy-2-methyl-propyl)-4-pyridyl]phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-[4-(3-methyltriazol-4-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-oxo-2-[4-(2H-tetrazol-5-yl)anilino]ethyl]-2-methyl-pyrazole-3-carboxamide;
tert-butyl N-[1-[[2-chloro-5-(1-isopropyl-6-oxo-3-pyridyl)phenyl]methyl]-2-[4-(2-methylimidazol-1-yl)anilino]-2-oxo-ethyl]carbamate;
N-[1-[[2-chloro-5-(1-isopropyl-6-oxo-3-pyridyl)phenyl]methyl]-2-[4-(2-methylimidazol-1-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-[3-hydroxy-4-(4-methyl-1,2,4-triazol-3-yl)anilino]-2-oxo-ethyl]-2-methyl-pyrazole-3-carboxamide;
N-[(1S)-1-[[2-chloro-5-(1-isopropyl-6-oxo-3-pyridyl)phenyl]methyl]-2-[3-methoxy-4-(2-methylpyrazol-3-yl)anilino]-2-oxo-ethyl]-3-methyl-isoxazole-4-carboxamide;
N-[(1S)-1-[[2-chloro-5-(1-isopropyl-6-oxo-3-pyridyl)phenyl]methyl]-2-[3-hydroxy-4-(2-methylpyrazol-3-yl)anilino]-2-oxo-ethyl]-3-methyl-isoxazole-4-carboxamide;
N-[1-[[2-chloro-5-(1-isopropyl-6-oxo-3-pyridyl)phenyl]methyl]-2-[4-(4-cyclopropyl-1,2,4-triazol-3-yl)anilino]-2-oxo-ethyl]-2-methyl-pyrazole-3-carboxamide;
N-[1-[[2-chloro-5-(2-methyl-4-pyridyl)phenyl]methyl]-2-[4-(4-cyclopropyl-1,2,4-triazol-3-yl)anilino]-2-oxo-ethyl]-2-methyl-pyrazole-3-carboxamide;
N-[1-[[2-chloro-5-(2-isopropyl-4-pyridyl)phenyl]methyl]-2-[4-(4-cyclopropyl-1,2,4-triazol-3-yl)anilino]-2-oxo-ethyl]-2-methyl-pyrazole-3-carboxamide;
N-[1-[[2-chloro-5-(2-isopropyl-4-pyridyl)phenyl]methyl]-2-[4-(4-methyl-1,2,4-triazol-3-yl)anilino]-2-oxo-ethyl]-2-methyl-pyrazole-3-carboxamide;
tert-butyl N-[1-[[2-chloro-5-(1-isopropyl-6-oxo-3-pyridyl)phenyl]methyl]-2-oxo-2-[4-(1H-triazol-5-yl)anilino]ethyl]carbamate;
N-[1-[[2-chloro-5-(1-isopropyl-6-oxo-3-pyridyl)phenyl]methyl]-2-[4-(3-methyl-1,2,4-triazol-4-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-[4-(3-methylisoxazol-4-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-[4-(3-methylimidazol-4-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-oxo-2-[4-(1H-pyrazol-4-yl)anilino]ethyl]-3-methyl-isoxazole-4-carboxamide;
N-[(1S)-1-[[2-chloro-5-[2-(1-hydroxy-2,2-dimethyl-propyl)-4-pyridyl]phenyl]methyl]-2-[4-(4-methyl-1,2,4-triazol-3-yl)anilino]-2-oxo-ethyl]-3-methyl-isoxazole-4-carboxamide;
N-[(1S)-1-[[2-chloro-5-[2-(1-hydroxy-2-methyl-propyl)-4-pyridyl]phenyl]methyl]-2-[4-(4-methyl-1,2,4-triazol-3-yl)anilino]-2-oxo-ethyl]-3-methyl-isoxazole-4-carboxamide;
N-[1-[[2-chloro-5-(1-isopropyl-6-oxo-3-pyridyl)phenyl]methyl]-2-oxo-2-[4-(1H-triazol-5-yl)anilino]ethyl]-2-methyl-pyrazole-3-carboxamide;
N-[(1S)-1-[[2-chloro-5-[2-(2,2-dimethylpropanoyl)-4-pyridyl]phenyl]methyl]-2-[4-(4-methyl-1,2,4-triazol-3-yl)anilino]-2-oxo-ethyl]-3-methyl-isoxazole-4-carboxamide;
N-[(1S)-1-[[2-chloro-5-[2-(cyclopropanecarbonyl)-4-pyridyl]phenyl]methyl]-2-[4-(4-methyl-1,2,4-triazol-3-yl)anilino]-2-oxo-ethyl]-3-methyl-isoxazole-4-carboxamide;
tert-butyl N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]carbamate;
benzyl N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]carbamate;
N-[1-[[2-chloro-5-(1-isopropyl-6-oxo-3-pyridyl)phenyl]methyl]-2-[4-(5-methyl-1,2,4-triazol-1-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-[4-(5-methyl-1H-pyrazol-4-yl)anilino]-2-oxo-ethyl]-2-methyl-pyrazole-3-carboxamide;
tert-butyl N-[1-[[2-chloro-5-(1-isopropyl-6-oxo-3-pyridyl)phenyl]methyl]-2-[4-(5-methyl-1H-pyrazol-4-yl)anilino]-2-oxo-ethyl]carbamate;
N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]-2-isopropyl-pyrazole-3-carboxamide;
N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]-1-fluoro-cyclopropanecarboxamide;
N-[1-[[2-chloro-5-(1-isopropyl-6-oxo-3-pyridyl)phenyl]methyl]-2-[4-(3,5-dimethylisoxazol-4-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-[4-(3,5-dimethyl-1H-pyrazol-4-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-[4-(4-methyl-1,2,4-triazol-3-yl)anilino]-2-oxo-ethyl]-3-(trifluoromethyl)isoxazole-4-carboxamide;
3-tert-butyl-N-[1-[[2-chloro-5-(1-isopropyl-6-oxo-3-pyridyl)phenyl]methyl]-2-[4-(4-methyl-1,2,4-triazol-3-yl)anilino]-2-oxo-ethyl]isoxazole-4-carboxamide;
3-[2-chloro-5-(1-isopropyl-6-oxo-3-pyridyl)phenyl]-2-[(2,2-difluoro-2-phenyl-acetyl)amino]-N-[4-(4-methyl-1,2,4-triazol-3-yl)phenyl]propenamide;
N-[1-[[2-chloro-5-(1-isopropyl-6-oxo-3-pyridyl)phenyl]methyl]-2-[4-(5-methyl-1H-triazol-4-yl)anilino]-2-oxo-ethyl]-2-methyl-pyrazole-3-carboxamide;
isopropyl N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]carbamate;
isobutyl N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]carbamate;
tert-butyl N-[1-[[2-chloro-5-(1-isopropyl-6-oxo-3-pyridyl)phenyl]methyl]-2-[3-chloro-4-(3-methylimidazol-4-yl)anilino]-2-oxo-ethyl]carbamate;
N-[1-[[2-chloro-5-(1-isopropyl-6-oxo-3-pyridyl)phenyl]methyl]-2-[3-chloro-4-(2-methylpyrazol-3-yl)anilino]-2-oxo-ethyl]-3-methyl-isoxazole-4-carboxamide;
N-[1-[[2-chloro-5-(1-isopropyl-6-oxo-3-pyridyl)phenyl]methyl]-2-[3-chloro-4-(3-methylimidazol-4-yl)anilino]-2-oxo-ethyl]-3-methyl-isoxazole-4-carboxamide;
N-[(1S)-1-[[2-chloro-5-(1-isopropyl-6-oxo-3-pyridyl)phenyl]methyl]-2-[4-(3-methylimidazol-4-yl)anilino]-2-oxo-ethyl]-1-fluoro-cyclopropanecarboxamide;
cyclobutyl N-[1-[[2-chloro-5-(1-isopropyl-6-oxo-3-pyridyl)phenyl]methyl]-2-[4-(4-methyl-1,2,4-triazol-3-yl)anilino]-2-oxo-ethyl]carbamate;
(1-methylcyclopropyl)methyl N-[1-[[2-chloro-5-(1-isopropyl-6-oxo-3-pyridyl)phenyl]methyl]-2-[4-(4-methyl-1,2,4-triazol-3-yl)anilino]-2-oxo-ethyl]carbamate;
N-[1-[[2-chloro-5-[3-[cyclopropyl(methyl)carbamoyl]phenyl]phenyl]methyl]-2-[4-(4-methyl-1,2,4-triazol-3-yl)anilino]-2-oxo-ethyl]-3-methyl-isoxazole-4-carboxamide;
N-[1-[[2-chloro-5-(3-ethylphenyl)phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-[3-(1-hydroxy-1-methyl-ethyl)phenyl]phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-(3-ethoxyphenyl)phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-[3-(trifluoromethoxy)phenyl]phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-[3-(difluoromethoxy)phenyl]phenyl]methyl]-2-[4-(4-methyl-1,2,4-triazol-3-yl)anilino]-2-oxo-ethyl]-2-methyl-pyrazole-3-carboxamide;
N-[1-[[5-[3-(azetidine-1-carbonyl)phenyl]-2-chloro-phenyl]methyl]-2-[4-(4-methyl-1,2,4-triazol-3-yl)anilino]-2-oxo-ethyl]-3-methyl-isoxazole-4-carboxamide;
N-[(1S)-1-[[2-chloro-5-(1-isopropyl-6-oxo-3-pyridyl)phenyl]methyl]-2-[4-(2-methylimidazol-1-yl)anilino]-2-oxo-ethyl]-1-fluoro-cyclopropanecarboxamide;
N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]-3-methyl-butanamide;
(2S)-3-[2-chloro-5-(1-isopropyl-6-oxo-3-pyridyl)phenyl]-2-[(2-cyclopropylacetyl)amino]-N-[4-(4-methyl-1,2,4-triazol-3-yl)phenyl]propenamide;
N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]-1-(trifluoromethyl)cyclopropanecarboxamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]-3-methyl-isoxazole-4-carboxamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]-1-fluoro-cyclopropanecarboxamide;
N-[1-[[2-chloro-5-[3-(3-fluoroazetidine-1-carbonyl)phenyl]phenyl]methyl]-2-[4-(4-methyl-1,2,4-triazol-3-yl)anilino]-2-oxo-ethyl]-3-methyl-isoxazole-4-carboxamide;
N-[1-[[2-chloro-5-(1-isopropyl-6-oxo-3-pyridyl)phenyl]methyl]-2-oxo-2-[4-[3-(trifluoromethyl)-1H-pyrazol-4-yl]anilino]ethyl]-2-methyl-pyrazole-3-carboxamide;
N-[1-[[2-chloro-5-(3-isopropoxyphenyl)phenyl]methyl]-2-[4-(4-methyl-1,2,4-triazol-3-yl)anilino]-2-oxo-ethyl]-2-methyl-pyrazole-3-carboxamide;
N-[(1S)-1-[[2-chloro-5-[2-[cyclopropyl(hydroxy)methyl]-4-pyridyl]phenyl]methyl]-2-[4-(3-methylimidazol-4-yl)anilino]-2-oxo-ethyl]-3-methyl-isoxazole-4-carboxamide;
N-[(1S)-1-[[2-chloro-5-[2-[cyclopropyl(hydroxy)methyl]-4-pyridyl]phenyl]methyl]-2-[4-(3-methylimidazol-4-yl)anilino]-2-oxo-ethyl]-1-fluoro-cyclopropanecarboxamide;
N-[(1S)-1-[[2-chloro-5-[2-[cyclopropyl(hydroxy)methyl]-4-pyridyl]phenyl]methyl]-2-[4-(2-methylimidazol-1-yl)anilino]-2-oxo-ethyl]-3-methyl-isoxazole-4-carboxamide;
N-[(1S)-1-[[2-chloro-5-[2-[cyclopropyl(hydroxy)methyl]-4-pyridyl]phenyl]methyl]-2-[4-(2-methylimidazol-1-yl)anilino]-2-oxo-ethyl]-1-fluoro-cyclopropanecarboxamide;
(2S)-3-[2-chloro-5-(1-isopropyl-6-oxo-3-pyridyl)phenyl]-2-[(2-cyclobutylacetyl)amino]-N-[4-(4-methyl-1,2,4-triazol-3-yl)phenyl]propenamide;
N-[(1S)-1-[[5-(2-tert-butyl-4-pyridyl)-2-chloro-phenyl]methyl]-2-[4-(4-methyl-1,2,4-triazol-3-yl)anilino]-2-oxo-ethyl]-3-methyl-isoxazole-4-carboxamide;
N-[1-[[2-chloro-5-(1-isopropyl-6-oxo-3-pyridyl)phenyl]methyl]-2-[4-(5-methyl-1H-pyrazol-4-yl)anilino]-2-oxo-ethyl]-1-fluoro-cyclopropanecarboxamide;
N-[1-[[2-chloro-5-[4-hydroxy-3-(morpholinomethyl)phenyl]phenyl]methyl]-2-[4-(4-methyl-1,2,4-triazol-3-yl)anilino]-2-oxo-ethyl]-2-methyl-pyrazole-3-carboxamide;
cyclopropyl N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]carbamate;
N-[(1S)-1-[[2-chloro-5-(1-isopropyl-6-oxo-3-pyridyl)phenyl]methyl]-2-[3-hydroxy-4-(3-methylimidazol-4-yl)anilino]-2-oxo-ethyl]-3-methyl-isoxazole-4-carboxamide;
N-[(1S)-1-[[2-chloro-5-(1-isopropyl-6-oxo-3-pyridyl)phenyl]methyl]-2-[3-hydroxy-4-(3-methylimidazol-4-yl)anilino]-2-oxo-ethyl]-1-fluoro-cyclopropanecarboxamide;
tert-butyl N-[(1S)-1-[[2-chloro-5-(1-isopropyl-6-oxo-3-pyridyl)phenyl]methyl]-2-[3-fluoro-4-(3-methylimidazol-4-yl)anilino]-2-oxo-ethyl]carbamate;
N-[(1S)-1-[[2-chloro-5-(1-isopropyl-6-oxo-3-pyridyl)phenyl]methyl]-2-[3-fluoro-4-(3-methylimidazol-4-yl)anilino]-2-oxo-ethyl]-2-methyl-pyrazole-3-carboxamide;
N-[(1S)-1-[[2-chloro-5-(1-isopropyl-6-oxo-3-pyridyl)phenyl]methyl]-2-[4-(3-methyl-1H-pyrazol-4-yl)anilino]-2-oxo-ethyl]-1-fluoro-cyclopropanecarboxamide;
N-[(1S)-1-[[2-chloro-5-(1-isopropyl-6-oxo-3-pyridyl)phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[2-chloro-5-(1-isopropyl-6-oxo-3-pyridyl)phenyl]methyl]-2-[3-fluoro-4-(1H-pyrazol-4-yl)anilino]-2-oxo-ethyl]-2-methyl-pyrazole-3-carboxamide;
N-[1-[[2-chloro-5-[3-(dimethylaminomethyl)-4-hydroxy-phenyl]phenyl]methyl]-2-[4-(4-methyl-1,2,4-triazol-3-yl)anilino]-2-oxo-ethyl]-2-methyl-pyrazole-3-carboxamide, formic acid;
N-[(1S)-1-[[2-chloro-5-[2-[cyclopropyl(hydroxy)methyl]-4-pyridyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-[2-(1-cyclopropyl-1-hydroxy-ethyl)-4-pyridyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-[2-(1-cyclopropyl-1-hydroxy-ethyl)-4-pyridyl]phenyl]methyl]-2-[4-(3-methylimidazol-4-yl)anilino]-2-oxo-ethyl]-1-fluoro-cyclopropanecarboxamide;
N-[(1S)-1-[[2-chloro-5-(1-isopropyl-6-oxo-3-pyridyl)phenyl]methyl]-2-[4-(3,5-dimethylimidazol-4-yl)anilino]-2-oxo-ethyl]-2-methyl-pyrazole-3-carboxamide;
N-[1-[[2-chloro-5-[3-(1-hydroxycyclobutyl)phenyl]phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-[3-(cyclobutoxy)phenyl]phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-(2-pyrrolidin-1-yl-4-pyridyl)phenyl]methyl]-2-[4-(3-methylimidazol-4-yl)anilino]-2-oxo-ethyl]-2-methyl-pyrazole-3-carboxamide;
N-[(1S)-1-[[2-chloro-5-(1-isopropyl-6-oxo-3-pyridyl)phenyl]methyl]-2-[4-(3-methylimidazol-4-yl)anilino]-2-oxo-ethyl]-1-fluoro-cyclobutanecarboxamide;
N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]-1-fluoro-cyclobutanecarboxamide;
N-[1-[[5-[2-(azetidin-1-yl)-4-pyridyl]-2-chloro-phenyl]methyl]-2-[4-(3-methylimidazol-4-yl)anilino]-2-oxo-ethyl]-2-methyl-pyrazole-3-carboxamide;
N-[1-[[2-chloro-5-[2-[cyclopropyl(methyl)amino]-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-(2-ethoxy-4-pyridyl)phenyl]methyl]-2-[4-(3-methylimidazol-4-yl)anilino]-2-oxo-ethyl]-2-methyl-pyrazole-3-carboxamide;
N-[1-[[2-chloro-5-[2-(2,2,2-trifluoroethoxy)-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-[cyclopropylmethyl(methyl)amino]-4-pyridyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]-2,2-dimethyl-cyclopropanecarboxamide;
N-[1-[[2-chloro-5-[3-(dimethylcarbamoyl)-4-(methoxymethoxy)phenyl]phenyl]methyl]-2-[4-(4-methyl-1,2,4-triazol-3-yl)anilino]-2-oxo-ethyl]-2-methyl-pyrazole-3-carboxamide;
N-[1-[[5-[3-(azetidine-1-carbonyl)-4-(methoxymethoxy)phenyl]-2-chloro-phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-[4-hydroxy-3-(piperazin-1-ylmethyl)phenyl]phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-[2-(cyclobutoxy)-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-[isopropyl(methyl)amino]-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-(1-piperidyl)-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-(difluoromethoxy)-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-(dimethylamino)-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[1-isopropyl-6-oxo-5-(trifluoromethyl)-3-pyridyl]phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-[3-(2-hydroxy-1,1-dimethyl-ethyl)phenyl]phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-(2-morpholino-4-pyridyl)phenyl]methyl]-2-[4-(3-methylimidazol-4-yl)anilino]-2-oxo-ethyl]-2-methyl-pyrazole-3-carboxamide;
N-[1-[[2-chloro-5-[2-(cyclopropylmethoxy)-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[3-(dimethylcarbamoyl)-4-hydroxy-phenyl]phenyl]methyl]-2-[4-(4-methyl-1,2,4-triazol-3-yl)anilino]-2-oxo-ethyl]-2-methyl-pyrazole-3-carboxamide;
N-[1-[[5-[3-(azetidin-1-ylmethyl)-4-hydroxy-phenyl]-2-chloro-phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-[3-(cyclopropylmethyl)phenyl]phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-(3-cyclopropylphenyl) phenyl]methyl]-2-[4-(4-methyl-1,2,4-triazol-3-yl)anilino]-2-oxo-ethyl]-2-methyl-pyrazole-3-carboxamide;
N-[(1S)-1-[[5-(1-tert-butylpyrazol-4-yl)-2-chloro-phenyl]methyl]-2-[4-(3-methylimidazol-4-yl)anilino]-2-oxo-ethyl]-2-methyl-pyrazole-3-carboxamide;
N-[1-[[5-[3-(azetidine-1-carbonyl)-4-hydroxy-phenyl]-2-chloro-phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-[4-hydroxy-3-(piperazine-1-carbonyl)phenyl]phenyl]methyl]-2-[4-(4-methyl-1,2,4-triazol-3-yl)anilino]-2-oxo-ethyl]-2-methyl-pyrazole-3-carboxamide;
N-[(1S)-1-[[2-chloro-5-(1-isopropyl-6-oxo-3-pyridyl)phenyl]methyl]-2-[3-hydroxy-4-(3-methyl-1H-pyrazol-4-yl)anilino]-2-oxo-ethyl]-2-methyl-pyrazole-3-carboxamide;
N-[(1S)-1-[[2-chloro-5-(1-isopropyl-6-oxo-3-pyridyl)phenyl]methyl]-2-[3-hydroxy-4-(1H-pyrazol-4-yl)anilino]-2-oxo-ethyl]-2-methyl-pyrazole-3-carboxamide;
N-[1-[[5-[3-(1-amino-1-methyl-ethyl)phenyl]-2-chloro-phenyl]methyl]-2-[4-(3-methylimidazol-4-yl)anilino]-2-oxo-ethyl]-2-methyl-pyrazole-3-carboxamide;
N-[(1S)-1-[[2-chloro-5-(1-isopropyl-6-oxo-3-pyridyl)phenyl]methyl]-2-[4-(3,5-dimethyl-1H-pyrazol-4-yl)-3-hydroxy-anilino]-2-oxo-ethyl]-2-methyl-pyrazole-3-carboxamide;
N-[1-[[2-chloro-5-[3-(5,6-dihydro-4H-1,3-oxazin-2-yl)-4-hydroxy-phenyl]phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-[3-(1-hydroxy-1-methyl-ethyl)phenyl]phenyl]methyl]-2-[4-(3-methylimidazol-4-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-[4-(3-chloro-1H-pyrazol-4-yl)anilino]-2-oxo-ethyl]-2-methyl-pyrazole-3-carboxamide;
N-[(1S)-1-[[2-chloro-5-[3-(1-hydroxy-1-methyl-ethyl)phenyl]phenyl]methyl]-2-[4-(3,5-dimethylimidazol-4-yl)anilino]-2-oxo-ethyl]-2-methyl-pyrazole-3-carboxamide;
N-[(1S)-1-[[5-(1-tert-butylpyrazol-4-yl)-2-chloro-phenyl]methyl]-2-[4-(3,5-dimethylimidazol-4-yl)anilino]-2-oxo-ethyl]-2-methyl-pyrazole-3-carboxamide;
N-[1-[[2-chloro-5-[2-(6-oxa-2-azaspiro[3.3]heptan-2-yl)-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-[(3S)-3-(dimethylamino)pyrrolidin-1-yl]-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-[(3S)-3-(hydroxymethyl)pyrrolidin-1-yl]-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-[(3R)-3-(hydroxymethyl)pyrrolidin-1-yl]-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-[2-(hydroxymethyl)azetidin-1-yl]-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-[(2S)-2-(hydroxymethyl)azetidin-1-yl]-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-[(2R)-2-(hydroxymethyl)pyrrolidin-1-yl]-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-[3-(hydroxymethyl)azetidin-1-yl]-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-[(3S)-3-hydroxypyrrolidin-1-yl]-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-[(3R)-3-hydroxypyrrolidin-1-yl]-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-(3-hydroxyazetidin-1-yl)-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-[2-(2,6-diazaspiro[3.3]heptan-6-yl)-4-pyridyl]phenyl]methyl]-2-[4-(3-methylimidazol-4-yl)anilino]-2-oxo-ethyl]-2-methyl-pyrazole-3-carboxamide;
N-[1-[[5-[2-(3-aminoazetidin-1-yl)-4-pyridyl]-2-chloro-phenyl]methyl]-2-[4-(3-methylimidazol-4-yl)anilino]-2-oxo-ethyl]-2-methyl-pyrazole-3-carboxamide;
N-[1-[[2-chloro-5-[2-[3-(methylamino)azetidin-1-yl]-4-pyridyl]phenyl]methyl]-2-[4-(3-methylimidazol-4-yl)anilino]-2-oxo-ethyl]-2-methyl-pyrazole-3-carboxamide;
N-[1-[[2-chloro-5-(2-piperazin-1-yl-4-pyridyl)phenyl]methyl]-2-[4-(3-methylimidazol-4-yl)anilino]-2-oxo-ethyl]-2-methyl-pyrazole-3-carboxamide;
N-[1-[[2-chloro-5-(4-cyano-3-ethyl-phenyl)phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-(4-cyano-3,5-dimethyl-phenyl)phenyl]methyl]-2-[4-(4-methyl-1,2,4-triazol-3-yl)anilino]-2-oxo-ethyl]-2-methyl-pyrazole-3-carboxamide;
N-[(1S)-1-[[5-(1-tert-butylpyrazol-4-yl)-2-chloro-phenyl]methyl]-2-[4-(3,5-dimethyl-1H-pyrazol-4-yl)anilino]-2-oxo-ethyl]-2-methyl-pyrazole-3-carboxamide;
cyclopropyl N-[(1S)-1-[[2-chloro-5-(1-isopropyl-6-oxo-3-pyridyl)phenyl]methyl]-2-[4-(3,5-dimethylimidazol-4-yl)anilino]-2-oxo-ethyl]carbamate;
N-[1-[[2-chloro-5-(4-cyano-3-isopropyl-phenyl)phenyl]methyl]-2-[4-(4-methyl-1,2,4-triazol-3-yl)anilino]-2-oxo-ethyl]-2-methyl-pyrazole-3-carboxamide;
N-[(1S)-1-[[5-[3-(1-amino-1-methyl-ethyl)phenyl]-2-chloro-phenyl]methyl]-2-[4-(3,5-dimethylimidazol-4-yl)anilino]-2-oxo-ethyl]-2-methyl-pyrazole-3-carboxamide;
N-[(1S)-1-[[2-chloro-5-[1-(1-cyano-1-methyl-ethyl)pyrazol-4-yl]phenyl]methyl]-2-[4-(3,5-dimethylimidazol-4-yl)anilino]-2-oxo-ethyl]-2-methyl-pyrazole-3-carboxamide;
N-[(1S)-1-[[2-chloro-5-(6-isopropylpyrimidin-4-yl)phenyl]methyl]-2-[4-(3-methylimidazol-4-yl)anilino]-2-oxo-ethyl]-2-methyl-pyrazole-3-carboxamide;
N-[(1S)-1-[[2-chloro-5-[2-(4-methylpiperazin-1-yl)-4-pyridyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-[2-[(2R)-2-methylpiperazin-1-yl]-4-pyridyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-[2-[(2S)-2-methylpiperazin-1-yl]-4-pyridyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-[2-[(3S)-3-methylpiperazin-1-yl]-4-pyridyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-[2-[(3R)-3-methylpiperazin-1-yl]-4-pyridyl]phenyl]methyl]-2-[4-(3-methylimidazol-4-yl)anilino]-2-oxo-ethyl]-2-methyl-pyrazole-3-carboxamide;
cyclopropyl N-[(1S)-1-[[2-chloro-5-[2-(4-methylpiperazin-1-yl)-4-pyridyl]phenyl]methyl]-2-[4-(3,5-dimethylimidazol-4-yl)anilino]-2-oxo-ethyl]carbamate;
N-[(1S)-1-[[2-chloro-5-(1-isobutyltriazol-4-yl)phenyl]methyl]-2-[4-(3-methylimidazol-4-yl)anilino]-2-oxo-ethyl]-2-methyl-pyrazole-3-carboxamide;
N-[(1S)-1-[[2-chloro-5-[2-(4-methylpiperazin-1-yl)-4-pyridyl]phenyl]methyl]-2-[4-(3,5-dimethylimidazol-4-yl)anilino]-2-oxo-ethyl]-2-methyl-pyrazole-3-carboxamide;
cyclopropyl N-[(1S)-1-[[2-chloro-5-(1-isobutyltriazol-4-yl)phenyl]methyl]-2-[4-(3-methylimidazol-4-yl)anilino]-2-oxo-ethyl]carbamate;
N-[(1S)-1-[[2-chloro-5-[3-[(3S)-morpholin-3-yl]phenyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-[3-[(3R)-morpholin-3-yl]phenyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-[3-[(3S)-4-methylmorpholin-3-yl]phenyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-[3-[(3R)-4-methylmorpholin-3-yl]phenyl]phenyl]methyl]-2-[4-(3-methylimidazol-4-yl)anilino]-2-oxo-ethyl]-2-methyl-pyrazole-3-carboxamide;
N-[(1S)-1-[[2-chloro-5-(3-morpholin-2-ylphenyl)phenyl]methyl]-2-[4-(3-methylimidazol-4-yl)anilino]-2-oxo-ethyl]-2-methyl-pyrazole-3-carboxamide hydrochloride;
N-[(1S)-1-[[2-chloro-5-[3-[(2S)-morpholin-2-yl]phenyl]phenyl]methyl]-2-[4-(3-methylimidazol-4-yl)anilino]-2-oxo-ethyl]-2-methyl-pyrazole-3-carboxamide; N-ethylethanamine;
N-[(1S)-1-[[2-chloro-5-[3-[(2R)-morpholin-2-yl]phenyl]phenyl]methyl]-2-[4-(3-methylimidazol-4-yl)anilino]-2-oxo-ethyl]-2-methyl-pyrazole-3-carboxamide; N-ethylethanamine;
N-[(1S)-1-[[2-chloro-5-[2-[(1R,4R)-2-oxa-5-azabicyclo[2.2.1]heptan-5-yl]-4-pyridyl]phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[2-chloro-5-[2-[(1S,4S)-2,5-diazabicyclo[2.2.1]heptan-2-yl]-4-pyridyl]phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[2-chloro-5-(2-morpholino-4-pyridyl)phenyl]methyl]-2-[4-(3,5-dimethylimidazol-4-yl)anilino]-2-oxo-ethyl]-1-fluoro-cyclopropanecarboxamide;
N-[(1S)-1-[[2-chloro-5-(2-morpholino-4-pyridyl)phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[2-chloro-5-(2-cyclobutylpyrazol-3-yl)phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[2-chloro-5-(1,5-dimethylpyrazol-4-yl)phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[2-chloro-5-[4-[[(1R,4R)-2-oxa-5-azabicyclo[2.2.1]heptan-5-yl]methyl]phenyl]phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[2-chloro-5-[4-[[(1S,4S)-2,5-diazabicyclo[2.2.1]heptan-2-yl]methyl]phenyl]phenyl]methyl]-2-[4-(3,5-dimethyl-1H-pyrazol-4-yl)anilino]-2-oxo-ethyl]-1-fluoro-cyclopropanecarboxamide;
N-[1-[[3-(1-isopropyl-6-oxo-3-pyridyl)phenyl]-phenyl-methyl]-2-[4-(3-methylimidazol-4-yl)anilino]-2-oxo-ethyl]-2-methyl-pyrazole-3-carboxamide;
cyclopropyl N-[1-[[3-(1-isopropyl-6-oxo-3-pyridyl)phenyl]-phenyl-methyl]-2-[4-(3-methylimidazol-4-yl)anilino]-2-oxo-ethyl]carbamate;
N-[1-[[3-(1-isopropyl-6-oxo-3-pyridyl)phenyl]-phenyl-methyl]-2-[4-(3-methylimidazol-4-yl)anilino]-2-oxo-ethyl]-2-methyl-pyrazole-3-carboxamide;
N-[1-[[3-(1-isopropyl-6-oxo-3-pyridyl)phenyl]-phenyl-methyl]-2-[4-(3-methylimidazol-4-yl)anilino]-2-oxo-ethyl]-2-methyl-pyrazole-3-carboxamide;
N-[1-[[3-(1-isopropyl-6-oxo-3-pyridyl)phenyl]-phenyl-methyl]-2-[4-(3-methylimidazol-4-yl)anilino]-2-oxo-ethyl]-2-methyl-pyrazole-3-carboxamide;
N-[1-[[3-(1-isopropyl-6-oxo-3-pyridyl)phenyl]-phenyl-methyl]-2-[4-(3-methyl-1H-pyrazol-4-yl)anilino]-2-oxo-ethyl]-2-methyl-pyrazole-3-carboxamide;
N-[1-[[3-(1-isopropyl-6-oxo-3-pyridyl)phenyl]-phenyl-methyl]-2-[4-(3-methyl-1H-pyrazol-4-yl)anilino]-2-oxo-ethyl]-2-methyl-pyrazole-3-carboxamide;
N-[1-[[3-(1-isopropyl-6-oxo-3-pyridyl)phenyl]-phenyl-methyl]-2-[4-(3-methyl-1H-pyrazol-4-yl)anilino]-2-oxo-ethyl]-2-methyl-pyrazole-3-carboxamide;
N-[1-[[3-(1-isopropyl-6-oxo-3-pyridyl)phenyl]-phenyl-methyl]-2-[4-(3-methyl-1H-pyrazol-4-yl)anilino]-2-oxo-ethyl]-2-methyl-pyrazole-3-carboxamide;
cyclopropyl N-[1-[[3-(1-isopropyl-6-oxo-3-pyridyl)phenyl]-phenyl-methyl]-2-[4-(3-methylimidazol-4-yl)anilino]-2-oxo-ethyl]carbamate;
cyclopropyl N-[1-[[3-(1-isopropyl-6-oxo-3-pyridyl)phenyl]-phenyl-methyl]-2-[4-(3-methylimidazol-4-yl)anilino]-2-oxo-ethyl]carbamate;
cyclopropyl N-[1-[[3-(1-isopropyl-6-oxo-3-pyridyl)phenyl]-phenyl-methyl]-2-[4-(3-methylimidazol-4-yl)anilino]-2-oxo-ethyl]carbamate;
cyclopropyl N-[1-[[3-(1-isopropyl-6-oxo-3-pyridyl)phenyl]-phenyl-methyl]-2-[4-(3-methyl-1H-pyrazol-4-yl)anilino]-2-oxo-ethyl]carbamate;
cyclopropyl N-[1-[[3-(1-isopropyl-6-oxo-3-pyridyl)phenyl]-phenyl-methyl]-2-[4-(3-methyl-1H-pyrazol-4-yl)anilino]-2-oxo-ethyl]carbamate;
cyclopropyl N-[1-[[3-(1-isopropyl-6-oxo-3-pyridyl)phenyl]-phenyl-methyl]-2-[4-(3-methyl-1H-pyrazol-4-yl)anilino]-2-oxo-ethyl]carbamate;
cyclopropyl N-[1-[[3-(1-isopropyl-6-oxo-3-pyridyl)phenyl]-phenyl-methyl]-2-[4-(3-methyl-1H-pyrazol-4-yl)anilino]-2-oxo-ethyl]carbamate;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]isoxazole-3-carboxamide;
(2S)-3-[2-chloro-5-(1-isopropyl-6-oxo-3-pyridyl)phenyl]-2-[(2-cyclopropylacetyl)amino]-N-[4-(2,4-dimethylpyrazol-3-yl)phenyl]propanamide;
(2S)-3-[2-chloro-5-(1-isopropyl-6-oxo-3-pyridyl)phenyl]-2-[(2-cyclobutylacetyl)amino]-N-[4-(2,4-dimethylpyrazol-3-yl)phenyl]propanamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]-1-methyl-cyclopropanecarboxamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]oxazole-4-carboxamide;
(2S)-3-[2-chloro-5-(1-isopropyl-6-oxo-3-pyridyl)phenyl]-2-(2-cyclopropylpropanoylamino)-N-[4-(2,4-dimethylpyrazol-3-yl)phenyl]propanamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]-1-fluoro-cyclobutanecarboxamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]butanamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]cyclopropanecarboxamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]-2-methyl-propanamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]-4-methyl-pentanamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]-1H-imidazole-4-carboxamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]cyclobutanecarboxamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]isoxazole-5-carboxamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]-1H-pyrazole-4-carboxamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]-1H-1,2,4-triazole-3-carboxamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]oxazole-5-carboxamide;
N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]oxazole-2-carboxamide;
N-[(1S)-2-[4-(2,4-dimethylpyrazol-3-yl)anilino]-1-[(1R)-6-[6-[(1S,4S)-2-oxa-5-azabicyclo[2.2.1]heptan-5-yl]pyrimidin-4-yl]indan-1-yl]-2-oxo-ethyl]-2-methyl-pyrazole-3-carboxamide;
N-[(1S)-1-[(1R)-6-(6-cyclopropylpyridazin-4-yl)indan-1-yl]-2-[4-(2,4-dimethylpyrazol-3-yl)anilino]-2-oxo-ethyl]-1-fluoro-cyclopropanecarboxamide;
N-[(1S)-1-[(1R)-6-(5-cyclopropylpyridazin-3-yl)indan-1-yl]-2-[4-(3,5-dimethyl-1H-pyrazol-4-yl)anilino]-2-oxo-ethyl]-1-fluoro-cyclopropanecarboxamide;
N-[(1S)-2-[4-(2,4-dimethylpyrazol-3-yl)anilino]-1-[(1R)-6-[5-[(1S,4S)-2-oxa-5-azabicyclo[2.2.1]heptan-5-yl]pyridazin-3-yl]indan-1-yl]-2-oxo-ethyl]-1-fluoro-cyclopropanecarboxamide;
N-[(1S)-2-[4-(2,4-dimethylpyrazol-3-yl)anilino]-1-[(1R)-6-(2-isopropylpyrimidin-4-yl)indan-1-yl]-2-oxo-ethyl]-2-methyl-pyrazole-3-carboxamide;
N-[(1S)-1-[(1R)-6-(6-cyclopropylpyridazin-4-yl)indan-1-yl]-2-[4-(3,5-dimethyl-1H-pyrazol-4-yl)anilino]-2-oxo-ethyl]-1-fluoro-cyclopropanecarboxamide;
N-[(1S)-2-[4-(2,4-dimethylpyrazol-3-yl)anilino]-1-[(1R)-6-(6-isopropylpyrazin-2-yl)indan-1-yl]-2-oxo-ethyl]-1-fluoro-cyclopropanecarboxamide; N-[(1S)-2-[4-(2,4-dimethylpyrazol-3-yl)anilino]-2-oxo-1-[(1R)-6-(6-propylpyrazin-2-yl)indan-1-yl]ethyl]-1-fluoro-cyclopropanecarboxamide;
N-[(1S)-2-[4-(2,4-dimethylpyrazol-3-yl)anilino]-1-[(1R)-6-(6-isopropylpyrazin-2-yl)indan-1-yl]-2-oxo-ethyl]-2-methyl-pyrazole-3-carboxamide; N-[(1S)-2-[4-(2,4-dimethylpyrazol-3-yl)anilino]-2-oxo-1-[(1R)-6-(6-propylpyrazin-2-yl)indan-1-yl]ethyl]-2-methyl-pyrazole-3-carboxamide;
N-[(1S)-2-[4-(3,5-dimethyl-1H-pyrazol-4-yl)anilino]-1-[(1R)-6-(6-isopropylpyrazin-2-yl)indan-1-yl]-2-oxo-ethyl]-1-fluoro-cyclopropanecarboxamide; N-[(1S)-2-[4-(3,5-dimethyl-1H-pyrazol-4-yl)anilino]-2-oxo-1-[(1R)-6-(6-propylpyrazin-2-yl)indan-1-yl]ethyl]-1-fluoro-cyclopropanecarboxamide;
N-[(1S)-1-[(1R)-6-(6-cyclopropylpyrazin-2-yl)indan-1-yl]-2-[4-(2,4-dimethylpyrazol-3-yl)anilino]-2-oxo-ethyl]-2-methyl-pyrazole-3-carboxamide;
N-[(1S)-1-[(R)-cyclopropyl-[3-[6-[(1S,4S)-2-oxa-5-azabicyclo[2.2.1]heptan-5-yl]pyrimidin-4-yl]phenyl]methyl]-2-[4-(2,4-dimethylpyrazol-3-yl)anilino]-2-oxo-ethyl]-1-fluoro-cyclopropanecarboxamide;
N-[(1S)-1-[(R)-cyclopropyl-[3-[6-[(1S,4S)-2-oxa-5-azabicyclo[2.2.1]heptan-5-yl]pyrimidin-4-yl]phenyl]methyl]-2-[4-(2,4-dimethylpyrazol-3-yl)anilino]-2-oxo-ethyl]-2-methyl-pyrazole-3-carboxamide;
N-[(1S)-1-[(R)-cyclopropyl-[3-[6-[(1S,4S)-2-oxa-5-azabicyclo[2.2.1]heptan-5-yl]pyrazin-2-yl]phenyl]methyl]-2-[4-(2,4-dimethylpyrazol-3-yl)anilino]-2-oxo-ethyl]-1-fluoro-cyclopropanecarboxamide;
N-[(1S)-1-[(R)-cyclopropyl-[3-[6-[(1S,4S)-2-oxa-5-azabicyclo[2.2.1]heptan-5-yl]pyrazin-2-yl]phenyl]methyl]-2-[4-(2,4-dimethylpyrazol-3-yl)anilino]-2-oxo-ethyl]-2-methyl-pyrazole-3-carboxamide;
N-[(1S)-1-[(R)-cyclopropyl-[3-[5-[(1S,4S)-2-oxa-5-azabicyclo[2.2.1]heptan-5-yl]-3-pyridyl]phenyl]methyl]-2-[4-(2,4-dimethylpyrazol-3-yl)anilino]-2-oxo-ethyl]-2-methyl-pyrazole-3-carboxamide;
N-[(1S)-1-[(R)-cyclopropyl-[3-[5-[(1S,4S)-2-oxa-5-azabicyclo[2.2.1]heptan-5-yl]-3-pyridyl]phenyl]methyl]-2-[4-(2,4-dimethylpyrazol-3-yl)anilino]-2-oxo-ethyl]-1-fluoro-cyclopropanecarboxamide;
N-[(1S)-2-[4-(2,4-dimethylpyrazol-3-yl)anilino]-1-[(1R)-6-[6-[(1S,4S)-2-oxa-5-azabicyclo[2.2.1]heptan-5-yl]pyrazin-2-yl]indan-1-yl]-2-oxo-ethyl]-1-fluoro-cyclopropanecarboxamide;
N-[(1S)-1-[[2-chloro-5-[2-(2-oxa-8-azaspiro[3.5]nonan-8-yl)pyrimidin-4-yl]phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[5-[2-(1,3,3a,4,6,6a-hexahydrofuro[3,4-c]pyrrol-5-yl)pyrimidin-4-yl]-2-chloro-phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[2-chloro-5-[2-(2-oxa-7-azaspiro[3.5]nonan-7-yl)pyrimidin-4-yl]phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[2-chloro-5-[2-(2-oxa-7-azaspiro[3.4]octan-7-yl)pyrimidin-4-yl]phenyl]methyl]-2-[4-(3,5-dimethyl-1H-pyrazol-4-yl)anilino]-2-oxo-ethyl]-1-fluoro-cyclopropanecarboxamide;
N-[(1S,2R)-1-[[4-(2,4-dimethylpyrazol-3-yl)phenyl]carbamoyl]-2-[3-[6-[(1S,4S)-2-oxa-5-azabicyclo[2.2.1]heptan-5-yl]pyrazin-2-yl]phenyl]propyl]-1-fluoro-cyclopropanecarboxamide;
N-[(1S,2R)-1-[[4-(2,4-dimethylpyrazol-3-yl)phenyl]carbamoyl]-2-[3-[6-[(1S,4S)-2-oxa-5-azabicyclo[2.2.1]heptan-5-yl]pyrimidin-4-yl]phenyl]propyl]-1-fluoro-cyclopropanecarboxamide;
N-[(1S)-1-[[2-chloro-5-[2-(1-oxidothiomorpholin-1-ium-4-yl)pyrimidin-4-yl]phenyl]methyl]-2-[4-(3,5-dimethyl-1H-pyrazol-4-yl)anilino]-2-oxo-ethyl]-1-fluoro-cyclopropanecarboxamide;
N-[(1S)-1-[[2-chloro-5-(2-thiomorpholinopyrimidin-4-yl)phenyl]methyl]-2-[4-(3,5-dimethyl-1H-pyrazol-4-yl)anilino]-2-oxo-ethyl]-1-fluoro-cyclopropanecarboxamide;
N-[(1S)-2-[4-(2,4-dimethylpyrazol-3-yl)anilino]-1-[(1R)-6-[2-[(1S,4S)-2-oxa-5-azabicyclo[2.2.1]heptan-5-yl]-4-pyridyl]indan-1-yl]-2-oxo-ethyl]-2-methyl-pyrazole-3-carboxamide;
tert-butyl N-[(1S)-1-[(R)-cyclopropyl-[3-[2-[(1S,4S)-2-oxa-5-azabicyclo[2.2.1]heptan-5-yl]-4-pyridyl]phenyl]methyl]-2-[4-(2,4-dimethylpyrazol-3-yl)anilino]-2-oxo-ethyl]carbamate;
Diastereomer 1 of tert-butyl N-[1-[(R)-cyclopropyl-[3-[2-[(1S,4S)-2-oxa-5-azabicyclo[2.2.1]heptan-5-yl]-4-pyridyl]phenyl]methyl]-2-[4-(2,4-dimethylpyrazol-3-yl)anilino]-2-oxo-ethyl]carbamate;
Diastereomer 2 of tert-butyl N-[1-[(R)-cyclopropyl-[3-[2-[(1S,4S)-2-oxa-5-azabicyclo[2.2.1]heptan-5-yl]-4-pyridyl]phenyl]methyl]-2-[4-(2,4-dimethylpyrazol-3-yl)anilino]-2-oxo-ethyl]carbamate;
N-[(1S)-2-[4-(3,5-dimethyl-1H-pyrazol-4-yl)anilino]-1-[(1R)-6-[5-[(1S,4S)-2-oxa-5-azabicyclo[2.2.1]heptan-5-yl]-3-pyridyl]indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-[6-(3-fluoroazetidin-1-yl)pyrimidin-4-yl]indan-1-yl]-2-oxo-ethyl]-1-fluoro-cyclopropanecarboxamide;
N-[(1S)-1-[(1R)-6-[6-(3,3-difluoroazetidin-1-yl)pyrimidin-4-yl]indan-1-yl]-2-[4-(3,5-dimethyl-1H-pyrazol-4-yl)anilino]-2-oxo-ethyl]-1-fluoro-cyclopropanecarboxamide;
cyclopropyl N-[(1S)-2-[4-(3,5-dimethyl-1H-pyrazol-4-yl)anilino]-1-[(1R)-6-[6-[(1S,4S)-2-oxa-5-azabicyclo[2.2.1]heptan-5-yl]pyrimidin-4-yl]indan-1-yl]-2-oxo-ethyl]carbamate;
N-[(1S,2R)-1-[[4-(3,5-dimethyl-1H-pyrazol-4-yl)phenyl]carbamoyl]-2-[3-[2-[(1S,4S)-2-oxa-5-azabicyclo[2.2.1]heptan-5-yl]-4-pyridyl]phenyl]propyl]-1-fluoro-cyclopropanecarboxamide;
N-[(1R,2R)-1-[[4-(3,5-dimethyl-1H-pyrazol-4-yl)phenyl]carbamoyl]-2-[3-[2-[(1S,4S)-2-oxa-5-azabicyclo[2.2.1]heptan-5-yl]-4-pyridyl]phenyl]propyl]-1-fluoro-cyclopropanecarboxamide;
N-[1-[[2-chloro-5-[4-[1-[2-(2-methoxyethoxy)ethyl]triazol-4-yl]phenyl]phenyl]methyl]-2-[4-(3,5-dimethyl-1H-pyrazol-4-yl)anilino]-2-oxo-ethyl]-2-methyl-pyrazole-3-carboxamide;
N-[1-[[2-chloro-5-[3-[1-[2-(2-methoxyethoxy)ethyl]triazol-4-yl]phenyl]phenyl]methyl]-2-[4-(3,5-dimethyl-1H-pyrazol-4-yl)anilino]-2-oxo-ethyl]-2-methyl-pyrazole-3-carboxamide;
N-[(1S)-1-[(1R)-6-[3-[(1S,4S)-2,5-diazabicyclo[2.2.1]heptan-2-yl]phenyl]indan-1-yl]-2-[4-(3,5-dimethyl-1H-pyrazol-4-yl)anilino]-2-oxo-ethyl]-1-fluoro-cyclopropanecarboxamide;
N-[(1S)-2-[4-(1,3-dimethylpyrazol-4-yl)anilino]-1-[(1R)-6-[2-[(1S,4S)-2-oxa-5-azabicyclo[2.2.1]heptan-5-yl]-4-pyridyl]indan-1-yl]-2-oxo-ethyl]-1-fluoro-cyclopropanecarboxamide;
1-fluoro-N-[(1S)-1-[(1R)-6-[2-[(1S,4S)-2-oxa-5-azabicyclo[2.2.1]heptan-5-yl]-4-pyridyl]indan-1-yl]-2-oxo-2-[4-(1,3,5-trimethylpyrazol-4-yl)anilino]ethyl]cyclopropanecarboxamide;
N-[(1S)-2-[4-(3,5-dimethyl-1H-pyrazol-4-yl)anilino]-1-[(1R)-6-[6-methyl-2-[(1S,4S)-2-oxa-5-azabicyclo[2.2.1]heptan-5-yl]pyrimidin-4-yl]indan-1-yl]-2-oxo-ethyl]-1-fluoro-cyclopropanecarboxamide;
N-[(1S)-2-[4-(2,4-dimethylpyrazol-3-yl)-3-hydroxy-anilino]-1-[(1R)-6-[2-[(1S,4S)-2-oxa-5-azabicyclo[2.2.1]heptan-5-yl]-4-pyridyl]indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-[4-[(1S,4S)-2-oxa-5-azabicyclo[2.2.1]heptan-5-yl]-2-pyridyl]indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-[2-[(1S,4S)-2-oxa-5-azabicyclo[2.2.1]heptan-5-yl]pyrimidin-4-yl]indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-[2-methyl-6-[(1S,4S)-2-oxa-5-azabicyclo[2.2.1]heptan-5-yl]pyrimidin-4-yl]indan-1-yl]-2-oxo-ethyl]-1-fluoro-cyclopropanecarboxamide;
N-[(1S)-2-[4-(2,4-dimethylpyrazol-3-yl)anilino]-1-[(1R)-6-[6-[(1S,4S)-2-oxa-5-azabicyclo[2.2.1]heptan-5-yl]pyrimidin-4-yl]indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-[2-(6-oxa-2-azaspiro[3.3]heptan-2-yl)-4-pyridyl]indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-[2-[(1R,4R)-2-oxa-5-azabicyclo[2.2.1]heptan-5-yl]pyrimidin-4-yl]indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-[2-[(1R,4R)-2-oxa-5-azabicyclo[2.2.1]heptan-5-yl]-4-pyridyl]indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-(4-isopropylimidazol-1-yl)indan-1-yl]-2-oxo-ethyl]-2-methyl-pyrazole-3-carboxamide;
N-[(1S)-2-[4-(3,5-dimethyl-1H-pyrazol-4-yl)anilino]-1-[(1R)-6-(4-isopropylimidazol-1-yl)indan-1-yl]-2-oxo-ethyl]-1-fluoro-cyclopropanecarboxamide;
N-[(1S)-2-[4-(2,4-dimethylpyrazol-3-yl)anilino]-1-[(1R)-6-[2-[(3S)-3-hydroxypyrrolidin-1-yl]-4-pyridyl]indan-1-yl]-2-oxo-ethyl]-1-fluoro-cyclopropanecarboxamide;
N-[(1S)-2-[4-(3,5-dimethylimidazol-4-yl)-3-hydroxy-anilino]-1-[(1R)-6-[2-[(1S,4S)-2-oxa-5-azabicyclo[2.2.1]heptan-5-yl]-4-pyridyl]indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-(1-isopropyl-5-methyl-6-oxo-3-pyridyl)indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-[6-[(1S,4S)-2-oxa-5-azabicyclo[2.2.1]heptan-5-yl]pyrimidin-4-yl]indan-1-yl]-2-oxo-ethyl]-1-fluoro-cyclopropanecarboxamide;
N-[(1S)-2-[4-(2,4-dimethylpyrazol-3-yl)anilino]-1-[(1R)-6-(1-isopropyl-5-methyl-6-oxo-3-pyridyl)indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-4-fluoro-6-[2-[(1S,4S)-2-oxa-5-azabicyclo[2.2.1]heptan-5-yl]-4-pyridyl]indan-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-4-fluoro-6-(1-isopropyl-5-methyl-6-oxo-3-pyridyl)indan-1-yl]-2-oxo-ethyl]-1-fluoro-cyclopropanecarboxamide;
1-fluoro-N-[(1S)-1-[(1R)-7-(1-isopropyl-5-methyl-6-oxo-3-pyridyl)tetralin-1-yl]-2-[4-(2-methylimidazol-1-yl)anilino]-2-oxo-ethyl]cyclopropanecarboxamide;
1-fluoro-N-[(1S)-2-[4-(4-methyl-1,2,4-triazol-3-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]cyclopropanecarboxamide;
1-fluoro-N-[(1S)-2-[3-hydroxy-4-(3-methylimidazol-4-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]cyclopropanecarboxamide;
N-[(1S)-2-[4-(3,5-dimethyl-1H-pyrazol-4-yl)anilino]-1-[(1R)-6-[2-[(1S,4S)-2-oxa-5-azabicyclo[2.2.1]heptan-5-yl]-4-pyridyl]indan-1-yl]-2-oxo-ethyl]-1-fluoro-cyclopropanecarboxamide;
2-methyl-N-[(1S)-2-[4-(2-methylimidazol-1-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]pyrazole-3-carboxamide;
2-methyl-N-[(1S)-2-[4-(2-methylimidazol-1-yl)anilino]-1-[(1R)-7-[2-[(1S,4S)-2-oxa-5-azabicyclo[2.2.1]heptan-5-yl]-4-pyridyl]tetralin-1-yl]-2-oxo-ethyl]pyrazole-3-carboxamide;
1-fluoro-N-[(1S)-2-[4-(2-methylimidazol-1-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]cyclopropanecarboxamide;
1-fluoro-N-[(1S)-2-[4-(2-methylimidazol-1-yl)anilino]-1-[(1R)-7-[2-[(1S,4S)-2-oxa-5-azabicyclo[2.2.1]heptan-5-yl]-4-pyridyl]tetralin-1-yl]-2-oxo-ethyl]cyclopropanecarboxamide;
N-[(1S)-2-[4-(2,4-dimethylpyrazol-3-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]-1-fluoro-cyclopropanecarboxamide;
N-[(1S)-2-[4-(2,4-dimethylpyrazol-3-yl)anilino]-1-[(1R)-7-[2-[(1S,4S)-2-oxa-5-azabicyclo[2.2.1]heptan-5-yl]-4-pyridyl]tetralin-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-7-[6-[(1S,4S)-2-oxa-5-azabicyclo[2.2.1]heptan-5-yl]pyrimidin-4-yl]tetralin-1-yl]-2-oxo-ethyl]-1-fluoro-cyclopropanecarboxamide;
N-[(1S)-2-[4-(3,5-dimethyl-1H-pyrazol-4-yl)anilino]-2-oxo-1-[(1R)-7-(1-tetrahydropyran-4-ylpyrazol-4-yl)tetralin-1-yl]ethyl]-1-fluoro-cyclopropanecarboxamide;
N-[(1S)-2-[4-(3,5-dimethyl-1H-pyrazol-4-yl)anilino]-1-[(1R)-7-[2-[(1S,4S)-2-oxa-5-azabicyclo[2.2.1]heptan-5-yl]-4-pyridyl]tetralin-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]-2-methyl-pyrazole-3-carboxamide;
N-[(1S)-1-[(1R)-7-[1-(difluoromethyl)pyrazol-4-yl]tetralin-1-yl]-2-[4-(3,5-dimethyl-1H-pyrazol-4-yl)anilino]-2-oxo-ethyl]-1-fluoro-cyclopropanecarboxamide;
Diastereomer 1 of N-[(1S)-2-[2-chloro-5-(1-isopropyl-6-oxo-3-pyridyl)phenyl]-1-[[4-(3,5-dimethyl-1H-pyrazol-4-yl)phenyl]carbamoyl]propyl]-1-fluoro-cyclopropanecarboxamide;
N-[(1S)-1-[(1R)-7-[2-[(1S,4S)-2,5-diazabicyclo[2.2.1]heptan-2-yl]-4-pyridyl]tetralin-1-yl]-2-[4-(2,4-dimethylpyrazol-3-yl)anilino]-2-oxo-ethyl]-1-fluoro-cyclopropanecarboxamide;
N-[(1S)-2-[4-(3,5-dimethyl-1H-pyrazol-4-yl)anilino]-1-[(1R)-7-(1-isopropyl-5-methyl-6-oxo-3-pyridyl)tetralin-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-7-[4-[[(1R,4R)-2-oxa-5-azabicyclo[2.2.1]heptan-5-yl]methyl]phenyl]tetralin-1-yl]-2-oxo-ethyl]-1-fluoro-cyclopropanecarboxamide;
N-[(1S)-1-[(1R)-7-[2-[(1R,4R)-2,5-diazabicyclo[2.2.1]heptan-2-yl]-4-pyridyl]tetralin-1-yl]-2-[4-(3,5-dimethylimidazol-4-yl)anilino]-2-oxo-ethyl]-1-fluoro-cyclopropanecarboxamide;
Diastereomer 2 of N-[(1S)-2-[2-chloro-5-(1-isopropyl-6-oxo-3-pyridyl)phenyl]-1-[[4-(3,5-dimethyl-1H-pyrazol-4-yl)phenyl]carbamoyl]propyl]-1-fluoro-cyclopropanecarboxamide;
N-[(1S)-1-[(2-chloro-5-morpholino-phenyl)methyl]-2-[4-(3,5-dimethyl-1H-pyrazol-4-yl)anilino]-2-oxo-ethyl]-2-methyl-pyrazole-3-carboxamide;
N-[(1S)-1-[[2-chloro-5-[3-[[(1R,4R)-2,5-diazabicyclo[2.2.1]heptan-2-yl]methyl]phenyl]phenyl]methyl]-2-[4-(3,5-dimethyl-1H-pyrazol-4-yl)anilino]-2-oxo-ethyl]-1-fluoro-cyclopropanecarboxamide;
N-[(1S)-1-[(1R)-7-[3-(azetidin-1-ylmethyl)phenyl]tetralin-1-yl]-2-[4-(3,5-dimethyl-1H-pyrazol-4-yl)anilino]-2-oxo-ethyl]-2-methyl-pyrazole-3-carboxamide;
N-[(1S)-2-[4-(3,5-dimethyl-1H-pyrazol-4-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]-1-fluoro-cyclopropanecarboxamide;
N-[(1S)-2-[4-(3,5-dimethyl-1H-pyrazol-4-yl)anilino]-1-[(1R)-6-(6-isopropylpyrimidin-4-yl)indan-1-yl]-2-oxo-ethyl]-1-fluoro-cyclopropanecarboxamide;
N-[1-[[2-chloro-5-(4-cyano-3-methyl-phenyl)phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-(4-cyanophenyl)phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-[1-isopropyl-6-oxo-5-(trifluoromethyl)-3-pyridyl]phenyl]methyl]-2-[4-(4-methyl-1,2,4-triazol-3-yl)anilino]-2-oxo-ethyl]-1-fluoro-cyclopropanecarboxamide;
N-[1-[[2-chloro-5-[2-[cyclopropyl(methyl)carbamoyl]-4-pyridyl]phenyl]methyl]-2-[4-(4-methyl-1,2,4-triazol-3-yl)anilino]-2-oxo-ethyl]-3-methyl-isoxazole-4-carboxamide;
N-[1-[[5-[2-(azetidine-1-carbonyl)-4-pyridyl]-2-chloro-phenyl]methyl]-2-[4-(4-methyl-1,2,4-triazol-3-yl)anilino]-2-oxo-ethyl]-3-methyl-isoxazole-4-carboxamide;
N-[1-[[2-chloro-5-[1-methyl-6-oxo-5-(trifluoromethyl)-3-pyridyl]phenyl]methyl]-2-[4-(4-methyl-1,2,4-triazol-3-yl)anilino]-2-oxo-ethyl]-3-methyl-isoxazole-4-carboxamide;
N-[1-[[2-chloro-5-[3-(difluoromethyl)phenyl]phenyl]methyl]-2-[4-(4-methyl-1,2,4-triazol-3-yl)anilino]-2-oxo-ethyl]-2-methyl-pyrazole-3-carboxamide;
tert-butyl N-[1-[[2-chloro-5-(1-isopropyl-6-oxo-3-pyridyl)phenyl]methyl]-2-[4-(2-methyl-1,2,4-triazol-3-yl)anilino]-2-oxo-ethyl]carbamate;
N-[1-[[2-chloro-5-(1-isopropyl-6-oxo-3-pyridyl)phenyl]methyl]-2-[4-(2-methyl-1,2,4-triazol-3-yl)anilino]-2-oxo-ethyl]-2-methyl-pyrazole-3-carboxamide;
cyclopentyl N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]carbamate;
[2-fluoro-1-(fluoromethyl)ethyl] N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]carbamate;
(2,2,2-trifluoro-1-methyl-ethyl) N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]carbamate;
N-[1-[[2-chloro-5-[3-(trifluoromethyl)phenyl]phenyl]methyl]-2-[4-(4-methyl-1,2,4-triazol-3-yl)anilino]-2-oxo-ethyl]-2-methyl-pyrazole-3-carboxamide;
N-[1-[[2-chloro-5-[4-hydroxy-3-(morpholine-4-carbonyl)phenyl]phenyl]methyl]-2-[4-(4-methyl-1,2,4-triazol-3-yl)anilino]-2-oxo-ethyl]-2-methyl-pyrazole-3-carboxamide;
N-[(1S)-1-[[2-chloro-5-(1-isopropyl-6-oxo-3-pyridyl)phenyl]methyl]-2-[3-methoxy-4-(3-methylimidazol-4-yl)anilino]-2-oxo-ethyl]-3-methyl-isoxazole-4-carboxamide;
N-[(1S)-1-[[2-chloro-5-(1-isopropyl-6-oxo-3-pyridyl)phenyl]methyl]-2-[3-methoxy-4-(3-methylimidazol-4-yl)anilino]-2-oxo-ethyl]-1-fluoro-cyclopropanecarboxamide;
N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]cyclobutanecarboxamide;
N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]-1-hydroxy-cyclopropanecarboxamide;
N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]-3,3-difluoro-cyclobutanecarboxamide;
N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]-3-fluoro-bicyclo[1.1.1]pentane-1-carboxamide;
N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]bicyclo[1.1.1]pentane-3-carboxamide;
(1R)—N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]-2,2-difluoro-cyclopropanecarboxamide;
(1S)—N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]-2,2-difluoro-cyclopropanecarboxamide;
[(1R)-2,2,2-trifluoro-1-methyl-ethyl] N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]carbamate;
[1-(trifluoromethyl)cyclopropyl]methyl N-[1-[[2-chloro-5-(1-isopropyl-6-oxo-3-pyridyl)phenyl]methyl]-2-[4-(4-methyl-1,2,4-triazol-3-yl)anilino]-2-oxo-ethyl]carbamate;
tert-butyl N-[1-[[2-chloro-5-(1-isopropyl-6-oxo-3-pyridyl)phenyl]methyl]-2-[3-chloro-4-(1H-pyrazol-4-yl)anilino]-2-oxo-ethyl]carbamate;
N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]-2,2-difluoro-propanamide;
N-[(1S)-1-[[2-chloro-5-(1-isopropyl-6-oxo-3-pyridyl)phenyl]methyl]-2-[4-(4-methyl-1,2,4-triazol-3-yl)anilino]-2-oxo-ethyl]-2-fluoro-2-methyl-propanamide;
tert-butyl N-[1-[[2-chloro-5-(1-isopropyl-6-oxo-3-pyridyl)phenyl]methyl]-2-[4-(5-methyl-1H-triazol-4-yl)anilino]-2-oxo-ethyl]carbamate;
N-[1-[[2-chloro-5-(1-isopropyl-6-oxo-3-pyridyl)phenyl]methyl]-2-[4-(4-methyl-1,2,4-triazol-3-yl)anilino]-2-oxo-ethyl]-2,2-dimethyl-propanamide;
N-[1-[[2-chloro-5-(1-isopropyl-6-oxo-3-pyridyl)phenyl]methyl]-2-[4-(4-methyl-1,2,4-triazol-3-yl)anilino]-2-oxo-ethyl]-3,3-dimethyl-butanamide;
N-[1-[[2-chloro-5-(1-isopropyl-6-oxo-3-pyridyl)phenyl]methyl]-2-[4-(4-methyl-1,2,4-triazol-3-yl)anilino]-2-oxo-ethyl]-2-methyl-pyridine-3-carboxamide;
N-[1-[[2-chloro-5-(1-isopropyl-6-oxo-3-pyridyl)phenyl]methyl]-2-[4-(2-methyltriazol-4-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-[4-(1-methyltriazol-4-yl)anilino]-2-oxo-ethyl]-2-methyl-pyrazole-3-carboxamide;
tert-butyl N-[1-[[2-chloro-5-(1-isopropyl-6-oxo-3-pyridyl)phenyl]methyl]-2-[4-(5-methyl-1,2,4-triazol-1-yl)anilino]-2-oxo-ethyl]carbamate;
N-[(1S)-1-[[2-chloro-5-[2-[cyclopropyl(hydroxy)methyl]-4-pyridyl]phenyl]methyl]-2-[4-(4-methyl-1,2,4-triazol-3-yl)anilino]-2-oxo-ethyl]-3-methyl-isoxazole-4-carboxamide;
tert-butyl N-[1-[[2-chloro-5-(1-isopropyl-6-oxo-3-pyridyl)phenyl]methyl]-2-[4-(2-methyltriazol-4-yl)anilino]-2-oxo-ethyl]carbamate;
tert-butyl N-[1-[[2-chloro-5-(1-isopropyl-6-oxo-3-pyridyl)phenyl]methyl]-2-[4-(1-methyltriazol-4-yl)anilino]-2-oxo-ethyl]carbamate;
tert-butyl N-[1-[[2-chloro-5-(1-isopropyl-6-oxo-3-pyridyl)phenyl]methyl]-2-[4-(3-methyl-1,2,4-triazol-1-yl)anilino]-2-oxo-ethyl]carbamate;
N-[1-[[2-chloro-5-(1-isopropyl-6-oxo-3-pyridyl)phenyl]methyl]-2-[4-(3-methyl-1,2,4-triazol-1-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-oxo-2-[4-(1,2,4-triazol-4-yl)anilino]ethyl]-2-methyl-pyrazole-3-carboxamide;
N-[1-[[2-chloro-5-(1-methyl-6-oxo-3-pyridyl)phenyl]methyl]-2-[4-(4-cyclopropyl-1,2,4-triazol-3-yl)anilino]-2-oxo-ethyl]-2-methyl-pyrazole-3-carboxamide;
tert-butyl N-[1-[[2-chloro-5-(1-isopropyl-6-oxo-3-pyridyl)phenyl]methyl]-2-oxo-2-[4-(1,2,4-triazol-4-yl)anilino]ethyl]carbamate;
N-[1-[[2-chloro-5-[2-[1-(hydroxymethyl)cyclopropyl]-4-pyridyl]phenyl]methyl]-2-[4-(4-methyl-1,2,4-triazol-3-yl)anilino]-2-oxo-ethyl]-2-methyl-pyrazole-3-carboxamide;
tert-butyl N-[1-[[2-chloro-5-(1-isopropyl-6-oxo-3-pyridyl)phenyl]methyl]-2-[4-(3-methyltriazol-4-yl)anilino]-2-oxo-ethyl]carbamate;
tert-butyl N-[1-[[2-chloro-5-(1-isopropyl-6-oxo-3-pyridyl)phenyl]methyl]-2-[4-(2-methylpyrazol-3-yl)anilino]-2-oxo-ethyl]carbamate;
methyl N-[(1S)-1-[[2-chloro-5-(1-cyclopropyl-6-oxo-3-pyridyl)phenyl]methyl]-2-[4-(4-methyl-1,2,4-triazol-3-yl)anilino]-2-oxo-ethyl]carbamate;
N-[(1S)-1-[[2-chloro-5-(1-cyclopropyl-6-oxo-3-pyridyl)phenyl]methyl]-2-[4-(4-methyl-1,2,4-triazol-3-yl)anilino]-2-oxo-ethyl]-1-methyl-cyclopropanecarboxamide;
tert-butyl N-[(1S)-1-[[2-chloro-5-(1-cyclopropyl-6-oxo-3-pyridyl)phenyl]methyl]-2-[4-(4-methyl-1,2,4-triazol-3-yl)anilino]-2-oxo-ethyl]carbamate;
tert-butyl N-[1-[[2-chloro-5-(1-isopropyl-6-oxo-3-pyridyl)phenyl]methyl]-2-[4-(1-methylpyrazol-3-yl)anilino]-2-oxo-ethyl]carbamate;
tert-butyl N-[1-[[2-chloro-5-(1-isopropyl-6-oxo-3-pyridyl)phenyl]methyl]-2-oxo-2-[4-(1H-pyrazol-3-yl)anilino]ethyl]carbamate;
tert-butyl N-[1-[[2-chloro-5-(1-isopropyl-6-oxo-3-pyridyl)phenyl]methyl]-2-[4-(4-methyl-1,2,4-triazol-3-yl)anilino]-2-oxo-ethyl]carbamate;
tert-butyl N-[1-[[2-chloro-5-(1-isopropyl-6-oxo-3-pyridyl)phenyl]methyl]-2-(4-imidazol-1-ylanilino)-2-oxo-ethyl]carbamate;
tert-butyl N-[1-[[2-chloro-5-(1-isopropyl-6-oxo-3-pyridyl)phenyl]methyl]-2-oxo-2-[4-(1,2,4-triazol-1-yl)anilino]ethyl]carbamate;
tert-butyl N-[1-[[2-chloro-5-(1-isopropyl-6-oxo-3-pyridyl)phenyl]methyl]-2-[4-(1H-imidazol-2-yl)anilino]-2-oxo-ethyl]carbamate;
tert-butyl N-[1-[[2-chloro-5-(1-isopropyl-6-oxo-3-pyridyl)phenyl]methyl]-2-oxo-2-[4-(1H-pyrazol-4-yl)anilino]ethyl]carbamate;
tert-butyl N-[1-[[2-chloro-5-(1-isopropyl-6-oxo-3-pyridyl)phenyl]methyl]-2-oxo-2-[4-(2H-tetrazol-5-yl)anilino]ethyl]carbamate;
tert-butyl N-[1-[[2-chloro-5-(1-isopropyl-6-oxo-3-pyridyl)phenyl]methyl]-2-[4-(1H-imidazol-4-yl)anilino]-2-oxo-ethyl]carbamate;
N-[1-[[2-chloro-5-(1-isopropyl-6-oxo-3-pyridyl)phenyl]methyl]-2-oxo-2-[4-(1,2,4-triazol-1-yl)anilino]ethyl]-2-methyl-pyrazole-3-carboxamide;
N-[1-[[2-chloro-5-(1-isopropyl-6-oxo-3-pyridyl)phenyl]methyl]-2-[4-(1H-imidazol-4-yl)anilino]-2-oxo-ethyl]-2-methyl-pyrazole-3-carboxamide;
N-[1-[[2-chloro-5-(1-isopropyl-6-oxo-3-pyridyl)phenyl]methyl]-2-oxo-2-(4-pyrazol-1-ylanilino)ethyl]-2-methyl-pyrazole-3-carboxamide;
N-[1-[[2-chloro-5-(1-isopropyl-6-oxo-3-pyridyl)phenyl]methyl]-2-(4-imidazol-1-ylanilino)-2-oxo-ethyl]-2-methyl-pyrazole-3-carboxamide;
tert-butyl N-[1-[[2-chloro-5-(1-methylpyrazol-4-yl)phenyl]methyl]-2-[4-(1H-imidazol-4-yl)anilino]-2-oxo-ethyl]carbamate;
tert-butyl N-[1-[[2-chloro-5-(1-methylpyrazol-4-yl)phenyl]methyl]-2-(4-imidazol-1-ylanilino)-2-oxo-ethyl]carbamate;

or pharmaceutically acceptable salts, hydrates or solvates thereof.

1. A compound according to formula I, 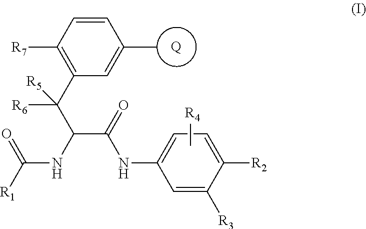
wherein:
R₁ is selected from the group consisting of 5- or 6-membered heteroaryl, 9- or 10-membered bicyclic heteroaryl, phenyl, 4-6-membered heterocycloalkyl, (C₁-C₆)alkoxy, (C₃-C₇)cycloalkoxy, (C₃-C₇)cycloalkyl(C₁-C₆)alkyl, (C₃-C₇)cycloalkyl(C₁-C₆)alkoxy, (C₁-C₆)alkyl, phenyl-(C₁-C₄)alkyl, (C₃-C₇)cycloalkyl, and —NR_{c}R_{d}, wherein said 5- or 6-membered heteroaryl, 9- or 10-membered bicyclic heteroaryl, phenyl, 4-6-membered heterocycloalkyl, (C₁-C₆)alkoxy, (C₃-C₇)cycloalkoxy, (C₃-C₇)cycloalkyl(C₁-C₆)alkyl, (C₃-C₇)cycloalkyl(C₁-C₆)alkoxy, (C₁-C₆)alkyl, phenyl-(C₁-C₄)alkyl, or (C₃-C₇)cycloalkyl is optionally substituted with one or more substituents independently selected from Rₐ;
Rₐ is selected from the group consisting of deuterium, halogen, hydroxy —NR_{c}R_{d}, (C₁-C₆)alkyl, (C₁-C₆)alkylcarbonyl, (C₃-C₇)cycloalkyl, phenyl, 4-6-membered heterocycloalkyl, and 5- or 6-membered heteroaryl, wherein said (C₁-C₆)alkyl, (C₁-C₆)alkylcarbonyl, (C₃-C₇)cycloalkyl, phenyl, 4-6-membered heterocycloalkyl, or 5- or 6-membered heteroaryl is optionally substituted with one or more substituents independently selected from the group consisting of deuterium, halogen, hydroxy, (C₁-C₄)alkyl, (C₃-C₇)cycloalkyl, (C₁-C₄)alkoxy, —SO₂—(C₁-C₄)alkyl, and —NR_{c}R_{d};
R₂ is a 5-membered heteroaryl, wherein said 5-membered heteroaryl is optionally substituted with one or more substituents independently selected from R_{b};
R_{b} is selected from the group consisting of deuterium, halogen, cyano, hydroxy, —NR_{c}R_{d}, (C₁-C₆)alkyl, (C₁-C₆)alkoxy, and (C₃-C₇)cycloalkyl, wherein said (C₁-C₆)alkyl, (C₁-C₆)alkoxy, or (C₃-C₇)cycloalkyl is optionally substituted with one or more substituents independently selected from the group consisting of deuterium, halogen, hydroxy, —NR_{c}R_{d}, and (C₁-C₄)alkoxy;
R_{c} and R_{d} are each independently hydrogen or (C₁-C₄)alkyl;
R₃ is selected from the group consisting of hydrogen, deuterium, hydroxy, (C₁-C₆)alkoxy, and halogen;
or R₂ and R₃ together with the phenyl to which they are attached form a 9- or 10-membered bicyclic heteroaromatic ring system, wherein said 9- or 10-membered bicyclic heteroaromatic ring system is optionally substituted with one or more substituents independently selected from Rₑ;
Rₑ is selected from the group consisting of deuterium, halogen, (C₁-C₄)alkyl, hydroxy(C₁-C₄)alkyl, (C₃-C₇)cycloalkyl, and —NR_{c}R_{d};
R₄ is selected from the group consisting of hydrogen, deuterium, and halogen;
R₅ and R₆ are each independently selected from the group consisting of hydrogen, deuterium, (C₁-C₄)alkyl, (C₃-C₇)cycloalkyl, and phenyl, with the proviso that at least one of R₅ and R₆ is hydrogen or deuterium;
R₇ is selected from the group consisting of hydrogen and halogen, with the proviso that R₇ is not hydrogen when both R₅ and R₆ are hydrogen or deuterium;
or R₆ and R₇ together with the phenyl to which R₇ is attached form a fused bicyclic ring system selected from the group consisting of tetralin and indane, wherein said tetralin or indane is optionally substituted with one more substituents independently selected from the group consisting of deuterium, halogen, and (C₁-C₄)alkyl;
Q is selected from the group consisting of phenyl, 4-8 membered heterocycloalkyl, 5- or 6-membered heteroaryl, 9- or 10-membered bicyclic heteroaryl, pyridonyl, indolinyl, isoindolinyl, tetrahydroquinolinyl, and tetrahydroisoquinolinyl, wherein said pyridonyl, phenyl, 4-8 membered heterocycloalkyl, 5- or 6-membered heteroaryl, 9- or 10-membered bicyclic heteroaryl, indolinyl, isoindolinyl, tetrahydroquinolinyl, or tetrahydroisoquinolinyl is optionally substituted with one or more substituents independently selected from R₈;
R₈ is selected from the group consisting of deuterium, oxo, hydroxy, —S(O)₂R_{f}, —S(O)R_{f}, —NR_{g}Rₕ, —C(O)NR_{g}Rₕ, —C(O)R_{f}, cyano, (C₁-C₆)alkyl, (C₃-C₇)cycloalkyl, 4-8 membered heterocycloalkyl, (C₃-C₇)cycloalkyl(C₁-C₄)alkyl, (C₃-C₇)cycloalkyl(C₁-C₄)alkoxy, (C₁-C₄)alkoxy(C₁-C₄)alkyl, (C₁-C₄)alkoxy, (C₃-C₇)cycloalkoxy, 5- or 6-membered heteroaryl, (5- or 6-membered heteroaryl)-(C₁-C₄)alkyl, and (4-8 membered heterocycloalkyl)-(C₁-C₄)alkyl, wherein said (C₁-C₆)alkyl, (C₃-C₇)cycloalkyl, 4-8 membered heterocycloalkyl, (C₃-C₇)cycloalkyl(C₁-C₄)alkyl, (C₃-C₇)cycloalkyl(C₁-C₄)alkoxy, (C₁-C₄)alkoxy(C₁-C₄)alkyl, (C₁-C₄)alkoxy, (C₃-C₇)cycloalkoxy, 5- or 6-membered heteroaryl, (5- or 6-membered heteroaryl)-(C₁-C₄)alkyl, or (4-8 membered heterocycloalkyl)-(C₁-C₄)alkyl is optionally substituted with one or more substituents independently selected from the group consisting of hydroxy, deuterium, halogen, cyano, oxo, (C₁-C₄)alkyl, (C₃-C₇)cycloalkyl, (C₁-C₄)alkoxy, hydroxy(C₁-C₄)alkyl, (C₁-C₄)alkoxy-(CH₂CH₂O)₀₋₂(C₁-C₄)alkyl, 4-7 membered heterocycloalkyl, —NR_{g}Rₕ, R_{g}RₕN—(C₁-C₄)alkyl, and —CO(O)Rᵢ;
R_{f} is (C₁-C₄)alkyl or (C₃-C₇)cycloalkyl;
R_{g} and Rₕ are each independently hydrogen, (C₁-C₄)alkyl, (C₃-C₇)cycloalkyl, (C₃-C₇)cycloalkyl(C₁-C₄)alkyl, or hydroxy(C₁-C₄)alkyl;
or R_{g} and Rₕ together with the nitrogen to which they are attached form a 4-8 membered heterocycloalkyl wherein said 4-8 membered heterocycloalkyl is optionally substituted with one or more substituents independently selected from the group consisting of deuterium, halogen, hydroxy, (C₁-C₄)alkyl, hydroxy(C₁-C₄)alkyl, and halo(C₁-C₄)alkyl;
Rᵢ is hydrogen or (C₁-C₄)alkyl;
or pharmaceutically acceptable salts thereof.

2. The compound according to claim 1 having formula (Ia)

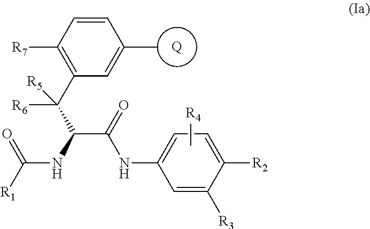

3. The compound according to claim 1, wherein:
R₁ is selected from the group consisting of (C₃-C₇)cycloalkyl, (C₃-C₇)cycloalkyl(C₁-C₆)alkyl, (C₃-C₇)cycloalkyl(C₁-C₆)alkoxy, (C₃-C₇)cycloalkoxy, and 5- or 6 membered heteroaryl, wherein said (C₃-C₇)cycloalkyl, (C₃-C₇)cycloalkyl(C₁-C₆)alkyl, (C₃-C₇)cycloalkyl(C₁-C₆)alkoxy, (C₃-C₇)cycloalkoxy, or 5- or 6 membered heteroaryl is optionally substituted with one or more substituents independently selected from Rₐ;
R₂ is a 5-membered heteroaryl, wherein said 5-membered heteroaryl is optionally substituted with one or more substituents independently selected from R_{b};
R₅ and R₆ are each independently selected from the group consisting of hydrogen, deuterium, (C₁-C₄)alkyl, (C₃-C₇)cycloalkyl, and phenyl, with the proviso that at least one of R₅ and R₆ is hydrogen or deuterium;
R₇ is hydrogen or halogen, with the proviso that R₇ is not hydrogen when both R₅ and R₆ are hydrogen or deuterium;
or R₆ and R₇ together with the phenyl to which R₇ is attached form a fused bicyclic ring system selected from the group consisting of tetralin and indane, wherein said tetralin or indane is optionally substituted with one more substituents independently selected from the group consisting of deuterium, halogen, and (C₁-C₄)alkyl;
Q is selected from the group consisting of phenyl, 4-8 membered heterocycloalkyl, 5- or 6-membered heteroaryl, 9- or 10-membered bicyclic heteroaryl, pyridonyl, indolinyl, isoindolinyl, tetrahydroquinolinyl, and tetrahydroisoquinolinyl, wherein said pyridonyl, phenyl, 4-8 membered heterocycloalkyl, 5- or 6-membered heteroaryl, 9- or 10-membered bicyclic heteroaryl, indolinyl, isoindolinyl, tetrahydroquinolinyl, or tetrahydroisoquinolinyl is optionally substituted with one or more substituents independently selected from R₈;
and pharmaceutically acceptable salts thereof

4. The compound according to claim 1, wherein:
R₁ is a 5- or 6 membered heteroaryl optionally substituted with one or more substituents independently selected from Rₐ;
R₂ is a 5-membered heteroaryl optionally substituted with one or more substituents independently selected from R_{b};
R₆ and R₇ together with the phenyl to which R₇ is attached form a fused bicyclic ring system selected from the group consisting of tetralin and indane, wherein said tetralin or indane is optionally substituted with one or more substituents independently selected from the group consisting of deuterium, halogen, and C₁₋₄ alkyl;
Q is selected from the group consisting of phenyl, 5- or 6-membered heteroaryl, pyridonyl, pyrrolidinyl, indolinyl, isoindolinyl, tetrahydroisoquinolinyl, and tetrahydroquinolinyl, wherein said phenyl, 5- or 6-membered heteroaryl, pyridonyl, pyrrolidinyl, indolinyl, isoindolinyl, tetrahydroisoquinolinyl, or tetrahydroquinolinyl is optionally substituted with one or more substituents independently selected from R₈.

5. The compound according to claim 1, wherein:
R₁ is selected from the group consisting of (C₃-C₇)cycloalkyl, (C₃-C₇)cycloalkyl(C₁-C₆)alkyl, (C₃-C₇)cycloalkyl(C₁-C₆)alkoxy, and (C₃-C₇)cycloalkoxy wherein said (C₃-C₇)cycloalkyl, (C₃-C₇)cycloalkyl(C₁-C₆)alkyl, (C₃-C₇)cycloalkyl(C₁-C₆)alkoxy, or (C₃-C₇)cycloalkoxy is optionally substituted with one or more substituents independently selected from Rₐ;
R₂ is a 5-membered heteroaryl optionally substituted with one or more substituents independently selected from R_{b};
R₆ and R₇ together with the phenyl to which R₇ is attached form a fused bicyclic ring system selected from the group consisting of tetralin and indane, wherein said tetralin or indane is optionally substituted with one or more substituents independently selected from the group consisting of deuterium, halogen, and C₁₋₄ alkyl;
Q is selected from the group consisting of phenyl, 5- or 6-membered heteroaryl, pyridonyl, indolinyl, isoindolinyl, tetrahydroisoquinolinyl, and tetrahydroquinolinyl, wherein said phenyl, 5- or 6-membered heteroaryl, pyridonyl, indolinyl, isoindolinyl, tetrahydroisoquinolinyl, or tetrahydroquinolinyl is optionally substituted with one or more substituents independently selected from R₈.

6. The compound according to claim 1, wherein:
R₁ is a 5- or 6 membered heteroaryl optionally substituted with one or more substituents independently selected from Rₐ;
R₂ is a 5-membered heteroaryl optionally substituted with one or more substituents independently selected from R_{b};
R₅ and R₆ are each independently selected from the group consisting of hydrogen, deuterium, (C₁-C₄)alkyl, (C₃-C₇)cycloalkyl, and phenyl, with the proviso that at least one of R₅ and R₆ is hydrogen or deuterium and at least one of R₅ and R₆ is selected from the group consisting of (C₁-C₄)alkyl, (C₃-C₇)cycloalkyl, and phenyl;
R₇ is hydrogen or halogen;
Q is selected from the group consisting of phenyl, 5- or 6-membered heteroaryl, pyridonyl, indolinyl, isoindolinyl, tetrahydroisoquinolinyl, and tetrahydroquinolinyl, wherein said phenyl, 5- or 6-membered heteroaryl, pyridonyl, indolinyl, isoindolinyl, tetrahydroisoquinolinyl, or tetrahydroquinolinyl is optionally substituted with one or more substituents independently selected from R₈.

7. The compound according to claim 1, wherein:
R₁ is selected the group consisting of (C₃-C₇)cycloalkyl, (C₃-C₇)cycloalkyl(C₁-C₆)alkyl, (C₃-C₇)cycloalkyl(C₁-C₆)alkoxy, and (C₃-C₇)cycloalkoxy, wherein said (C₃-C₇)cycloalkyl, (C₃-C₇)cycloalkyl(C₁-C₆)alkyl, (C₃-C₇)cycloalkyl(C₁-C₆)alkoxy, or (C₃-C₇)cycloalkoxy is optionally substituted with one or more substituents independently selected independently from Rₐ;
R₂ is a 5-membered heteroaryl optionally substituted with one or more substituents independently selected from R_{b};
R₅ and R₆ are each independently selected from the group consisting of hydrogen, deuterium, (C₁-C₄)alkyl, (C₃-C₇)cycloalkyl, and phenyl, with the proviso that at least one of R₅ and R₆ is hydrogen or deuterium and at least one of R₅ and R₆ is selected from the group consisting of (C₁-C₄)alkyl, (C₃-C₇)cycloalkyl, and phenyl;
R₇ is hydrogen or halogen;
Q is selected from the group consisting of phenyl, 5- or 6-membered heteroaryl, pyridonyl, indolinyl, isoindolinyl, tetrahydroisoquinolinyl, and tetrahydroquinolinyl, wherein said phenyl, 5- or 6-membered heteroaryl, pyridonyl, indolinyl, isoindolinyl, tetrahydroisoquinolinyl, or tetrahydroquinolinyl is optionally substituted with one or more substituents independently selected from R₈.

8. The compound according to claim 1, wherein:
R₁ is pyrazolyl or isoxazolyl, wherein said pyrazolyl or isoxazolyl is optionally substituted with one or more substituents independently selected from Rₐ;
R₂ is pyrazolyl or imidazolyl, wherein said pyrazolyl or imidazolyl is optionally substituted with one or more substituents independently selected from R_{b};
Q is selected from the group consisting of phenyl, 5- or 6-membered heteroaryl, pyridonyl, indolinyl, isoindolinyl, tetrahydroisoquinolinyl, and tetrahydroquinolinyl, wherein said phenyl, 5- or 6-membered heteroaryl, pyridonyl, indolinyl, isoindolinyl, tetrahydroisoquinolinyl, or tetrahydroquinolinyl is optionally substituted with one or more substituents independently selected from R₈.

9. The compound according to claim 8, wherein:
R₁ is pyrazol-3-yl or isoxazol-4-yl, wherein said pyrazol-3-yl or isoxazol-4-yl is optionally substituted with one or more substituents independently selected from Rₐ;
R₂ is pyrazol-4-yl, pyrazol-3-yl, or imidazo-4-yl, wherein said pyrazol-4-yl, pyrazol-3-yl, or imidazo-4-yl is optionally substituted with one or more substituents independently selected from R_{b};
Q is selected from the group consisting of phenyl, 5- or 6-membered heteroaryl, pyridonyl, indolinyl, isoindolinyl, tetrahydroisoquinolinyl, and tetrahydroquinolinyl, wherein said phenyl, 5- or 6-membered heteroaryl, pyridonyl, indolinyl, isoindolinyl, tetrahydroisoquinolinyl, or tetrahydroquinolinyl is optionally substituted with one or more substituents independently selected from R₈.

10. The compound according to claim 1, wherein:
R₁ is (C₃-C₇)cycloalkyl, wherein said (C₃-C₇)cycloalkyl is optionally substituted with one or more substituents independently selected from Rₐ;
R₂ is pyrazolyl or imidazolyl, wherein said pyrazolyl or imidazolyl is optionally substituted with one or more substituents independently selected from R_{b};
Q is selected from the group consisting of phenyl, 5- or 6-membered heteroaryl, pyridonyl, indolinyl, isoindolinyl, tetrahydroisoquinolinyl, and tetrahydroquinolinyl, wherein said phenyl, 5- or 6-membered heteroaryl, pyridonyl, indolinyl, isoindolinyl, tetrahydroisoquinolinyl, or tetrahydroquinolinyl is optionally substituted with one or more substituents independently selected from R₈.

11. The compound according to claim 10 wherein
R₁ is cyclopropyl optionally substituted with one or more substituents independently selected from Rₐ;
R₂ is pyrazol-4-yl or imidazo-4-yl, wherein said pyrazol-4-yl or imidazo-4-yl is optionally substituted with one or more substituents independently selected from R_{b};
Q is selected from the group consisting of phenyl, 5- or 6-membered heteroaryl, pyridonyl, indolinyl, isoindolinyl, tetrahydroisoquinolinyl, and tetrahydroquinolinyl, wherein said phenyl, 5- or 6-membered heteroaryl, pyridonyl, indolinyl, isoindolinyl, tetrahydroisoquinolinyl, or tetrahydroquinolinyl is optionally substituted with one or more substituents independently selected from R₈.

12. The compound according to claim 1, wherein:
R₁ is unsubstituted, or Rₐ is deuterium or (C₁-C₆)alkyl;
R₂ is unsubstituted, or R_{b} is deuterium or (C₁-C₆)alkyl;
Q is unsubstituted, or R₈ is deuterium or (C₁-C₆)alkyl;
wherein when Rₐ, R_{b}, and/or R₈ is (C₁-C₆)alkyl, wherein said (C₁-C₆)alkyl is optionally substituted with one or more substituents independently selected from the group consisting of hydroxy, (C₁-C₄)alkyl, (C₃-C₇)cycloalkyl, and —NR_{g}Rₕ, wherein R_{g} and Rₕ is independently selected from the group consisting of hydrogen, (C₁-C₄)alkyl, (C₃-C₇)cycloalkyl, (C₃-C₇)cycloalkyl(C₁-C₄)alkyl, and hydroxy(C₁-C₄)alkyl.

13. The compound according to claim 1, wherein:
R₁ is unsubstituted, or Rₐ is deuterium or (C₁-C₆)alkyl;
R₂ is unsubstituted, or R_{b} is deuterium or (C₁-C₆)alkyl;
R₈ is —NR_{g}Rₕ, or (C₁-C₆)alkyl substituted with —NR_{g}Rₕ, wherein R_{g} and Rₕ together with the nitrogen to which they are attached form a 4-8 membered heterocycloalkyl wherein said 4-8 membered heterocycloalkyl is optionally substituted with one or more substituents independently selected from the group consisting of deuterium, halogen, hydroxy, (C₁-C₄)alkyl, hydroxy(C₁-C₄)alkyl, and halo(C₁-C₄)alkyl.

14. A compound according to claim 1 selected from the group consisting of:
(i) N-[(1S)-2-[4-(3,5-dimethyl-1H-pyrazol-4-yl)anilino]-1-[(1R)-6-isoindolin-5-ylindan-1-yl]-2-oxo-ethyl]-2-methyl-pyrazole-3-carboxamide;
(ii) N-[(1S)-2-[4-(3,5-dimethyl-1H-pyrazol-4-yl)anilino]-1-[(1R)-6-[4-(morpholinomethyl)phenyl]indan-1-yl]-2-oxo-ethyl]-2-methyl-pyrazole-3-carboxamide;
(iii) N-[(1S)-1-[(1R)-6-(1-tert-butylpyrazol-4-yl)indan-1-yl]-2-[4-(3,5-dimethyl-1H-pyrazol-4-yl)anilino]-2-oxo-ethyl]-2-methyl-pyrazole-3-carboxamide;
(iv) N-[(1S)-1-[(1R)-6-(1-cyclobutylpyrazol-4-yl)indan-1-yl]-2-[4-(3,5-dimethyl-1H-pyrazol-4-yl)anilino]-2-oxo-ethyl]-2-methyl-pyrazole-3-carboxamide;
(v) N-[(1S)-2-[4-(3,5-dimethyl-1H-pyrazol-4-yl)anilino]-1-[(1R)-6-(1-isopropyl-6-oxo-3-pyridyl)indan-1-yl]-2-oxo-ethyl]-1-fluoro-cyclopropanecarboxamide;
(vi) N-[(1S)-2-[4-(3,5-dimethyl-1H-pyrazol-4-yl)anilino]-1-[(1R)-6-(1-isopropylpyrazol-4-yl)indan-1-yl]-2-oxo-ethyl]-2-methyl-pyrazole-3-carboxamide;
(vii) N-[(1S)-1-[(1R)-6-(1-cyclopropylpyrazol-4-yl)indan-1-yl]-2-[4-(3,5-dimethyl-1H-pyrazol-4-yl)anilino]-2-oxo-ethyl]-2-methyl-pyrazole-3-carboxamide;
(viii) N-[(1S)-2-[4-(3,5-dimethyl-1H-pyrazol-4-yl)anilino]-2-oxo-1-[(1R)-6-(2-piperazin-1-yl-4-pyridyl)indan-1-yl]ethyl]-1-fluoro-cyclopropanecarboxamide;
(ix) N-[(1S)-2-[4-(3,5-dimethyl-1H-pyrazol-4-yl)anilino]-1-[(1R)-6-(2-methylisoindolin-5-yl)indan-1-yl]-2-oxo-ethyl]-2-methyl-pyrazole-3-carboxamide;
(x) N-[(1S)-2-[4-(3,5-dimethyl-1H-pyrazol-4-yl)anilino]-1-[(1R)-6-[1-(2-hydroxy-1,1-dimethyl-ethyl)pyrazol-4-yl]indan-1-yl]-2-oxo-ethyl]-2-methyl-pyrazole-3-carboxamide;
(xi) N-[(1S)-2-[4-(3,5-dimethyl-1H-pyrazol-4-yl)anilino]-1-[(1R)-6-[1-(2-hydroxy-2-methyl-propyl)pyrazol-4-yl]indan-1-yl]-2-oxo-ethyl]-2-methyl-pyrazole-3-carboxamide;
(xii) N-[(1S)-2-[4-(3,5-dimethyl-1H-pyrazol-4-yl)anilino]-1-[(1R)-6-(2-isopropyl-4-pyridyl)indan-1-yl]-2-oxo-ethyl]-2-methyl-pyrazole-3-carboxamide;
(xiii) N-[(1S)-2-[4-(3,5-dimethyl-1H-pyrazol-4-yl)anilino]-1-[(1R)-6-(2-methyl-3,4-dihydro-1H-isoquinolin-6-yl)indan-1-yl]-2-oxo-ethyl]-2-methyl-pyrazole-3-carboxamide;
(xiv) N-[(1S)-2-[4-(3,5-dimethyl-1H-pyrazol-4-yl)anilino]-1-[(1R)-6-[2-(1-hydroxy-1-methyl-ethyl)-4-pyridyl]indan-1-yl]-2-oxo-ethyl]-2-methyl-pyrazole-3-carboxamide;
(xv) N-[(1S)-2-[4-(3,5-dimethyl-1H-pyrazol-4-yl)anilino]-2-oxo-1-[(1R)-6-(1,2,3,4-tetrahydroisoquinolin-6-yl)indan-1-yl]ethyl]-2-methyl-pyrazole-3-carboxamide;
(xvi) N-[(1S)-2-[4-(3,5-dimethyl-1H-pyrazol-4-yl)anilino]-1-[(1R)-6-[3-methyl-4-(morpholinomethyl)phenyl]indan-1-yl]-2-oxo-ethyl]-2-methyl-pyrazole-3-carboxamide;
(xvii) N-[(1S)-2-[4-(3,5-dimethyl-1H-pyrazol-4-yl)anilino]-2-oxo-1-[(1R)-6-[4-(pyrrolidin-1-ylmethyl)phenyl]indan-1-yl]ethyl]-2-methyl-pyrazole-3-carboxamide;
(xviii) N-[(1S)-1-[(1R)-6-[4-(azetidin-1-ylmethyl)phenyl]indan-1-yl]-2-[4-(3,5-dimethyl-1H-pyrazol-4-yl)anilino]-2-oxo-ethyl]-2-methyl-pyrazole-3-carboxamide;
(xix) N-[(1S)-1-[(1R)-6-[2-[(1S,4S)-2,5-diazabicyclo[2.2.1]heptan-2-yl]-4-pyridyl]indan-1-yl]-2-[4-(3,5-dimethyl-1H-pyrazol-4-yl)anilino]-2-oxo-ethyl]-1-fluoro-cyclopropanecarboxamide;
(xx) N-[(1S)-1-[(1R)-7-(1-cyclopropylpyrazol-4-yl)tetralin-1-yl]-2-[4-(3,5-dimethyl-1H-pyrazol-4-yl)anilino]-2-oxo-ethyl]-2-methyl-pyrazole-3-carboxamide;
(xxi) N-[(1S)-2-[4-(3,5-dimethyl-1H-pyrazol-4-yl)anilino]-1-[(1R)-7-(1-isopropyl-6-oxo-3-pyridyl)tetralin-1-yl]-2-oxo-ethyl]-1-fluoro-cyclopropanecarboxamide;
(xxii) N-[(1S)-1-[(1R)-7-(1-isopropyl-6-oxo-3-pyridyl)tetralin-1-yl]-2-[4-(3-methylimidazol-4-yl)anilino]-2-oxo-ethyl]-2-methyl-pyrazole-3-carboxamide;
(xxiii) cyclopropyl N-[(1S)-2-[3-hydroxy-4-(3-methylimidazol-4-yl)anilino]-1-[(1R)-7-(1-isopropyl-6-oxo-3-pyridyl)tetralin-1-yl]-2-oxo-ethyl]carbamate;
(xxiv) N-[(1S)-2-[4-(3,5-dimethyl-1H-pyrazol-4-yl)-3-hydroxy-anilino]-1-[(1R)-7-(1-isopropyl-6-oxo-3-pyridyl)tetralin-1-yl]-2-oxo-ethyl]-2-methyl-pyrazole-3-carboxamide;
(xxv) N-[(1S)-1-[(1R)-7-(1-tert-butylpyrazol-4-yl)tetralin-1-yl]-2-[4-(3,5-dimethyl-1H-pyrazol-4-yl)anilino]-2-oxo-ethyl]-2-methyl-pyrazole-3-carboxamide;
(xxvi) N-[(1S)-2-[4-(3,5-dimethyl-1H-pyrazol-4-yl)anilino]-1-[(1R)-7-isoindolin-5-yltetralin-1-yl]-2-oxo-ethyl]-2-methyl-pyrazole-3-carboxamide;
(xxvii) N-[(1S)-2-[4-(3,5-dimethyl-1H-pyrazol-4-yl)anilino]-1-[(1R)-7-(2-methyl-3,4-dihydro-1H-isoquinolin-6-yl)tetralin-1-yl]-2-oxo-ethyl]-2-methyl-pyrazole-3-carboxamide;
(xxviii) N-[(1S)-2-[4-(3,5-dimethyl-1H-pyrazol-4-yl)anilino]-1-[(1R)-7-(2-methylisoindolin-5-yl)tetralin-1-yl]-2-oxo-ethyl]-2-methyl-pyrazole-3-carboxamide;
(xxix) N-[(1S)-2-[4-(3,5-dimethyl-1H-pyrazol-4-yl)anilino]-2-oxo-1-[(1R)-7-(1,2,3,4-tetrahydroisoquinolin-6-yl)tetralin-1-yl]ethyl]-2-methyl-pyrazole-3-carboxamide;
(xxx) N-[(1S)-2-[4-(3,5-dimethyl-1H-pyrazol-4-yl)anilino]-2-oxo-1-[(1R)-7-(2-piperazin-1-yl-4-pyridyl)tetralin-1-yl]ethyl]-1-fluoro-cyclopropanecarboxamide;
(xxxi) N-[(1S)-1-[(1R)-7-[2-[(1S,4S)-2,5-diazabicyclo[2.2.1]heptan-2-yl]-4-pyridyl]tetralin-1-yl]-2-[4-(3,5-dimethylimidazol-4-yl)anilino]-2-oxo-ethyl]-1-fluoro-cyclopropanecarboxamide;
(xxxii) N-[(1S)-2-[4-(3,5-dimethylimidazol-4-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]-1-fluoro-cyclopropanecarboxamide;
(xxxiii) N-[(1S)-2-[4-(3,5-dimethylimidazol-4-yl)anilino]-1-[(1R)-7-isoindolin-5-yltetralin-1-yl]-2-oxo-ethyl]-2-methyl-pyrazole-3-carboxamide;
(xxxiv) N-[(1S)-2-[4-(3,5-dimethyl-1H-pyrazol-4-yl)-3-hydroxy-anilino]-1-[(1R)-7-(1-isopropyl-6-oxo-3-pyridyl)tetralin-1-yl]-2-oxo-ethyl]-2-methyl-pyrazole-3-carboxamide;
(xxxv) N-[(1S)-2-[4-(3,5-dimethyl-1H-pyrazol-4-yl)-3-hydroxy-anilino]-1-[(1R)-7-(1-isopropyl-6-oxo-3-pyridyl)tetralin-1-yl]-2-oxo-ethyl]-1-fluoro-cyclopropanecarboxamide;
(xxxvi) N-[(1S)-2-[3-hydroxy-4-(3-methylimidazol-4-yl)anilino]-1-[(1R)-7-(1-isopropyl-6-oxo-3-pyridyl)tetralin-1-yl]-2-oxo-ethyl]-2-methyl-pyrazole-3-carboxamide;
(xxxvii) N-[(1S)-1-[(1R)-7-[3-(1-amino-1-methyl-ethyl)phenyl]tetralin-1-yl]-2-[4-(3,5-dimethylimidazol-4-yl)anilino]-2-oxo-ethyl]-2-methyl-pyrazole-3-carboxamide;
(xxxviii) N-[(1S)-1-[[2-chloro-5-[3-(1-hydroxy-1-methyl-ethyl)phenyl]phenyl]methyl]-2-[4-(3,5-dimethyl-1H-pyrazol-4-yl)anilino]-2-oxo-ethyl]-2-methyl-pyrazole-3-carboxamide;
(xxxix) N-[(1S)-1-[[5-[3-(1-amino-1-methyl-ethyl)phenyl]-2-chloro-phenyl]methyl]-2-[4-(3,5-dimethyl-1H-pyrazol-4-yl)anilino]-2-oxo-ethyl]-2-methyl-pyrazole-3-carboxamide;
(xl) N-[(1S)-1-[[2-chloro-5-[3-(dimethylaminomethyl)phenyl]phenyl]methyl]-2-[4-(3,5-dimethyl-1H-pyrazol-4-yl)anilino]-2-oxo-ethyl]-2-methyl-pyrazole-3-carboxamide;
(xli) N-[(1S)-1-[[2-chloro-5-(1-isopropyl-6-oxo-3-pyridyl)phenyl]methyl]-2-[3-hydroxy-4-(2-methylpyrazol-3-yl)anilino]-2-oxo-ethyl]-3-methyl-isoxazole-4-carboxamide;
(xlii) N-[1-[[2-chloro-5-(1-isopropyl-6-oxo-3-pyridyl)phenyl]methyl]-2-[4-(3,5-dimethyl-1H-pyrazol-4-yl)anilino]-2-oxo-ethyl]-2-methyl-pyrazole-3-carboxamide;
(xliii) N-[(1S)-1-[[2-chloro-5-(1-isopropyl-6-oxo-3-pyridyl)phenyl]methyl]-2-[4-(2,4-dimethylpyrazol-3-yl)anilino]-2-oxo-ethyl]-3-methyl-isoxazole-4-carboxamide;
(xliv) N-[(1S)-1-[[2-chloro-5-[2-[cyclopropyl(hydroxy)methyl]-4-pyridyl]phenyl]methyl]-2-[4-(3-methylimidazol-4-yl)anilino]-2-oxo-ethyl]-3-methyl-isoxazole-4-carboxamide;
(xlv) N-[(1S)-1-[[2-chloro-5-(1-isopropyl-6-oxo-3-pyridyl)phenyl]methyl]-2-[3-hydroxy-4-(3-methylimidazol-4-yl)anilino]-2-oxo-ethyl]-3-methyl-isoxazole-4-carboxamide;
(xlvi) N-[(1S)-1-[[2-chloro-5-(1-isopropyl-6-oxo-3-pyridyl)phenyl]methyl]-2-[4-(3,5-dimethyl-1H-pyrazol-4-yl)anilino]-2-oxo-ethyl]-1-fluoro-cyclopropanecarboxamide;
(xlvii) N-[(1S)-1-[[2-chloro-5-[2-(1-cyclopropyl-1-hydroxy-ethyl)-4-pyridyl]phenyl]methyl]-2-[4-(3-methylimidazol-4-yl)anilino]-2-oxo-ethyl]-2-methyl-pyrazole-3-carboxamide;
(xlviii) N-[(1S)-1-[[2-chloro-5-[2-(1-cyclopropyl-1-hydroxy-ethyl)-4-pyridyl]phenyl]methyl]-2-[4-(3-methylimidazol-4-yl)anilino]-2-oxo-ethyl]-1-fluoro-cyclopropanecarboxamide;
(xlix) N-[1-[[2-chloro-5-[2-[cyclopropyl(methyl)amino]-4-pyridyl]phenyl]methyl]-2-[4-(3-methylimidazol-4-yl)anilino]-2-oxo-ethyl]-2-methyl-pyrazole-3-carboxamide;
(l) N-[1-[[2-chloro-5-[2-[isopropyl(methyl)amino]-4-pyridyl]phenyl]methyl]-2-[4-(3-methylimidazol-4-yl)anilino]-2-oxo-ethyl]-2-methyl-pyrazole-3-carboxamide;
(li) N-[(1S)-1-[[2-chloro-5-(1-isopropyl-6-oxo-3-pyridyl)phenyl]methyl]-2-[3-hydroxy-4-(3-methyl-1H-pyrazol-4-yl)anilino]-2-oxo-ethyl]-2-methyl-pyrazole-3-carboxamide;
(lii) N-[(1S)-1-[[2-chloro-5-(1-isopropyl-6-oxo-3-pyridyl)phenyl]methyl]-2-[3-hydroxy-4-(1H-pyrazol-4-yl)anilino]-2-oxo-ethyl]-2-methyl-pyrazole-3-carboxamide;
(liii) N-[1-[[5-[3-(1-amino-1-methyl-ethyl)phenyl]-2-chloro-phenyl]methyl]-2-[4-(3-methylimidazol-4-yl)anilino]-2-oxo-ethyl]-2-methyl-pyrazole-3-carboxamide;
(liv) N-[(1S)-1-[[2-chloro-5-(1-isopropyl-6-oxo-3-pyridyl)phenyl]methyl]-2-[4-(3,5-dimethyl-1H-pyrazol-4-yl)-3-hydroxy-anilino]-2-oxo-ethyl]-2-methyl-pyrazole-3-carboxamide;
(lv) N-[1-[[2-chloro-5-[3-(1-hydroxy-1-methyl-ethyl)phenyl]phenyl]methyl]-2-[4-(3-methylimidazol-4-yl)anilino]-2-oxo-ethyl]-2-methyl-pyrazole-3-carboxamide;
(lvi) N-[1-[[2-chloro-5-(1-isopropyl-6-oxo-3-pyridyl)phenyl]methyl]-2-[4-(3-chloro-1H-pyrazol-4-yl)anilino]-2-oxo-ethyl]-2-methyl-pyrazole-3-carboxamide;
(lvii) N-[(1S)-1-[[2-chloro-5-[3-(1-hydroxy-1-methyl-ethyl)phenyl]phenyl]methyl]-2-[4-(3,5-dimethylimidazol-4-yl)anilino]-2-oxo-ethyl]-2-methyl-pyrazole-3-carboxamide;
(lviii) N-[(1S)-1-[[5-(1-tert-butyl pyrazol-4-yl)-2-chloro-phenyl]methyl]-2-[4-(3,5-dimethylimidazol-4-yl)anilino]-2-oxo-ethyl]-2-methyl-pyrazole-3-carboxamide;
(lix) N-[(1S)-1-[[5-(1-tert-butyl pyrazol-4-yl)-2-chloro-phenyl]methyl]-2-[4-(3,5-dimethyl-1H-pyrazol-4-yl)anilino]-2-oxo-ethyl]-2-methyl-pyrazole-3-carboxamide;
(lx) N-[(1S)-1-[[2-chloro-5-[2-[(2R)-2-methylpiperazin-1-yl]-4-pyridyl]phenyl]methyl]-2-[4-(3-methylimidazol-4-yl)anilino]-2-oxo-ethyl]-2-methyl-pyrazole-3-carboxamide;
(lxi) N-[(1S)-1-[[2-chloro-5-[2-[(1R,4R)-2-oxa-5-azabicyclo[2.2.1]heptan-5-yl]-4-pyridyl]phenyl]methyl]-2-[4-(3,5-dimethyl-1H-pyrazol-4-yl)anilino]-2-oxo-ethyl]-1-fluoro-cyclopropanecarboxamide;
(lxii) N-[(1S)-1-[[2-chloro-5-[2-[(1S,4S)-2,5-diazabicyclo[2.2.1]heptan-2-yl]-4-pyridyl]phenyl]methyl]-2-[4-(3,5-dimethyl-1H-pyrazol-4-yl)anilino]-2-oxo-ethyl]-1-fluoro-cyclopropanecarboxamide;
(lxiii) N-[(1S)-1-[[2-chloro-5-(2-morpholino-4-pyridyl)phenyl]methyl]-2-[4-(3,5-dimethylimidazol-4-yl)anilino]-2-oxo-ethyl]-1-fluoro-cyclopropanecarboxamide;
(lxiv) N-[(1S)-1-[[2-chloro-5-(2-morpholino-4-pyridyl)phenyl]methyl]-2-[4-(3,5-dimethyl-1H-pyrazol-4-yl)anilino]-2-oxo-ethyl]-1-fluoro-cyclopropanecarboxamide;
(lxv) N-[1-[[3-(1-isopropyl-6-oxo-3-pyridyl)phenyl]-phenyl-methyl]-2-[4-(3-methyl-1H-pyrazol-4-yl)anilino]-2-oxo-ethyl]-2-methyl-pyrazole-3-carboxamide;
(lxvi) cyclopropyl N-[1-[[3-(1-isopropyl-6-oxo-3-pyridyl)phenyl]-phenyl-methyl]-2-[4-(3-methyl-1H-pyrazol-4-yl)anilino]-2-oxo-ethyl]carbamate;
(lxvii) N-[(1S)-2-[4-(3,5-dimethyl-1H-pyrazol-4-yl)anilino]-1-[(1R)-7-[2-[(1R,4R)-2-oxa-5-azabicyclo[2.2.1]heptan-5-yl]-4-pyridyl]tetralin-1-yl]-2-oxo-ethyl]-1-fluoro-cyclopropanecarboxamide; and
(lxviii) N-[(1S)-2-[4-(3,5-dimethyl-1H-pyrazol-4-yl)anilino]-1-[(1R)-7-[2-[(1S,4S)-2-oxa-5-azabicyclo[2.2.1]heptan-5-yl]-4-pyridyl]tetralin-1-yl]-2-oxo-ethyl]-1-fluoro-cyclopropanecarboxamide;
or pharmaceutically acceptable salts thereof.

15. A method of treating a disease, disorder, or condition that is responsive to modulation of IL-17, the method comprising administering to a subject in need thereof a therapeutically effective amount of a compound according to claim 1.

16. The method according to claim 15, wherein the disease, disorder, or condition is an autoimmune disease.

17. The method according to claim 16, wherein the autoimmune disease is psoriasis, ankylosing spondylitis, spondyloarthritis, or psoriatic arthritis.

18. A pharmaceutical composition comprising a compound according to claim 1 and one or more pharmaceutically acceptable vehicles, excipients, or carriers.

19. A method of treating a disease, disorder, or condition that is responsive to modulation of IL-17, the method comprising administering to a subject in need thereof a therapeutically effective amount of a pharmaceutical composition according to claim 18.

20. The method according to claim 17, wherein the autoimmune disease is psoriasis.
